(19) United States
(12) Patent Application Publication
(10) Pub. No.: US 2023/0116201 A1
(43) Pub. Date: Apr. 13, 2023

(54) COLLAGEN 1 TRANSLATION INHIBITORS AND METHODS OF USE THEREOF

(71) Applicant: Anima Biotech Inc., Bernardsville, NJ (US)

(72) Inventors: David William SHEPPARD, Ramat Gan (IL); Jason Paul TIERNEY, Ramat Gan (IL); Wolfgang SCHMIDT, Essex (GB); William Rameshchandra ESMIEU, Essex (GB); Julie Nicole HAMBLIN, Essex (GB); Richard James BULL, Essex (GB); Iris ALROY, Ramat Gan (IL); Wissam MANSOUR, Ramat Gan (IL); Moty KLEPFISH, Ramat Gan (IL); Aviad MANDABI, Ramat Gan (IL)

(73) Assignee: Anima Biotech Inc., Bernardsville, NJ (US)

(21) Appl. No.: 17/790,961

(22) PCT Filed: Jan. 28, 2021
(86) PCT No.: PCT/US2021/015353

§ 371 Date: Jul. 6, 2022

Related U.S. Application Data
(60) Provisional application No. 62/967,645, filed on Jan. 30, 2020.

(51) Int. Cl. C07D 405/14; C07D 209/10; C07D 403/12; C07D 401/12; C07D 401/14; C07D 403/14; C07D 417/14; C07D 471/04; C07D 413/14; C07D 487/04; C07D 498/04; A61P 19/04

(52) U.S. Cl. CPC C07D 405/14; C07D 209/10; C07D 403/12; C07D 401/12; C07D 401/14; C07D 403/14; C07D 417/14; C07D 471/04; C07D 413/14; C07D 487/04; C07D 498/04; A61P 19/04

(57) ABSTRACT

The present invention relates to novel Collagen 1 translation inhibitors, composition and methods of preparation thereof, and uses thereof for treating Fibrosis including lung, liver, kidney, cardiac and dermal fibrosis, IPF, wound healing, scarring and Gingival fibromatosis, Systemic Sclerosis, and alcoholic and non-alcoholic steatohepatitis (NASH).

### FIELD OF THE INVENTION

The present invention relates to novel Collagen 1 translation inhibitors, composition and methods of preparation thereof, and uses thereof for treating Fibrosis including lung, liver, kidney, cardiac and dermal fibrosis, IPF, wound healing, scarring and Gingival fibromatosis, Systemic Sclerosis, and alcoholic and non-alcoholic steatohepatitis (NASH).

### BACKGROUND OF THE INVENTION

The formation of fibrous connective tissue is part of the normal healing process following tissue damage due to injury or inflammation. During this process, activated immune cells including macrophages stimulate the proliferation and activation of fibroblasts, which in turn deposit connective tissue. However, abnormal or excessive production of connective tissue may lead to accumulation of fibrous material such that it interferes with the normal function of the tissue. Fibrotic growth can proliferate and invade healthy surrounding tissue, even after the original injury heals. Such abnormal formation of excessive connective tissue, occurring in a reparative or reactive process, is referred to as fibrosis.

Many agents cause activation of the fibrotic process and are released in response to tissue injury, inflammation and oxidative stress. Regardless of the initiating events, a feature common to all fibrotic diseases is the conversion of tissue resident fibroblast into ECM-producing myofibroblasts that secret collagen type I. Current programs indirectly target myofibroblast activation and collagen secretion by inhibiting a single fibrosis inducing signal.

Physiologically, fibrosis acts to deposit connective tissue, which can obliterate the architecture and function of the underlying organ or tissue. Defined by the pathological accumulation of extracellular matrix (ECM) proteins, fibrosis results in scarring and thickening of the affected tissue, which interferes with normal organ function. In various conditions, the formation of fibrotic tissue is characterized by the deposition of abnormally large amounts of collagen. The synthesis of collagen is also involved in a number of other pathological conditions. For example, clinical conditions and disorders associated with primary or secondary fibrosis, such as systemic sclerosis, graft-versus host disease (GVHD), pulmonary fibrosis and autoimmune disorders, are distinguished by excessive production of connective tissue, which results in the destruction of normal tissue architecture and function. These diseases can best be interpreted in terms of perturbations in cellular functions, a major manifestation of which is excessive collagen synthesis and deposition. The role of collagen in fibrosis has prompted attempts to develop drugs that inhibit its accumulation.

Excessive accumulation of collagen is the major pathologic feature in a variety of clinical conditions characterized by tissue fibrosis. These conditions include localized processes, as for example, pulmonary fibrosis and liver cirrhosis, or more generalized processes, like progressive systemic sclerosis. Collagen deposition is a feature of different forms of dermal fibrosis, which in addition to scleroderma, include localized and generalized morphea, keloids, hypertrophic scars, familial cutaneous collagenoma and connective tissue nevi of the collagen type. Recent advances in the understanding of the normal biochemistry of collagen have allowed us to define specific levels of collagen biosynthesis and degradation at which a pharmacologic intervention could lead to reduced collagen deposition in the tissues. Such compounds could potentially provide us with novel means to reduce the excessive collagen accumulation in diseases.

Fibrosis of the liver, also referred to herein as hepatic fibrosis, may be caused by various types of chronic liver injury, especially if an inflammatory component is involved. Self-limited, acute liver injury (e.g., acute viral hepatitis A), even when fulminant, does not necessarily distort the scaffolding architecture and hence does not typically cause fibrosis, despite loss of hepatocytes. However, factors such as chronic alcoholism, malnutrition, hemochromatosis, and exposure to poisons, toxins or drugs, may lead to chronic liver injury and hepatic fibrosis due to exposure to hepatotoxic chemical substances. Hepatic scarring, caused by surgery or other forms of injury associated with mechanical biliary obstruction, may also result in liver fibrosis.

Fibrosis itself is not necessarily symptomatic, however it can lead to the development of portal hypertension, in which scarring distorts blood flow through the liver, or cirrhosis, in which scarring results in disruption of normal hepatic architecture and liver dysfunction. The extent of each of these pathologies determines the clinical manifestation of hepato-fibrotic disorders. For example, congenital hepatic fibrosis affects portal vein branches, largely sparing the parenchyma. The result is portal hypertension with sparing of hepatocellular function.

### Treatment

Attempts to develop anti-fibrotic agents for the treatment of various disorders have been reported. However, treatment of established fibrosis, formed after months or years of chronic or repeated injury, still remains a challenge.

Treatments aimed at reversing the fibrosis are usually too toxic for long-term use (e.g. corticosteroids, penicillamine) or have no proven efficacy (e.g. colchicine).

Many patients do not respond to available treatments for fibrotic disorders, and long-term treatment is limited by toxicity and side effects. Therefore, a need remains for developing therapeutic modalities aimed at reducing fibrosis. The development of safe and effective treatments for established cirrhosis and portal hypertension and for attenuating fibrosis would be highly beneficial.

Attempts to treat Idiopathic pulmonary fibrosis (IPF) with a combination of anti-inflammatory drugs (prednisone, azathioprine and N-acetyl-1-cysteine (NAC)), failed to improve outcomes, and instead increased mortality. In 2014, two drugs, pirfenidone, a drug with poorly understood mechanisms, and nintedanib, a tyrosine kinase inhibitor, were approved for the treatment of IPF mainly on the basis of their ability to reduce the decrease in forced vital capacity (FVC) and to slow the pace of disease progression. To date, however, it is unclear whether these drugs improve symptoms such as dyspnoea and cough, or whether their beneficial effect on functional decline translates to increased survival.

The compounds of this invention target activated fibroblasts and collagen over production and can therefore be used for treating fibrosis, including primary or secondary fibrosis, such as systemic sclerosis, graft-versus host disease (GVHD), pulmonary fibrosis and autoimmune disorders, lung fibrosis and Idiopathic pulmonary fibrosis (IPF), as well as localized processes, as for example, pulmonary fibrosis and liver cirrhosis, or more generalized processes, like progressive systemic sclerosis. The compounds can be further useful in the treatment of different forms of dermal fibrosis, which in addition to scleroderma, include localized and generalized morphea, keloids, hypertrophic scars, familial cutaneous collagenoma and connective tissue nevi of the collagen type. The compounds can be further useful in the treatment of lung fibrosis and Idiopathic pulmonary fibrosis (IPF), as well as hepatic fibrosis, resulting from hepatic scarring, caused by surgery or other forms of injury associated with mechanical biliary obstruction. Such fibrosis can lead to portal hypertension, in which scarring distorts blood flow through the liver, or cirrhosis as well as other hepato-fibrotic disorders including Non-alcoholic steatohepatitis (NASH), and alcoholic steatohepatitis (ASH), non-alcoholic fatty liver disease (NAFLD) and alcoholic fatty liver disease (AFLD), which can be similarly be treated by compounds of the invention.

### SUMMARY OF THE INVENTION

This invention provides a compound or its pharmaceutically acceptable salt, optical isomer, tautomer, hydrate, N-oxide, reverse amide analog, prodrug, isotopic variants (e.g., deuterated analog), PROTAC, pharmaceutical product or any combination thereof, represented by the structure of formula I, II and I(a)-I(f), and by the structures listed in Table 1, as defined herein below. In various embodiments, the compound is Collagen I translation inhibitor.

This invention further provides a pharmaceutical composition comprising a compound or its pharmaceutically acceptable salt, optical isomer, tautomer, hydrate, N-oxide, prodrug, isotopic variants (e.g., deuterated analog), PROTAC, pharmaceutical product or any combination thereof, represented by the structure of formula I, II and I(a)-I(f), and by the structures listed in Table 1, as defined herein below, and a pharmaceutically acceptable carrier.

This invention further provides a method of treating, suppressing, reducing the severity, reducing the risk of developing or inhibiting fibrosis in a subject, comprising administering a compound represented by the structure of formula I, II and I(a)-I(f), and by the structures listed in Table 1, as defined herein below, to a subject suffering from fibrosis under conditions effective to treat, suppress, reduce the severity, reduce the risk of developing, or inhibit fibrosis in said subject. In some embodiments, the fibrosis is a systemic fibrotic disease. In some embodiments, the systemic fibrotic disease is systemic sclerosis, multifocal fibrosclerosis (IgG4-associated fibrosis), nephrogenic systemic fibrosis, sclerodermatous graft vs. host disease, or any combination thereof. In some embodiments, the fibrosis is an organ-specific fibrotic disease. In some embodiments, the organ-specific fibrotic disease is lung fibrosis, cardiac fibrosis, kidney fibrosis, pulmonary fibrosis, liver and portal vein fibrosis, radiation-induced fibrosis, bladder fibrosis, intestinal fibrosis, peritoneal sclerosis, diffuse fasciitis, wound healing, scaring, or any combination thereof. In some embodiments, the lung fibrosis is Idiopathic pulmonary fibrosis (IPF). In some embodiments, the cardiac fibrosis is hypertension-associated cardiac fibrosis, Post-myocardial infarction, Chagas disease-induced myocardial fibrosis or any combination thereof. In some embodiments, the kidney fibrosis is diabetic and hypertensive nephropathy, urinary tract obstruction-induced kidney fibrosis, inflammatory/autoimmune-induced kidney fibrosis, aristolochic acid nephropathy, polycystic kidney disease, or any combination thereof. In some embodiments, the pulmonary fibrosis is idiopathic pulmonary fibrosis, silica-induced pneumoconiosis (silicosis), asbestos-induced pulmonary fibrosis (asbestosis), chemotherapeutic agent-induced pulmonary fibrosis, or any combination thereof. In some embodiments, the liver and portal vein fibrosis is alcoholic and nonalcoholic liver fibrosis, hepatitis C-induced liver fibrosis, primary biliary cirrhosis, parasite-induced liver fibrosis (schistosomiasis), or any combination thereof. In some embodiments, the diffuse fasciitis is localized scleroderma, keloids, dupuytren's disease, peyronie's disease, myelofibrosis, oral submucous fibrosis, or any combination thereof. In some embodiments, the fibrosis is primary or secondary fibrosis. In some embodiments, the fibrosis is a result of systemic sclerosis, graft-versus host disease (GVHD), pulmonary fibrosis, autoimmune disorder, tissue injury, inflammation, oxidative stress or any combination thereof. In some embodiments, the fibrosis is hepatic fibrosis, lung fibrosis or dermal fibrosis. In some embodiments, the subject has a liver cirrhosis. In some embodiments, the dermal fibrosis is scleroderma. In some embodiments, the dermal fibrosis is a result of a localized or generalized morphea, keloids, hypertrophic scars, familial cutaneous collagenoma, connective tissue nevi of the collagen type, or any combination thereof. In some embodiments, the hepatic fibrosis is a result of hepatic scarring or chronic liver injury. In some embodiments, the chronic liver injury results from alcoholism, malnutrition, hemochromatosis, exposure to poisons, toxins or drugs.

This invention further provides a method of treating, suppressing, reducing the severity, reducing the risk of developing or inhibiting lung fibrosis in a subject, comprising administering a compound represented by the structure of formula I, II and I(a)-I(f), and by the structures listed in Table 1, as defined herein below, to a subject suffering from lung fibrosis under conditions effective to treat, suppress, reduce the severity, reduce the risk of developing, or inhibit lung fibrosis in said subject. In some embodiments, the lung fibrosis is Idiopathic pulmonary fibrosis (IPF).

This invention further provides a method of treating, suppressing, reducing the severity, reducing the risk of developing or inhibiting Idiopathic pulmonary fibrosis (IPF) in a subject, comprising administering a compound represented by the structure of formula I, II and I(a)-I(f), and by the structures listed in Table 1, as defined herein below, to a subject suffering from Idiopathic pulmonary fibrosis (IPF) under conditions effective to treat, suppress, reduce the severity, reduce the risk of developing, or inhibit Idiopathic pulmonary fibrosis (IPF) in said subject.

This invention further provides a method of treating, suppressing, reducing the severity, reducing the risk of developing or inhibiting hepato-fibrotic disorder in a subject, comprising administering a compound represented by the structure of formula I, II and I(a)-I(f), and by the structures listed in Table 1, as defined herein below, to a subject suffering from hepato-fibrotic disorder under conditions effective to treat, suppress, reduce the severity, reduce the risk of developing, or inhibit hepato-fibrotic disorder in said subject. In some embodiments, the hepato-fibrotic disorder is a portal hypertension, cirrhosis, congenital hepatic fibrosis or any combination thereof.

This invention further provides a method of treating, suppressing, reducing the severity, reducing the risk of developing or inhibiting cirrhosis in a subject, comprising administering a compound represented by the structure of formula I, II and I(a)-I(f), and by the structures listed in Table 1, as defined herein below, to a subject suffering from cirrhosis under conditions effective to treat, suppress, reduce the severity, reduce the risk of developing, or inhibit cirrhosis in said subject. In some embodiments, the cirrhosis is a result of hepatitis or alcoholism.

This invention further provides a method of treating, suppressing, reducing the severity, reducing the risk of developing or inhibiting a alcoholic steatohepatitis (ASH) in a subject, comprising administering a compound represented by the structure of formula I, II and I(a)-I(f), and by the structures listed in Table 1, as defined herein below, to a subject suffering from alcoholic steatohepatitis (ASH) under conditions effective to treat, suppress, reduce the severity, reduce the risk of developing, or inhibit the alcoholic steatohepatitis (ASH) in said subject.

This invention further provides a method of treating, suppressing, reducing the severity, reducing the risk of developing or inhibiting a non-alcoholic steatohepatitis (NASH) in a subject, comprising administering a compound represented by the structure of formula I, II and I(a)-I(f), and by the structures listed in Table 1, as defined herein below, to a subject suffering from non-alcoholic steatohepatitis (NASH) under conditions effective to treat, suppress, reduce the severity, reduce the risk of developing, or inhibit the non-alcoholic steatohepatitis (NASH) in said subject.

This invention further provides a method of treating, suppressing, reducing the severity, reducing the risk of developing or inhibiting a alcoholic fatty liver disease (AFLD) in a subject, comprising administering a compound represented by the structure of formula I, II and I(a)-I(f), and by the structures listed in Table 1, as defined herein below, to a subject suffering from alcoholic fatty liver disease (AFLD) under conditions effective to treat, suppress, reduce the severity, reduce the risk of developing, or inhibit the alcoholic fatty liver disease (AFLD) in said subject.

This invention further provides a method of treating, suppressing, reducing the severity, reducing the risk of developing or inhibiting a non alcoholic fatty liver disease (NAFLD) in a subject, comprising administering a compound represented by the structure of formula I, II and I(a)-I(f), and by the structures listed in Table 1, as defined herein below, to a subject suffering from non alcoholic fatty liver disease (NAFLD) under conditions effective to treat, suppress, reduce the severity, reduce the risk of developing, or inhibit the non alcoholic fatty liver disease (NAFLD) in said subject.

This invention further provides a method of treating, suppressing, reducing the severity, reducing the risk of developing or inhibiting an autoimmune disease or disorder in a subject, comprising administering a compound represented by the structure of formula I, II and I(a)-I(f), and by the structures listed in Table 1, as defined herein below, to a subject suffering from an autoimmune disease or disorder under conditions effective to treat, suppress, reduce the severity, reduce the risk of developing, or inhibit the autoimmune disease or disorder in said subject.

This invention further provides a method of treating, suppressing, reducing the severity, reducing the risk of developing or inhibiting an autoimmune disease or disorder in a subject, comprising administering a compound represented by the structure of formula I, II and I(a)-I(f), and by the structures listed in Table 1, as defined herein below, to a subject suffering from an autoimmune disease or disorder under conditions effective to treat, suppress, reduce the severity, reduce the risk of developing, or inhibit the autoimmune disease or disorder in said subject.

### BRIEF DESCRIPTION OF THE DRAWINGS

The patent of application file contains at least one drawing executed in color. Copies of this patent or patent application publication with color drawing(s) will be provided by the office upon request and payment of the necessary fee.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 demonstrates how Protein synthesis monitoring (PSM) specifically monitors collagen 1 synthesis. The assay system comprises human lung fibroblast cell line, WI-38 cells, which are activated to produce higher levels of collagen. Two tRNAs (di-tRNA) which decode one specific glycine codon and one specific proline codon were transfected with control RNAi or an RNAi directed to Collagen 1. The FRET signal specifically monitors collagen 1 translation, as the FRET signal in collagen 1-targeted siRNA treated cells is inhibited by 90%. In blue, cell nuclei stained with DAPI; In Cyan, FRET signals from tRNA pair which decodes glycine-proline di-codons.

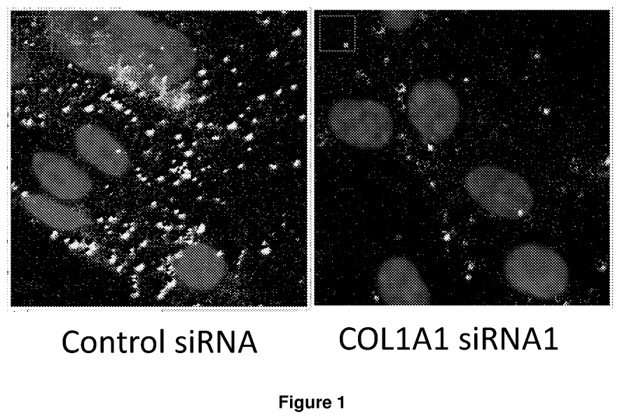

FIG. 2 depicts that hits selectively regulate collagen translation. In the upper panel, the Y-axis depicts normalized values of metabolic labeling in control cells. Only compounds which showed minimal effects on global protein synthesis (±20% of control) and minimal effects on collagen 1 protein accumulation in W138 cells by di-tRNA Collagen FRET and by Collagen 1 specific immunofluorescence were selected as compounds which selectively regulate collagen synthesis; In the lower panel, Y axis shows the FRET score for the collagen specific di-tRNA (PSM score) and the X-axis shows the normalized immunofluorescence values (relative to control). Compounds that show high PSM score are marked by dot size; compounds that increase collagen content are marked as red, and compounds that decrease collagen content are marked as green.

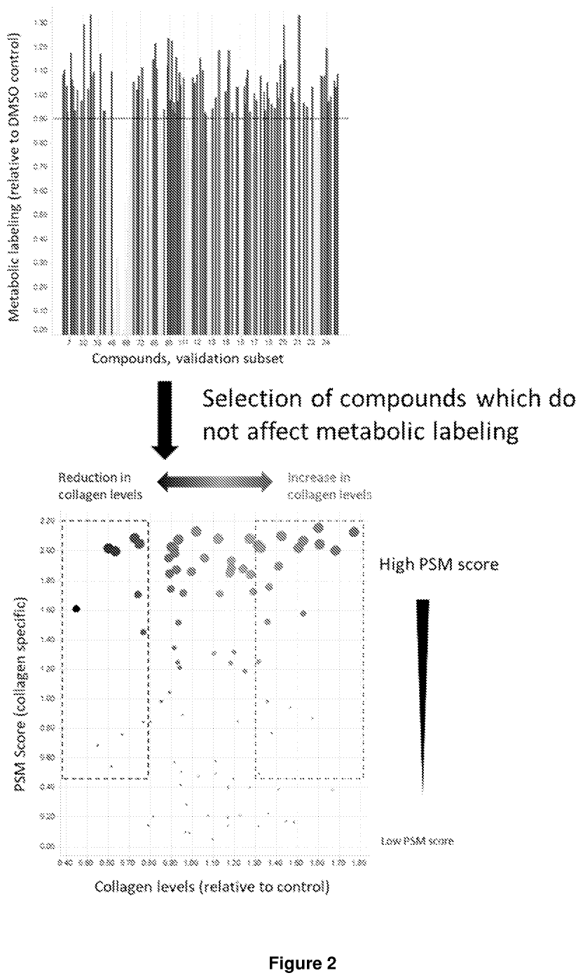

FIG. 3 demonstrates that compounds act at the level of translation. Upper panel: WI-38 Human Lung Fibroblasts, 96 hours incubation with compounds. Immunofluorescence. In blue, cell nuclei stained with DAPI; In green, Collagen protein detected with anti-collagen antibody. Lower panel: WI-38 Human Lung Fibroblasts, 24 hours incubation with compounds. FISH analysis. In blue, cell nuclei stained with DAPI; In red, collagen mRNA detected with fluorescent in situ hybridization using collagen 1 mRNA specific probes.

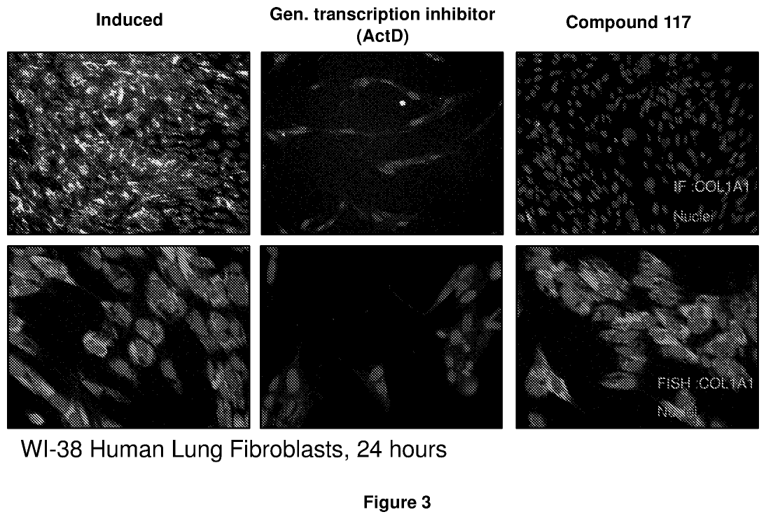

FIG. 4 demonstrates the efficacy and toxicity of compounds 124, 133, 110 and 131. FIG. 4A depicts the logged EC₅₀ of efficacy plotted against logged EC₅₀ of toxicity. Dashed lines represent ×10 or ×100 window between efficacy and toxicity. FIG. 4B depicts representative images from compound 110. Images were taken with ×20 objective in Operetta machine (Perkin-Elmer). Green: Collagen type-I; Grey: DAPI.

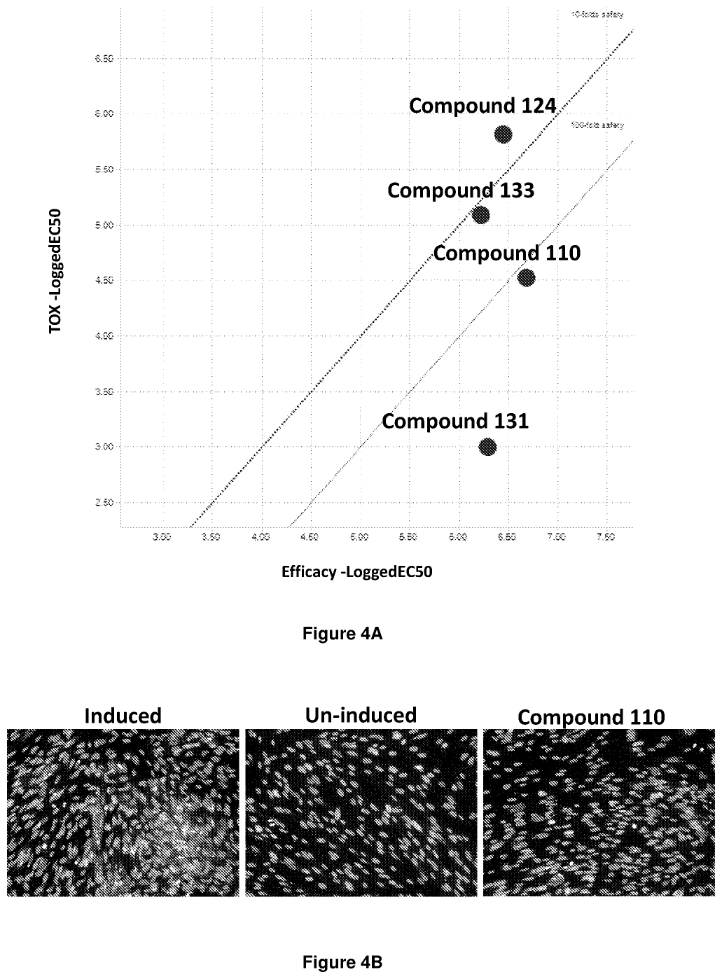

### DETAILED DESCRIPTION OF THE INVENTION

In various embodiments, this invention is directed to a compound represented by the structure of formula (I):

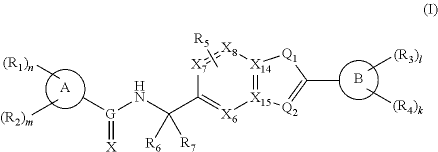

wherein

A and B rings are each independently a single or fused aromatic or heteroaromatic ring system (e.g., A: phenyl, pyrimidine, 2-, 3- or 4-pyridine, pyridazine, pyrazine, isothiazole, thiadiazole, imidazole, triazole, triazolopyrimidine, thiazole, oxazole, isoxazole, 1-methylimidazole, pyrrole, furane, thiophene, oxadiazole, or pyrazole; B: phenyl, pyrimidine, 2-, 3- or 4-pyridine, pyridazine or pyrazine, thiazole, imidazole, indazole), or a single or fused C₃-C₁₀ cycloalkyl (e.g. cyclopentyl) or a single or fused C₃-C₁₀ heterocyclic ring (e.g., piperidine, A, B: tetrahydro-2H-pyran, thiane 1,1-dioxide, tetrahydrofurane, oxazolone, oxazolidone, thiazolone, isothiazolynone, isoxazolidinone, imidazolidinone, pyrazolone, 2H-pyrrol-2-one, furanone, thiophenone, 6,7-dihydro-5H-pyrazolo[5,1-b][1,3]oxazine);

R₁ and R₂ are each independently H, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

or R₂ and R₁ are joint together to form a 5 or 6 membered substituted or unsubstituted, aliphatic or aromatic, carbocyclic or heterocyclic ring;

R₃ and R₄ are each independently H, F, Cl, Br, I, OH, SH, R₈—OH, R₈—SH, —R₈—O—R₁₀ (e.g., CH₂—O—CH₃)—O—R₈—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁) (e.g., CH₂—NH—CH₃, CH₂—N(CH₃)₂, CH₂—NH—C(═O)—CH₃), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl, CH₂—OCH₂—CH₂—O—CH₃), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂—O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

or R₃ and R₄ are joint together to form a 5 or 6 membered substituted or unsubstituted, aliphatic or aromatic, carbocyclic or heterocyclic ring;

R₅ is absent or is H, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

R₆ and R₇ are each independently H, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

or R₆ and R₇ are joint together to form a 3 to 6 membered substituted or unsubstituted, aliphatic or aromatic, carbocyclic or heterocyclic ring (e.g. cyclopropyl);

Q₁ is NH, N(R), S, O, N—OH or N—OMe;

Q₂ is N, or C(R);

R is H, F, Cl, Br, I, OH, SH, OH, alkoxy, N(R)₂, CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂O—CH₃), —R₈—O—R₁₀, —R₈—R₁₀ (e.g., (CH₂)₂O—CH₃), substituted or unsubstituted aryl (e.g., phenyl), substituted or unsubstituted heteroaryl (e.g., pyridine (2, 3, and 4-pyridine), (wherein substitutions include: F, Cl, Br, I, OH, SH, C₁-C₅ linear or branched alkyl, OH, alkoxy, N(R)₂, CF₃, phenyl, halophenyl, (benzyloxy)phenyl, CN, NO₂ or any combination thereof);

G=X is C═O, C═S, S═O, SO₂, CH₂, CHR, or C(R)₂;

each R₈ is independently [CH₂]ₚ
wherein p is between 1 and 10;

R₉ is [CH]_{q}, [C]_{q}
wherein q is between 2 and 10;

R₁₀ and R₁₁ are each independently H, C₁-C₅ substituted or unsubstituted linear or branched alkyl (e.g., methyl, ethyl, CH₂—CH₂—O—CH₃), C₁-C₅ linear or branched alkoxy (e.g., O—CH₃), C(O)R, or S(O)₂R; or R₁₀ and R₁₁ are joint to form a substituted or unsubstituted C₃-C₈ heterocyclic ring (e.g., piperazine, piperidine),
wherein substitutions include: F, Cl, Br, I, OH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof)

m, n, l and k are each independently an integer between 0 and 4 (e.g., 0, 1 or 2);

X₆, X₇ and X₈ are each independently C or N;

X₁₄ and X₁₅ are each independently C or N;

or its pharmaceutically acceptable salt, optical isomer, tautomer, hydrate, N-oxide, reverse amide analog, prodrug, isotopic variant (e.g., deuterated analog), PROTAC, pharmaceutical product or any combination thereof;

wherein R₅ can only be attached to X₆, X₇ and/or X₈ that are C.

In various embodiments, this invention is directed to a compound represented by the structure of formula (II):

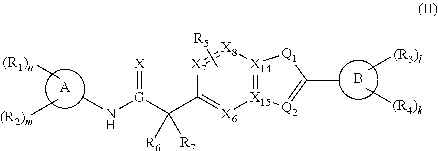

wherein

A and B rings are each independently a single or fused aromatic or heteroaromatic ring system (e.g., A: phenyl, pyrimidine, 2-, 3- or 4-pyridine, pyridazine, pyrazine, isothiazole, thiadiazole, imidazole, triazole, triazolopyrimidine, thiazole, oxazole, isoxazole, 1-methylimidazole, pyrrole furane, thiophene, 1 oxadiazole, or pyrazole; B: phenyl, pyrimidine, 2-, 3- or 4-pyridine, pyridazine or pyrazine, thiazole, imidazole, indazole), or a single or fused C₃-C₁₀ cycloalkyl (e.g. cyclopentyl) or a single or fused C₃-C₁₀ heterocyclic ring (e.g., piperidine, A, B: tetrahydro-2H-pyran, thiane 1,1-dioxide, tetrahydrofurane, oxazolone, oxazolidone, thiazolone, isothiazolynone, isoxazolidinone, imidazolidinone, pyrazolone, 2H-pyrrol-2-one, furanone, thiophenone, 6,7-dihydro-5H-pyrazolo[5,1-b][1,3]oxazine);

R₁ and R₂ are each independently H, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

or R₂ and R₁ are joint together to form a 5 or 6 membered substituted or unsubstituted, aliphatic or aromatic, carbocyclic or heterocyclic ring;

R₃ and R₄ are each independently H, F, Cl, Br, I, OH, SH, R₈—OH, R₈—SH, —R₈—O—R₁₀, —O—R₈—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl, CH₂—OCH₂—CH₂—O—CH₃), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

or R₃ and R₄ are joint together to form a 5 or 6 membered substituted or unsubstituted, aliphatic or aromatic, carbocyclic or heterocyclic ring;

R₅ is absent or is H, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

R₆ and R₇ are each independently H, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

or R₆ and R₇ are joint together to form a 3 to 6 membered substituted or unsubstituted, aliphatic or aromatic, carbocyclic or heterocyclic ring (e.g. cyclopropyl);

Q₁ is NH, N(R), S, O, N—OH, or N—OMe;

Q₂ is N, or C(R);

R is H, F, Cl, Br, I, OH, SH, OH, alkoxy, N(R)₂, CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂O—CH₃), —R₈—O—R₁₀, —R₈—R₁₀ (e.g., (CH₂)₂O—CH₃), substituted or unsubstituted aryl (e.g., phenyl), substituted or unsubstituted heteroaryl (e.g., pyridine (2, 3, and 4-pyridine), (wherein substitutions include: F, Cl, Br, I, OH, SH, C₁-C₅ linear or branched alkyl, OH, alkoxy, N(R)₂, CF₃, phenyl, halophenyl, (benzyloxy)phenyl, CN, NO₂ or any combination thereof);

G=X is C═O, C═S, S═O, SO₂, CH₂, CHR, or C(R)₂;

each R₈ is independently [CH₂]ₚ
wherein p is between 1 and 10;

R₉ is [CH]_{q}, [C]_{q}
wherein q is between 2 and 10;

R₁₀ and R₁₁ are each independently H, C₁-C₈ substituted or unsubstituted linear or branched alkyl (e.g., methyl, ethyl, CH₂—CH₂—O—CH₃), C₁-C₅ linear or branched alkoxy (e.g., O—CH₃), C(O)R, or S(O)₂R; or R₁₀ and R₁₁ are joint to form a substituted or unsubstituted C₃-C₈ heterocyclic ring (e.g., piperazine, piperidine),
wherein substitutions include: F, Cl, Br, I, OH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof)

m, n, l and k are each independently an integer between 0 and 4 (e.g., 0, 1 or 2);

X₆, X₇ and X₈ are each independently C or N;

X₁₄ and X₁₅ are each independently C or N;

or its pharmaceutically acceptable salt, optical isomer, tautomer, hydrate, N-oxide, reverse amide analog, prodrug, isotopic variant (e.g., deuterated analog), PROTAC, pharmaceutical product or any combination thereof;

wherein R₅ can only be attached to X₆, X₇ and/or X₈ that are C.

In various embodiments, this invention is directed to a compound represented by the structure of formula I(a)

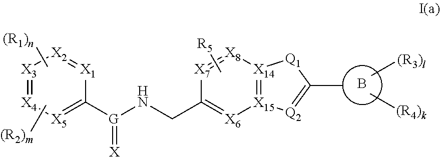

wherein

B ring is a single or fused aromatic or heteroaromatic ring system (e.g., phenyl, pyrimidine, 2-, 3- or 4-pyridine, pyridazine or pyrazine, thiazole, imidazole, indazole), or a single or fused C₃-C₁₀ cycloalkyl (e.g. cyclopentyl) or a single or fused C₃-C₁₀ heterocyclic ring (e.g., piperidine, tetrahydropyran, thiane 1,1-dioxide);

R₁ and R₂ are each independently H, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

or R₂ and R₁ are joint together to form a 5 or 6 membered substituted or unsubstituted, aliphatic or aromatic, carbocyclic or heterocyclic ring;

R₃ and R₄ are each independently H, F, Cl, Br, I, OH, SH, R₈—OH, R₈—SH, —R₈—O—R₁₀, —O—R₈—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl, CH₂—OCH₂—CH₂—O—CH₃), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

or R₃ and R₄ are joint together to form a 5 or 6 membered substituted or unsubstituted, aliphatic or aromatic, carbocyclic or heterocyclic ring;

R₅ is absent or is H, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

Q₁ is NH, N(R), S, O, N—OH, or N—OMe;

Q₂ is N, or C(R);

R is H, F, Cl, Br, I, OH, SH, OH, alkoxy, N(R)₂, CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈—R₁₀ (e.g., (CH₂)₂—O—CH₃), substituted or unsubstituted aryl (e.g., phenyl), substituted or unsubstituted heteroaryl (e.g., pyridine (2, 3, and 4-pyridine), (wherein substitutions include: F, Cl, Br, I, OH, SH, C₁-C₅ linear or branched alkyl, OH, alkoxy, N(R)₂, CF₃, phenyl, halophenyl, (benzyloxy)phenyl, CN, NO₂ or any combination thereof);

G=X is C═O, C═S, S═O, SO₂, CH₂, CHR, or C(R)₂;

each R₈ is independently is [CH₂]ₚ
wherein p is between 1 and 10;

R₉ is [CH]_{q}, [C]_{q}
wherein q is between 2 and 10;

R₁₀ and R₁₁ are each independently H, C₁-C₅ substituted or unsubstituted linear or branched alkyl (e.g., methyl, ethyl, CH₂—CH₂—O—CH₃), C₁-C₅ linear or branched alkoxy (e.g., O—CH₃), C(O)R, or S(O)₂R; or R₁₀ and R₁₁ are joint to form a substituted or unsubstituted C₃-C₈ heterocyclic ring (e.g., piperazine, piperidine),
wherein substitutions include: F, Cl, Br, I, OH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof)

m, n, l and k are each independently an integer between 0 and 4 (e.g., 0, 1 or 2);

X₁, X₂, X₃, X₄, X₅, X₆, X₇ and X₈ are each independently C or N;

X₁₄ and X₁₅ are each independently C or N;

or its pharmaceutically acceptable salt, optical isomer, tautomer, hydrate, N-oxide, reverse amide analog, prodrug, isotopic variant (e.g., deuterated analog), PROTAC, pharmaceutical product or any combination thereof;

wherein R₅ can only be attached to X₆, X₇ and/or X₈ that are C.

In various embodiments, this invention is directed to a compound represented by the structure of formula I(b):

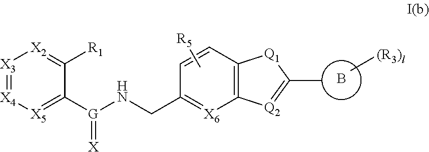

wherein

B ring is a single or fused aromatic or heteroaromatic ring system (e.g., phenyl, pyrimidine, 2-, 3- or 4-pyridine, pyridazine or pyrazine, thiazole, imidazole, indazole), or a single or fused C₃-C₁₀ cycloalkyl (e.g. cyclopentyl) or a single or fused C₃-C₁₀ heterocyclic ring (e.g., piperidine, tetrahydro-2H-pyran, thiane 1,1-dioxide);

R₁ is H, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

R₃ is H, F, Cl, Br, I, OH, SH, R₈—OH, R₈—SH, —R₈—O—R₁₀, —O—R₈—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl, CH₂—OCH₂—CH₂—O—CH₃), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂—O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

R₅ is absent or is H, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

Q₁ is NH, N(R), S, O, N—OH, or N—OMe;

Q₂ is N, or C(R);

R is H, F, Cl, Br, I, OH, SH, OH, alkoxy, N(R)₂, CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂O—CH₃), —R₈—O—R₁₀, —R₈—R₁₀ (e.g., (CH₂)₂O—CH₃), substituted or unsubstituted aryl (e.g., phenyl), substituted or unsubstituted heteroaryl (e.g., pyridine (2, 3, and 4-pyridine), (wherein substitutions include: F, Cl, Br, I, OH, SH, C₁-C₅ linear or branched alkyl, OH, alkoxy, N(R)₂, CF₃, phenyl, halophenyl, (benzyloxy)phenyl, CN, NO₂ or any combination thereof);

G=X is C═O, C═S, S═O, SO₂, CH₂, CHR, or C(R)₂;

each R₈ is independently is [CH₂]ₚ

wherein p is between 1 and 10;

R₉ is [CH]_{q}, [C]_{q}
wherein q is between 2 and 10;

R₁₀ and R₁₁ are each independently H, C₁-C₅ substituted or unsubstituted linear or branched alkyl (e.g., methyl, ethyl, CH₂—CH₂—O—CH₃), C₁-C₅ linear or branched alkoxy (e.g., O—CH₃), C(O)R, or S(O)₂R; or R₁₀ and R₁₁ are joint to form a substituted or unsubstituted C₃-C₈ heterocyclic ring (e.g., piperazine, piperidine),

wherein substitutions include: F, Cl, Br, I, OH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof)

l is an integer between 0 and 4 (e.g., 0, 1 or 2);

X₂, X₃, X₄, X₅, and X₆ are each independently C or N;

or its pharmaceutically acceptable salt, optical isomer, tautomer, hydrate, N-oxide, reverse amide analog, prodrug, isotopic variant (e.g., deuterated analog), PROTAC, pharmaceutical product or any combination thereof;

wherein R₅ can only be attached to carbon atoms.

In various embodiments, this invention is directed to a compound represented by the structure of formula I(c):

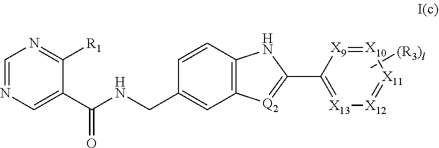

wherein

R₁ is H, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

R₃ is H, F, Cl, Br, I, OH, SH, R₈—OH, R₈—SH, —R₈—O—R₁₀, —O—R₈—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl, CH₂—OCH₂—CH₂—O—CH₃), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂—O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

Q₂ is N, or C(R);

R is H, F, Cl, Br, I, OH, SH, OH, alkoxy, N(R)₂, CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈—R₁₀ (e.g., (CH₂)₂—O—CH₃), substituted or unsubstituted aryl (e.g., phenyl), substituted or unsubstituted heteroaryl (e.g., pyridine (2, 3, and 4-pyridine), (wherein substitutions include: F, Cl, Br, I, OH, SH, C₁-C₅ linear or branched alkyl, OH, alkoxy, N(R)₂, CF₃, phenyl, halophenyl, (benzyloxy)phenyl, CN, NO₂ or any combination thereof);

each R₈ is independently is [CH₂]ₚ
wherein p is between 1 and 10;

R₉ is [CH]_{q}, [C]_{q}
wherein q is between 2 and 10;

R₁₀ and R₁₁ are each independently H, C₁-C₅ substituted or unsubstituted linear or branched alkyl (e.g., methyl, ethyl, CH₂—CH₂—O—CH₃), C₁-C₅ linear or branched alkoxy (e.g., O—CH₃), C(O)R, or S(O)₂R; or R₁₀ and R₁₁ are joint to form a substituted or unsubstituted C₃-C₈ heterocyclic ring (e.g., piperazine, piperidine),

wherein substitutions include: F, Cl, Br, I, OH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof)

l is an integer between 0 and 4 (e.g., 0, 1 or 2);

X₉, X₁₀, X₁₁, X₁₂, and X₁₃ are each independently C or N;

or its pharmaceutically acceptable salt, optical isomer, tautomer, hydrate, N-oxide, reverse amide analog, prodrug, isotopic variant (e.g., deuterated analog), PROTAC, pharmaceutical product or any combination thereof.

In various embodiments, this invention is directed to a compound represented by the structure of formula I(d):

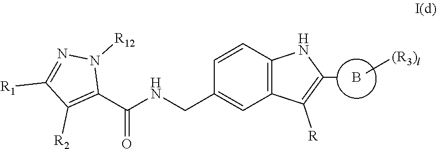

wherein

B ring is a single or fused aromatic or heteroaromatic ring system (e.g., phenyl, pyrimidine, 2-, 3- or 4-pyridine, pyridazine or pyrazine, thiazole, imidazole, indazole), or a single or fused C₃-C₁₀ cycloalkyl (e.g. cyclopentyl) or a single or fused C₃-C₁₀ heterocyclic ring (e.g., piperidine, tetrahydro-2H-pyran, thiane 1,1-dioxide);

R₁ and R₂ are each independently H, F, Cl, Br, I, OH, SH, R₈—OH, R₈—S, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

or R₂ and R₁ are joint together to form a 5 or 6 membered substituted or unsubstituted, aliphatic or aromatic, carbocyclic or heterocyclic ring;

R₃ is H, F, Cl, Br, I, OH, SH, R₈—OH, R₈—SH, —R₈—O—R₁₀, —O—R₈—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl, CH₂—OCH₂—CH₂—O—CH₃), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂—O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

R is H, F, Cl, Br, I, OH, SH, OH, alkoxy, N(R)₂, CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈—R₁₀ (e.g., (CH₂)₂—O—CH₃), substituted or unsubstituted aryl (e.g., phenyl), substituted or unsubstituted heteroaryl (e.g., pyridine (2, 3, and 4-pyridine), (wherein substitutions include: F, Cl, Br, I, OH, SH, C₁-C₅ linear or branched alkyl, OH, alkoxy, N(R)₂, CF₃, phenyl, halophenyl, (benzyloxy)phenyl, CN, NO₂ or any combination thereof);

each R₈ is independently is [CH₂]ₚ
wherein p is between 1 and 10;

R₉ is [CH]_{q}, [C]_{q}
wherein q is between 2 and 10;

R₁₀ and R₁₁ are each independently H, C₁-C₅ substituted or unsubstituted linear or branched alkyl (e.g., methyl, ethyl, CH₂—CH₂—O—CH₃), C₁-C₅ linear or branched alkoxy (e.g., O—CH₃), C(O)R, or S(O)₂R; or R₁₀ and R₁₁ are joint to form a substituted or unsubstituted C₃-C₈ heterocyclic ring (e.g., piperazine, piperidine),

wherein substitutions include: F, Cl, Br, I, OH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof)

R₁₂ is H, F, Cl, Br, I, OH, SH, OH, N(R)₂, CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂) or substituted or unsubstituted C₃-C₈ cycloalkyl (e.g. cyclopropyl) (wherein substitutions include: F, Cl, Br, I, OH, SH, C₁-C₅ linear or branched alkyl, OH, alkoxy, N(R)₂, CF₃, phenyl, halophenyl, CN, NO₂ or any combination thereof);

l is an integer between 0 and 4 (e.g., 0, 1 or 2);

or its pharmaceutically acceptable salt, optical isomer, tautomer, hydrate, N-oxide, reverse amide analog, prodrug, isotopic variant (e.g., deuterated analog), PROTAC, pharmaceutical product or any combination thereof.

In various embodiments, this invention is directed to a compound represented by the structure of formula I(d(i)):

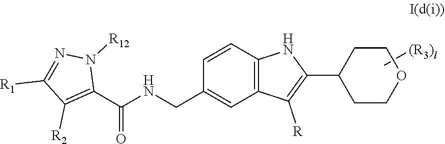

wherein

R₁ and R₂ are each independently H, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

or R₂ and R₁ are joint together to form a 5 or 6 membered substituted or unsubstituted, aliphatic or aromatic, carbocyclic or heterocyclic ring;

R₃ is H, F, Cl, Br, I, OH, SH, R₈—OH, R₈—SH, —R₈—O—R₁₀, —O—R₈—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl, CH₂—OCH₂—CH₂—O—CH₃), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂—O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

R is H, F, Cl, Br, I, OH, SH, OH, alkoxy, N(R)₂, CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈—R₁₀ (e.g., (CH₂)₂—O—CH₃), substituted or unsubstituted aryl (e.g., phenyl), substituted or unsubstituted heteroaryl (e.g., pyridine (2, 3, and 4-pyridine), (wherein substitutions include: F, Cl, Br, I, OH, SH, C₁-C₅ linear or branched alkyl, OH, alkoxy, N(R)₂, CF₃, phenyl, halophenyl, (benzyloxy)phenyl, CN, NO₂ or any combination thereof);

each R₈ is independently is [CH₂]ₚ
wherein p is between 1 and 10;

R₉ is [CH]_{q}, [C]_{q}
wherein q is between 2 and 10;

R₁₀ and R₁₁ are each independently H, C₁-C₅ substituted or unsubstituted linear or branched alkyl (e.g., methyl, ethyl, CH₂—CH₂—O—CH₃), C₁-C₅ linear or branched alkoxy (e.g., O—CH₃), C(O)R, or S(O)₂R; or R₁₀ and R₁₁ are joint to form a substituted or unsubstituted C₃-C₈ heterocyclic ring (e.g., piperazine, piperidine),

wherein substitutions include: F, Cl, Br, I, OH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof)

R₁₂ is H, F, Cl, Br, I, OH, SH, OH, N(R)₂, CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂) or substituted or unsubstituted C₃-C₈ cycloalkyl (e.g. cyclopropyl) (wherein substitutions include: F, Cl, Br, I, OH, SH, C₁-C₅ linear or branched alkyl, OH, alkoxy, N(R)₂, CF₃, phenyl, halophenyl, CN, NO₂ or any combination thereof);

l is an integer between 0 and 4 (e.g., 0, 1 or 2);

or its pharmaceutically acceptable salt, optical isomer, tautomer, hydrate, N-oxide, reverse amide analog, prodrug, isotopic variant (e.g., deuterated analog), PROTAC, pharmaceutical product or any combination thereof.

In various embodiments, this invention is directed to a compound represented by the structure of formula I(e):

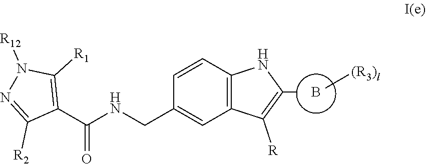

wherein

B ring is a single or fused aromatic or heteroaromatic ring system (e.g., phenyl, pyrimidine, 2-, 3- or 4-pyridine, pyridazine or pyrazine, thiazole, imidazole, indazole), or a single or fused C₃-C₁₀ cycloalkyl (e.g. cyclopentyl) or a single or fused C₃-C₁₀ heterocyclic ring (e.g., piperidine, tetrahydro-2H-pyran, thiane 1,1-dioxide);

R₁ and R₂ are each independently H, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

R₃ is H, F, Cl, Br, I, OH, SH, R₈—OH, R₈—SH, —R₈—O—R₁₀, —O—R₈—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl, CH₂—OCH₂—CH₂—O—CH₃), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂—O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

R is H, F, Cl, Br, I, OH, SH, OH, alkoxy, N(R)₂, CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈—R₁₀ (e.g., (CH₂)₂—O—CH₃), substituted or unsubstituted aryl (e.g., phenyl), substituted or unsubstituted heteroaryl (e.g., pyridine (2, 3, and 4-pyridine), (wherein substitutions include: F, Cl, Br, I, OH, SH, C₁-C₅ linear or branched alkyl, OH, alkoxy, N(R)₂, CF₃, phenyl, halophenyl, (benzyloxy)phenyl, CN, NO₂ or any combination thereof);

each R₈ is independently is [CH₂]ₚ
wherein p is between 1 and 10;

R₉ is [CH]_{q}, [C]_{q}
wherein q is between 2 and 10;

R₁₀ and R₁₁ are each independently H, C₁-C₅ substituted or unsubstituted linear or branched alkyl (e.g., methyl, ethyl, CH₂—CH₂—O—CH₃), C₁-C₅ linear or branched alkoxy (e.g., O—CH₃), C(O)R, or S(O)₂R; or R₁₀ and R₁₁ are joint to form a substituted or unsubstituted C₃-C₈ heterocyclic ring (e.g., piperazine, piperidine),

wherein substitutions include: F, Cl, Br, I, OH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof)

R₁₂ is H, F, Cl, Br, I, OH, SH, OH, N(R)₂, CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂) or substituted or unsubstituted C₃-C₈ cycloalkyl (e.g. cyclopropyl) (wherein substitutions include: F, Cl, Br, I, OH, SH, C₁-C₅ linear or branched alkyl, OH, alkoxy, N(R)₂, CF₃, phenyl, halophenyl, CN, NO₂ or any combination thereof);

l is an integer between 0 and 4 (e.g., 0, 1 or 2);

or its pharmaceutically acceptable salt, optical isomer, tautomer, hydrate, N-oxide, reverse amide analog, prodrug, isotopic variant (e.g., deuterated analog), PROTAC, pharmaceutical product or any combination thereof.

In various embodiments, this invention is directed to a compound represented by the structure of formula I(e(i)):

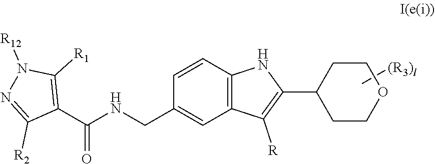

wherein

R₁ and R₂ are each independently H, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

R₃ is H, F, Cl, Br, I, OH, SH, R₈—OH, R₈—SH, —R₈—O—R₁₀, —O—R₈—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl, CH₂—OCH₂—CH₂—O—CH₃), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂—O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

R is H, F, Cl, Br, I, OH, SH, OH, alkoxy, N(R)₂, CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈—R₁₀ (e.g., (CH₂)₂—O—CH₃), substituted or unsubstituted aryl (e.g., phenyl), substituted or unsubstituted heteroaryl (e.g., pyridine (2, 3, and 4-pyridine), (wherein substitutions include: F, Cl, Br, I, OH, SH, C₁-C₅ linear or branched alkyl, OH, alkoxy, N(R)₂, CF₃, phenyl, halophenyl, (benzyloxy)phenyl, CN, NO₂ or any combination thereof);

each R₈ is independently is [CH₂]ₚ
wherein p is between 1 and 10;

R₉ is [CH]_{q}, [C]_{q}
wherein q is between 2 and 10;

R₁₀ and R₁₁ are each independently H, C₁-C₅ substituted or unsubstituted linear or branched alkyl (e.g., methyl, ethyl, CH₂—CH₂—O—CH₃), C₁-C₅ linear or branched alkoxy (e.g., O—CH₃), C(O)R, or S(O)₂R; or R₁₀ and R₁₁ are joint to form a substituted or unsubstituted C₃-C₈ heterocyclic ring (e.g., piperazine, piperidine),

wherein substitutions include: F, Cl, Br, I, OH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof)

R₁₂ is H, F, Cl, Br, I, OH, SH, OH, N(R)₂, CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂) or substituted or unsubstituted C₃-C₈ cycloalkyl (e.g. cyclopropyl) (wherein substitutions include: F, Cl, Br, I, OH, SH, C₁-C₅ linear or branched alkyl, OH, alkoxy, N(R)₂, CF₃, phenyl, halophenyl, CN, NO₂ or any combination thereof);

l is an integer between 0 and 4 (e.g., 0, 1 or 2);

or its pharmaceutically acceptable salt, optical isomer, tautomer, hydrate, N-oxide, reverse amide analog, prodrug, isotopic variant (e.g., deuterated analog), PROTAC, pharmaceutical product or any combination thereof.

In various embodiments, this invention is directed to a compound represented by the structure of formula I(f):

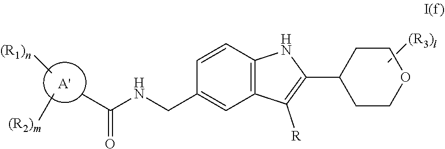

wherein

A′ ring is a 5 (five) membered heteroaromatic, or a heterocyclic ring (e.g. thiazolyl, isothiazolyl, oxazolyl, isoxazolyl, imidazolyl, 1-methylimidazolyl, pyrazolyl, pyrrolyl, furanyl, thiophene-yl, triazolyl, thiadiazolyl, oxadiazolyl, pyrrolidine, 2-oxo-pyrrolidine, tetrahydrofuranyl, oxazolonyl, oxazolidonyl, thiazolonyl, isothiazolynonyl, isoxazolidinonyl, imidazolidinonyl, pyrazolonyl, 2H-pyrrol-2-onyl, furanonyl, and thiophenonyl);

R₁ and R₂ are each independently H, F, Cl, Br, I, OH, SH, R₈—OH, R₈—S, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

or R₂ and R₁ are joint together to form a 5 or 6 membered substituted or unsubstituted, aliphatic or aromatic, carbocyclic or heterocyclic ring;

R₃ is H, F, Cl, Br, I, OH, SH, R₈—OH, R₈—SH, —R₈—O—R₁₀, —O—R₈—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl, CH₂—OCH₂—CH₂—O—CH₃), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂—O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof);

R is H, F, Cl, Br, I, OH, SH, OH, alkoxy, N(R)₂, CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈—R₁₀ (e.g., (CH₂)₂—O—CH₃), substituted or unsubstituted aryl (e.g., phenyl), substituted or unsubstituted heteroaryl (e.g., pyridine (2, 3, and 4-pyridine), (wherein substitutions include: F, Cl, Br, I, OH, SH, C₁-C₅ linear or branched alkyl, OH, alkoxy, N(R)₂, CF₃, phenyl, halophenyl, (benzyloxy)phenyl, CN, NO₂ or any combination thereof);

each R₈ is independently is [CH₂]ₚ
wherein p is between 1 and 10;

R₉ is [CH]_{q}, [C]_{q}
wherein q is between 2 and 10;

R₁₀ and R₁₁ are each independently H, C₁-C₅ substituted or unsubstituted linear or branched alkyl (e.g., methyl, ethyl, CH₂—CH₂—O—CH₃), C₁-C₅ linear or branched alkoxy (e.g., O—CH₃), C(O)R, or S(O)₂R; or R₁₀ and R₁₁ are joint to form a substituted or unsubstituted C₃-C₈ heterocyclic ring (e.g., piperazine, piperidine),

wherein substitutions include: F, Cl, Br, I, OH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof)

m, n, 1 are each independently an integer between 0 and 4 (e.g., 0, 1 or 2);

or its pharmaceutically acceptable salt, optical isomer, tautomer, hydrate, N-oxide, reverse amide analog, prodrug, isotopic variant (e.g., deuterated analog), PROTAC, pharmaceutical product or any combination thereof.

In some embodiments, A of formula I, II, I(a), I(b), and/or I(c) is a phenyl. In other embodiments, A is pyridinyl. In other embodiments, A is 2-pyridinyl. In other embodiments, A is 3-pyridinyl. In other embodiments, A is 4-pyridinyl. In other embodiments, A is pyrimidine. In other embodiments, A is pyridazine. In other embodiments, A is pyrazine. In other embodiments, A is pyrazole. In other embodiments, A is naphthyl. In other embodiments, A is benzothiazolyl. In other embodiments, A is benzimidazolyl. In other embodiments, A is quinolinyl. In other embodiments, A is isoquinolinyl. In other embodiments, A is indolyl. In other embodiments, A is tetrahydronaphthyl. In other embodiments, A is indenyl. In other embodiments, A is benzofuran-2(3H)-one. In other embodiments, A is benzo[d][1,3]dioxole. In other embodiments, A is tetrahydrothiophenel, 1-dioxide. In other embodiments, A is thiazole. In other embodiments, A is benzimidazole. In others embodiment, A is piperidine. In other embodiments, A is 1-methylpiperidine. In other embodiments, A is imidazole. In other embodiments, A is 1-methylimidazole. In other embodiments, A is thiophene. In other embodiments, A is isoquinoline. In other embodiments, A is indole. In other embodiments, A is 1,3-dihydroisobenzofuran. In other embodiments, A is benzofuran. In other embodiments, A is tetrahydro-2H-pyran. In other embodiments, A is single or fused C₃-C₁₀ cycloalkyl ring. In other embodiments, A is cyclohexyl. In other embodiments, A is cyclopentyl. In other embodiments, A is, cyclopentenyl. In other embodiments, A is cyclopentadienyl. In other embodiments, A is isothiazolyl. In other embodiments, A is thiadiazolyl. In other embodiments, A is triazolyl. In other embodiments, A is thiazolyl. In other embodiments, A is oxazolyl. In other embodiments, A is isoxazolyl. In other embodiments, A is pyrrolyl. In other embodiments, A is furanyl. In other embodiments, A is oxadiazolyl. In other embodiments, A is oxadiazolyl. In other embodiments, A is 1,2,3-, 1,2,4-, 1,2,5- or 1,3,4-oxadiazolyl; each is a separate embodiment according to this invention. In other embodiments, A is tetrahydrofuranyl. In other embodiments, A is oxazolonyl. In other embodiments, A is oxazolidonyl. In other embodiments, A is thiazolonyl. In other embodiments, A is isothiazolinonyl. In other embodiments, A is isoxazolidinonyl. In other embodiments, A is imidazolidinonyl. In other embodiments, A is pyrazolonyl. In other embodiments, A is 2H-pyrrol-2-onyl. In other embodiments, A is furanonyl. In other embodiments, A is thiophenonyl. In other embodiments, A is thiane 1,1 dioxide. In other embodiments, A is triazolopyrimidine. In other embodiments, A is 3H-[1,2,3]triazolo[4,5-d]pyrimidine, 1H-[1,2,3]triazolo[4,5-d]pyrimidine, [1,2,4]triazolo[4,3-c]pyrimidine, [1,2,4]triazolo[4,3-a]pyrimidine, [1,2,3]triazolo[1,5-a]pyrimidine, [1,2,3]triazolo[1,5-c]pyrimidine, [1,2,4]triazolo[1,5-a]pyrimidine or [1,2,4]triazolo[1,5-c]pyrimidine; each is a separate embodiment according to this invention. In other embodiments, A is 6,7-dihydro-5H-pyrazolo[5,1-b][1,3]oxazine. In some embodiments, the A′ ring of formula I(f) is a 5 (five) membered heteroaromatic ring. In some embodiments, the A′ ring of formula I(f) is thiazolyl. In other embodiments, A′ is isothiazolyl. In other embodiments, A′ is oxazolyl. In other embodiments, A′ is isoxazolyl. In other embodiments, A′ is imidazolyl. In other embodiments, A′ is 1-methylimidazolyl. In other embodiments, A′ is pyrazolyl. In other embodiments, A′ is pyrrolyl. In other embodiments, A′ is furanyl. In other embodiments, A′ is thiophene-yl. In other embodiments, A′ is triazolyl. In other embodiments, A′ is thiadiazolyl. In other embodiments, A′ is oxadiazolyl. In other embodiments, A′ is 1,2,3-, 1,2,4-, 1,2,5- or 1,3,4- oxadiazolyl; each represents a separate embodiment according to this invention. In some embodiments, the A′ ring of formula I(f) is a 5 (five) membered heterocyclic ring. In other embodiments, A′ is pyrrolidine. In other embodiments, A′ is 2-oxo-pyrrolidine. In other embodiments, A′ is tetrahydrofuranyl. In other embodiments, A′ is oxazolonyl. In other embodiments, A′ is oxazolidonyl. In other embodiments, A′ is oxazolidonyl. In other embodiments, A′ is thiazolonyl. In other embodiments, A′ is isothiazolynonyl. In other embodiments, A′ is isoxazolidinonyl. In other embodiments, A′ is imidazolidinonyl. In other embodiments, A′ is pyrazolonyl. In other embodiments, A′ is 2H-pyrrol-2-onyl. In other embodiments, A′ is furanonyl. In other embodiments, A′ is thiophenonyl.

In some embodiments, B of formula I, II, I(a), I(b), I(c), I(d) and/or I(e) is a phenyl ring. In other embodiments, B is pyridinyl. In other embodiments, B is 2-pyridinyl. In other embodiments, B is 3-pyridinyl. In other embodiments, B is 4-pyridinyl. In other embodiments, B is pyrimidine. In other embodiments, B is pyridazine. In other embodiments, B is pyrazine. In other embodiments, B is thiazole. In other embodiments, B is imidazole. In other embodiments, B is indazole. In other embodiments, B is naphthyl. In other embodiments, B is indolyl. In other embodiments, B is benzimidazolyl. In other embodiments, B is benzothiazolyl. In other embodiments, B is quinoxalinyl. In other embodiments, B is tetrahydronaphthyl. In other embodiments, B is quinolinyl. In other embodiments, B is isoquinolinyl. In other embodiments, B is indenyl. In other embodiments, B is naphthalene. In other embodiments, B is tetrahydrothiophenel, 1-dioxide. In other embodiments, B is benzimidazole. In other embodiments, B is piperidine. In other embodiments, B is 1-methylpiperidine. In other embodiments, B is 1-methylimidazole. In other embodiments, B is thiophene. In other embodiments, B is isoquinoline. In other embodiments, B is indole. In other embodiments, B is 1,3-dihydroisobenzofuran. In other embodiments, B is benzofuran. In other embodiments, B is tetrahydro-2H-pyran. In other embodiments, B is single or fused C₃-C₁₀ cycloalkyl ring. In other embodiments, B is cyclohexyl. In other embodiments, B is cyclopentyl. In other embodiments, B is thiane 1,1-dioxide.

In some embodiments, X₁ of compound of formula I(a) is C. In other embodiments, X₁ is N.

In some embodiments, X₂ of compound of formula I(a) and/or I(b) is C. In other embodiments, X₂ is N.

In some embodiments, X₃ of compound of formula I(a) and/or I(b) is C. In other embodiments, X₃ is N.

In some embodiments, X₄ of compound of formula I(a) and/or I(b) is C. In other embodiments, X₄ is N.

In some embodiments, X₅ of compound of formula I(a) and/or I(b) is C. In other embodiments, X₅ is N.

In some embodiments, X₆ of compound of formula I, I(a) and/or I(b) is C. In other embodiments, X₆ is N.

In some embodiments, X₇ of compound of formula I and/or I(a) is C. In other embodiments, X₇ is N.

In some embodiments, X₈ of compound of formula I and/or I(a) is C. In other embodiments, X₈ is N.

In some embodiments, X₉ of compound of formula I(c) is C. In other embodiments, X₉ is N.

In some embodiments, X₁₀ of compound of formula I(c) is C. In other embodiments, X₁₀ is N.

In some embodiments, X₁₁ of compound of formula I(c) is C. In other embodiments, X₁₁ is N.

In some embodiments, X₁₂ of compound of formula I(c) is C. In other embodiments, X₁₂ is N.

In some embodiments, X₁₃ of compound of formula I(c) is C. In other embodiments, X₁₃ is N.

In some embodiments, X₁₄ of compound of formula I, II is C. In other embodiments, X₁₂ is N.

In some embodiments, X₁₅ of compound of formula I, II is C. In other embodiments, X₁₃ is N.

In some embodiments, at least one of X₁-X₅ is N. In some embodiments, at least two of X₁-X₅ are N. In some embodiments, at least one of X₉-X₁₃ is N. It is understood that if any of X₁-X₁₃ are N, then any of R₁—R₃ cannot be attached thereto.

In some embodiments, R₁ of formula I, II, I(a), I(b), I(c), I(d), I(d(i)), (Ie), I(e(i)) and I(f) is H. In some embodiments, R₁ is C₁-C₅ linear or branched, substituted or unsubstituted alkyl. In other embodiments, R₁ is methyl. In other embodiments, R₁ is ethyl. In other embodiments, R₁ is iso-propyl. In other embodiments, R₁ is t-Bu. In other embodiments, R₁ is iso-butyl. In other embodiments, R₁ is pentyl. In other embodiments, R₁ is propyl. In other embodiments, R₁ is benzyl. In other embodiments, R₁ is in the ortho position. In other embodiments, R₁ is an ortho-methyl.

In other embodiments, R₁ of formula I, II, I(a), I(b), I(c), I(d), I(d(i)), (Ie), I(e(i)) and I(f) is F. In other embodiments, R₁ is Cl. In other embodiments, R₁ is Br. In other embodiments, R₁ is I. In other embodiments, R₁ is R₈—(C₃-C₈ cycloalkyl). In other embodiments, R₁ is CH₂-cyclohexyl. In other embodiments, R₁ is R₈—(C₃-C₈ heterocyclic ring). In other embodiments, R₁ is CH₂-imidazole. In other embodiments, R₁ is CH₂-indazole. In other embodiments, R₁ is CF₃. In other embodiments, R₁ is C₁-C₅ linear or branched, or C₃-C₈ cyclic haloalkyl. In other embodiments, R₁ is CHF₂. In other embodiments, R₁ is CN. In other embodiments, R₁ is CF₂CH₂CH₃. In other embodiments, R₁ is CH₂CH₂CF₃. In other embodiments, R₁ is CF₂CH(CH₃)₂. In other embodiments, R₁ is CF(CH₃)—CH(CH₃)₂. In other embodiments, R₁ is OCD₃. In other embodiments, R₁ is NO₂. In other embodiments, R₁ is NH₂. In other embodiments, R₁ is R₈—N(R₁₀)(R₁₁). In other embodiments, R₁ is CH₂—NH₂. In other embodiments, R₁ is CH₂—N(CH₃)₂). In other embodiments, R₁ is R₉—R₈—N(R₁₀)(R₁₁). In other embodiments, R₁ is C≡C—CH₂—NH₂. In other embodiments, R₁ is B(OH)₂. In other embodiments, R₁ is NHC(O)—R₁₀. In other embodiments, R₁ is NHC(O)CH₃. In other embodiments, R₁ is NHCO—N(R₁₀)(R₁₁). In other embodiments, R₁ is NHC(O)N(CH₃)₂. In other embodiments, R₁ is COOH. In other embodiments, R₁ is C(O)O—R₁₀. In other embodiments, R₁ is C(O)O—CH(CH₃)₂. In other embodiments, R₁ is C(O)O—CH₃. In other embodiments, R₁ is SO₂N(R₁₀)(R₁₁). In other embodiments, R₁ is SO₂N(CH₃)₂. In other embodiments, R₁ is SO₂NHC(O)CH₃. In other embodiments, R₁ is C₁-C₅ linear or branched, substituted or unsubstituted alkyl. In other embodiments, R₁ is methyl. In other embodiments, R₁ is ethyl. In other embodiments, R₁ is iso-propyl. In other embodiments, R₁ is t-Bu. In other embodiments, R₁ is iso-butyl. In other embodiments, R₁ is pentyl. In other embodiments, R₁ is propyl. In other embodiments, R₁ is benzyl. In other embodiments, R₁ is C₁-C₅ linear or branched, substituted or unsubstituted alkenyl. In other embodiments, R₁ is CH═C(Ph)₂. In other embodiments, R₁ is 2-CH₂—C₆H₄—Cl. In other embodiments, R₁ is 3-CH₂—C₆H₄—Cl. In other embodiments, R₁ is 4-CH₂—C₆H₄—Cl. In other embodiments, R₁ is ethyl. In other embodiments, R₁ is iso-propyl. In other embodiments, R₁ is t-Bu. In other embodiments, R₁ is iso-butyl. In other embodiments, R₁ is pentyl. In other embodiments, R₁ is substituted or unsubstituted C₃-C₈ cycloalkyl (e.g., cyclopropyl, cyclopentyl). In other embodiments, R₁ is C₁-C₅ linear or branched, or C₃-C₈ cyclic alkoxy. In other embodiments, R₁ is methoxy. In other embodiments, R₁ is ethoxy. In other embodiments, R₁ is propoxy. In other embodiments, R₁ is isopropoxy. In other embodiments, R₁ is O—CH₂-cyclopropyl. In other embodiments, R₁ is O-cyclobutyl. In other embodiments, R₁ is O-cyclopentyl. In other embodiments, R₁ is O-cyclohexyl. In other embodiments, R₁ is O-1-oxacyclobutyl. In other embodiments, R₁ is O-2-oxacyclobutyl. In other embodiments, R₁ is 1-butoxy. In other embodiments, R₁ is 2-butoxy. In other embodiments, R₁ is O-tBu. In other embodiments, R₁ is C₁-C₅ linear or branched, or C₃-C₈ cyclic alkoxy wherein at least one methylene group (CH₂) in the alkoxy is replaced with an oxygen atom (O). In other embodiments, R₁ is O-1-oxacyclobutyl. In other embodiments, R₁ is O-2-oxacyclobutyl. In other embodiments, R₁ is C₁-C₅ linear or branched haloalkoxy. In other embodiments, R₁ is OCF₃. In other embodiments, R₁ is OCHF₂. In other embodiments, R₁ is substituted or unsubstituted C₃-C₈ heterocyclic ring. In other embodiments, R₁ is oxazole. In other embodiments, R₁ is methyl substituted oxazole. In other embodiments, R₁ is oxadiazole. In other embodiments, R₁ is methyl substituted oxadiazole. In other embodiments, R₁ is imidazole. In other embodiments, R₁ is methyl substituted imidazole. In other embodiments, R₁ is pyridine. In other embodiments, R₁ is 2-pyridine. In other embodiments, R₁ is 3-pyridine. In other embodiments, R₁ is 4-pyridine. In other embodiments, R₁ is tetrazole. In other embodiments, R₁ is pyrimidine. In other embodiments, R₁ is pyrazine. In other embodiments, R₁ is oxacyclobutane. In other embodiments, R₁ is 1-oxacyclobutane. In other embodiments, R₁ is 2-oxacyclobutane. In other embodiments, R₁ is indole. In other embodiments, R₁ is pyridine oxide. In other embodiments, R₁ is protonated pyridine oxide. In other embodiments, R₁ is deprotonated pyridine oxide. In other embodiments, R₁ is 3-methyl-4H-1,2,4-triazole. In other embodiments, R₁ is 5-methyl-1,2,4-oxadiazole. In other embodiments, R₁ is substituted or unsubstituted aryl. In other embodiments, R₁ is phenyl. In other embodiments, R₁ is bromophenyl. In other embodiments, R₁ is 2-bromophenyl. In other embodiments, R₁ is 3-bromophenyl. In other embodiments, R₁ is 4-bromophenyl. In other embodiments, R₁ is substituted or unsubstituted benzyl. In other embodiments, R₁ is benzyl. In other embodiments, R₁ is R₈—N(R₁₀)(R₁₁). In other embodiments, R₁ is CH₂—NH₂. In other embodiments, substitutions include: C₁-C₅ linear or branched alkyl (e.g. methyl), aryl, phenyl, heteroaryl (e.g., imidazole), and/or C₃-C₈ cycloalkyl, each is a separate embodiment according to this invention.

In some embodiments, R₂ of formula I, I(a), I(b), I(c), I(d), I(d(i)), I(e), I(e(i)) and/or I(f) is H. In some embodiments, R₂ is C₁-C₅ linear or branched, substituted or unsubstituted alkyl. In other embodiments, R₂ is methyl. In other embodiments, R₂ is ethyl. In other embodiments, R₂ is iso-propyl. In other embodiments, R₂ is t-Bu. In other embodiments, R₂ is iso-butyl. In other embodiments, R₂ is pentyl. In other embodiments, R₂ is propyl. In other embodiments, R₂ is benzyl. In other embodiments, R₂ is in the ortho position. In other embodiments, R₂ is an ortho-methyl.

In some embodiments, R₂ of formula I, II, I(a), I(b), I(c), I(d), I(d(i)), I(e), I(e(i)) and/or I(f) is F. In other embodiments, R₂ is Cl. In other embodiments, R₂ is Br. In other embodiments, R₂ is I. In other embodiments, R₂ is R₈—(C₃-C₈ cycloalkyl). In other embodiments, R₂ is CH₂-cyclohexyl. In other embodiments, R₂ is R₈—(C₃-C₈ heterocyclic ring). In other embodiments, R₂ is CH₂-imidazole. In other embodiments, R₂ is CF₃. In other embodiments, R₂ is C₁-C₅ linear or branched, or C₃-C₈ cyclic haloalkyl. In other embodiments, R₂ is CHF₂. In other embodiments, R₂ is CN. In other embodiments, R₂ is CF₂CH₂CH₃. In other embodiments, R₂ is CH₂CH₂CF₃. In other embodiments, R₂ is CF₂CH(CH₃)₂. In other embodiments, R₂ is CF(CH₃)—CH(CH₃)₂. In other embodiments, R₂ is OCD₃. In other embodiments, R₂ is NO₂. In other embodiments, R₂ is NH₂. In other embodiments, R₂ is R₈—N(R₁₀)(R₁₁). In other embodiments, R₂ is CH₂—NH₂. In other embodiments, R₂ is CH₂—N(CH₃)₂). In other embodiments, R₂ is R₉—R₈—N(R₁₀)(R₁₁). In other embodiments, R₂ is C≡C—CH₂—NH₂. In other embodiments, R₂ is B(OH)₂. In other embodiments, R₂ is NHC(O)—R₁₀. In other embodiments, R₂ is NHC(O)CH₃. In other embodiments, R₂ is NHCO—N(R₁₀)(R₁₁). In other embodiments, R₂ is NHC(O)N(CH₃)₂. In other embodiments, R₂ is COOH. In other embodiments, R₂ is C(O)O—R₁₀. In other embodiments, R₂ is C(O)O—CH(CH₃)₂. In other embodiments, R₂ is C(O)O—CH₃. In other embodiments, R₂ is SO₂N(R₁₀)(R₁₁). In other embodiments, R₂ is SO₂N(CH₃)₂. In other embodiments, R₂ is SO₂NHC(O)CH₃. In other embodiments, R₂ is C₁-C₅ linear or branched, substituted or unsubstituted alkyl. In other embodiments, R₂ is methyl. In other embodiments, R₂ is ethyl. In other embodiments, R₂ is iso-propyl. In other embodiments, R₂ is t-Bu. In other embodiments, R₂ is iso-butyl. In other embodiments, R₂ is pentyl. In other embodiments, R₂ is propyl. In other embodiments, R₂ is benzyl. In other embodiments, R₂ is C₁-C₅ linear or branched, substituted or unsubstituted alkenyl. In other embodiments, R₂ is CH═C(Ph)₂. In other embodiments, R₂ is 2-CH₂—C₆H₄—Cl. In other embodiments, R₂ is 3-CH₂—C₆H₄—Cl. In other embodiments, R₂ is 4-CH₂—C₆H₄—Cl. In other embodiments, R₂ is ethyl. In other embodiments, R₂ is iso-propyl. In other embodiments, R₂ is t-Bu. In other embodiments, R₂ is iso-butyl. In other embodiments, R₂ is pentyl. In other embodiments, R₂ is substituted or unsubstituted C₃-C₈ cycloalkyl (e.g., cyclopropyl, cyclopentyl). In other embodiments, R₂ is C₁-C₅ linear or branched, or C₃-C₈ cyclic alkoxy. In other embodiments, R₂ is methoxy. In other embodiments, R₂ is ethoxy. In other embodiments, R₂ is propoxy. In other embodiments, R₂ is isopropoxy. In other embodiments, R₂ is O—CH₂-cyclopropyl. In other embodiments, R₂ is O-cyclobutyl. In other embodiments, R₂ is O-cyclopentyl. In other embodiments, R₂ is O-cyclohexyl. In other embodiments, R₂ is O-1-oxacyclobutyl. In other embodiments, R₂ is O-2-oxacyclobutyl. In other embodiments, R₂ is 1-butoxy. In other embodiments, R₂ is 2-butoxy. In other embodiments, R₂ is O-tBu. In other embodiments, R₂ is C₁-C₅ linear or branched haloalkoxy. In other embodiments, R₂ is OCF₃. In other embodiments, R₂ is OCHF₂. In other embodiments, R₂ is substituted or unsubstituted C₃-C₈ heterocyclic ring. In other embodiments, R₂ is oxazole or methyl substituted oxazole. In other embodiments, R₂ is oxadiazole or methyl substituted oxadiazole. In other embodiments, R₂ is imidazole or methyl substituted imidazole. In other embodiments, R₂ is pyridine. In other embodiments, R₂ is 2-pyridine. In other embodiments, R₂ is 3-pyridine. In other embodiments, R₂ is 4-pyridine. In other embodiments, R₂ is tetrazole. In other embodiments, R₂ is pyrimidine. In other embodiments, R₂ is pyrazine. In other embodiments, R₂ is oxacyclobutane. In other embodiments, R₂ is 1-oxacyclobutane. In other embodiments, R₂ is 2-oxacyclobutane. In other embodiments, R₂ is indole. In other embodiments, R₂ is pyridine oxide. In other embodiments, R₂ is protonated pyridine oxide. In other embodiments, R₂ is deprotonated pyridine oxide. In other embodiments, R₂ is 3-methyl-4H-1,2,4-triazole. In other embodiments, R₂ is 5-methyl-1,2,4-oxadiazole. In other embodiments, R₂ is substituted or unsubstituted aryl. In other embodiments, R₂ is phenyl. In other embodiments, R₂ is bromophenyl. In other embodiments, R₂ is 2-bromophenyl. In other embodiments, R₂ is 3-bromophenyl. In other embodiments, R₂ is 4-bromophenyl. In other embodiments, R₂ is substituted or unsubstituted benzyl. In other embodiments, R₂ is benzyl. In other embodiments, R₂ is R₈—N(R₁₀)(R₁₁). In other embodiments, R₂ is CH₂—NH₂. In other embodiments, substitutions include: C₁-C₅ linear or branched alkyl (e.g. methyl), aryl, phenyl, heteroaryl (e.g., imidazole), and/or C₃-C₈ cycloalkyl, each is a separate embodiment according to this invention.

In some embodiments, R₁ and R₂ of formula I, II, I(a), I(d), I(d(i)), and/or I(f) are joint together to form a pyrrol ring. In some embodiments, R₁ and R₂ are joint together to form a [1,3]dioxole ring. In some embodiments, R₁ and R₂ are joint together to form a furanone ring (e.g., furan-2(3H)-one). In some embodiments, R₁ and R₂ are joint together to form a benzene ring. In some embodiments, R₁ and R₂ are joint together to form a pyridine ring. In some embodiments, R₁ and R₂ are joint together to form an oxazine ring. In some embodiments, R₁ and R₂ are joint together to form a pyrimidine ring.

In some embodiments, R₃ of formula I, II, I(a), I(b), I(c), I(d), I(d(i)), I(e), I(e(i)) and/or I(f) is H. In other embodiments, R₃ is Cl. In other embodiments, R₃ is I. In other embodiments, R₃ is F. In other embodiments, R₃ is Br. In other embodiments, R₃ is OH. In other embodiments, R₃ is CD₃. In other embodiments, R₃ is OCD₃. In other embodiments, R₃ is R₈—OH. In other embodiments, R₃ is CH₂—OH. In other embodiments, R₃ is —R₈—O—R₁₀. In other embodiments, R₃ is CH₂—O—CH₃. In other embodiments, R₃ is —O—R₈—R₁₀. In other embodiments, R₃ is CH₂—O—CH₃. In other embodiments, R₃ is R₈—N(R₁₀)(R₁₁). In other embodiments, R₃ is CH₂—NH—CH₃. In other embodiments, R₃ is CH₂—NH₂. In other embodiments, R₃ is CH₂—N(CH₃)₂. In other embodiments, R₃ is COOH. In other embodiments, R₃ is C(O)O—R₁₀. In other embodiments, R₃ is C(O)O—CH₂CH₃. In other embodiments, R₃ is R₈—C(O)—R₁₀. In other embodiments, R₃ is CH₂C(O)CH₃. In other embodiments, R₃ is C(O)—R₁₀. In other embodiments, R₃ is C(O)—CH₃. In other embodiments, R₃ is C(O)—CH₂CH₃. In other embodiments, R₃ is C(O)—CH₂CH₂CH₃. In other embodiments, R₃ is C₁-C₅ linear or branched C(O)-haloalkyl. In other embodiments, R₃ is C(O)—CF₃. In other embodiments, R₃ is C(O)N(R₁₀)(R₁₁). In other embodiments, R₃ is C(O)N(CH₃)₂). In other embodiments, R₃ is SO₂N(R₁₀)(R₁₁). In other embodiments, R₃ is SO₂N(CH₃)₂. In other embodiments, R₃ is C₁-C₅ linear or branched, substituted or unsubstituted alkyl. In other embodiments, R₃ is methyl. In other embodiments, R₃ is ethyl. In other embodiments, R₃ is CH₂—OCH₂—CH₂—O—CH₃. In other embodiments, R₃ is propyl. In other embodiments, R₃ is iso-propyl. In other embodiments, R₃ is t-Bu. In other embodiments, R₃ is iso-butyl. In other embodiments, R₃ is pentyl. In other embodiments, R₃ is C(OH)(CH₃)(Ph). In other embodiments, R₃ is C₁-C₅ linear or branched, or C₃-C₈ cyclic haloalkyl. In other embodiments, R₃ is CF₂CH₃. In other embodiments, R₃ is CF₂-cyclobutyl. In other embodiments, R₃ is CH₂CF₃. In other embodiments, R₃ is CF₂CH₂CH₃. In other embodiments, R₃ is CF₃. In other embodiments, R₃ is CF₂CH₂CH₃. In other embodiments, R₃ is CH₂CH₂CF₃. In other embodiments, R₃ is CF₂CH(CH₃)₂. In other embodiments, R₃ is CF(CH₃)—CH(CH₃)₂. In other embodiments, R₃ is substituted or unsubstituted C₁-C₅ linear or branched, or C₃-C₈ cyclic alkoxy. In other embodiments, R₃ is substituted C₁-C₅ linear or branched, or C₃-C₈ cyclic alkoxy. In other embodiments, R₃ is O—(CH₂)₂O—CH₃. In other embodiments, R₃ is unsubstituted C₁-C₅ linear or branched, or C₃-C₈ cyclic alkoxy. In other embodiments, R₃ is methoxy. In other embodiments, R₃ is isopropoxy. In other embodiments, R₃ is substituted or unsubstituted C₃-C₈ cycloalkyl. In other embodiments, R₃ is cyclopropyl. In other embodiments, R₃ is cyclopentyl. In other embodiments, R₃ is substituted or unsubstituted C₃-C₈ heterocyclic ring. In other embodiments, R₃ is thiophene. In other embodiments, R₃ is oxazole. In other embodiments, R₃ is isoxazole. In other embodiments, R₃ is imidazole. In other embodiments, R₃ is furane. In other embodiments, R₃ is triazole. In other embodiments, R₃ is pyridine. In other embodiments, R₃ is 2-pyridine. In other embodiments, R₃ is 3-pyridine. In other embodiments, R₃ is 4-pyridine. In other embodiments, R₃ is pyrimidine. In other embodiments, R₃ is pyrazine. In other embodiments, R₃ is oxacyclobutane. In other embodiments, R₃ is 1-oxacyclobutane. In other embodiments, R₃ is 2-oxacyclobutane. In other embodiments, R₃ is indole. In other embodiments, R₃ is 3-methyl-4H-1,2,4-triazole. In other embodiments, R₃ is 5-methyl-1,2,4-oxadiazole. In other embodiments, R₃ is substituted or unsubstituted aryl. In other embodiments, R₃ is phenyl. In other embodiments, R₃ is CH(CF₃)(NH—R₁₀).

In some embodiments, R₄ of formula I, II, I(a), I(b), and/or I(c) is H. In other embodiments, R₄ is Cl. In other embodiments, R₄ is I. In other embodiments, R₄ is F. In other embodiments, R₄ is Br. In other embodiments, R₄ is OH. In other embodiments, R₄ is CD₃. In other embodiments, R₄ is OCD₃. In other embodiments, R₄ is R₈—OH. In other embodiments, R₄ is CH₂—OH. In other embodiments, R₄ is —R₈—O—R₁₀. In other embodiments, R₄ is CH₂—O—CH₃. In other embodiments, R₄ is —O—R₈—R₁₀. In other embodiments, R₄ is CH₂—O—CH₃. In other embodiments, R₄ is R₈—N(R₁₀)(R₁₁). In other embodiments, R₄ is CH₂—NH—CH₃. In other embodiments, R₄ is CH₂—NH₂. In other embodiments, R₄ is CH₂—N(CH₃)₂. In other embodiments, R₄ is COOH. In other embodiments, R₄ is C(O)O—R₁₀. In other embodiments, R₄ is C(O)O—CH₂CH₃. In other embodiments, R₄ is R₈—C(O)—R₁₀. In other embodiments, R₄ is CH₂C(O)CH₃. In other embodiments, R₄ is C(O)—R₁₀. In other embodiments, R₄ is C(O)—CH₃. In other embodiments, R₄ is C(O)—CH₂CH₃. In other embodiments, R₄ is C(O)—CH₂CH₂CH₃. In other embodiments, R₄ is C₁-C₅ linear or branched C(O)-haloalkyl. In other embodiments, R₄ is C(O)—CF₃. In other embodiments, R₄ is C(O)N(R₁₀)(R₁₁). In other embodiments, R₄ is C(O)N(CH₃)₂). In other embodiments, R₄ is SO₂N(R₁₀)(R₁₁). In other embodiments, R₄ is SO₂N(CH₃)₂. In other embodiments, R₄ is C₁-C₅ linear or branched, substituted or unsubstituted alkyl. In other embodiments, R₄ is methyl. In other embodiments, R₄ is C(OH)(CH₃)(Ph). In other embodiments, R₄ is ethyl. In other embodiments, R₄ is CH₂—OCH₂—CH₂—O—CH₃. In other embodiments, R₄ is propyl. In other embodiments, R₄ is iso-propyl. In other embodiments, R₄ is t-Bu. In other embodiments, R₄ is iso-butyl. In other embodiments, R₄ is pentyl. In other embodiments, R₄ is C₁-C₅ linear or branched, or C₃-C₈ cyclic haloalkyl. In other embodiments, R₃ is CF₂CH₃. In other embodiments, R₃ is CF₂-cyclobutyl. In other embodiments, R₄ is CH₂CF₃. In other embodiments, R₄ is CF₂CH₂CH₃. In other embodiments, R₄ is CF₃. In other embodiments, R₄ is CF₂CH₂CH₃. In other embodiments, R₄ is CH₂CH₂CF₃. In other embodiments, R₄ is CF₂CH(CH₃)₂. In other embodiments, R₄ is CF(CH₃)—CH(CH₃)₂. In other embodiments, R₄ is C₁-C₅ linear or branched, or C₃-C₈ cyclic alkoxy. In other embodiments, R₄ is substituted C₁-C₅ linear or branched, or C₃-C₈ cyclic alkoxy. In other embodiments, R₄ is O—(CH₂)₂O—CH₃. In other embodiments, R₄ is unsubstituted C₁-C₅ linear or branched, or C₃-C₈ cyclic alkoxy. In other embodiments, R₄ is methoxy. In other embodiments, R₄ is isopropoxy. In other embodiments, R₄ is substituted or unsubstituted C₃-C₈ cycloalkyl. In other embodiments, R₄ is cyclopropyl. In other embodiments, R₄ is cyclopentyl. In other embodiments, R₄ is substituted or unsubstituted C₃-C₈ heterocyclic ring. In other embodiments, R₄ is thiophene. In other embodiments, R₄ is oxazole. In other embodiments, R₄ is isoxazole. In other embodiments, R₄ is imidazole. In other embodiments, R₄ is furane. In other embodiments, R₄ is triazole. In other embodiments, R₄ is pyridine. In other embodiments, R₄ is 2-pyridine. In other embodiments, R₄ is 3-pyridine. In other embodiments, R₄ is 4-pyridine. In other embodiments, R₄ is pyrimidine. In other embodiments, R₄ is pyrazine. In other embodiments, R₄ is oxacyclobutane. In other embodiments, R₄ is 1-oxacyclobutane. In other embodiments, R₄ is 2-oxacyclobutane. In other embodiments, R₄ is indole. In other embodiments, R₄ is 3-methyl-4H-1,2,4-triazole. In other embodiments, R₄ is 5-methyl-1,2,4-oxadiazole. In other embodiments, R₄ is substituted or unsubstituted aryl. In other embodiments, R₄ is phenyl. In other embodiments, R₄ is CH(CF₃)(NH—R₁₀).

In some embodiments, R₃ and R₄ of formula I, II, I(a), I(b), and/or I(c) are joint together to form a [1,3]dioxole ring. In some embodiments, R₃ and R₄ are joint together to form a furanone ring (e.g., furan-2(3H)-one). In some embodiments, R₃ and R₄ are joint together to form a benzene ring. In some embodiments, R₃ and R₄ are joint together to form a cyclopentene ring. In some embodiments, R₃ and R₄ are joint together to form an imidazole ring.

In some embodiments, R₅ of formula I, II, I(a), and/or I(b) is absent. In some embodiments, R₅ is H. In some embodiments, R₅ is F. In some embodiments, R₅ is Cl. In some embodiments, R₅ is Br. In some embodiments, R₅ is I. In some embodiments, R₅ is OH. In some embodiments, R₅ is SH. In some embodiments, R₅ is R₈—OH. In some embodiments, R₅ is R₈—SH. In some embodiments, R₅ is —R₈—O—R₁₀. In some embodiments, R₅ is R₈—(C₃-C₈ cycloalkyl). In some embodiments, R₅ is R₈—(C₃-C₈ heterocyclic ring). In some embodiments, R₅ is CF₃. In some embodiments, R₅ is CD₃. In some embodiments, R₅ is OCD₃. In some embodiments, R₅ is CN. In some embodiments, R₅ is NO₂. In some embodiments, R₅ is —CH₂CN. In some embodiments, R₅ is —R₈CN. In some embodiments, R₅ is NH₂. In some embodiments, R₅ is NHR. In some embodiments, R₅ is N(R)₂. In some embodiments, R₅ is R₈—N(R₁₀)(R₁₁). In some embodiments, R₅ is R₉—R₈—N(R₁₀)(R₁₁). In some embodiments, R₅ is B(OH)₂. In some embodiments, R₅ is —OC(O)CF₃. In some embodiments, R₅ is —OCH₂Ph. In some embodiments, R₅ is NHC(O)—R₁₀. In some embodiments, R₅ is NHCO—N(R₁₀)(R₁₁). In some embodiments, R₅ is COOH. In some embodiments, R₅ is —C(O)Ph. In some embodiments, R₅ is C(O)O—R₁₀. In some embodiments, R₅ is R₈—C(O)—R₁₀. In some embodiments, R₅ is C(O)H. In some embodiments, R₅ is C(O)—R₁₀. In some embodiments, R₅ is C₁-C₅ linear or branched C(O)-haloalkyl. In some embodiments, R₅ is —C(O)NH₂. In some embodiments, R₅ is C(O)NHR. In some embodiments, R₅ is C(O)N(R₁₀)(R₁₁). In some embodiments, R₅ is SO₂R, SO₂N(R₁₀)(R₁₁). In some embodiments, R₅ is CH(CF₃)(NH—R₁₀). In some embodiments, R₅ is, C₁-C₅ linear or branched, substituted or unsubstituted alkyl. In some embodiments, R₅ is methyl. In some embodiments, R₅ is ethyl. In some embodiments, R₅ is C₁-C₅ linear or branched, substituted or unsubstituted alkenyl. In some embodiments, R₅ is C₁-C₅ linear or branched, or C₃-C₈ cyclic haloalkyl. In some embodiments, R₅ is CHF₂. In some embodiments, R₅ is C₁-C₅ linear or branched, or C₃-C₈ cyclic alkoxy. In some embodiments, R₅ is methoxy. In some embodiments, R₅ is C₁-C₅ linear or branched thioalkoxy. In some embodiments, R₅ is C₁-C₅ linear or branched haloalkoxy. In some embodiments, R₅ is C₁-C₅ linear or branched alkoxyalkyl. In some embodiments, R₅ is substituted or unsubstituted C₃-C₈ cycloalkyl. In some embodiments, R₅ is cyclopropyl. In some embodiments, R₅ is substituted or unsubstituted C₃-C₈ heterocyclic ring. In some embodiments, R₅ is substituted or unsubstituted aryl. In some embodiments, R₅ is substituted or unsubstituted benzyl.

In some embodiments, R₆ of formula I or II is H. In some embodiments, R₆ is F. In some embodiments, R₆ is Cl. In some embodiments, R₆ is Br. In some embodiments, R₆ is I. In some embodiments, R₆ is OH. In some embodiments, R₆ is SH. In some embodiments, R₆ is R₈—OH. In some embodiments, R₆ is R₈—SH. In some embodiments, R₆ is —R₈—O—R₁₀. In some embodiments, R₆ is R₈—(C₃-C₈ cycloalkyl). In some embodiments, R₆ is R₈—(C₃-C₈ heterocyclic ring). In some embodiments, R₆ is CF₃. In some embodiments, R₆ is CD₃. In some embodiments, R₆ is OCD₃. In some embodiments, R₆ is CN. In some embodiments, R₆ is NO₂. In some embodiments, R₆ is —CH₂CN. In some embodiments, R₆ is —R₈CN. In some embodiments, R₆ is NH₂. In some embodiments, R₆ is NHR. In some embodiments, R₆ is N(R)₂. In some embodiments, R₆ is R₈—N(R₁₀)(R₁₁). In some embodiments, R₆ is R₉—R₈—N(R₁₀)(R₁₁). In some embodiments, R₆ is B(OH)₂. In some embodiments, R₆ is —OC(O)CF₃. In some embodiments, R₆ is —OCH₂Ph. In some embodiments, R₆ is NHC(O)—R₁₀. In some embodiments, R₆ is NHCO—N(R₁₀)(R₁₁). In some embodiments, R₆ is COOH. In some embodiments, R₆ is —C(O)Ph. In some embodiments, R₆ is C(O)O—R₁₀. In some embodiments, R₆ is R₈—C(O)—R₁₀. In some embodiments, R₆ is C(O)H. In some embodiments, R₆ is C(O)—R₁₀. In some embodiments, R₆ is C₁-C₅ linear or branched C(O)-haloalkyl. In some embodiments, R₆ is —C(O)NH₂. In some embodiments, R₆ is C(O)NHR. In some embodiments, R₆ is C(O)N(R₁₀)(R₁). In some embodiments, R₆ is SO₂R. In some embodiments, R₆ is SO₂N(R₁₀)(R₁₁). In some embodiments, R₆ is CH(CF₃)(NH—R₁₀). In some embodiments, R₆ is C₁-C₅ linear or branched, substituted or unsubstituted alkyl. In some embodiments, R₆ is methyl. In some embodiments, R₆ is ethyl. In some embodiments, R₆ is C₁-C₅ linear or branched, substituted or unsubstituted alkenyl. In some embodiments, R₆ is C₁-C₅ linear or branched, or C₃-C₈ cyclic haloalkyl. In some embodiments, R₆ is CHF₂. In some embodiments, R₆ is C₁-C₅ linear or branched, or C₃-C₈ cyclic alkoxy. In some embodiments, R₆ is methoxy. In some embodiments, R₆ is C₁-C₅ linear or branched thioalkoxy. In some embodiments, R₆ is C₁-C₅ linear or branched haloalkoxy. In some embodiments, R₆ is C₁-C₅ linear or branched alkoxyalkyl. In some embodiments, R₆ is substituted or unsubstituted C₃-C₈ cycloalkyl. In some embodiments, R₆ is cyclopropyl. In some embodiments, R₆ is substituted or unsubstituted C₃-C₈ heterocyclic ring. In some embodiments, R₆ is substituted or unsubstituted aryl. In some embodiments, R₆ is substituted or unsubstituted benzyl.

In some embodiments, R₇ of formula I or II is H. In some embodiments, R₇ is F. In some embodiments, R₇ is Cl. In some embodiments, R₇ is Br. In some embodiments, R₇ is I. In some embodiments, R₇ is OH. In some embodiments, R₇ is SH. In some embodiments, R₇ is R₈—OH. In some embodiments, R₇ is R₈—SH. In some embodiments, R₇ is —R₈—O—R₁₀. In some embodiments, R₇ is R₈—(C₃-C₈ cycloalkyl). In some embodiments, R₇ is R₈—(C₃-C₈ heterocyclic ring). In some embodiments, R₇ is CF₃. In some embodiments, R₇ is CD₃. In some embodiments, R₇ is OCD₃. In some embodiments, R₇ is CN. In some embodiments, R₇ is NO₂. In some embodiments, R₇ is —CH₂CN. In some embodiments, R₇ is —R₈CN. In some embodiments, R₇ is NH₂. In some embodiments, R₇ is NHR. In some embodiments, R₇ is N(R)₂. In some embodiments, R₇ is R₈—N(R₁₀)(R₁₁). In some embodiments, R₇ is R₉—R₈—N(R₁₀)(R₁₁). In some embodiments, R₇ is B(OH)₂. In some embodiments, R₇ is —OC(O)CF₃. In some embodiments, R₆ is —OCH₂Ph. In some embodiments, R₇ is NHC(O)—R₁₀. In some embodiments, R₇ is NHCO—N(R₁₀)(R₁₁). In some embodiments, R₆ is COOH. In some embodiments, R₆ is —C(O)Ph. In some embodiments, R₇ is C(O)O—R₁₀. In some embodiments, R₆ is R₈—C(O)—R₁₀. In some embodiments, R₆ is C(O)H. In some embodiments, R₇ is C(O)—R₁₀. In some embodiments, R₇ is C₁-C₅ linear or branched C(O)-haloalkyl. In some embodiments, R₇ is —C(O)NH₂. In some embodiments, R₇ is C(O)NHR. In some embodiments, R₇ is C(O)N(R₁₀)(R₁). In some embodiments, R₇ is SO₂R. In some embodiments, R₇ is SO₂N(R₁₀)(R₁₁). In some embodiments, R₇ is CH(CF₃)(NH—R₁₀). In some embodiments, R₇ is C₁-C₅ linear or branched, substituted or unsubstituted alkyl. In some embodiments, R₇ is methyl. In some embodiments, R₇ is ethyl. In some embodiments, R₇ is C₁-C₅ linear or branched, substituted or unsubstituted alkenyl. In some embodiments, R₇ is C₁-C₅ linear or branched, or C₃-C₈ cyclic haloalkyl. In some embodiments, R₇ is CHF₂. In some embodiments, R₇ is C₁-C₅ linear or branched, or C₃-C₈ cyclic alkoxy. In some embodiments, R₇ is methoxy. In some embodiments, R₇ is C₁-C₅ linear or branched thioalkoxy. In some embodiments, R₇ is C₁-C₅ linear or branched haloalkoxy. In some embodiments, R₇ is C₁-C₅ linear or branched alkoxyalkyl. In some embodiments, R₇ is substituted or unsubstituted C₃-C₈ cycloalkyl. In some embodiments, R₇ is cyclopropyl. In some embodiments, R₇ is substituted or unsubstituted C₃-C₈ heterocyclic ring. In some embodiments, R₇ is substituted or unsubstituted aryl. In some embodiments, R₇ is substituted or unsubstituted benzyl.

In some embodiments, R₆ and R₇ of formula I, II, I(a), I(b), and I(c) are joint together to form a 3 to 6 membered substituted or unsubstituted, aliphatic or aromatic, carbocyclic or heterocyclic ring. In some embodiments, R₆ and R₇ are joint together to form a 3 to 6 membered aliphatic carbocyclic ring. In some embodiments, R₆ and R₇ are joint together to form a cyclopropyl.

In some embodiments, R₈ of formula I, II, I(a), I(b), I(c), I(d), I(d(i)), I(e), I(e(i)) and/or I(f) is CH₂. In other embodiments, R₈ is CH₂CH₂. In other embodiments, R₈ is CH₂CH₂CH₂.

In some embodiments, p of formula I, II, I(a), I(b), I(c), I(d), I(d(i)), I(e), I(e(i)) and/or I(f) is 1. In other embodiments, p is 2. In other embodiments, p is 3.

In some embodiments, R₉ of formula I, II, I(a), I(b), I(c), I(d), I(d(i)), I(e), I(e(i)) and/or I(f) is C≡C.

In some embodiments, q of formula I, II, I(a), I(b), I(c), I(d), I(d(i)), I(e), I(e(i)) and/or I(f) is 2.

In some embodiments, R₁₀ of formula I, II, I(a), I(b), I(c), I(d), I(d(i)), I(e), I(e(i)) and/or I(f) is substituted or unsubstituted C₁-C₅ linear or branched alkyl. In other embodiments, R₁₀ is H. In other embodiments, R₁₀ is CH₃. In other embodiments, R₁₀ is CH₂CH₃. In other embodiments, R₁₀ is CH₂CH₂CH₃. In other embodiments, R₁₀ is CH₂—CH₂—O—CH₃. In other embodiments, R₁₀ is C₁-C₅ linear or branched alkoxy. In other embodiments, R₁₀ is O—CH₃.

In some embodiments, R₁₁ of formula I, II, I(a), I(b), I(c), I(d), I(d(i)), I(e), I(e(i)) and/or I(f) is substituted or unsubstituted C₁-C₅ linear or branched alkyl. In other embodiments, R₁₀ is H. In other embodiments, R₁₁ is CH₃. In other embodiments, R₁₁ is CH₂—CH₂—O—CH₃. In other embodiments, R₁₁ is C₁-C₅ linear or branched alkoxy. In other embodiments, R₁₁ is O—CH₃.

In some embodiments, R₁₀ and R₁₁ of formula I, II, I(a), I(b), I(c), I(d), I(d(i)), (Ie), I(e(i)) and/or I(f) are joint to form a substituted or unsubstituted C₃-C₈ heterocyclic ring. In other embodiments, R₁₀ and R₁₁ are joint to form a piperazine ring. In other embodiments, R₁₀ and R₁₁ are joint to form a piperidine ring. In some embodiments, substitutions include: F, Cl, Br, I, OH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof; each represents a separate embodiment according to this invention.

In some embodiments, R₁₂ of formula I(d), I(d(i)), I(e) and/or I(e(i)) is H. In other embodiments, R₁₂ is C₁-C₅ linear or branched alkyl. In other embodiments, R₁₂ is methyl. In other embodiments, R₁₂ is ethyl. In other embodiments, R₁₂ is C₁-C₅ linear or branched haloalkyl. In other embodiments, R₁₂ is CHF₂. In other embodiments, R₁₂ is CF₃. In other embodiments, R₁₂ is Cl. In other embodiments, R₁₂ is CN. In other embodiments, R₁₂ is substituted or unsubstituted C₃-C₈ cycloalkyl. In other embodiments, R₁₂ is cyclopropyl.

In some embodiments, R of formula I, II, I(a), I(b), I(c), I(d), I(d(i)), I(e), I(e(i)) and/or I(f) is H. In other embodiments, R is C₁-C₅ linear or branched alkyl. In other embodiments, R is methyl. In other embodiments, R is ethyl. In other embodiments, R is CH₂—CH₂—O—CH₂—CH₂—O—CH₃. In other embodiments, R is CH₂—O—CH₂—CH₂—O—CH₃. In other embodiments, R is C₁-C₅ linear or branched haloalkyl. In other embodiments, R is CHF₂. In other embodiments, R is CF₃. In other embodiments, R is —R₈—O—R₈—O—R₁₀. In other embodiments, R is (CH₂)₂—O—(CH₂)₂O—CH₃. In other embodiments, R is —R₈—O—R₁₀. In other embodiments, R is —R₈—R₁₀. In other embodiments, R is (CH₂)₂O—CH₃. In other embodiments, R is Cl. In other embodiments, R is CN.

In some embodiments, m of formula I, II, I(a) and I(f) is 0. In some embodiments, m is 1.

In some embodiments, n of formula I, II, I(a) and I(f), is 0. In other embodiments, n is 1.

In some embodiments, k of formula I, II and I(a), is 0. In other embodiments, k is 1.

In some embodiments, 1 of formula I, II I(a), I(b), I(c), I(d), I(d(i)), I(e), I(e(i)) and I(f) is 0. In other embodiments, l is 1. In other embodiments, l is 2.

In some embodiments, Q₁ of formula I, II, I(a) and I(b) is NH. In other embodiments, Q₁ is NR. In other embodiments, Q₁ is N—CH₃. In other embodiments, Q₁ is N—(CH₂)₂—O—(CH₂)₂O—CH₃. In other embodiments, Q₁ is S. In other embodiments, Q₁ is O. In other embodiments, Q₁ is N—OH. In other embodiments, Q₁ is N—OMe.

In some embodiments, Q₂ of formula I, II, I(a), I(b), and I(c) is C(R). In other embodiments, Q₂ is C—CH₃. In other embodiments, Q₂ is CH. In other embodiments, Q₂ is C—Cl. In other embodiments, Q₂ is C—CN. In other embodiments, Q₂ is C—CF₃. In other embodiments, Q₂ is C—CHF₂. In other embodiments, Q₂ is C—CH₂—O—(CH₂)₂O—CH₃. In other embodiments, Q₂ is N.

In some embodiments, G=X of formula I, II, I(a) and I(b) is C═O. In other embodiments, G=X is CH₂.

In various embodiments, this invention is directed to the compounds presented in Table 1, pharmaceutical compositions and/or method of use thereof, each represents a separate embodiment according to this invention:

TABLE 1

| Compound | |
|---|---|
| Number | Compound Structure |
| 100 | 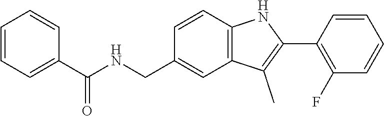 [SMILES: Cc1c(-c2ccccc2F)[nH]c2ccc(CNC(=O)c3ccccc3)cc12] |
| 101 | 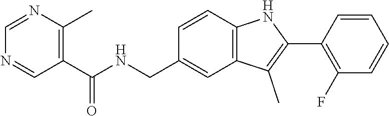 [SMILES: Cc1ncncc1C(=O)NCc1ccc2[nH]c(-c3ccccc3F)c(C)c2c1] |
| 102 | 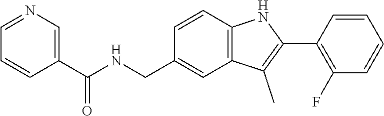 [SMILES: Cc1c(-c2ccccc2F)[nH]c2ccc(CNC(=O)c3cccnc3)cc12] |
| 103 | 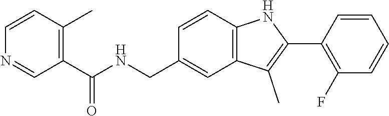 [SMILES: Cc1ccncc1C(=O)NCc1ccc2[nH]c(-c3ccccc3F)c(C)c2c1] |
| 104 | 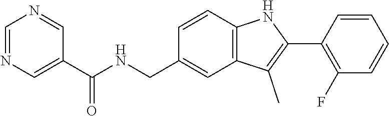 [SMILES: Cc1c(-c2ccccc2F)[nH]c2ccc(CNC(=O)c3cncnc3)cc12] |
| 105 | 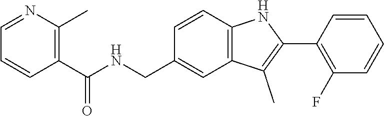 [SMILES: Cc1ncccc1C(=O)NCc1ccc2[nH]c(-c3ccccc3F)c(C)c2c1] |
| 106 | 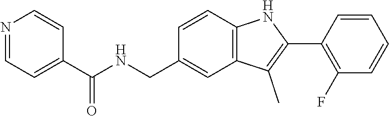 [SMILES: Cc1c(-c2ccccc2F)[nH]c2ccc(CNC(=O)c3ccncc3)cc12] |
| 109 | 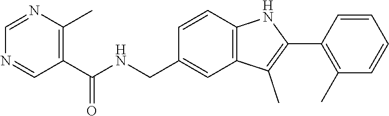 [SMILES: Cc1ccccc1-c1[nH]c2ccc(CNC(=O)c3cncnc3C)cc2c1C] |
| 110 | 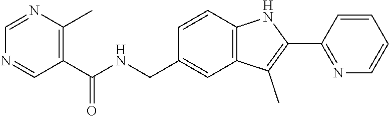 [SMILES: Cc1ncncc1C(=O)NCc1ccc2[nH]c(-c3ccccn3)c(C)c2c1] |
| 111 | 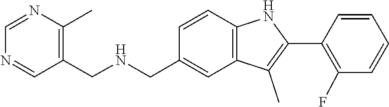 [SMILES: Cc1ncncc1CNCc1ccc2[nH]c(-c3ccccc3F)c(C)c2c1] |
| 112 | 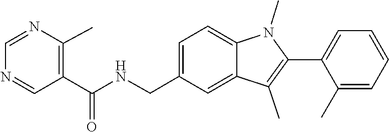 [SMILES: Cc1ccccc1-c1c(C)c2cc(CNC(=O)c3cncnc3C)ccc2n1C] |
| 113 | 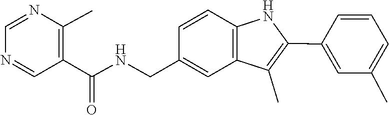 [SMILES: Cc1cccc(-c2[nH]c3ccc(CNC(=O)c4cncnc4C)cc3c2C)c1] |
| 114 | 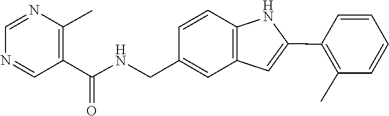 [SMILES: Cc1ccccc1-c1cc2cc(CNC(=O)c3cncnc3C)ccc2[nH]1] |
| 115 | 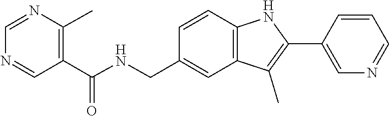 [SMILES: Cc1ncncc1C(=O)NCc1ccc2[nH]c(-c3cccnc3)c(C)c2c1] |
| 116 | 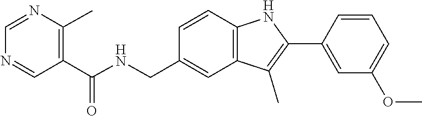 [SMILES: COc1cccc(-c2[nH]c3ccc(CNC(=O)c4cncnc4C)cc3c2C)c1] |
| 117 | 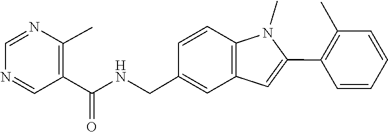 [SMILES: Cc1ccccc1-c1cc2cc(CNC(=O)c3cncnc3C)ccc2n1C] |
| 118 | 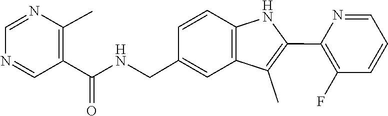 [SMILES: Cc1ncncc1C(=O)NCc1ccc2[nH]c(-c3ncccc3F)c(C)c2c1] |
| 119 | 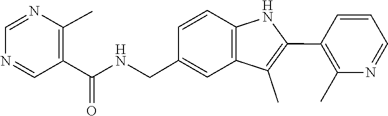 [SMILES: Cc1ncncc1C(=O)NCc1ccc2[nH]c(-c3cccnc3C)c(C)c2c1] |
| 120 | 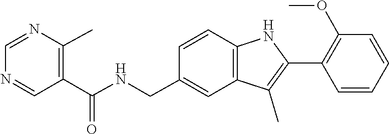 [SMILES: COc1ccccc1-c1[nH]c2ccc(CNC(=O)c3cncnc3C)cc2c1C] |
| 121 | 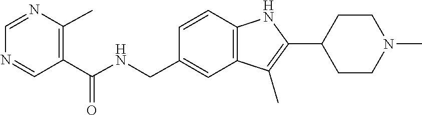 [SMILES: Cc1ncncc1C(=O)NCc1ccc2[nH]c(C3CCN(C)CC3)c(C)c2c1] |
| 122 | 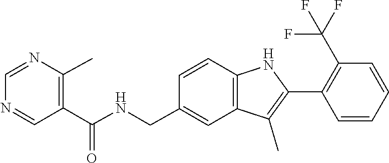 [SMILES: Cc1ncncc1C(=O)NCc1ccc2[nH]c(-c3ccccc3C(F)(F)F)c(C)c2c1] |
| 123 | 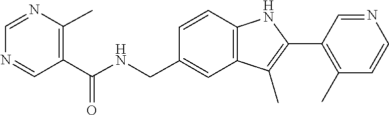 [SMILES: Cc1ccncc1-c1[nH]c2ccc(CNC(=O)c3cncnc3C)cc2c1C] |
| 124 | 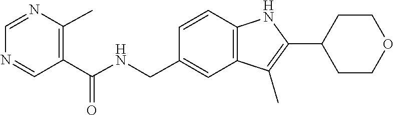 [SMILES: Cc1ncncc1C(=O)NCc1ccc2[nH]c(C3CCOCC3)c(C)c2c1] |
| 125 | 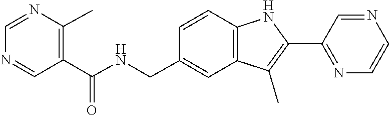 [SMILES: Cc1ncncc1C(=O)NCc1ccc2[nH]c(-c3cnccn3)c(C)c2c1] |
| 126 | 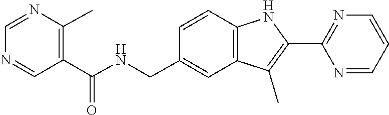 [SMILES: Cc1ncncc1C(=O)NCc1ccc2[nH]c(-c3ncccn3)c(C)c2c1] |
| 127 | 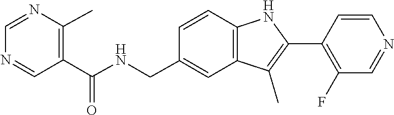 [SMILES: Cc1ncncc1C(=O)NCc1ccc2[nH]c(-c3ccncc3F)c(C)c2c1] |
| 128 | 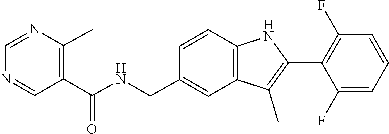 [SMILES: Cc1ncncc1C(=O)NCc1ccc2[nH]c(-c3c(F)cccc3F)c(C)c2c1] |
| 129 | 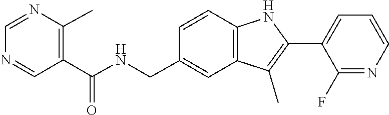 [SMILES: Cc1ncncc1C(=O)NCc1ccc2[nH]c(-c3cccnc3F)c(C)c2c1] |
| 130 | 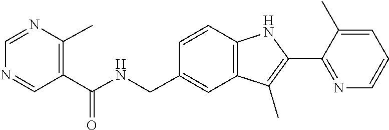 [SMILES: Cc1cccnc1-c1[nH]c2ccc(CNC(=O)c3cncnc3C)cc2c1C] |
| 131 | 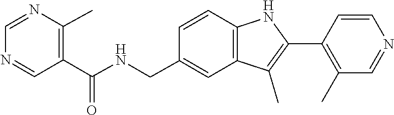 [SMILES: Cc1cnccc1-c1[nH]c2ccc(CNC(=O)c3cncnc3C)cc2c1C] |
| 132 (Isomer 2) | 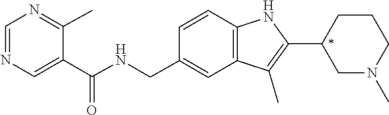 [SMILES: Cc1ncncc1C(=O)NCc1ccc2[nH]c(C3CCCN(C)C3)c(C)c2c1] |
| 133 | 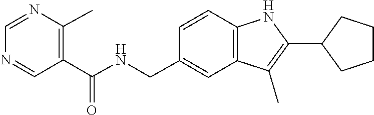 [SMILES: Cc1ncncc1C(=O)NCc1ccc2[nH]c(C3CCCC3)c(C)c2c1] |
| 134 (Isomer 1) | 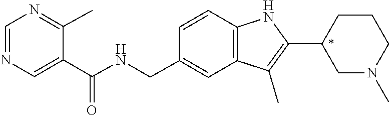 [SMILES: Cc1ncncc1C(=O)NCc1ccc2[nH]c(C3CCCN(C)C3)c(C)c2c1] |
| 135 | 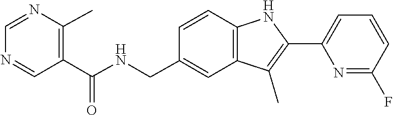 [SMILES: Cc1ncncc1C(=O)NCc1ccc2[nH]c(-c3cccc(F)n3)c(C)c2c1] |
| 136 | 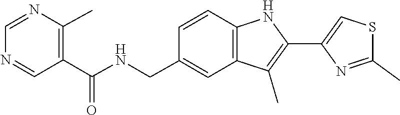 [SMILES: Cc1nc(-c2[nH]c3ccc(CNC(=O)c4cncnc4C)cc3c2C)cs1] |
| 137 | 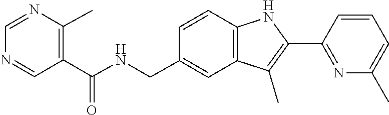 [SMILES: Cc1cccc(-c2[nH]c3ccc(CNC(=O)c4cncnc4C)cc3c2C)n1] |
| 138 | 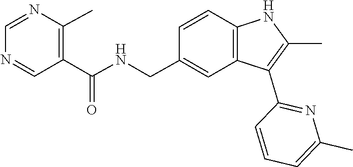 [SMILES: Cc1cccc(-c2c(C)[nH]c3ccc(CNC(=O)c4cncnc4C)cc23)n1] |
| 139 | 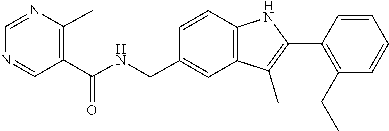 [SMILES: CCc1ccccc1-c1[nH]c2ccc(CNC(=O)c3cncnc3C)cc2c1C] |
| 140 | 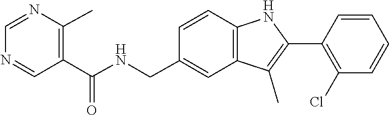 [SMILES: Cc1ncncc1C(=O)NCc1ccc2[nH]c(-c3ccccc3Cl)c(C)c2c1] |
| 141 | 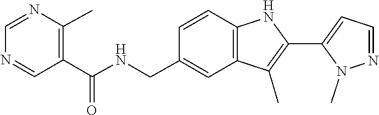 [SMILES: Cc1ncncc1C(=O)NCc1ccc2[nH]c(-c3ccnn3C)c(C)c2c1] |
| 142 | 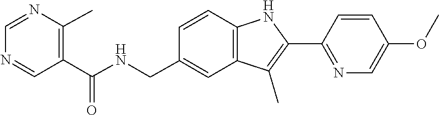 [SMILES: COc1ccc(-c2[nH]c3ccc(CNC(=O)c4cncnc4C)cc3c2C)nc1] |
| 143 | 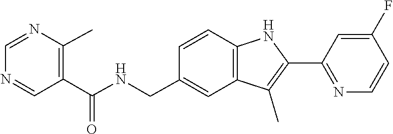 [SMILES: Cc1ncncc1C(=O)NCc1ccc2[nH]c(-c3cc(F)ccn3)c(C)c2c1] |
| 144 | 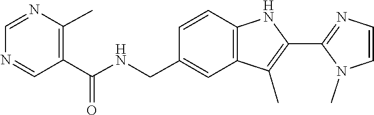 [SMILES: Cc1ncncc1C(=O)NCc1ccc2[nH]c(-c3nccn3C)c(C)c2c1] |
| 145 | 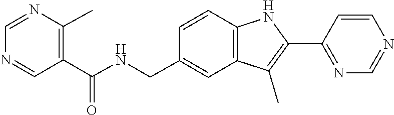 [SMILES: Cc1ncncc1C(=O)NCc1ccc2[nH]c(-c3ccncn3)c(C)c2c1] |
| 146 | 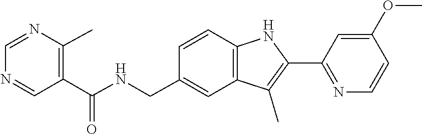 [SMILES: COc1ccnc(-c2[nH]c3ccc(CNC(=O)c4cncnc4C)cc3c2C)c1] |
| 147 | 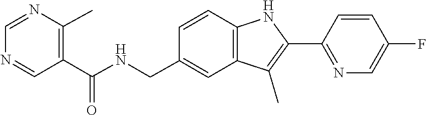 [SMILES: Cc1ncncc1C(=O)NCc1ccc2[nH]c(-c3ccc(F)cn3)c(C)c2c1] |
| 148 | 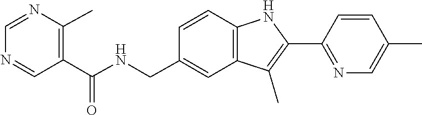 [SMILES: Cc1ccc(-c2[nH]c3ccc(CNC(=O)c4cncnc4C)cc3c2C)nc1] |
| 149 | 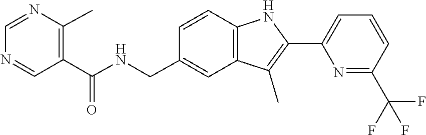 [SMILES: Cc1ncncc1C(=O)NCc1ccc2[nH]c(-c3cccc(C(F)(F)F)n3)c(C)c2c1] |
| 150 | 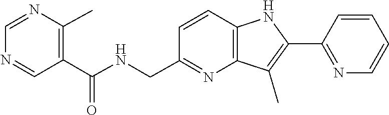 [SMILES: Cc1ncncc1C(=O)NCc1ccc2[nH]c(-c3ccccn3)c(C)c2n1] |
| 151 | 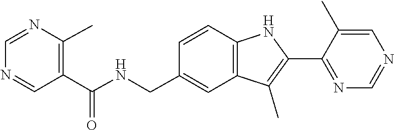 [SMILES: Cc1cncnc1-c1[nH]c2ccc(CNC(=O)c3cncnc3C)cc2c1C] |
| 152 | 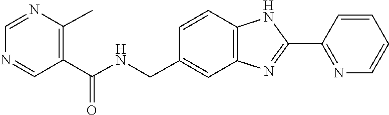 [SMILES: Cc1ncncc1C(=O)NCc1ccc2[nH]c(-c3ccccn3)nc2c1] |
| 153 | 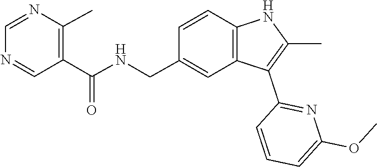 [SMILES: COc1cccc(-c2c(C)[nH]c3ccc(CNC(=O)c4cncnc4C)cc23)n1] |
| 154 | 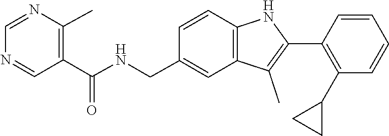 [SMILES: Cc1ncncc1C(=O)NCc1ccc2[nH]c(-c3ccccc3C3CC3)c(C)c2c1] |
| 155 | 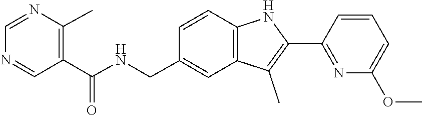 [SMILES: COc1cccc(-c2[nH]c3ccc(CNC(=O)c4cncnc4C)cc3c2C)n1] |
| 156 | 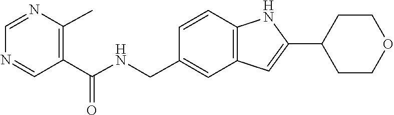 [SMILES: Cc1ncncc1C(=O)NCc1ccc2[nH]c(C3CCOCC3)cc2c1] |
| 157 | 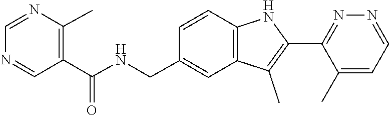 [SMILES: Cc1ccnnc1-c1[nH]c2ccc(CNC(=O)c3cncnc3C)cc2c1C] |
| 158 | 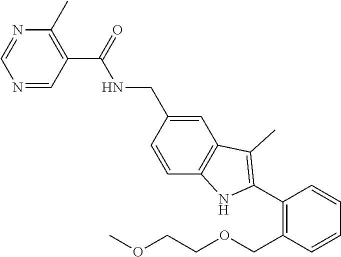 [SMILES: COCCOCc1ccccc1-c1[nH]c2ccc(CNC(=O)c3cncnc3C)cc2c1C] |
| 159 | 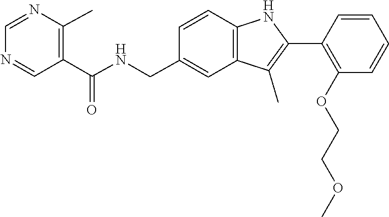 [SMILES: COCCOc1ccccc1-c1[nH]c2ccc(CNC(=O)c3cncnc3C)cc2c1C] |
| 160 | 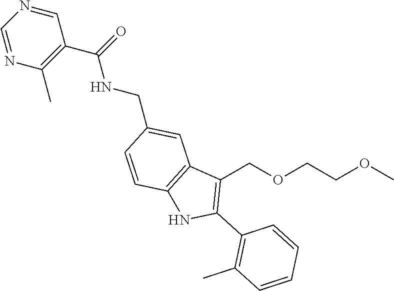 [SMILES: COCCOCc1c(-c2ccccc2C)[nH]c2ccc(CNC(=O)c3cncnc3C)cc12] |
| 161 | 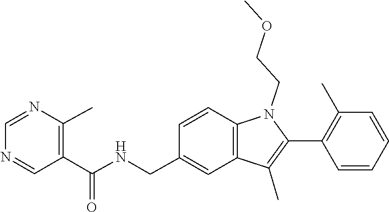 [SMILES: COCCn1c(-c2ccccc2C)c(C)c2cc(CNC(=O)c3cncnc3C)ccc21] |
| 162 | 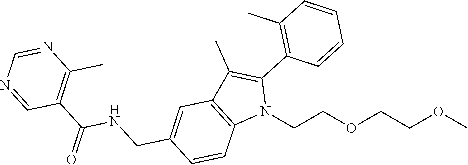 [SMILES: COCCOCCn1c(-c2ccccc2C)c(C)c2cc(CNC(=O)c3cncnc3C)ccc21] |
| 163 | 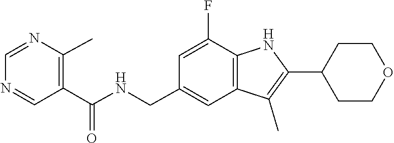 [SMILES: Cc1ncncc1C(=O)NCc1cc(F)c2[nH]c(C3CCOCC3)c(C)c2c1] |
| 164 | 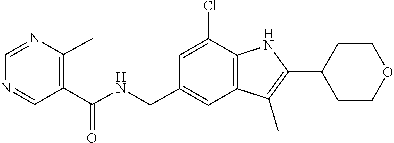 [SMILES: Cc1ncncc1C(=O)NCc1cc(Cl)c2[nH]c(C3CCOCC3)c(C)c2c1] |
| 165 | 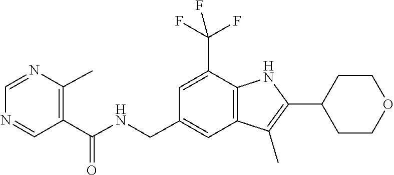 [SMILES: Cc1ncncc1C(=O)NCc1cc(C(F)(F)F)c2[nH]c(C3CCOCC3)c(C)c2c1] |
| 166 | 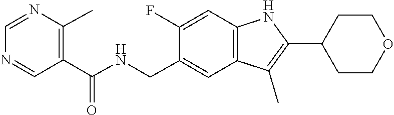 [SMILES: Cc1ncncc1C(=O)NCc1cc2c(C)c(C3CCOCC3)[nH]c2cc1F] |
| 167 | 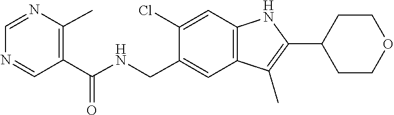 [SMILES: Cc1ncncc1C(=O)NCc1cc2c(C)c(C3CCOCC3)[nH]c2cc1Cl] |
| 168 | 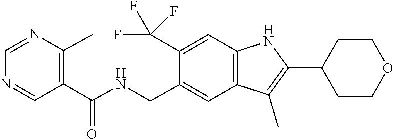 [SMILES: Cc1ncncc1C(=O)NCc1cc2c(C)c(C3CCOCC3)[nH]c2cc1C(F)(F)F] |
| 169 | 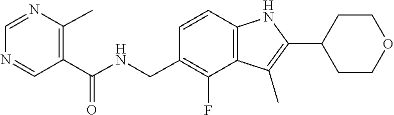 [SMILES: Cc1ncncc1C(=O)NCc1ccc2[nH]c(C3CCOCC3)c(C)c2c1F] |
| 170 | 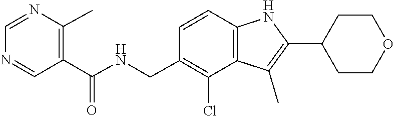 [SMILES: Cc1ncncc1C(=O)NCc1ccc2[nH]c(C3CCOCC3)c(C)c2c1Cl] |
| 171 | 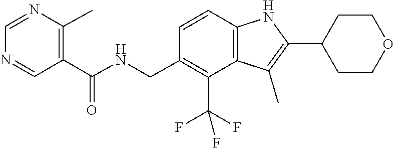 [SMILES: Cc1ncncc1C(=O)NCc1ccc2[nH]c(C3CCOCC3)c(C)c2c1C(F)(F)F] |
| 173 | 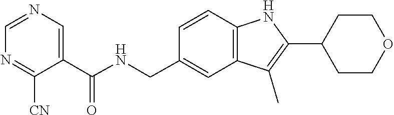 [SMILES: Cc1c(C2CCOCC2)[nH]c2ccc(CNC(=O)c3cncnc3C#N)cc12] |
| 174 | 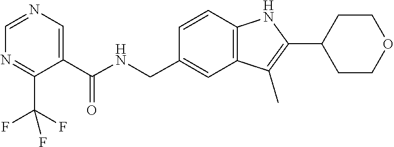 [SMILES: Cc1c(C2CCOCC2)[nH]c2ccc(CNC(=O)c3cncnc3C(F)(F)F)cc12] |
| 175 | 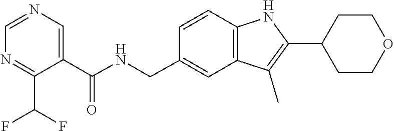 [SMILES: Cc1c(C2CCOCC2)[nH]c2ccc(CNC(=O)c3cncnc3C(F)F)cc12] |
| 176 | 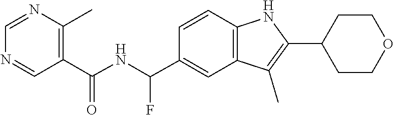 [SMILES: Cc1ncncc1C(=O)NC(F)c1ccc2[nH]c(C3CCOCC3)c(C)c2c1] |
| 177 | 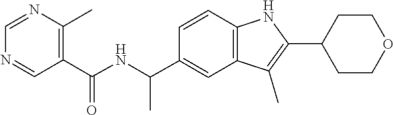 [SMILES: Cc1ncncc1C(=O)NC(C)c1ccc2[nH]c(C3CCOCC3)c(C)c2c1] |
| 178 | 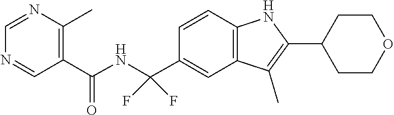 [SMILES: Cc1ncncc1C(=O)NC(F)(F)c1ccc2[nH]c(C3CCOCC3)c(C)c2c1] |
| 179 | 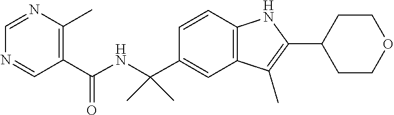 [SMILES: Cc1ncncc1C(=O)NC(C)(C)c1ccc2[nH]c(C3CCOCC3)c(C)c2c1] |
| 180 | 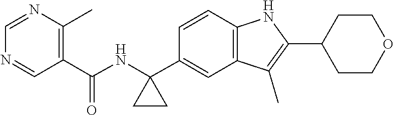 [SMILES: Cc1ncncc1C(=O)NC1(c2ccc3[nH]c(C4CCOCC4)c(C)c3c2)CC1] |
| 181 | 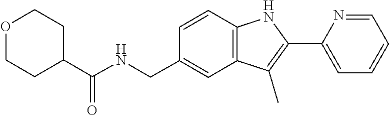 [SMILES: Cc1c(-c2ccccn2)[nH]c2ccc(CNC(=O)C3CCOCC3)cc12] |
| 182 | 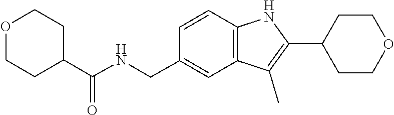 [SMILES: Cc1c(C2CCOCC2)[nH]c2ccc(CNC(=O)C3CCOCC3)cc12] |
| 183 | 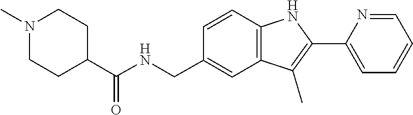 [SMILES: Cc1c(-c2ccccn2)[nH]c2ccc(CNC(=O)C3CCN(C)CC3)cc12] |
| 184 | 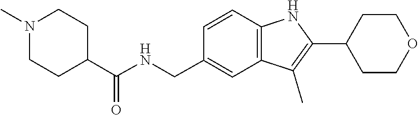 [SMILES: Cc1c(C2CCOCC2)[nH]c2ccc(CNC(=O)C3CCN(C)CC3)cc12] |
| 185 | 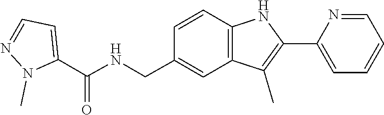 [SMILES: Cc1c(-c2ccccn2)[nH]c2ccc(CNC(=O)c3ccnn3C)cc12] |
| 186 | 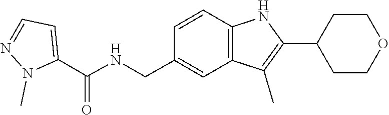 [SMILES: Cc1c(C2CCOCC2)[nH]c2ccc(CNC(=O)c3ccnn3C)cc12] |
| 187 | 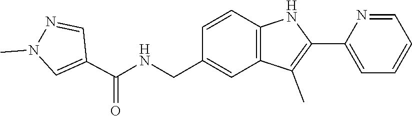 [SMILES: Cc1c(-c2ccccn2)[nH]c2ccc(CNC(=O)c3cnn(C)c3)cc12] |
| 188 | 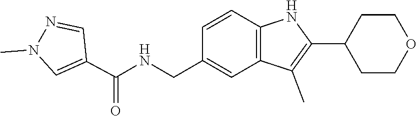 [SMILES: Cc1c(C2CCOCC2)[nH]c2ccc(CNC(=O)c3cnn(C)c3)cc12] |
| 189 | 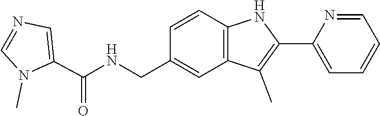 [SMILES: Cc1c(-c2ccccn2)[nH]c2ccc(CNC(=O)c3cncn3C)cc12] |
| 190 | 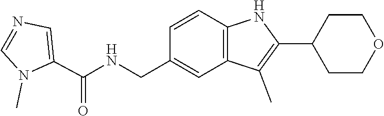 [SMILES: Cc1c(C2CCOCC2)[nH]c2ccc(CNC(=O)c3cncn3C)cc12] |
| 191 | 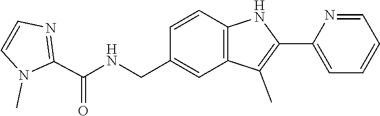 [SMILES: Cc1c(-c2ccccn2)[nH]c2ccc(CNC(=O)c3nccn3C)cc12] |
| 192 | 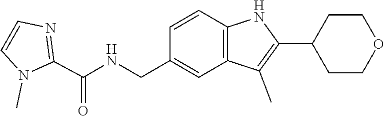 [SMILES: Cc1c(C2CCOCC2)[nH]c2ccc(CNC(=O)c3nccn3C)cc12] |
| 193 | 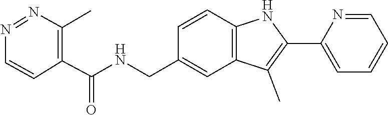 [SMILES: Cc1nnccc1C(=O)NCc1ccc2[nH]c(-c3ccccn3)c(C)c2c1] |
| 194 | 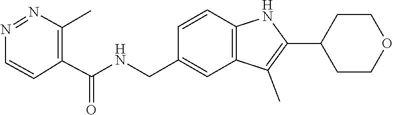 [SMILES: Cc1nnccc1C(=O)NCc1ccc2[nH]c(C3CCOCC3)c(C)c2c1] |
| 195 | 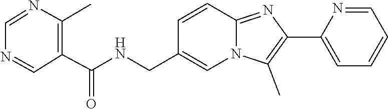 [SMILES: Cc1ncncc1C(=O)NCc1ccc2nc(-c3ccccn3)c(C)n2c1] |
| 196 | 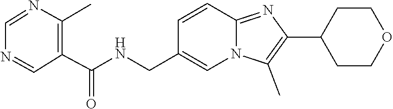 [SMILES: Cc1ncncc1C(=O)NCc1ccc2nc(C3CCOCC3)c(C)n2c1] |
| 197 | 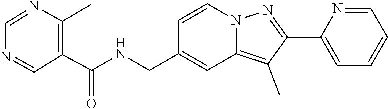 [SMILES: Cc1ncncc1C(=O)NCc1ccn2nc(-c3ccccn3)c(C)c2c1] |
| 198 | 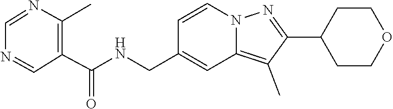 [SMILES: Cc1ncncc1C(=O)NCc1ccn2nc(C3CCOCC3)c(C)c2c1] |
| 199 | 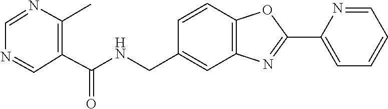 [SMILES: Cc1ncncc1C(=O)NCc1ccc2oc(-c3ccccn3)nc2c1] |
| 200 | 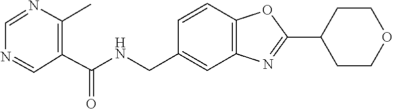 [SMILES: Cc1ncncc1C(=O)NCc1ccc2oc(C3CCOCC3)nc2c1] |
| 201 | 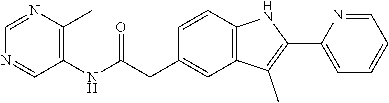 [SMILES: Cc1ncncc1NC(=O)Cc1ccc2[nH]c(-c3ccccn3)c(C)c2c1] |
| 202 | 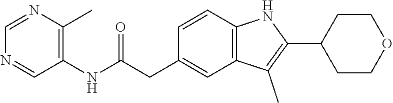 [SMILES: Cc1ncncc1NC(=O)Cc1ccc2[nH]c(C3CCOCC3)c(C)c2c1] |
| 203 | 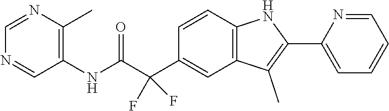 [SMILES: Cc1ncncc1NC(=O)C(F)(F)c1ccc2[nH]c(-c3ccccn3)c(C)c2c1] |
| 204 | 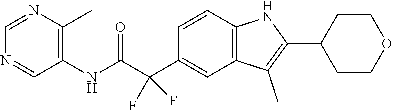 [SMILES: Cc1ncncc1NC(=O)C(F)(F)c1ccc2[nH]c(C3CCOCC3)c(C)c2c1] |
| 205 | 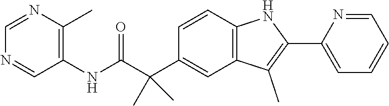 [SMILES: Cc1ncncc1NC(=O)C(C)(C)c1ccc2[nH]c(-c3ccccn3)c(C)c2c1] |
| 206 | 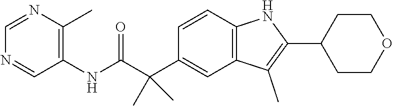 [SMILES: Cc1ncncc1NC(=O)C(C)(C)c1ccc2[nH]c(C3CCOCC3)c(C)c2c1] |
| 207 | 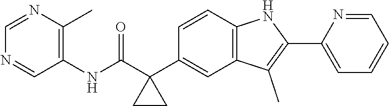 [SMILES: Cc1ncncc1NC(=O)C1(c2ccc3[nH]c(-c4ccccn4)c(C)c3c2)CC1] |
| 208 | 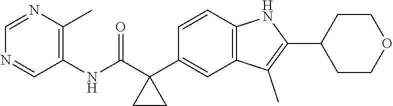 [SMILES: Cc1ncncc1NC(=O)C1(c2ccc3[nH]c(C4CCOCC4)c(C)c3c2)CC1] |
| 209 | 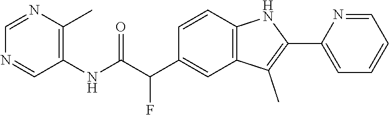 [SMILES: Cc1ncncc1NC(=O)C(F)c1ccc2[nH]c(-c3ccccn3)c(C)c2c1] |
| 210 | 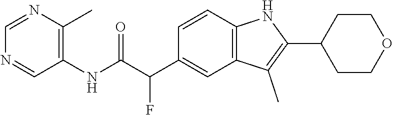 [SMILES: Cc1ncncc1NC(=O)C(F)c1ccc2[nH]c(C3CCOCC3)c(C)c2c1] |
| 211 | 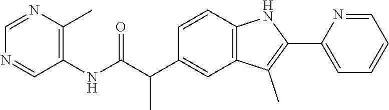 [SMILES: Cc1ncncc1NC(=O)C(C)c1ccc2[nH]c(-c3ccccn3)c(C)c2c1] |
| 212 | 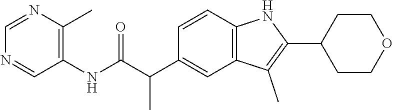 [SMILES: Cc1ncncc1NC(=O)C(C)c1ccc2[nH]c(C3CCOCC3)c(C)c2c1] |
| 213 | 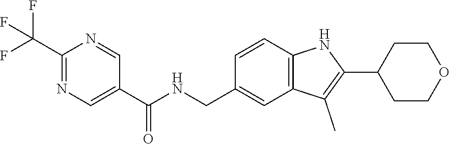 [SMILES: Cc1c(C2CCOCC2)[nH]c2ccc(CNC(=O)c3cnc(C(F)(F)F)nc3)cc12] |
| 214 | 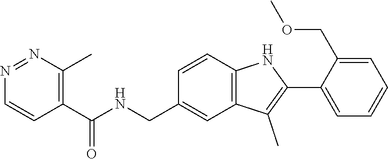 [SMILES: COCc1ccccc1-c1[nH]c2ccc(CNC(=O)c3ccnnc3C)cc2c1C] |
| 215 | 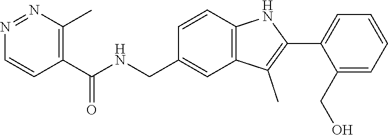 [SMILES: Cc1nnccc1C(=O)NCc1ccc2[nH]c(-c3ccccc3CO)c(C)c2c1] |
| 216 | 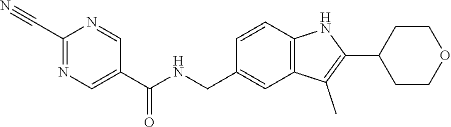 [SMILES: Cc1c(C2CCOCC2)[nH]c2ccc(CNC(=O)c3cnc(C#N)nc3)cc12] |
| 217 | 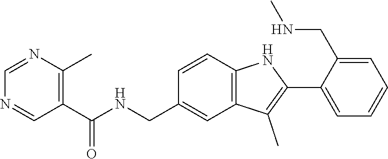 [SMILES: CNCc1ccccc1-c1[nH]c2ccc(CNC(=O)c3cncnc3C)cc2c1C] |
| 218 | 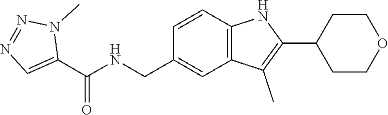 [SMILES: Cc1c(C2CCOCC2)[nH]c2ccc(CNC(=O)c3cnnn3C)cc12] |
| 219 | 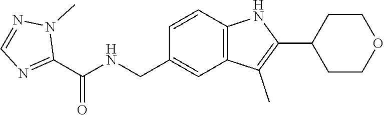 [SMILES: Cc1c(C2CCOCC2)[nH]c2ccc(CNC(=O)c3ncnn3C)cc12] |
| 220 | 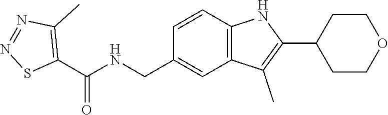 [SMILES: Cc1nnsc1C(=O)NCc1ccc2[nH]c(C3CCOCC3)c(C)c2c1] |
| 221 | 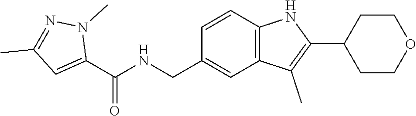 [SMILES: Cc1cc(C(=O)NCc2ccc3[nH]c(C4CCOCC4)c(C)c3c2)n(C)n1] |
| 222 | 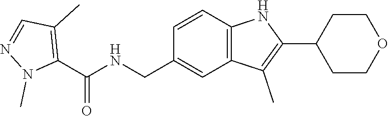 [SMILES: Cc1cnn(C)c1C(=O)NCc1ccc2[nH]c(C3CCOCC3)c(C)c2c1] |
| 223 | 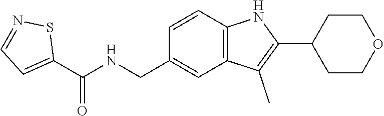 [SMILES: Cc1c(C2CCOCC2)[nH]c2ccc(CNC(=O)c3ccns3)cc12] |
| 224 | 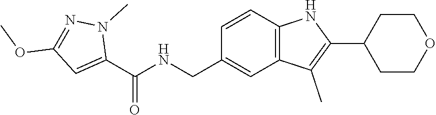 [SMILES: COc1cc(C(=O)NCc2ccc3[nH]c(C4CCOCC4)c(C)c3c2)n(C)n1] |
| 225 | 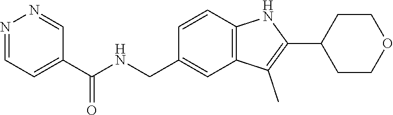 [SMILES: Cc1c(C2CCOCC2)[nH]c2ccc(CNC(=O)c3ccnnc3)cc12] |
| 227 | 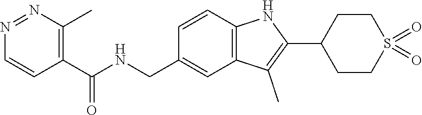 [SMILES: Cc1nnccc1C(=O)NCc1ccc2[nH]c(C3CCS(=O)(=O)CC3)c(C)c2c1] |
| 229 | 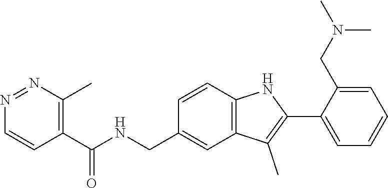 [SMILES: Cc1nnccc1C(=O)NCc1ccc2[nH]c(-c3ccccc3CN(C)C)c(C)c2c1] |
| 230 | 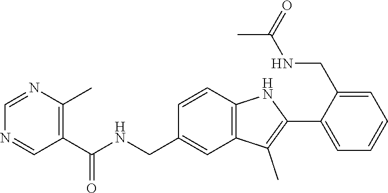 [SMILES: CC(=O)NCc1ccccc1-c1[nH]c2ccc(CNC(=O)c3cncnc3C)cc2c1C] |
| 231 | 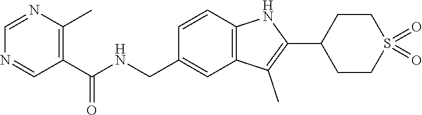 [SMILES: Cc1ncncc1C(=O)NCc1ccc2[nH]c(C3CCS(=O)(=O)CC3)c(C)c2c1] |
| 232 | 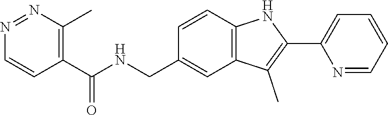 [SMILES: Cc1nnccc1C(=O)NCc1ccc2[nH]c(-c3ccccn3)c(C)c2c1] |
| 233 | 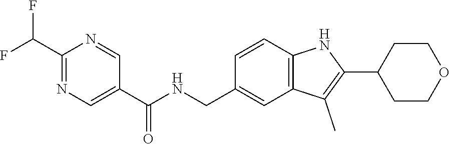 [SMILES: Cc1c(C2CCOCC2)[nH]c2ccc(CNC(=O)c3cnc(C(F)F)nc3)cc12] |
| 234 | 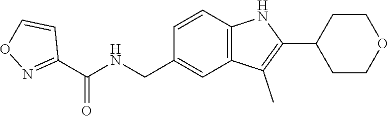 [SMILES: Cc1c(C2CCOCC2)[nH]c2ccc(CNC(=O)c3ccon3)cc12] |
| 235 | 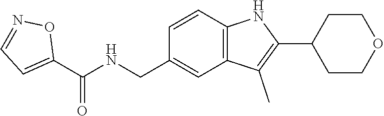 [SMILES: Cc1c(C2CCOCC2)[nH]c2ccc(CNC(=O)c3ccno3)cc12] |
| 236 | 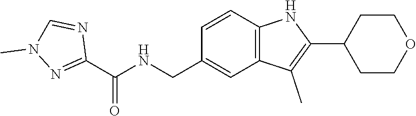 [SMILES: Cc1c(C2CCOCC2)[nH]c2ccc(CNC(=O)c3ncn(C)n3)cc12] |
| 237 | 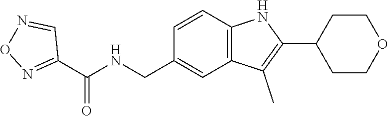 [SMILES: Cc1c(C2CCOCC2)[nH]c2ccc(CNC(=O)c3cnon3)cc12] |
| 238 | 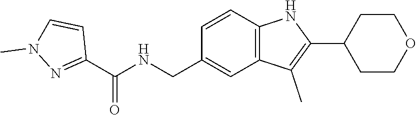 [SMILES: Cc1c(C2CCOCC2)[nH]c2ccc(CNC(=O)c3ccn(C)n3)cc12] |
| 239 | 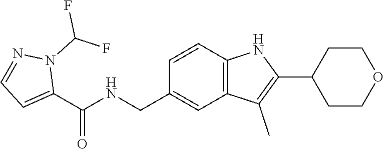 [SMILES: Cc1c(C2CCOCC2)[nH]c2ccc(CNC(=O)c3ccnn3C(F)F)cc12] |
| 240 | 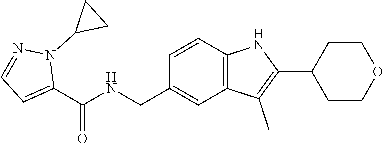 [SMILES: Cc1c(C2CCOCC2)[nH]c2ccc(CNC(=O)c3ccnn3C3CC3)cc12] |
| 241 | 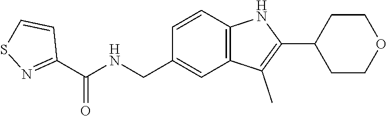 [SMILES: Cc1c(C2CCOCC2)[nH]c2ccc(CNC(=O)c3ccsn3)cc12] |
| 242 | 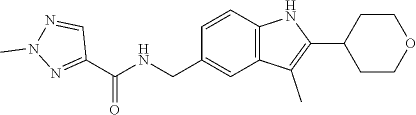 [SMILES: Cc1c(C2CCOCC2)[nH]c2ccc(CNC(=O)c3cnn(C)n3)cc12] |
| 243 | 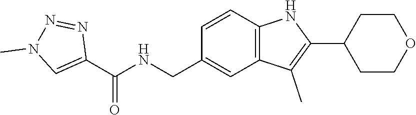 [SMILES: Cc1c(C2CCOCC2)[nH]c2ccc(CNC(=O)c3cn(C)nn3)cc12] |
| 244 | 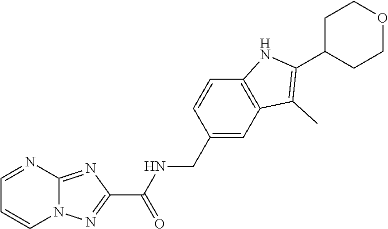 [SMILES: Cc1c(C2CCOCC2)[nH]c2ccc(CNC(=O)c3nc4ncccn4n3)cc12] |
| 245 | 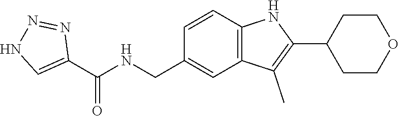 [SMILES: Cc1c(C2CCOCC2)[nH]c2ccc(CNC(=O)c3c[nH]nn3)cc12] |
| 246 | 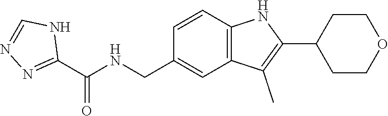 [SMILES: Cc1c(C2CCOCC2)[nH]c2ccc(CNC(=O)c3nnc[nH]3)cc12] |
| 247 | 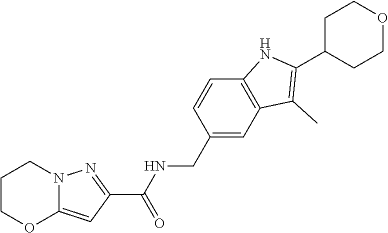 [SMILES: Cc1c(C2CCOCC2)[nH]c2ccc(CNC(=O)c3cc4n(n3)CCCO4)cc12] |
| 248 | 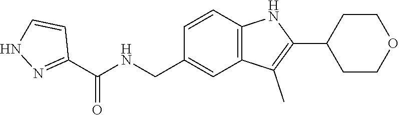 [SMILES: Cc1c(C2CCOCC2)[nH]c2ccc(CNC(=O)c3cc[nH]n3)cc12] |
| 249 | 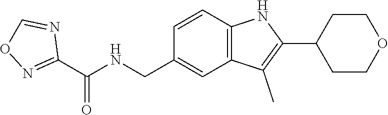 [SMILES: Cc1c(C2CCOCC2)[nH]c2ccc(CNC(=O)c3ncon3)cc12] |

It is well understood that in structures presented in this invention wherein the carbon atom has less than 4 bonds, H atoms are present to complete the valence of the carbon. It is well understood that in structures presented in this invention wherein the nitrogen atom has less than 3 bonds, H atoms are present to complete the valence of the nitrogen.

In some embodiments, this invention is directed to the compounds listed hereinabove, pharmaceutical compositions and/or method of use thereof, wherein the compound is pharmaceutically acceptable salt, optical isomer, tautomer, hydrate, N-oxide, reverse amide analog, prodrug, isotopic variant (deuterated analog), PROTAC, pharmaceutical product or any combination thereof. In some embodiments, the compounds are Collagen I translation inhibitors.

In various embodiments, the A ring of formula I and/or II is phenyl, naphthyl, pyridinyl, pyrimidinyl, pyridazinyl, pyrazinyl, triazinyl, tetrazinyl, thiazolyl, isothiazolyl, oxazolyl, isoxazolyl, imidazolyl, 1-methylimidazole, isoquinoline, pyrazolyl, pyrrolyl, furanyl, thiophene-yl, isoquinolinyl, indolyl, 1H-indole, isoindolyl, naphthyl, anthracenyl, benzimidazolyl, indazolyl, 2H-indazole, triazolyl, 4,5,6,7-tetrahydro-2H-indazole, 3H-indol-3-one, purinyl, benzoxazolyl, 1,3-benzoxazolyl, benzisoxazolyl, benzothiazolyl, 1,3-benzothiazole, 4,5,6,7-tetrahydro-1,3-benzothiazole, quinazolinyl, quinoxalinyl, cinnolinyl, phthalazinyl, quinolinyl, isoquinolinyl, 2,3-dihydroindenyl, indenyl, tetrahydronaphthyl, 3,4-dihydro-2H-benzo[b][1,4]dioxepine, benzo[d][1,3]dioxole, acridinyl, benzofuranyl, 1-benzofuran, isobenzofuranyl, benzofuran-2(3H)-one, benzothiophenyl, benzoxadiazole, benzo[c][1,2,5]oxadiazolyl, benzo[c]thiophenyl, benzodioxolyl, benzo[d][1,3]dioxole, thiadiazolyl, tetrahydrofuranyl, oxazolonyl, oxazolidonyl, thiazolonyl, isothiazolynonyl, isoxazolidinonyl, imidazolidinonyl, pyrazolonyl, 2H-pyrrol-2-onyl, furanonyl, thiophenonyl, pyrrolidine, 2-oxo-pyrrolidine, [1,3]oxazolo[4,5-b]pyridine, 1,2,3-, 1,2,4-, 1,2,5- or 1,3,4- oxadiazolyl, 3H-[1,2,3]triazolo[4,5-d]pyrimidine, 1H-[1,2,3]triazolo[4,5-d]pyrimidine, [1,2,4]triazolo[4,3-c]pyrimidine, [1,2,4]triazolo[4,3-a]pyrimidine, [1,2,3]triazolo[1,5-a]pyrimidine, [1,2,3]triazolo[1,5-c]pyrimidine, [1,2,4]triazolo[1,5-a]pyrimidine, [1,2,4]triazolo[1,5-c]pyrimidine, 6,7-dihydro-5H-pyrazolo[5,1-b][1,3]oxazine, imidazo[2,1-b][1,3]thiazole, 4H,5H,6H-cyclopenta[d][1,3]thiazole, 5H,6H,7H,8H-imidazo[1,2-a]pyridine, 7-oxo-6H,7H-[1,3]thiazolo[4,5-d]pyrimidine, [1,3]thiazolo[5,4-b]pyridine, 2H,3H-imidazo[2,1-b][1,3]thiazole, thieno[3,2-d]pyrimidin-4(3H)-one, 4-oxo-4H-thieno[3,2-d][1,3]thiazin, imidazo[1,2-a]pyridine, 1H-imidazo[4,5-b]pyridine, 1H-imidazo[4,5-c]pyridine, 3H-imidazo[4,5-c]pyridine, pyrazolo[1,5-a]pyridine, imidazo[1,2-a]pyrazine, imidazo[1,2-a]pyrimidine, 1H-pyrrolo[2,3-b]pyridine, pyrido[2,3-b]pyrazine, pyrido[2,3-b]pyrazin-3(4H)-one, 4H-thieno[3,2-b]pyrrole, quinoxalin-2(1H)-one, 1H-pyrrolo[3,2-b]pyridine, 7H-pyrrolo[2,3-d]pyrimidine, oxazolo[5,4-b]pyridine, thiazolo[5,4-b]pyridine, thieno[3,2-c]pyridine, each definition is a separate embodiment according to this invention; or A is C₃-C₈ cycloalkyl (e.g. cyclohexyl, cyclopentyl) or C₃-C₈ heterocyclic ring including but not limited to: tetrahydropyran, tetrahydro-2H-pyran, piperidine, 1-methylpiperidine, tetrahydrothiophene 1,1-dioxide, 1-(piperidin-1-yl)ethanone or morpholine. In some embodiments, A is a C₃-C₈ heterocyclic ring.

In some embodiments, if A is a phenyl, then at least one of R₁ and R₂ is not H, and at least one of n and m is not 0. In various embodiments, the A′ ring of formula I(f) is a 5 (five) membered heteroaromatic or a heterocyclic ring. In some embodiments, the A′ ring of formula I(f) is a 5 (five) membered heteroaromatic ring. In some embodiments, the A′ ring of formula I(f) is a 5 (five) membered heterocyclic ring. Non limiting examples of ring A′ are selected from: thiazolyl, isothiazolyl, oxazolyl, isoxazolyl, imidazolyl, 1-methylimidazolyl, pyrazolyl, pyrrolyl, furanyl, thiophene-yl, triazolyl, thiadiazolyl, 1,2,3-, 1,2,4-, 1,2,5- or 1,3,4- oxadiazolyl, tetrahydrofuranyl, oxazolonyl, oxazolidonyl, thiazolonyl, isothiazolynonyl, isoxazolidinonyl, imidazolidinonyl, pyrazolonyl, 2H-pyrrol-2-onyl, furanonyl, pyrrolidine, 2-oxo-pyrrolidine and thiophenonyl; each represents a separate embodiment according to this invention.

In various embodiments, the B ring of formula I, II, I(a), I(b), I(d) and/or I(e) is phenyl, naphthyl, pyridinyl, pyrimidinyl, pyridazinyl, pyrazinyl, triazinyl, tetrazinyl, thiazolyl, isothiazolyl, oxazolyl, isoxazolyl, imidazolyl, 1-methylimidazole, isoquinoline, pyrazolyl, pyrrolyl, furanyl, thiophene-yl, isoquinolinyl, indolyl, 1H-indole, isoindolyl, naphthyl, anthracenyl, benzimidazolyl, 2,3-dihydro-1H-benzo[d]imidazolyl, tetrahydronaphthyl 3,4-dihydro-2H-benzo[b][1,4]dioxepine, benzofuran-2(3H)-one, benzo[d][1,3]dioxole, indazolyl, 2H-indazole, triazolyl, 4,5,6,7-tetrahydro-2H-indazole, 3H-indol-3-one, purinyl, benzoxazolyl, 1,3-benzoxazolyl, benzisoxazolyl, benzothiazolyl, 1,3-benzothiazole, 4,5,6,7-tetrahydro-1,3-benzothiazole, quinazolinyl, quinoxalinyl, cinnolinyl, phthalazinyl, quinolinyl, isoquinolinyl, acridinyl, benzofuranyl, 1-benzofuran, isobenzofuranyl, benzothiophenyl, benzoxadiazole, benzo[c][1,2,5]oxadiazolyl, benzo[c]thiophenyl, benzodioxolyl, thiadiazolyl, [1,3]oxazolo[4,5-b]pyridine, oxadiazolyl, imidazo[2,1-b][1,3]thiazole, 4H,5H,6H-cyclopenta[d][1,3]thiazole, 5H,6H,7H,8H-imidazo[1,2-a]pyridine, 7-oxo-6H,7H-[1,3]thiazolo[4,5-d]pyrimidine, [1,3]thiazolo[5,4-b]pyridine, 2H,3H-imidazo[2,1-b][1,3]thiazole, thieno[3,2-d]pyrimidin-4(3H)-one, 4-oxo-4H-thieno[3,2-d][1,3]thiazin, imidazo[1,2-a]pyridine, 1H-imidazo[4,5-b]pyridine, 3H-imidazo[4,5-b]pyridine, 3H-imidazo[4,5-c]pyridine, pyrazolo[1,5-a]pyridine, imidazo[1,2-a]pyrazine, imidazo[1,2-a]pyrimidine, pyrido[2,3-b]pyrazin or pyrido[2,3-b]pyrazin-3(4H)-one, 4H-thieno[3,2-b]pyrrole, quinoxalin-2(1H)-one, 1,2,3,4-tetrahydroquinoxaline, 1-(pyridin-1(2H)-yl)ethanone, 1H-pyrrolo[2,3-b]pyridine, 1H-pyrrolo[3,2-b]pyridine, 7H-pyrrolo[2,3-d]pyrimidine, oxazolo[5,4-b]pyridine, thiazolo[5,4-b]pyridine, thieno[3,2-c]pyridine, C₃-C₈ cycloalkyl, or C₃-C₈ heterocyclic ring including but not limited to: tetrahydropyran, piperidine, 1-methylpiperidine, tetrahydrothiophene 1,1-dioxide, thiane 1,1-dioxide, 1-(piperidin-1-yl)ethanone or morpholine; each definition is a separate embodiment according to this invention. In some embodiments, B is a C₃-C₈ heterocyclic ring. In some embodiments, B is pyrimidine. In some embodiments, B is tetrahydro-2H-pyran. In other embodiments, B is C₃-C₈ cycloalkyl. In other embodiments, B is cyclopentyl.

In some embodiments, if B is a phenyl, then at least one of R₃ and R₄ is not H, and at least one of k and l is not 0.

In some embodiments, if A is a C₃-C₈ heterocyclic ring and B is a C₃-C₈ cycloalkyl, then at least one of R₁, R₂, R₃ and R₄ is not H, and at least one of n, m, k and l is not 0. In some embodiments, if A is a C₃-C₈ heterocyclic ring and B is a C₃-C₈ cycloalkyl, then at least one of R₁ and R₂ is not H, and at least one of n and m is not 0. In some embodiments, if A is a C₃-C₈ heterocyclic ring and B is a C₃-C₈ cycloalkyl, then at least one of R₃ and R₄ is not H, and at least one of k and l is not 0.

In some embodiments, if A is a C₃-C₈ heterocyclic ring and B is 2, 3, or 4-pyridinyl, then at least one of R₁, R₂, R₃ and R₄ is not H, and at least one of n, m, k and l is not 0. In some embodiments, if A is a C₃-C₈ heterocyclic ring and B is 3-pyridinyl, then at least one of R₁, R₂, R₃ and R₄ is not H, and at least one of n, m, k and l is not 0. In some embodiments, if A is a C₃-C₈ heterocyclic ring and B is pyridinyl, then at least one of R₁ and R₂ is not H, and at least one of n and m is not 0. In some embodiments, if A is a C₃-C₈ heterocyclic ring and B is pyridinyl, then at least one of R₃ and R₄ is not H, and at least one of k and l is not 0.

In various embodiments, compound of formula I, II, I(a), I(b), I(c), I(d), I(d(i)), I(e), I(e(i)) and/or I(f) is substituted by R₁, R₂ R₃ and R₄. Single substituents can be present at the ortho, meta, or para positions.

In various embodiments, R₁ and/or R₂ of formula I, II, I(a), I(b), I(c), I(d), I(d(i)), I(e), I(e(i)) and/or I(f) are each independently H.

In various embodiments, R₁ and/or R₂ of formula I, II, I(a), I(b), I(c), I(d), I(d(i)), I(e), I(e(i)) and/or I(f) are each independently H, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl; each represents a separate embodiment of this invention; wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof).

In some embodiments, R₁ and R₂ are joint together to form a 5 or 6 membered substituted or unsubstituted, aliphatic or aromatic, carbocyclic or heterocyclic ring. In some embodiments, R₁ and R₂ are joined together to form a 5 or 6 membered heterocyclic ring. In some embodiments, R₁ and R₂ are joined together to form a pyrrol ring. In some embodiments, R₁ and R₂ are joined together to form a [1,3]dioxole ring. In some embodiments, R₁ and R₂ are joined together to form a furan-2(3H)-one ring. In some embodiments, R₁ and R₂ are joint together to form a benzene ring. In some embodiments, R₁ and R₂ are joined together to form a pyridine ring. In some embodiments, R₁ and R₂ are joined together to form a morpholine ring. In some embodiments, R₁ and R₂ are joined together to form a piperazine ring. In some embodiments, R₁ and R₂ are joined together to form an imidazole ring. In some embodiments, R₁ and R₂ are joined together to form a pyrrole ring. In some embodiments, R₁ and R₂ are joined together to form a cyclohexene ring. In some embodiments, R₁ and R₂ are joined together to form a pyrazine ring.

In various embodiments, compound of formula I, II, I(a), I(b), I(c), I(d), I(d(i)), I(e), I(e(i)) and/or I(f) is substituted by R₃ and R₄. Single substituents can be present at the ortho, meta, or para positions.

In various embodiments, R₃ and R₄ of formula I, II, I(a), I(b), I(c), I(d), I(d(i)), I(e), I(e(i)) and/or I(f) are each independently H, F, Cl, Br, I, OH, SH, R₈—OH, R₈—SH, —R₈—O—R₁₀, —O—R₈—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl, CH₂—OCH₂—CH₂—O—CH₃), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, substituted or unsubstituted benzyl, (wherein substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof); each represents a separate embodiment of this invention.

In some embodiments, R₃ and R₄ are joint together to form a 5 or 6 membered substituted or unsubstituted, aliphatic or aromatic, carbocyclic or heterocyclic ring. In some embodiments, R₃ and R₄ are joint together to form a 5 or 6 membered carbocyclic ring. In some embodiments, R₃ and R₄ are joined together to form a 5 or 6 membered heterocyclic ring. In some embodiments, R₃ and R₄ are joined together to form a dioxole ring. [1,3]dioxole ring. In some embodiments, R₃ and R₄ are joined together to form a dihydrofuran-2(3H)-one ring. In some embodiments, R₃ and R₄ are joined together to form a furan-2(3H)-one ring. In some embodiments, R₃ and R₄ are joined together to form a benzene ring. In some embodiments, R₃ and R₄ are joint together to form an imidazole ring. In some embodiments, R₃ and R₄ are joined together to form a pyridine ring. In some embodiments, R₃ and R₄ are joined together to form a pyrrole ring. In some embodiments, R₃ and R₄ are joined together to form a cyclohexene ring. In some embodiments, R₃ and R₄ are joined together to form a cyclopentene ring. In some embodiments, R₄ and R₃ are joint together to form a dioxepine ring.

In various embodiments, R₅ of compound of formula I, II, I(a) and I(b) is absent. In other embodiments, R₅ is H, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, or substituted or unsubstituted benzyl; each represents a separate embodiment of this invention. In some embodiments, substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof; each represents a separate embodiment of this invention.

In various embodiments, R₆ and R₇ of compound of formula I and/or II, are each independently H, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted C₃-C₈ heterocyclic ring, substituted or unsubstituted aryl, or substituted or unsubstituted benzyl; each represents a separate embodiment of this invention. In various embodiments, substitutions include: F, Cl, Br, I, C₁-C₅ linear or branched alkyl, OH, alkoxy, N(R)₂, CF₃, aryl, phenyl, heteroaryl, C₃-C₈ cycloalkyl, halophenyl, (benzyloxy)phenyl, CN, NO₂ or any combination thereof; each represents a separate embodiment of this invention.

In various embodiments, n of compound of formula I, II, I(a) and/or I(f) is 0. In some embodiments, n is 0 or 1. In some embodiments, n is between 1 and 3. In some embodiments, n is between 1 and 4. In some embodiments, n is between 0 and 2. In some embodiments, n is between 0 and 3. In some embodiments, n is between 0 and 4. In some embodiments, n is 1. In some embodiments, n is 2. In some embodiments, n is 3. In some embodiments, n is 4.

In various embodiments, m of compound of formula I, II, I(a) and/or I(f) is 0. In some embodiments, m is 0 or 1. In some embodiments, m is between 1 and 3. In some embodiments, m is between 1 and 4. In some embodiments, m is between 0 and 2. In some embodiments, m is between 0 and 3. In some embodiments, m is between 0 and 4. In some embodiments, m is 1. In some embodiments, m is 2. In some embodiments, m is 3. In some embodiments, m is 4.

In various embodiments, 1 of compound of formula I, II, I(a), I(b), I(c), I(d), I(d(i)), I(e), I(e(i)) and/or I(f) is 0. In some embodiments, l is 0 or 1. In some embodiments, l is between 1 and 3. In some embodiments, l is between 1 and 4. In some embodiments, l is 1 or 2. In some embodiments, l is between 0 and 3. In some embodiments, l is between 0 and 4. In some embodiments, l is 1. In some embodiments, l is 2. In some embodiments, l is 3. In some embodiments, l is 4.

In various embodiments, k of compound of formula I, II and I(a) is 0. In some embodiments, k is 0 or 1. In some embodiments, k is between 1 and 3. In some embodiments, k is between 1 and 4. In some embodiments, k is between 0 and 2. In some embodiments, k is between 0 and 3. In some embodiments, k is between 0 and 4. In some embodiments, k is 1. In some embodiments, k is 2. In some embodiments, k is 3. In some embodiments, k is 4.

It is understood that for heterocyclic rings, n, m, l and/or k are limited to the number of available positions for substitution, i.e. to the number of CH or NH groups minus one. Accordingly, if A and/or B rings are, for example, furanyl, thiophenyl or pyrrolyl, n, m, l and k are between 0 and 2; and if A and/or B rings are, for example, oxazolyl, imidazolyl or thiazolyl, n, m, l and k are either 0 or 1; and if A and/or B rings are, for example, oxadiazolyl or thiadiazolyl, n, m, l and k are 0.

In various embodiments, each R₈ of compound of formula I, II, I(a), I(b), I(c), I(d), I(d(i)), I(e), I(e(i)) and/or I(f) is independently CH₂. In some embodiments, R₈ is CH₂CH₂. In some embodiments, R₈ is CH₂CH₂CH₂. In some embodiments, R₈ is CH₂CH₂CH₂CH₂.

In various embodiments, p of compound of formula I, II, I(a), I(b), I(c), I(d), I(d(i)), I(e), I(e(i)) and/or I(f) is 1. In some embodiments, p is 2. In some embodiments, p is 3. In some embodiments, p is 4. In some embodiments, p is 5. In some embodiments, p is between 1 and 3. In some embodiments, p is between 1 and 5. In some embodiments, p is between 1 and 10.

In some embodiments, R₉ of compound of formula I, II, I(a), I(b), I(c), I(d), I(d(i)), I(e), I(e(i)) and/or I(f) is C≡C. In some embodiments, R₉ is C≡C—C≡C. In some embodiments, R₉ is CH═CH. In some embodiments, R₉ is CH═CH—CH═CH.

In some embodiments, q of compound of formula I, II, I(a), I(b), I(c), I(d), I(d(i)), I(e), I(e(i)) and/or I(f) is 2. In some embodiments, q is 4. In some embodiments, q is 6. In some embodiments, q is 8. In some embodiments, q is between 2 and 6.

In various embodiments, R₁₀ of compound of formula I, II, I(a), I(b), I(c), I(d), I(d(i)), I(e), I(e(i)) and/or I(f) is H. In some embodiments, R₁₀ is C₁-C₅ linear or branched alkyl. In some embodiments, R₁₀ is methyl. In some embodiments, R₁₀ is ethyl. In some embodiments, R₁₀ is propyl. In some embodiments, R₁₀ is isopropyl. In some embodiments, R₁₀ is butyl. In some embodiments, R₁₀ is isobutyl. In some embodiments, R₁₀ is t-butyl. In some embodiments, R₁₀ is cyclopropyl. In some embodiments, R₁₀ is pentyl. In some embodiments, R₁₀ is isopentyl. In some embodiments, R₁₀ is neopentyl. In some embodiments, R₁₀ is benzyl. In some embodiments, R₁₀ is C(O)R. In some embodiments, R₁₀ is S(O)₂R.

In various embodiments, R₁₁ of compound of formula I, II, I(a), I(b), I(c), I(d), I(d(i)), I(e), I(e(i)) and/or I(f) is H. In some embodiments, R₁₁ is C₁-C₅ linear or branched alkyl. In some embodiments, R₁₁ is methyl. In some embodiments, R₁₁ is ethyl. In some embodiments, R₁₀ is propyl. In some embodiments, R₁₁ is isopropyl. In some embodiments, R₁₁ is butyl. In some embodiments, R₁₁ is isobutyl. In some embodiments, R₁₁ is t-butyl. In some embodiments, R₁₁ is cyclopropyl. In some embodiments, R₁₁ is pentyl. In some embodiments, R₁₁ is isopentyl. In some embodiments, R₁₁ is neopentyl. In some embodiments, R₁₁ is benzyl. In some embodiments, R₁₁ is C(O)R. In some embodiments, R₁₁ is S(O)₂R.

In some embodiments, R₁₀ and R₁₁ of formula I, II, I(a), I(b), I(c), I(d), I(d(i)), I(e), I(e(i)) and/or I(f) are joint to form a substituted or unsubstituted C₃-C₈ heterocyclic ring. In other embodiments, R₁₀ and R₁₁ are joint to form a piperazine ring. In other embodiments, R₁₀ and R₁₁ are joint to form a piperidine ring. In some embodiments, substitutions include: F, Cl, Br, I, OH, C₁-C₅ linear or branched alkyl, C₁-C₅ linear or branched alkyl-OH (e.g., C(CH₃)₂CH₂—OH, CH₂CH₂—OH), C₃-C₈ heterocyclic ring (e.g., piperidine), alkoxy, N(R)₂, CF₃, aryl, phenyl, halophenyl, (benzyloxy)phenyl, CN, NO₂ or any combination thereof; each represents a separate embodiment according to this invention.

In various embodiments, R₂ of compound of formula I(d), I(d(i)), I(e), and/or I(e(i)) is H. In other embodiments, R₁₂ is F. In other embodiments, R₁₂ is Cl. In other embodiments, R₁₂ is Br. In other embodiments, R₁₂ is I. In other embodiments, R₁₂ is OH. In other embodiments, R₁₂ is SH. In other embodiments, R₁₂ is N(R)₂. In other embodiments, R₁₂ is CF₃. In other embodiments, R₁₂ is CN. In other embodiments, R₁₂ is NO₂. In other embodiments, R₁₂ is C₁-C₅ linear or branched alkyl. In other embodiments, R₁₂ is methyl. In other embodiments, R₁₂ is ethyl. In other embodiments, R₁₂ is CH₂—CH₂—O—CH₂—CH₂—O—CH₃. In other embodiments, R₁₂ is CH₂—O—CH₂—CH₂—O—CH₃. In other embodiments, R₁₂ is C₁-C₅ linear or branched alkoxy. In other embodiments, R₁₂ is C₁-C₅ linear or branched haloalkyl. In other embodiments, R₁₂ is CHF₂. In other embodiments, R₁₂ is CF₃. In other embodiments, R₁₂ is CF₂CH₃. In other embodiments, R₁₂ is CH₂CF₃, CF₂CH₂CH₃. In other embodiments, R₁₂ is CH₂CH₂CF₃, CF₂CH(CH₃)₂. In other embodiments, R₁₂ is CF(CH₃)—CH(CH₃)₂. In other embodiments, R₁₂ is C₃-C₈ substituted or unsubstituted cycloalkyl. In other embodiments, R₁₂ is cyclopropyl. In other embodiments, R₁₂ may be further substituted by at least one selected from: F, Cl, Br, I, OH, SH, C₁-C₅ linear or branched alkyl, OH, alkoxy, N(R)₂, CF₃, phenyl, halophenyl, CN, and NO₂.

In various embodiments, R of compound of formula I, II, I(a), I(b), I(c), I(d), I(d(i)), I(e), I(e(i)) and/or I(f) is H. In other embodiments, R is F. In other embodiments, R is Cl. In other embodiments, R is Br. In other embodiments, R is I. In other embodiments, R is OH. In other embodiments, R is SH. In other embodiments, R is OH. In other embodiments, R is alkoxy. In other embodiments, R is N(R)₂. In other embodiments, R is CF₃. In other embodiments, R is CN. In other embodiments, R is NO₂. In other embodiments, R is C₁-C₅ linear or branched alkyl. In other embodiments, R is methyl. In other embodiments, R is ethyl. In other embodiments, R is CH₂—CH₂—O—CH₂—CH₂—O—CH₃. In other embodiments, R is CH₂—O—CH₂—CH₂—O—CH₃. In other embodiments, R is C₁-C₅ linear or branched alkoxy. In other embodiments, R is C₁-C₅ linear or branched haloalkyl. In other embodiments, R is CHF₂. In other embodiments, R is CF₃. In other embodiments, R is CF₂CH₃. In other embodiments, R is CH₂CF₃, CF₂CH₂CH₃. In other embodiments, R is CH₂CH₂CF₃, CF₂CH(CH₃)₂. In other embodiments, R is CF(CH₃)—CH(CH₃)₂. In other embodiments, R is R₈-aryl. In other embodiments, R is CH₂-Ph. In other embodiments, R is —R₈—O—R₈—O—R₁₀. In other embodiments, R is (CH₂)₂—O—(CH₂)₂—O—CH₃). In other embodiments, R is —R₈—O—R₁₀. In other embodiments, R is —R₈—R₁₀. In other embodiments, R is (CH₂)₂O—CH₃. In other embodiments, R is C₁-C₅ linear or branched alkoxy. In other embodiments, R is phenyl. In other embodiments, R is aryl. In other embodiments, R is heteroaryl. In other embodiments, two gem R substituents are joint together to form a 5 or 6 membered heterocyclic ring.

In various embodiments, Q₁ of compound of formula I, II, I(a) and I(b) is NH. In other embodiments, Q₁ is NR. In other embodiments, Q₁ is N—CH₃. In other embodiments, Q₁ is N—(CH₂)₂—O—CH₃. In other embodiments, Q₁ is N—(CH₂)₂—O—(CH₂)₂O—CH₃. In other embodiments, Q₁ O. In other embodiments, Q₁ is S. In other embodiments, Q₁ is N—OH. In other embodiments, Q₁ is N—OMe.

In various embodiments, Q₂ of compound of formula I, II, I(a), I(b), and I(c) is N. In other embodiments, Q₂ is C(R). In other embodiments, Q₂ is C—CH₃. In other embodiments, Q₂ is C—CHF₂. In other embodiments, Q₂ is C—CF₃. In other embodiments, Q₂ is C—CN. In other embodiments, Q₂ is C—Cl. In other embodiments, Q₂ is C—CH₂—O—CH₂—CH₂—O—CH₃. In other embodiments, Q₂ is C—C—.

In some embodiments, each of X₁, X₂, X₃, X₄, X₅, X₆, X₇, X₈, X₉, X₁₀, X₁₁, X₁₂, X₁₃, X₁₄, and/or X₁₅ of compound of formula I-I(c) or II is independently C. In other embodiments, N.

In some embodiments, at least one of X₁-X₅ is N. In some embodiments, at least two of X₁-X₅ are N. In some embodiments, at least one of X₉-X₁₃ is N. It is understood that if any of X₁-X₁₃ are N, then any of R₁—R₃ cannot be attached thereto.

In various embodiments, G=X of compound of formula I, II, I(a) and I(b) is C═O. In other embodiments, G=X is C═S. In other embodiments, G=X is S═O. In other embodiments, G=X is SO₂. In other embodiments, G=X is CH₂. In other embodiments, G=X is CHR. In other embodiments, G=X is C(R)₂. As used herein, “single or fused aromatic or heteroaromatic ring systems” can be any such ring, including but not limited to phenyl, naphthyl, pyridinyl, (2-, 3-, and 4-pyridinyl), quinolinyl, pyrimidinyl, pyridazinyl, pyrazinyl, triazinyl, tetrazinyl, thiazolyl, isothiazolyl, oxazolyl, isoxazolyl, imidazolyl, 1-methylimidazole, pyrazolyl, pyrrolyl, furanyl, thiophene-yl, quinolinyl, isoquinolinyl, 2,3-dihydroindenyl, indenyl, tetrahydronaphthyl, 3,4-dihydro-2H-benzo[b][1,4]dioxepine benzodioxolyl, benzo[d][1,3]dioxole, tetrahydronaphthyl, indolyl, 1H-indole, isoindolyl, anthracenyl, benzimidazolyl, 2,3-dihydro-1H-benzo[d]imidazolyl, indazolyl, 2H-indazole, triazolyl, 4,5,6,7-tetrahydro-2H-indazole, 3H-indol-3-one, purinyl, benzoxazolyl, 1,3-benzoxazolyl, benzisoxazolyl, benzothiazolyl, 1,3-benzothiazole, 4,5,6,7-tetrahydro-1,3-benzothiazole, quinazolinyl, quinoxalinyl, 1,2,3,4-tetrahydroquinoxaline, 1-(pyridin-1(2H)-yl)ethanone, cinnolinyl, phthalazinyl, quinolinyl, isoquinolinyl, acridinyl, benzofuranyl, 1-benzofuran, isobenzofuranyl, benzofuran-2(3H)-one, benzothiophenyl, benzoxadiazole, benzo[c][1,2,5]oxadiazolyl, benzo[c]thiophenyl, benzodioxolyl, thiadiazolyl, [1,3]oxazolo[4,5-b]pyridine, 1,2,3-, 1,2,4-, 1,2,5- or 1,3,4- oxadiazolyl, imidazo[2,1-b][1,3]thiazole, 4H,5H,6H-cyclopenta[d][1,3]thiazole, 5H,6H,7H,8H-imidazo[1,2-a]pyridine, 7-oxo-6H,7H-[1,3]thiazolo[4,5-d]pyrimidine, [1,3]thiazolo[5,4-b]pyridine, 2H,3H-imidazo[2,1-b][1,3]thiazole, thieno[3,2-d]pyrimidin-4(3H)-one, 4-oxo-4H-thieno[3,2-d][1,3]thiazin, imidazo[1,2-a]pyridine, 1H-imidazo[4,5-b]pyridine, 1H-imidazo[4,5-c]pyridine, 3H-imidazo[4,5-c]pyridine, pyrazolo[1,5-a]pyridine, imidazo[1,2-a]pyrazine, imidazo[1,2-a]pyrimidine, 1H-pyrrolo[2,3-b]pyridine, pyrido[2,3-b]pyrazine, pyrido[2,3-b]pyrazin-3(4H)-one, 4H-thieno[3,2-b]pyrrole, quinoxalin-2(1H)-one, 1H-pyrrolo[3,2-b]pyridine, 7H-pyrrolo[2,3-d]pyrimidine, oxazolo[5,4-b]pyridine, thiazolo[5,4-b]pyridine, thieno[3,2-c]pyridine, 3-methyl-4H-1,2,4-triazole, 5-methyl-1,2,4-oxadiazole etc.

As used herein, the term “alkyl” can be any straight- or branched-chain alkyl group containing up to about 30 carbons unless otherwise specified. In various embodiments, an alkyl includes C₁-C₅ carbons. In some embodiments, an alkyl includes C₁-C₆ carbons. In some embodiments, an alkyl includes C₁-C₅ carbons. In some embodiments, an alkyl includes C₁-C₁₀ carbons. In some embodiments, an alkyl is a C₁-C₁₂ carbons. In some embodiments, an alkyl is a C₁-C₂₀ carbons. In some embodiments, branched alkyl is an alkyl substituted by alkyl side chains of 1 to 5 carbons. In various embodiments, the alkyl group may be unsubstituted. In some embodiments, the alkyl group may be substituted by a halogen, haloalkyl, hydroxyl, alkoxy, carbonyl, amido, alkylamido, dialkylamido, cyano, nitro, CO₂H, amino, alkylamino, dialkylamino, carboxyl, thio, thioalkyl, C₁-C₅ linear or branched haloalkoxy, CF₃, phenyl, halophenyl, (benzyloxy)phenyl, —CH₂CN, NH₂, NH-alkyl, N(alkyl)₂, —OC(O)CF₃, —OCH₂Ph, —NHCO-alkyl, —C(O)Ph, C(O)O-alkyl, C(O)H, —C(O)NH₂ or any combination thereof.

The alkyl group can be a sole substituent, or it can be a component of a larger substituent, such as in an alkoxy, alkoxyalkyl, haloalkyl, arylalkyl, alkylamino, dialkylamino, alkylamido, alkylurea, etc. Preferred alkyl groups are methyl, ethyl, and propyl, and thus halomethyl, dihalomethyl, trihalomethyl, haloethyl, dihaloethyl, trihaloethyl, halopropyl, dihalopropyl, trihalopropyl, methoxy, ethoxy, propoxy, arylmethyl, arylethyl, arylpropyl, methylamino, ethylamino, propylamino, dimethylamino, diethylamino, methylamido, acetamido, propylamido, halomethylamido, haloethylamido, halopropylamido, methyl-urea, ethyl-urea, propyl-urea, 2, 3, or 4-CH₂—C₆H₄—Cl, C(OH)(CH₃)(Ph), etc.

As used herein, the term “aryl” refers to any aromatic ring that is directly bonded to another group and can be either substituted or unsubstituted. The aryl group can be a sole substituent, or the aryl group can be a component of a larger substituent, such as in an arylalkyl, arylamino, arylamido, etc. In some embodiments, the term aryl according to this invention, includes also heteroaryl. Exemplary aryl groups include, without limitation, phenyl, tolyl, xylyl, furanyl, naphthyl, pyridinyl, pyrimidinyl, pyridazinyl, pyrazinyl, triazinyl, thiazolyl, oxazolyl, isooxazolyl, pyrazolyl, imidazolyl, thiophene-yl, pyrrolyl, indolyl, phenylmethyl, phenylethyl, phenylamino, phenylamido, 3-methyl-4H-1,2,4-triazolyl, oxadiazolyl, 5-methyl-1,2,4-oxadiazolyl, isothiazolyl, thiadiazolyl, triazolyl, etc. Substitutions include but are not limited to: F, Cl, Br, I, C₁-C₅ linear or branched alkyl, C₁-C₅ linear or branched haloalkyl, C₁-C₅ linear or branched alkoxy, C₁-C₅ linear or branched haloalkoxy, CF₃, phenyl, halophenyl, CN, NO₂, —CH₂CN, NH₂, NH-alkyl, N(alkyl)₂, hydroxyl, —OC(O)CF₃, —OCH₂Ph, —NHCO-alkyl, COOH, —C(O)Ph, C(O)O-alkyl, C(O)H, —C(O)NH₂ or any combination thereof.

As used herein, the term “alkoxy” refers to an ether group substituted by an alkyl group as defined above. Alkoxy refers both to linear and to branched alkoxy groups. Nonlimiting examples of alkoxy groups are methoxy, ethoxy, propoxy, iso-propoxy, tert-butoxy.

As used herein, the term “aminoalkyl” refers to an amine group substituted by an alkyl group as defined above. Aminoalkyl refers to monoalkylamine, dialkylamine or trialkylamine. Nonlimiting examples of aminoalkyl groups are —N(Me)₂, —NHMe, —NH₃.

A “haloalkyl” group refers, in some embodiments, to an alkyl group as defined above, which is substituted by one or more halogen atoms, e.g. by F, Cl, Br or I. The term “haloalkyl” include but is not limited to fluoroalkyl, i.e., to an alkyl group bearing at least one fluorine atom. Nonlimiting examples of haloalkyl groups are CF₃, CF₂CF₃, CF₂CH₃, CH₂CF₃, CF₂CH₂CH₃, CH₂CH₂CF₃, CF₂CH(CH₃)₂ and CF(CH₃)—CH(CH₃)₂.

A “halophenyl” group refers, in some embodiments, to a phenyl substitutent which is substituted by one or more halogen atoms, e.g. by F, Cl, Br or I. In one embodiment, the halophenyl is 4-chlorophenyl.

An “alkoxyalkyl” group refers, in some embodiments, to an alkyl group as defined above, which is substituted by alkoxy group as defined above, e.g. by methoxy, ethoxy, propoxy, i-propoxy, t-butoxy etc. Nonlimiting examples of alkoxyalkyl groups are —CH₂—O—CH₃, —CH₂—O—CH(CH₃)₂, —CH₂—O—C(CH₃)₃, —CH₂—CH₂—O—CH₃, —CH₂—CH₂—O—CH(CH₃)₂, —CH₂—CH₂—O—C(CH₃)₃.

A “cycloalkyl” or “carbocyclic” group refers, in various embodiments, to a ring structure comprising carbon atoms as ring atoms, which may be either saturated or unsaturated, substituted or unsubstituted, single or fused. In some embodiments the cycloalkyl is a 3-10 membered ring. In some embodiments the cycloalkyl is a 3-12 membered ring. In some embodiments the cycloalkyl is a 6 membered ring. In some embodiments the cycloalkyl is a 5-7 membered ring. In some embodiments the cycloalkyl is a 3-8 membered ring. In some embodiments, the cycloalkyl group may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thio, thioalkyl, C₁-C₅ linear or branched haloalkoxy, CF₃, phenyl, halophenyl, (benzyloxy)phenyl, —CH₂CN, NH₂, NH-alkyl, N(alkyl)₂, —OC(O)CF₃, —OCH₂Ph, —NHCO-alkyl, —C(O)Ph, C(O)O-alkyl, C(O)H, —C(O)NH₂ or any combination thereof. In some embodiments, the cycloalkyl ring may be fused to another saturated or unsaturated cycloalkyl or heterocyclic 3-8 membered ring. In some embodiments, the cycloalkyl ring is a saturated ring. In some embodiments, the cycloalkyl ring is an unsaturated ring. Non limiting examples of a cycloalkyl group comprise cyclohexyl, cyclohexenyl, cyclopropyl, cyclopropenyl, cyclopentyl, cyclopentenyl, cyclopentadienyl, cyclobutyl, cyclobutenyl, cycloctyl, cycloctadienyl (COD), cycloctaene (COE) etc.

A “heterocycle” or “heterocyclic” group refers, in various embodiments, to a ring structure comprising in addition to carbon atoms, sulfur, oxygen, nitrogen or any combination thereof, as part of the ring. A “heteroaromatic ring” refers in various embodiments, to an aromatic ring structure comprising in addition to carbon atoms, sulfur, oxygen, nitrogen or any combination thereof, as part of the ring. In some embodiments the heterocycle or heteroaromatic ring is a 3-10 membered ring. In some embodiments the heterocycle or heteroaromatic ring is a 3-12 membered ring. In some embodiments the heterocycle or heteroaromatic ring is a 6 membered ring. In some embodiments the heterocycle or heteroaromatic ring is a 5-7 membered ring. In some embodiments the heterocycle or heteroaromatic ring is a 3-8 membered ring. In some embodiments, the heterocycle group or heteroaromatic ring may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thio, thioalkyl, C₁-C₅ linear or branched haloalkoxy, CF₃, phenyl, halophenyl, (benzyloxy)phenyl, —CH₂CN, NH₂, NH-alkyl, N(alkyl)₂, —OC(O)CF₃, —OCH₂Ph, —NHCO-alkyl, —C(O)Ph, C(O)O-alkyl, C(O)H, —C(O)NH₂ or any combination thereof. In some embodiments, the heterocycle ring or heteroaromatic ring may be fused to another saturated or unsaturated cycloalkyl or heterocyclic 3-8 membered ring. In some embodiments, the heterocyclic ring is a saturated ring. In some embodiments, the heterocyclic ring is an unsaturated ring. Non limiting examples of a heterocyclic ring or heteroaromatic ring systems comprise pyridine, piperidine, morpholine, piperazine, thiophene, pyrrole, benzodioxole, benzofuran-2(3H)-one, benzo[d][1,3]dioxole, indole, oxazole, isoxazole, imidazole and 1-methylimidazole, furane, triazole, pyrimidine, pyrazine, oxacyclobutane (1 or 2-oxacyclobutane), naphthalene, tetrahydrothiophene 1,1-dioxide, thiazole, benzimidazole, piperidine, 1-methylpiperidine, isoquinoline, 1,3-dihydroisobenzofuran, benzofuran, 3-methyl-4H-1,2,4-triazole, oxadiazolyl, 5-methyl-1,2,4-oxadiazole, pyrazole, isothiazole, thiadiazole, tetrahydrofurane, oxazolone, oxazolidone, thiazolone, isothiazolinone, isoxazolidinone, imidazolidinone, pyrazolone, 2H-pyrrol-2-one, furanone, thiophenone, thiane 1,1-dioxide, triazolopyrimidine, 6,7-dihydro-5H-pyrazolo[5,1-b][1,3]oxazine or indole.

In various embodiments, this invention provides a compound of this invention or its isomer, metabolite, pharmaceutically acceptable salt, pharmaceutical product, tautomer, hydrate, N-oxide, reverse amide analog, prodrug, isotopic variant (deuterated analog), PROTAC, polymorph, or crystal or combinations thereof. In various embodiments, this invention provides an isomer of the compound of this invention. In some embodiments, this invention provides a metabolite of the compound of this invention. In some embodiments, this invention provides a pharmaceutically acceptable salt of the compound of this invention. In some embodiments, this invention provides a pharmaceutical product of the compound of this invention. In some embodiments, this invention provides a tautomer of the compound of this invention. In some embodiments, this invention provides a hydrate of the compound of this invention. In some embodiments, this invention provides an N-oxide of the compound of this invention. In some embodiments, this invention provides a reverse amide analog of the compound of this invention. In some embodiments, this invention provides a prodrug of the compound of this invention. In some embodiments, this invention provides an isotopic variant (including but not limited to deuterated analog) of the compound of this invention. In some embodiments, this invention provides a PROTAC (Proteolysis targeting chimera) of the compound of this invention. In some embodiments, this invention provides a polymorph of the compound of this invention. In some embodiments, this invention provides a crystal of the compound of this invention. In some embodiments, this invention provides composition comprising a compound of this invention, as described herein, or, In some embodiments, a combination of an isomer, metabolite, pharmaceutically acceptable salt, pharmaceutical product, tautomer, hydrate, N-oxide, reverse amide analog, prodrug, isotopic variant (deuterated analog), PROTAC, polymorph, or crystal of the compound of this invention.

In various embodiments, the term “isomer” includes, but is not limited to, stereoisomers including optical isomers and analogs, structural isomers and analogs, conformational isomers and analogs, and the like. In some embodiments, the isomer is a stereoisomer. In another embodiment, the isomer is an optical isomer.

Certain compounds of the present invention may exist in particular geometric or stereoisomeric forms. The present invention contemplates all such compounds, including cis- and trans-isomers, R- and S-enantiomers, diastereomers, the racemic mixtures thereof, and other mixtures thereof, as falling within the scope of the invention. Additional asymmetric carbon atoms may be present in a substituent such as an alkyl group. All such isomers, as well as mixtures thereof, are included in this invention.

In various embodiments, this invention encompasses the use of various stereoisomers of the compounds of the invention. It will be appreciated by those skilled in the art that the compounds of the present invention may contain at least one chiral center. Accordingly, the compounds used in the methods of the present invention may exist in, and be isolated in, optically-active or racemic forms. The compounds according to this invention may further exist as stereoisomers which may be also optically-active isomers (e.g., enantiomers such as (R) or (S)), as enantiomerically enriched mixtures, racemic mixtures, or as single diastereomers, diastereomeric mixtures, or any other stereoisomers, including but not limited to: (R)(R), (R)(S), (S)(S), (S)(R), (R)(R)(R), (R)(R)(S), (R)(S)(R), (S)(R)(R), (R)(S)(S), (S)(R)(S), (S)(S)(R) or (S)(S)(S) stereoisomers. Some compounds may also exhibit polymorphism. It is to be understood that the present invention encompasses any racemic, optically-active, polymorphic, or stereoisomeric form, or mixtures thereof, which form possesses properties useful in the treatment of the various conditions described herein.

In various embodiments, Compounds 132 and 134 are stereoisomers. In various embodiments, Compounds 132 and 134 are optical isomers. In one embodiment, Compounds 132 and 134 are enantiomers. In one embodiment, Compounds 132 and 134 are optically active. In one embodiment, Compounds 132 is different enantiomer than compound 134. In one embodiment, compound 132 is the R isomer and compound 134 is the S isomer. In another embodiment, compound 132 is the S isomer and compound 134 is the R isomer. In one embodiment, the * on the piperidine carbon of compounds 132 and 134 represents a chiral center, which in some embodiments refer to the R isomer in compound 132 and the S isomer in compound 134, and in other embodiments refers to the S isomer in compound 132 and the R isomer in compound 134. The chiral center of compound 132 can be either in R or S configuation as long as the corresponding chiral center in compound 134 is in S or R configuration respectively.

It is well known in the art how to prepare optically active forms (for example, by resolution of the racemic form by recrystallization techniques, by synthesis from optically-active starting materials, by chiral synthesis, or by chromatographic separation using a chiral stationary phase).

The compounds of the present invention can also be present in the form of a racemic mixture, containing substantially equivalent amounts of stereoisomers. In some embodiments, the compounds of the present invention can be prepared or otherwise isolated, using known procedures, to obtain a stereoisomer substantially free of its corresponding stereoisomer (i.e., substantially pure). By substantially pure, it is intended that a stereoisomer is at least about 95% pure, more preferably at least about 98% pure, most preferably at least about 99% pure.

Compounds of the present invention can also be in the form of a hydrate, which means that the compound further includes a stoichiometric or non-stoichiometric amount of water bound by non-covalent intermolecular forces.

As used herein, when some chemical functional group (e.g. alkyl or aryl) is said to be “substituted”, it is herein defined that one or more substitutions are possible.

Compounds of the present invention may exist in the form of one or more of the possible tautomers and depending on the conditions it may be possible to separate some or all of the tautomers into individual and distinct entities. It is to be understood that all of the possible tautomers, including all additional enol and keto tautomers and/or isomers are hereby covered. For example, the following tautomers, but not limited to these, are included: Tautomerization of the imidazole ring

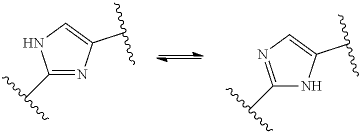 [SMILES: *c1c[nH]c(*)n1 ; *c1cnc(*)[nH]1]

### Tautomerization of the Pyrazolone Ring:

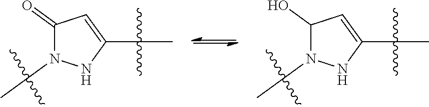 [SMILES: *c1cc(=O)n(*)[nH]1 ; *C1=CC(O)N(*)N1]

The invention includes “pharmaceutically acceptable salts” of the compounds of this invention, which may be produced, by reaction of a compound of this invention with an acid or base. Certain compounds, particularly those possessing acid or basic groups, can also be in the form of a salt, preferably a pharmaceutically acceptable salt. The term “pharmaceutically acceptable salt” refers to those salts that retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxylic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. Other salts are known to those of skill in the art and can readily be adapted for use in accordance with the present invention.

Suitable pharmaceutically acceptable salts of amines of compounds the compounds of this invention may be prepared from an inorganic acid or from an organic acid. In various embodiments, examples of inorganic salts of amines are bisulfates, borates, bromides, chlorides, hemisulfates, hydrobromates, hydrochlorates, 2-hydroxyethylsulfonates (hydroxyethanesulfonates), iodates, iodides, isothionates, nitrates, persulfates, phosphate, sulfates, sulfamates, sulfanilates, sulfonic acids (alkylsulfonates, arylsulfonates, halogen substituted alkylsulfonates, halogen substituted arylsulfonates), sulfonates and thiocyanates.

In various embodiments, examples of organic salts of amines may be selected from aliphatic, cycloaliphatic, aromatic, araliphatic, heterocyclic, carboxylic and sulfonic classes of organic acids, examples of which are acetates, arginines, aspartates, ascorbates, adipates, anthranilates, algenates, alkane carboxylates, substituted alkane carboxylates, alginates, benzenesulfonates, benzoates, bisulfates, butyrates, bicarbonates, bitartrates, citrates, camphorates, camphorsulfonates, cyclohexylsulfamates, cyclopentanepropionates, calcium edetates, camsylates, carbonates, clavulanates, cinnamates, dicarboxylates, digluconates, dodecylsulfonates, dihydrochlorides, decanoates, enanthuates, ethanesulfonates, edetates, edisylates, estolates, esylates, fumarates, formates, fluorides, galacturonates gluconates, glutamates, glycolates, glucorate, glucoheptanoates, glycerophosphates, gluceptates, glycollylarsanilates, glutarates, glutamate, heptanoates, hexanoates, hydroxymaleates, hydroxycarboxlic acids, hexylresorcinates, hydroxybenzoates, hydroxynaphthoates, hydrofluorates, lactates, lactobionates, laurates, malates, maleates, methylenebis(beta-oxynaphthoate), malonates, mandelates, mesylates, methane sulfonates, methylbromides, methylnitrates, methylsulfonates, monopotassium maleates, mucates, monocarboxylates, naphthalenesulfonates, 2-naphthalenesulfonates, nicotinates, nitrates, napsylates, N-methylglucamines, oxalates, octanoates, oleates, pamoates, phenylacetates, picrates, phenylbenzoates, pivalates, propionates, phthalates, phenylacetate, pectinates, phenylpropionates, palmitates, pantothenates, polygalacturates, pyruvates, quinates, salicylates, succinates, stearates, sulfanilate, subacetates, tartrates, theophyllineacetates, p-toluenesulfonates (tosylates), trifluoroacetates, terephthalates, tannates, teoclates, trihaloacetates, triethiodide, tricarboxylates, undecanoates and valerates.

In various embodiments, examples of inorganic salts of carboxylic acids or hydroxyls may be selected from ammonium, alkali metals to include lithium, sodium, potassium, cesium; alkaline earth metals to include calcium, magnesium, aluminium; zinc, barium, cholines, quaternary ammoniums.

In some embodiments, examples of organic salts of carboxylic acids or hydroxyl may be selected from arginine, organic amines to include aliphatic organic amines, alicyclic organic amines, aromatic organic amines, benzathines, t-butylamines, benethamines (N-benzylphenethylamine), dicyclohexylamines, dimethylamines, diethanolamines, ethanolamines, ethylenediamines, hydrabamines, imidazoles, lysines, methylamines, meglamines, N-methyl-D-glucamines, N,N′-dibenzylethylenediamines, nicotinamides, organic amines, ornithines, pyridines, picolies, piperazines, procain, tris(hydroxymethyl)methylamines, triethylamines, triethanolamines, trimethylamines, tromethamines and ureas.

In various embodiments, the salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of a existing salt for another ion or suitable ion-exchange resin.

### Pharmaceutical Composition

Another aspect of the present invention relates to a pharmaceutical composition including a pharmaceutically acceptable carrier and a compound according to the aspects of the present invention. The pharmaceutical composition can contain one or more of the above-identified compounds of the present invention. Typically, the pharmaceutical composition of the present invention will include a compound of the present invention or its pharmaceutically acceptable salt, as well as a pharmaceutically acceptable carrier. The term “pharmaceutically acceptable carrier” refers to any suitable adjuvants, carriers, excipients, or stabilizers, and can be in solid or liquid form such as, tablets, capsules, powders, solutions, suspensions, or emulsions.

Typically, the composition will contain from about 0.01 to 99 percent, preferably from about 20 to 75 percent of active compound(s), together with the adjuvants, carriers and/or excipients. While individual needs may vary, determination of optimal ranges of effective amounts of each component is within the skill of the art. Typical dosages comprise about 0.01 to about 100 mg/kg body wt. The preferred dosages comprise about 0.1 to about 100 mg/kg body wt. The most preferred dosages comprise about 1 to about 100 mg/kg body wt. Treatment regimen for the administration of the compounds of the present invention can also be determined readily by those with ordinary skill in art. That is, the frequency of administration and size of the dose can be established by routine optimization, preferably while minimizing any side effects.

The solid unit dosage forms can be of the conventional type. The solid form can be a capsule and the like, such as an ordinary gelatin type containing the compounds of the present invention and a carrier, for example, lubricants and inert fillers such as, lactose, sucrose, or cornstarch. In some embodiments, these compounds are tabulated with conventional tablet bases such as lactose, sucrose, or cornstarch in combination with binders like acacia, cornstarch, or gelatin, disintegrating agents, such as cornstarch, potato starch, or alginic acid, and a lubricant, like stearic acid or magnesium stearate.

The tablets, capsules, and the like can also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it can contain, in addition to materials of the above type, a liquid carrier such as a fatty oil.

Various other materials may be present as coatings or to modify the physical form of the dosage unit. For instance, tablets can be coated with shellac, sugar, or both. A syrup can contain, in addition to active ingredient, sucrose as a sweetening agent, methyl and propylparabens as preservatives, a dye, and flavoring such as cherry or orange flavor.

For oral therapeutic administration, these active compounds can be incorporated with excipients and used in the form of tablets, capsules, elixirs, suspensions, syrups, and the like. Such compositions and preparations should contain at least 0.1% of active compound. The percentage of the compound in these compositions can, of course, be varied and can conveniently be between about 2% to about 60% of the weight of the unit. The amount of active compound in such therapeutically useful compositions is such that a suitable dosage will be obtained. Preferred compositions according to the present invention are prepared so that an oral dosage unit contains between about 1 mg and 800 mg of active compound.

The active compounds of the present invention may be orally administered, for example, with an inert diluent, or with an assimilable edible carrier, or they can be enclosed in hard or soft shell capsules, or they can be compressed into tablets, or they can be incorporated directly with the food of the diet.

The pharmaceutical forms suitable for injectable use include sterile aqueous solutions or dispersions and sterile powders for the extemporaneous preparation of sterile injectable solutions or dispersions. In all cases, the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds or pharmaceutical compositions of the present invention may also be administered in injectable dosages by solution or suspension of these materials in a physiologically acceptable diluent with a pharmaceutical adjuvant, carrier or excipient. Such adjuvants, carriers and/or excipients include, but are not limited to, sterile liquids, such as water and oils, with or without the addition of a surfactant and other pharmaceutically and physiologically acceptable components. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

These active compounds may also be administered parenterally. Solutions or suspensions of these active compounds can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

In various embodiments, the compounds of this invention are administered in combination with an agent treating fibrosis. In some embodiment, the agent treating lung fibrosis is at least one selected from: pirfenidone and Nintedanib. Other examples of agents which can be useful in treating lung fibrosis including IPF, in combination with compound of the invention, include but are not limited to: Pioglitazone, Tralokinumab, Lebrikizumab, FG-3019, Simtuzumab, STX-100, BMS-986020, Rituximab, Carbon Monoxide, Azithromycin, and Cotrimoxazole. In various embodiments, the compounds of this invention are administered in combination with an agent treating NASH.

When administering the compounds of the present invention, they can be administered systemically or, alternatively, they can be administered directly to a specific site where fibrosis is present. Thus, administering can be accomplished in any manner effective for delivering the compounds or the pharmaceutical compositions to the fibrotic cells. Exemplary modes of administration include, without limitation, administering the compounds or compositions orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes.

### Biological Activity

In various embodiments, the invention provides compounds and compositions, including any embodiment described herein, for use in any of the methods of this invention. In various embodiments, use of a compound of this invention or a composition comprising the same, will have utility in inhibiting, suppressing, enhancing or stimulating a desired response in a subject, as will be understood by one skilled in the art. In some embodiments, the compositions may further comprise additional active ingredients, whose activity is useful for the particular application for which the compound of this invention is being administered.

The invention relates to the treatment, inhibition and reduction of fibrosis, including lung and hepatic fibrosis. More specifically, embodiments of the invention provide compositions and methods useful for the treatment and inhibition of fibrotic disorders, lung fibrosis, Idiotypic pulmonary fibrosis (IPF), hepato-fibrotic conditions associated with Non-Alcoholic Fatty Liver Disease (NAFLD) and Non-Alcoholic Steatohepatitis (NASH), employing the use of a compound according to this invention or a pharmaceutically acceptable salt thereof. In another embodiment, the human subject is afflicted with lung fibrosis. In another embodiment, the human subject is afflicted with Idiotypic pulmonary fibrosis (IPF). In another embodiment, the human subject is afflicted with Non-Alcoholic Fatty Liver Disease (NAFLD). In another embodiment, the human subject is afflicted with Non-Alcoholic Steatohepatitis (NASH). In another embodiment, the human subject is not afflicted with Non-Alcoholic Steatohepatitis (NASH).

In various conditions, the formation of fibrotic tissue is characterized by the deposition of abnormally large amounts of collagen. The synthesis of collagen is also involved in a number of other pathological conditions. For example, clinical conditions and disorders associated with primary or secondary fibrosis, such as systemic sclerosis, graft-versus host disease (GVHD), pulmonary fibrosis and autoimmune disorders, are distinguished by excessive production of connective tissue, which results in the destruction of normal tissue architecture and function. These diseases can best be interpreted in terms of perturbations in cellular functions, a major manifestation of which is excessive collagen synthesis and deposition. The role of collagen in fibrosis has prompted attempts to develop drugs that inhibit its accumulation.

Excessive accumulation of collagen is the major pathologic feature in a variety of clinical conditions characterized by tissue fibrosis. These conditions include localized processes, as for example, pulmonary fibrosis and liver cirrhosis, or more generalized processes, like progressive systemic sclerosis. Collagen deposition is a feature of different forms of dermal fibrosis, which in addition to scleroderma, include localized and generalized morphea, keloids, hypertrophic scars, familial cutaneous collagenoma and connective tissue nevi of the collagen type. Recent advances in the understanding of the normal biochemistry of collagen have allowed us to define specific levels of collagen biosynthesis and degradation at which a pharmacologic intervention could lead to reduced collagen deposition in the tissues. Such compounds could potentially provide us with novel means to reduce the excessive collagen accumulation in diseases.

Accordingly, in various embodiments, this invention is directed to a method of treating, suppressing, reducing the severity, reducing the risk of developing or inhibiting fibrosis in a subject, comprising administering a compound according to this invention, to a subject suffering from fibrosis under conditions effective to treat, suppress, reduce the severity, reduce the risk of developing, or inhibit fibrosis in said subject. In some embodiments, the fibrosis is systemic. In some embodiments, the fibrosis is organ specific. In some embodiments, the fibrosis is a result of wound healing. In some embodiments, the fibrosis is a result of scarring. In some embodiments, the fibrosis is primary or secondary fibrosis. In some embodiments, the fibrosis is a result of systemic sclerosis, progressive systemic sclerosis, graft-versus host disease (GVHD), pulmonary fibrosis, autoimmune disorders, or any combination thereof; each represents a separate embodiment according to this invention. In another embodiment, the human subject is afflicted with lung fibrosis. In another embodiment, the human subject is afflicted with Idiotypic pulmonary fibrosis (IPF). In some embodiments, the fibrosis is pulmonary fibrosis. In some embodiments, the subject has a liver cirrhosis. In some embodiments, the fibrosis is hepatic fibrosis, lung fibrosis or dermal fibrosis. In some embodiments, the dermal fibrosis is scleroderma. In some embodiments, the dermal fibrosis is a result of a localized or generalized morphea, keloids, hypertrophic scars, familial cutaneous collagenoma, connective tissue nevi of the collagen type, or any combination thereof; each represents a separate embodiment according to this invention. In some embodiments, the fibrosis results from tissue injury, inflammation, oxidative stress or any combination thereof; each represents a separate embodiment according to this invention. In some embodiments, the fibrosis is gingival fibromatosis. In some embodiments, the compound is a Collagen I translation inhibitor. In some embodiments, the compound is any one of the compounds listed in Table 1; each compound represents a separate embodiment according to this invention.

Human fibrotic diseases constitute a major health problem worldwide owing to the large number of affected individuals, the incomplete knowledge of the fibrotic process pathogenesis, the marked heterogeneity in their etiology and clinical manifestations, the absence of appropriate and fully validated biomarkers, and, most importantly, the current void of effective disease-modifying therapeutic agents. The fibrotic disorders encompass a wide spectrum of clinical entities including systemic fibrotic diseases such as systemic sclerosis (SSc), sclerodermatous graft vs. host disease, and nephrogenic systemic fibrosis, as well as numerous organ-specific disorders including radiation-induced fibrosis and cardiac, pulmonary, lung, liver, and kidney fibrosis. Although their causative mechanisms are quite diverse and, in several instances have remained elusive, these diseases share the common feature of an uncontrolled and progressive accumulation of fibrotic tissue in affected organs causing their dysfunction and ultimate failure. Despite the remarkable heterogeneity in the etiologic mechanisms responsible for the development of fibrotic diseases and in their clinical manifestations, numerous studies have identified activated myofibroblasts as the common cellular element ultimately responsible for the replacement of normal tissues with nonfunctional fibrotic tissue.

In various embodiments, this invention is directed to a method of treating, suppressing, reducing the severity, reducing the risk of developing or inhibiting systemic fibrotic disease in a subject, comprising administering a compound according to this invention, to a subject suffering from a systemic fibrotic disease under conditions effective to treat, suppress, reduce the severity, reduce the risk of developing, or inhibit the systemic fibrotic disease in said subject. In some embodiments, the systemic fibrotic disease is systemic sclerosis. In some embodiments, the systemic fibrotic disease is multifocal fibrosclerosis (IgG4-associated fibrosis). In some embodiments, the systemic fibrotic disease is nephrogenic systemic fibrosis. In some embodiments, the systemic fibrotic disease is sclerodermatous graft vs. host disease.

In various embodiments, this invention is directed to a method of treating, suppressing, reducing the severity, reducing the risk of developing or inhibiting an organ-specific fibrotic disease in a subject, comprising administering a compound according to this invention, to a subject suffering from an organ-specific fibrotic disease under conditions effective to treat, suppress, reduce the severity, reduce the risk of developing, or inhibit the organ-specific fibrotic disease in said subject.

In some embodiments, the organ-specific fibrotic disease is lung fibrosis. In some embodiments, the organ-specific fibrotic disease is Idiotypic pulmonary fibrosis (IPF).

In some embodiments, the organ-specific fibrotic disease is cardiac fibrosis. In some embodiments, the cardiac fibrosis is hypertension-associated cardiac fibrosis. In some embodiments, the cardiac fibrosis is post-myocardial infarction. In some embodiments, the cardiac fibrosis is chagas disease-induced myocardial fibrosis.

In some embodiments, the organ-specific fibrotic disease is kidney fibrosis. In some embodiments, the kidney fibrosis is diabetic and hypertensive nephropathy. In some embodiments, the kidney fibrosis is urinary tract obstruction-induced kidney fibrosis. In some embodiments, the kidney fibrosis is inflammatory/autoimmune-induced kidney fibrosis. In some embodiments, the kidney fibrosis is aristolochic acid nephropathy. In some embodiments, the kidney fibrosis is polycystic kidney disease.

In various embodiments, this invention is directed to a method of treating, suppressing, reducing the severity, reducing the risk of developing or inhibiting cardiac fibrosis in a subject, comprising administering a compound of this invention, to a subject suffering from cardiac fibrosis under conditions effective to treat, suppress, reduce the severity, reduce the risk of developing, or inhibit cardiac fibrosis in said subject. In some embodiments, the compound is a Collagen I translation inhibitor. In some embodiments, the compound is any one of the compounds listed in Table 1; each compound represents a separate embodiment according to this invention.

In some embodiments, the organ-specific fibrotic disease is pulmonary fibrosis. In some embodiments, the pulmonary fibrosis is idiopathic pulmonary fibrosis. In some embodiments, the pulmonary fibrosis is silica-induced pneumoconiosis (silicosis). In some embodiments, the pulmonary fibrosis is asbestos-induced pulmonary fibrosis (asbestosis). In some embodiments, the pulmonary fibrosis is chemotherapeutic agent-induced pulmonary fibrosis.

In some embodiments, the organ-specific fibrotic disease is liver and portal vein fibrosis. In some embodiments, the liver and portal vein fibrosis is alcoholic and nonalcoholic liver fibrosis. In some embodiments, the liver and portal vein fibrosis is hepatitis C-induced liver fibrosis. In some embodiments, the liver and portal vein fibrosis is primary biliary cirrhosis. In some embodiments, the liver and portal vein fibrosis is parasite-induced liver fibrosis (schistosomiasis).

In some embodiments, the organ-specific fibrotic disease is radiation-induced fibrosis (various organs). In some embodiments, the organ-specific fibrotic disease is bladder fibrosis. In some embodiments, the organ-specific fibrotic disease is intestinal fibrosis. In some embodiments, the organ-specific fibrotic disease is peritoneal sclerosis.

In some embodiments, the organ-specific fibrotic disease is diffuse fasciitis. In some embodiments, the diffuse fasciitis is localized scleroderma, keloids. In some embodiments, the diffuse fasciitis is dupuytren's disease. In some embodiments, the diffuse fasciitis is peyronie's disease. In some embodiments, the diffuse fasciitis is myelofibrosis. In some embodiments, the diffuse fasciitis is oral submucous fibrosis.

In some embodiments, the organ-specific fibrotic disease is a result of wound healing. In some embodiments, the organ-specific fibrotic disease is a result of scarring.

Fibrosis of the liver, also referred to herein as hepatic fibrosis, may be caused by various types of chronic liver injury, especially if an inflammatory component is involved. Self-limited, acute liver injury (e.g., acute viral hepatitis A), even when fulminant, does not necessarily distort the scaffolding architecture and hence does not typically cause fibrosis, despite loss of hepatocytes. However, factors such as chronic alcoholism, malnutrition, hemochromatosis, and exposure to poisons, toxins or drugs, may lead to chronic liver injury and hepatic fibrosis due to exposure to hepatotoxic chemical substances. Hepatic scarring, caused by surgery or other forms of injury associated with mechanical biliary obstruction, may also result in liver fibrosis.

In various embodiments, this invention is directed to a method of treating, suppressing, reducing the severity, reducing the risk of developing or inhibiting hepatic fibrosis in a subject, comprising administering a compound of this invention, to a subject suffering from hepatic fibrosis under conditions effective to treat, suppress, reduce the severity, reduce the risk of developing, or inhibit hepatic fibrosis in said subject. In some embodiments, the hepatic fibrosis results from hepatic scarring. In some embodiments, the hepatic fibrosis results from chronic liver injury. In some embodiments, the chronic liver injury results from chronic alcoholism, malnutrition, hemochromatosis, exposure to poisons, toxins or drugs; each represents a separate embodiment according to this invention. In some embodiments, the subject has a liver cirrhosis. In some embodiments, the compound is a Collagen I translation inhibitor. In some embodiments, the compound is any one of the compounds listed in Table 1; each compound represents a separate embodiment according to this invention.

Fibrosis itself is not necessarily symptomatic, however it can lead to the development of portal hypertension, in which scarring distorts blood flow through the liver, or cirrhosis, in which scarring results in disruption of normal hepatic architecture and liver dysfunction. The extent of each of these pathologies determines the clinical manifestation of hepato-fibrotic disorders. For example, congenital hepatic fibrosis affects portal vein branches, largely sparing the parenchyma. The result is portal hypertension with sparing of hepatocellular function.

In various embodiments, this invention is directed to a method of treating, suppressing, reducing the severity, reducing the risk of developing or inhibiting an hepato-fibrotic disorder in a subject, comprising administering a compound of this invention, to a subject suffering from hepato-fibrotic disorder under conditions effective to treat, suppress, reduce the severity, reduce the risk of developing, or inhibit the hepato-fibrotic disorder in said subject. In some embodiments, the hepato-fibrotic disorder is: portal hypertension, cirrhosis, congenital hepatic fibrosis or any combination thereof; each represents a separate embodiment according to this invention. In some embodiments, the compound is a Collagen I translation inhibitor. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting portal hypertension in a subject, comprising administering a compound of this invention, to a subject suffering from portal hypertension under conditions effective to treat, suppress, reduce the severity, reduce the risk of developing, or inhibit portal hypertension in said subject. In some embodiments, the compound is a Collagen I translation inhibitor. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting cirrhosis in a subject, comprising administering a compound of this invention, to a subject suffering from cirrhosis under conditions effective to treat, suppress, reduce the severity, reduce the risk of developing, or inhibit cirrhosis in said subject. In some embodiments, the cirrhosis is a result of hepatitis. In some embodiments, the cirrhosis is a result of alcoholism. In some embodiments, the compound is a Collagen I translation inhibitor. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting human alcoholism in a subject, comprising administering a compound of this invention, to a subject suffering from alcoholism under conditions effective to treat, suppress, reduce the severity, reduce the risk of developing, or inhibit alcoholism in said subject. In some embodiments, the compound is a Collagen I translation inhibitor. In some embodiments, the compound is any one of the compounds listed in Table 1; each compound represents a separate embodiment according to this invention.

Non-alcoholic steatohepatitis (NASH) and alcoholic steatohepatitis (ASH) have a similar pathogenesis and histopathology but a different etiology and epidemiology. NASH and ASH are advanced stages of non-alcoholic fatty liver disease (NAFLD) and alcoholic fatty liver disease (AFLD). NAFLD is characterized by excessive fat accumulation in the liver (steatosis), without any other evident causes of chronic liver diseases (viral, autoimmune, genetic, etc.), and with an alcohol consumption Z 20-30 g/day. On the contrary, AFLD is defined as the presence of steatosis and alcohol consumption >20-30 g/day.

In various embodiments, this invention is directed to a method of treating, suppressing, reducing the severity, reducing the risk of developing or inhibiting Non-alcoholic steatohepatitis (NASH) in a subject, comprising administering a compound of this invention, to a subject suffering from Non-alcoholic steatohepatitis (NASH) under conditions effective to treat, suppress, reduce the severity, reduce the risk of developing, or inhibit Non-alcoholic steatohepatitis (NASH) in said subject. In some embodiments, the compound is a Collagen I translation inhibitor. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting alcoholic steatohepatitis (ASH) in a subject, comprising administering a compound of this invention, to a subject suffering from alcoholic steatohepatitis (ASH) under conditions effective to treat, suppress, reduce the severity, reduce the risk of developing, or inhibit alcoholic steatohepatitis (ASH) in said subject. In some embodiments, the compound is a Collagen I translation inhibitor. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting non-alcoholic fatty liver disease (NAFLD) in a subject, comprising administering a compound of this invention, to a subject suffering from non-alcoholic fatty liver disease (NAFLD) under conditions effective to treat, suppress, reduce the severity, reduce the risk of developing, or inhibit non-alcoholic fatty liver disease (NAFLD) in said subject. In some embodiments, the compound is a Collagen I translation inhibitor. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting alcoholic fatty liver disease (AFLD) in a subject, comprising administering a compound of this invention, to a subject suffering from alcoholic fatty liver disease (AFLD) under conditions effective to treat, suppress, reduce the severity, reduce the risk of developing, or inhibit alcoholic fatty liver disease (AFLD) in said subject. In some embodiments, the compound is a Collagen I translation inhibitor. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting lung fibrosis in a subject, comprising administering a compound of this invention, to a subject suffering from lung fibrosis under conditions effective to treat, suppress, reduce the severity, reduce the risk of developing, or inhibit lung fibrosis in said subject. In some embodiments, the compound is a Collagen I translation inhibitor. In some embodiments, the compound is any one of the compounds listed in Table 1; each compound represents a separate embodiment according to this invention.

Idiopathic pulmonary fibrosis (IPF) is an aging-associated recalcitrant lung disease with historically limited therapeutic options. The recent approval of two drugs, pirfenidone and nintedanib, by the United States Food and Drug Administration (FDA) in 2014 has heralded a new era in its management. Both drugs demonstrated efficacy in Phase III clinical trials by retarding the rate of progression of IPF; neither drug appears to be able to completely arrest disease progression. Advances in the understanding of IPF pathobiology have led to an unprecedented expansion in the number of potential therapeutic targets. Drugs targeting several of these are under investigation in various stages of clinical development.

In various embodiments, this invention is directed to a method of treating, suppressing, reducing the severity, reducing the risk of developing or inhibiting Idiopathic pulmonary fibrosis (IPF) in a subject, comprising administering a compound of this invention, to a subject suffering from Idiopathic pulmonary fibrosis (IPF) under conditions effective to treat, suppress, reduce the severity, reduce the risk of developing, or inhibit Idiopathic pulmonary fibrosis (IPF) in said subject. In some embodiments, the compound is a Collagen I translation inhibitor. In some embodiments, the compound is any one of the compounds listed in Table 1; each compound represents a separate embodiment according to this invention. In some embodiments, the compound is administered in combination with an agent treating IPF. In some embodiments, the compound is administered in combination with pirfenidone, nintedanib, or combination thereof; each represents a separate embodiment according to this invention.

In various embodiments, this invention is directed to a method of treating, suppressing, reducing the severity, reducing the risk of developing or inhibiting dermal fibrosis in a subject, comprising administering a compound of this invention, to a subject suffering from dermal fibrosis under conditions effective to treat, suppress, reduce the severity, reduce the risk of developing, or inhibit dermal fibrosis in said subject. In some embodiments, the dermal fibrosis is scleroderma. In some embodiments, the dermal fibrosis is a result of a localized or generalized morphea, keloids, hypertrophic scars, familial cutaneous collagenoma, connective tissue nevi of the collagen type, or any combination thereof; each represents a separate embodiment according to this invention. In some embodiments, the compound is a Collagen I translation inhibitor. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting scleroderma in a subject, comprising administering a compound of this invention, to a subject suffering from scleroderma under conditions effective to treat, suppress, reduce the severity, reduce the risk of developing, or inhibit scleroderma in said subject. In some embodiments, the compound is a Collagen I translation inhibitor. In some embodiments, the compound is any one of the compounds listed in Table 1; each compound represents a separate embodiment according to this invention.

In various embodiments, this invention is directed to a method of inhibiting Collagen I (Col I) over production in a subject, comprising administering a compound of this invention, to a subject suffering from Collagen I (Col I) over production under conditions effective to inhibit Collagen I (Col I) over production in said subject. In some embodiments, the compound is a Collagen I translation inhibitor. In some embodiments, the compound is any one of the compounds listed in Table 1; each compound represents a separate embodiment according to this invention.

In some embodiments, this invention is directed to a method of treating, suppressing, reducing the severity, reducing the risk of developing or inhibiting an autoimmune disease or disorder in a subject, comprising administering a compound of this invention, to a subject suffering from an autoimmune disease or disorder under conditions effective to treat, suppress, reduce the severity, reduce the risk of developing, or inhibit the autoimmune disease or disorder in said subject. In some embodiments, the compound is a Collagen I translation inhibitor. In some embodiments, the compound is any one of the compounds listed in Table 1; each compound represents a separate embodiment according to this invention.

As used herein, subject or patient refers to any mammalian patient, including without limitation, humans and other primates, dogs, cats, horses, cows, sheep, pigs, rats, mice, and other rodents. In various embodiments, the subject is male. In some embodiments, the subject is female. In some embodiments, while the methods as described herein may be useful for treating either males or females.

The following examples are presented in order to more fully illustrate the preferred embodiments of the invention. They should in no way, however, be construed as limiting the broad scope of the invention.

### EXAMPLES

### General

All compounds were profiled for cellular potency in inhibition of collagen 1 (COL1) protein translation using a phenotypic screening platform.

### Example 1

### Synthetic Details for Compounds of the Invention (Schemes 1-16)

### General Methods

All reagents were commercial grade and were used as received without further purification, unless otherwise specified. Reagent grade solvents were used in all cases, unless otherwise specified. Thin layer chromatography was carried out using pre-coated silica gel F-254 plates (thickness 0.25 mm). ¹H-NMR and ¹⁹F-NMR spectra were recorded on a Bruker Bruker Avance 400 MHz or Avance III 400 MHz spectrometer. The chemical shifts are expressed in ppm using the residual solvent as internal standard. Splitting patterns are designated as s (singlet), d (doublet), dd (doublet of doublets), t (triplet), dt (doublet of triplets), q (quartet), m (multiplet) and br s (broad singlet).

### Abbreviations

AcOH Acetic acid
amphos Bis(di-tert-butyl(4-dimethylaminophenyl)phosphine
n-BuLi n-butyllithium
t-BuLi tert-butyllithium
DBU 1,8-Diazabicyclo[5.4.0]undec-7-ene
dppb 1,4-Bis(diphenylphosphino)butane
dppf 1,1′-Bis(diphenylphosphino)ferrocene

### DCM Dichloromethane

DIBAL-H Diisobutylaluminum hydride

### DIPEA N,N-Diisopropylethylamine

### DMAP 4-(Dimethylamino)pyridine

### DMF N,N-Dimethylformamide

### DMA Dimethylacetamide

### DMSO Dimethylsulfonamide

HATU [O-(7-Azabenzotriazol-1-yl)-N,N,N′,N′-tetramethyluronium-hexafluorphosphat]
HPLC High performance liquid chromatography
MsCl Methanesulfonyl chloride

### NBS N-Bromosuccinimide

rt Room temperature
T3P Propylphosphonic anhydride
TBAF Tetrabutylammonium fluoride
TCFH N,N,N′,N′-tetramethylchloroformamidinium hexafluorophosphate

### THF Tetrahydrofuran

TMS-OTf Trimethylsilyl trifluoromethanesulfonate

### General Synthesis of Compounds of the Invention

The original general synthesis towards RHS-modified compounds (Compound 110 analogues, see Table 1 for structures) is shown in Scheme 1. This route features a first step indole formation reaction with simultaneous introduction of the right-hand side (RHS) group R₂. During the following steps, the left-hand side (LHS) of the molecule is manipulated and the amide group introduced at the end of the sequence.

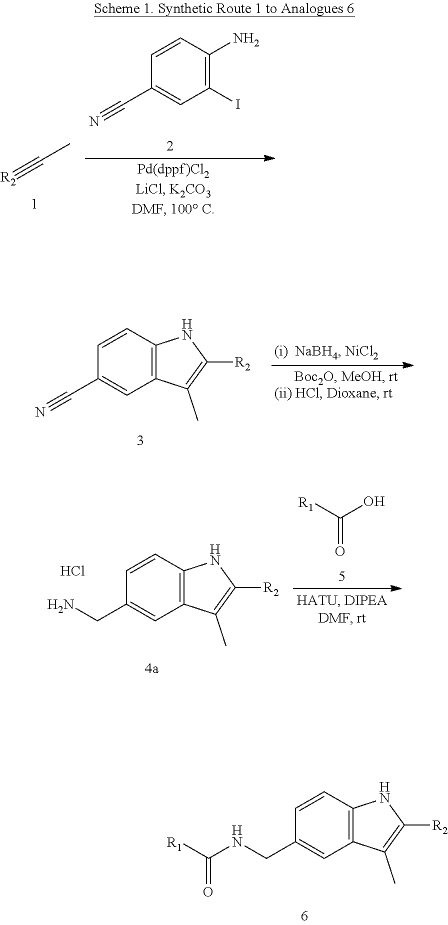

The first synthetic route involved the reaction of substituted alkynes 1 with commercial 4-amino-3-iodobenzonitrile 2 under Larock palladium-catalysed cyclisation conditions to deliver the 5-cyano indoles 3. Conversion of the nitrile group to the corresponding aminomethyl functionality was achieved by subjecting 3 to a one-pot reduction in situ Boc-protection sequence followed by acid deprotection of the N-Boc amine. The resulting key amine intermediate 4a was isolated as a hydrochloride salt. The final amide analogues 6 were prepared using heterocyclic carboxylic acids 5 as an array, under HATU amide coupling conditions from the amine hydrochloride intermediate 4a.

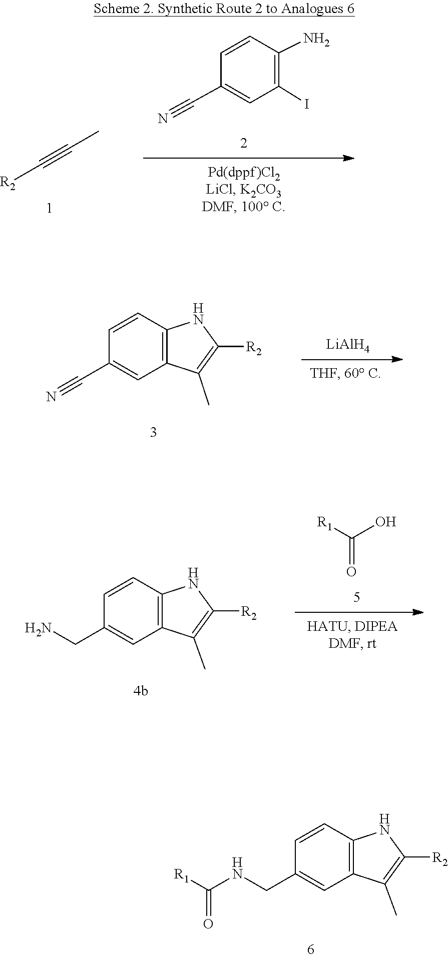

An alternative and slightly shorter synthesis to Route 1 is depicted in Scheme 2, as Route 2. Following this route, the nitrile intermediate 3 was reduced using lithium aluminium hydride in THF at 60° C. The first step of Scheme 2 involves Larock indole cyclisation; R₂-substituted alkynes 1 were reacted with commercial 4-amino-3-iodobenzonitrile 2 under palladium-catalysed cyclisation conditions to afford 5-cyano indole intermediates 3. Conversion of the cyano group to the corresponding aminomethyl functionality was achieved by subjecting intermediates 3 to lithium aluminium hydride reduction affording amine intermediates 4b. In the final step, intermediates 4b were reacted with heterocyclic carboxylic acids 5 under HATU amide coupling conditions and provided the final compounds 6.

An alternative route (Route 3) towards RHS-modified analogues 6 is shown below in Scheme 3 and allows the late stage introduction of the R₂ group.

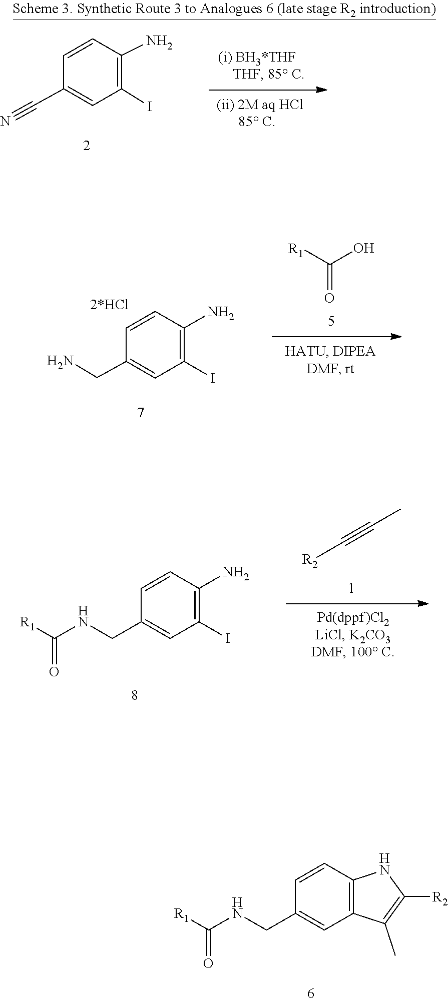

Synthetic route 3 commences with a borane reduction of 4-amino-3-iodobenzonitrile 2 to afford, after acidic work-up, aminomethyl intermediate 7 as a dihydrochloride salt. Intermediate 7 was then subjected to HATU amide coupling conditions using heterocyclic carboxylic acids 5 affording amide intermediates 8. The synthesis route was then completed by a Larock indole cyclisation reaction. R₂-substituted alkynes 1 were reacted with amide intermediates 8 under palladium-catalysed cyclisation conditions to furnish the final compound analogues 6.

R₂-substituted acetylene intermediates 1 were precursors to prepare final compound 6 analogues, as described above. The R₂-substituted acetylene intermediates 1 were prepared by one of three approaches (A, B and C) as shown in Scheme 4.

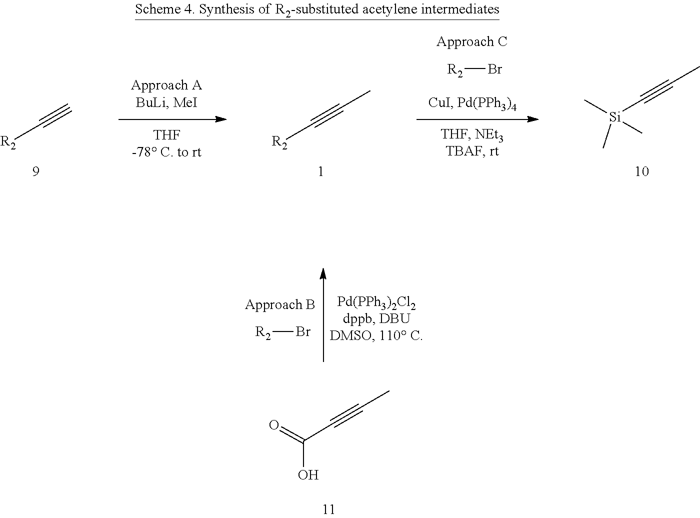

For approach A, R₂-substituted acetylene intermediates 1 were prepared via methylation of commercial acetylenes 9, using n-butyllithium in THF for deprotonation and methyl iodide for subsequent alkylation. Approach B was based on a decarboxylative alkynylation of haloheteroaryl precursors using 2-butynoic 11 as the alkyne building block. Finally, approach C proceeded via alkynylation of haloheteroaryl precursors using 1-TMS-propyne 10 to introduce the methylacetylene moiety.

The synthesis of the amine-linked analogues 14 is displayed below in Scheme 5.

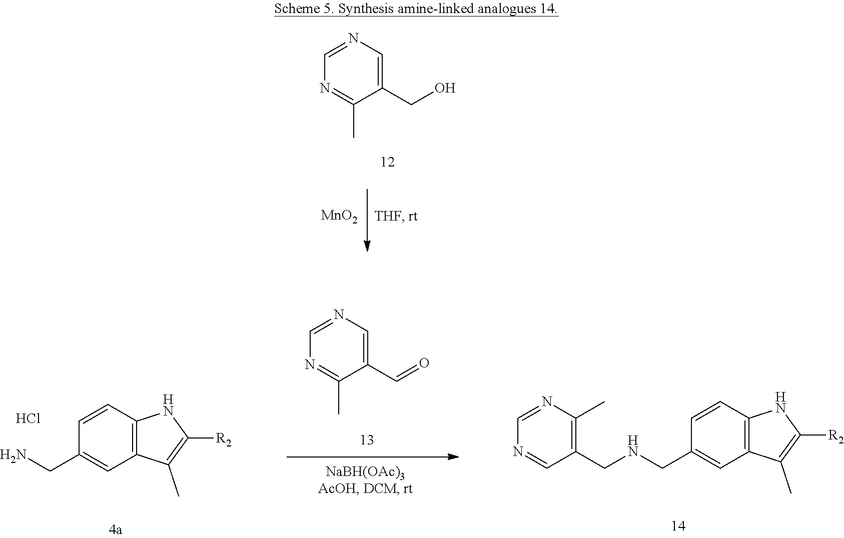

Commercial (4-methylpyrimidin-5-yl)methanol 12 was oxidised to 4-methylpyrimidine-5-carbaldehyde 13 in good yield, by treatment with manganese(IV) oxide in THF at room temperature. The final step involves reductive amination of aldehyde 13 with the indole amine hydrochloride intermediates 4a to afford the amine-linked analogues 14, using sodium triacetoxyborohydride and acetic acid in DCM at room temperature.

### Scaffold Modifications

The synthesis of the benzimidazole analogues 17 is shown below in Scheme 6.

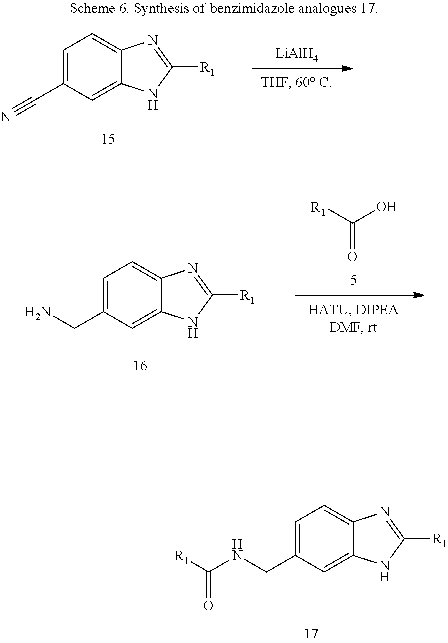

Commercial 6-cyano benzimidazole 15 was reduced to aminomethyl intermediates 16, using lithium aluminium hydride in THF at 60° C. Amine intermediates 16 were then converted to the amide benzimidazole analogues 17 by a HATU coupling using heterocyclic carboxylic acids 5 in the presence of DIPEA and DMF.

The synthesis of the 1H-pyrrolo[3,2-b]pyridine analogues 22 is shown below in Scheme 7.

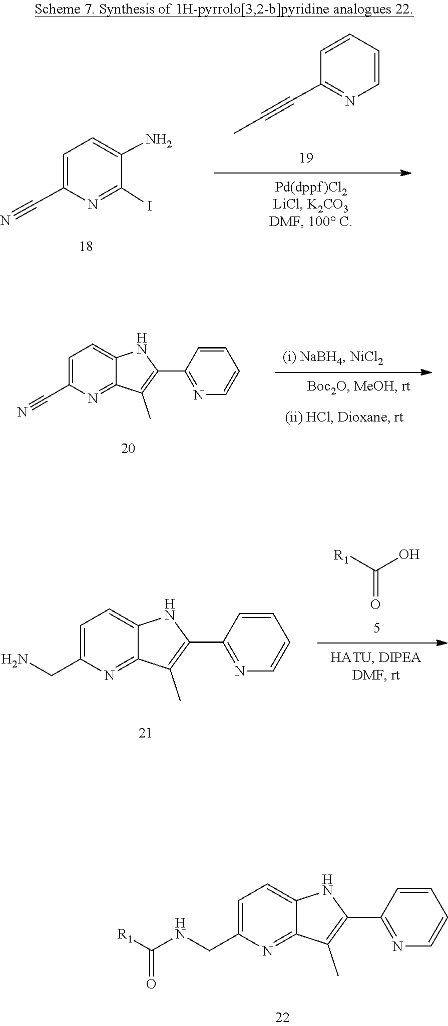

Commercial 5-amino-6-iodopicolinonitrile 18 was reacted with 2-(prop-1-yn-1-yl)pyridine 19 via a palladium-catalyzed Larock cyclisation reaction to deliver the azaindole intermediate 20. Reduction of the nitrile function in 20 was accomplished by a two-step process. In the first step, the cyano group of 20 was reduced under mild conditions, using sodium borohydride in the presence of nickel(II) chloride and Boc-anhydride affording the N-Boc-protected aminomethyl intermediate. This N-Boc-protected aminomethyl intermediate was subjected in a second step to acidic N-Boc deprotection to furnish the aminomethyl azaindole intermediate 21. The amine intermediate 21 was isolated as the free base rather than than the hydrochloride salt. Amidation of the amine intermediate 21 using heterocyclic carboxylic acids 5 under standard HATU amide coupling conditions gave the final amide 1H-pyrrolo[3,2-b]pyridine analogues 22.

The 3-des-methylindole analogues 26 were synthesized as shown below in Scheme 8.

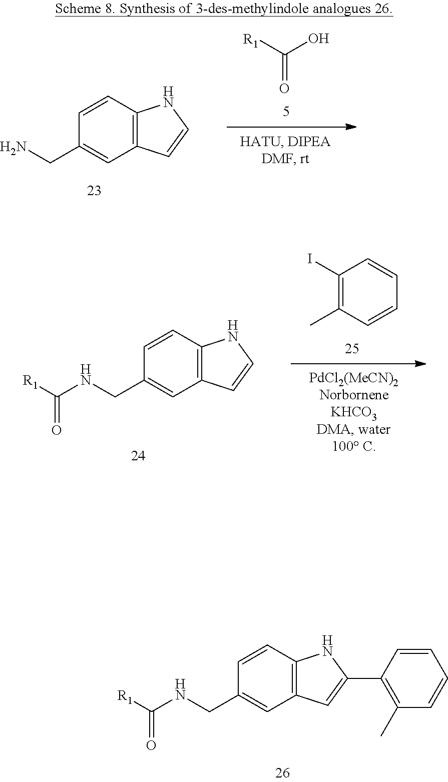

Commercial 5-aminomethylindole 23 was coupled with heterocyclic carboxylic acids 5, using standard HATU conditions providing amide intermediates 24 in good yield. The amide intermediates 24 were reacted with 2-iodotoluene 25 in the presence of bis(acetonitrile)dichloropalladium(II), norbornene and potassium hydrogen carbonate affording the C2-arylated-3-des-methylindole analogues 26. The 2-Tetrahydro-2H-pyran-substituted 3-des-methylindole analogues 29 were synthesized via a modified route as shown below in Scheme 9.

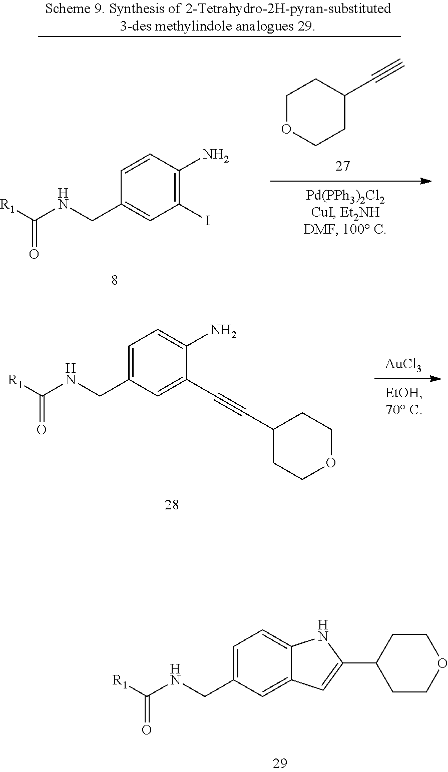

The earlier key aminoiodo intermediates 8 were subjected to a Sonogashira reaction, using 4-ethynyltetrahydro-2H-pyran 27 in the presence of bis(triphenylphosphine)palladium(II) dichloride, copper(I) iodide and diethylamine in DMF to afford the aminoalkyne intermediates 28. Subsequent cyclisation of the aminoalkynes 28 to the final 2-tetrahydro-2H-pyran-indoles 29 was achieved by heating in refluxing ethanol in the presence of gold(III) chloride.

The synthesis of the N-methylated indole analogues 34 is detailed below in Scheme 10.

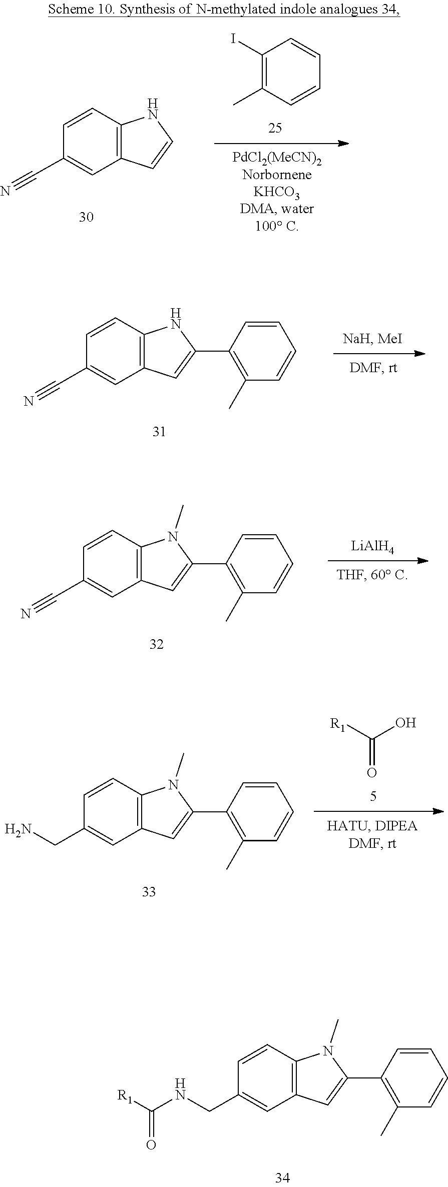

Commercial 5-cyanoindole 30 underwent a palladium-catalysed direct C2-arylation reaction with 2-iodotoluene 25, in the presence of bis(acetonitrile)dichloropalladium(II), norbornene and potassium hydrogen carbonate affording indole intermediate 31. The indole intermediate 31 was N-alkylated using methyl iodide in the presence of sodium hydride and provided the N-methylindole 32. The 5-nitrile moiety of the N-methylindole intermediate 32 was then converted to the corresponding aminomethyl group by reduction with lithium aluminium hydride in THF at 60° C. affording amine intermediate 33. Amine intermediate 33 was then subjected to standard HATU amide coupling with heteroaryl carboxylic acids 5 providing the final amide N-methylated indole analogues 34.

The bis-methylated indole analogues 40 were synthesised as described below in Scheme 11.

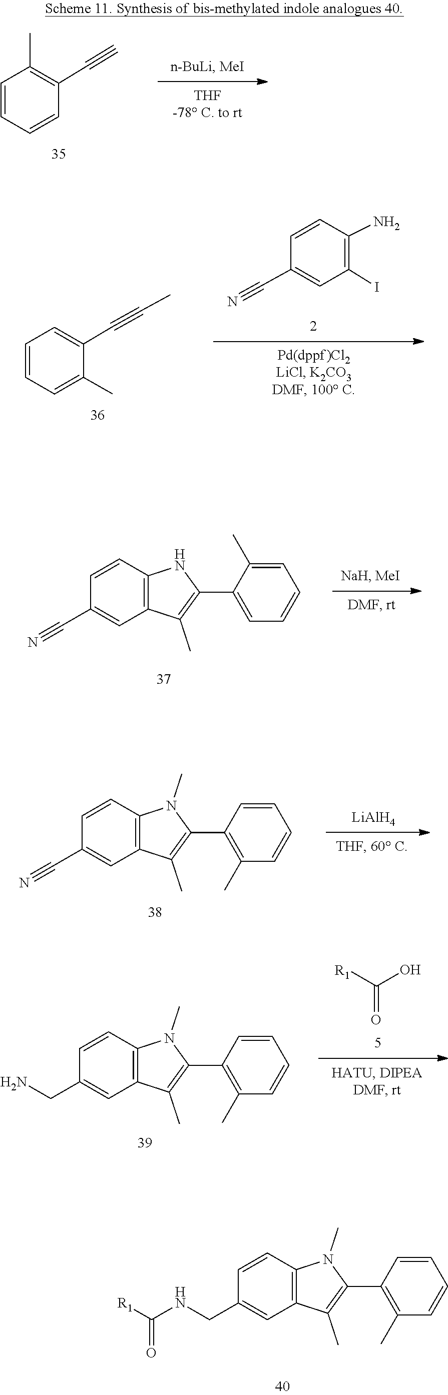

Commercial 1-ethynyl-2-methylbenzene 35 was methylated via a lithiation-alkylation sequence (Approach A in Scheme 4) to give 1-methyl-2-(prop-1-yn-1-yl)benzene 36. Larock cyclisation of intermediate 36 in the presence of 4-amino-3-iodobenzonitrile 2, under palladium-catalyzed conditions afforded the 2-tolyl indole-5-nitrile intermediate 37. Indole intermediate 37 was then N-alkylated using methyl iodide in the presence of sodium hydride to provide the N-methylindole 38. The 5-nitrile moiety in the N-methylindole intermediate 38 was reduced to the corresponding aminomethyl group employing lithium aluminium hydride in THF at 60° C. to afford amine intermediate 39. Amine intermediate 39 was subjected to a standard HATU amide coupling with heteroaryl carboxylic acids 5 providing the final bis-methylated indole analogues 40.

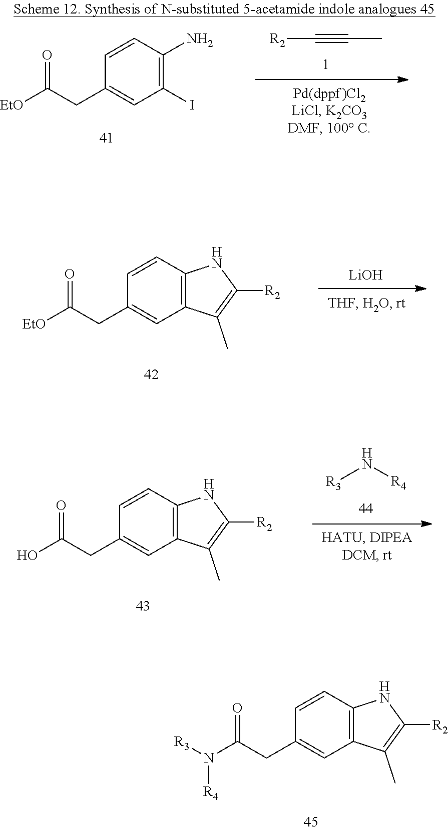

Commercially available ethyl 2-(4-amino-3-iodophenyl)acetate 41 was reacted with various 2-substituted 1-methylalkynes 1 via a Larock indole cyclisation, under palladium-catalyzed conditions at elevated temperature to afford the 5-substituted ethyl acetate indole intermediate 42. The ethyl ester moiety of intermediate 42 was hydrolyzed under basic conditions to give the carboxylic acid intermediate 43. HATU amide coupling of carboxylic acid intermediate 43 with various primary/secondary amines 44 furnished the final N-substituted 5-acetamide indole analogues 45.

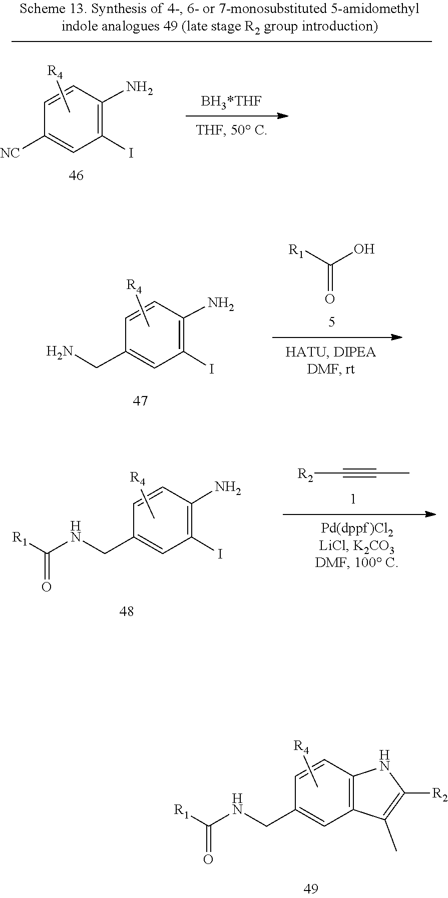

The synthetic scheme described in Scheme 13 is similar to the earlier described Scheme 3, which includes the same sequence of steps and the late stage R₂ group introduction. Commercially available substituted 4-amino-5-iodobenzonitriles 46 were reduced to the corresponding substituted 4-(aminomethyl)-2-iodoanilines 47 employing borane in tetrahydrofuran at elevated temperature. Standard HATU amide coupling of the 4-(aminomethyl) moiety of intermediate 47 with various heteroaryl carboxylic acids 5 afforded the amide intermediates 48. The final step involves late stage R₂ group introduction via a Larock indole cyclisation. The 2-iodoaniline moiety of intermediate 48 was reacted with 2-substituted 1-methylalkynes 1, under palladium-catalyzed conditions affording the final 4-, 6- or 7-monosubstituted 5-amidomethyl indole analogues 49.

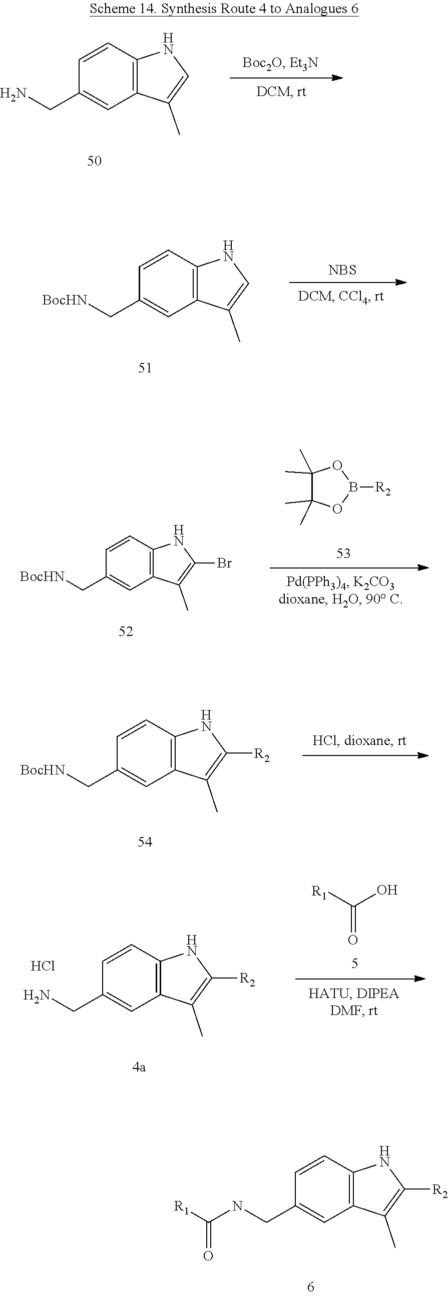

The amino group of (3-methyl-1H-indol-5-yl)methanamine 50 was protected as a N-Boc group to afford intermediate tert-butyl ((3-methyl-1H-indol-5-yl)methyl)carbamate 51, using di-tert-butyl dicarbonate at ambient temperature in the presence of triethylamine. Intermediate 51 was brominated at the 2- position of the indole ring, using N-bromosuccinimide in carbon tetrachloride and DCM to provide the key intermediate tert-butyl ((2-bromo-3-methyl-1H-indol-5-yl)methyl)carbamate 52. The key heteroaryl bromide intermediate 52 was subjected to Suzuki cross-couplings with various heterocyclic boronic esters to generate intermediate 54. The N-Boc protecting group of intermediate 54 was removed under acidic conditions, using a solution of hydrogen chloride in dioxane to afford the benzylic amine intermediate 4a as a hydrochloride salt. The synthetic approach in Scheme 14 is complimentary to the synthesis in Scheme 1 and was compatible with different R₂ groups). The final amide analogues 6 were prepared using heterocyclic carboxylic acids 5 as an array, under HATU amide coupling conditions from the benzylic amine hydrochloride intermediate 4a.

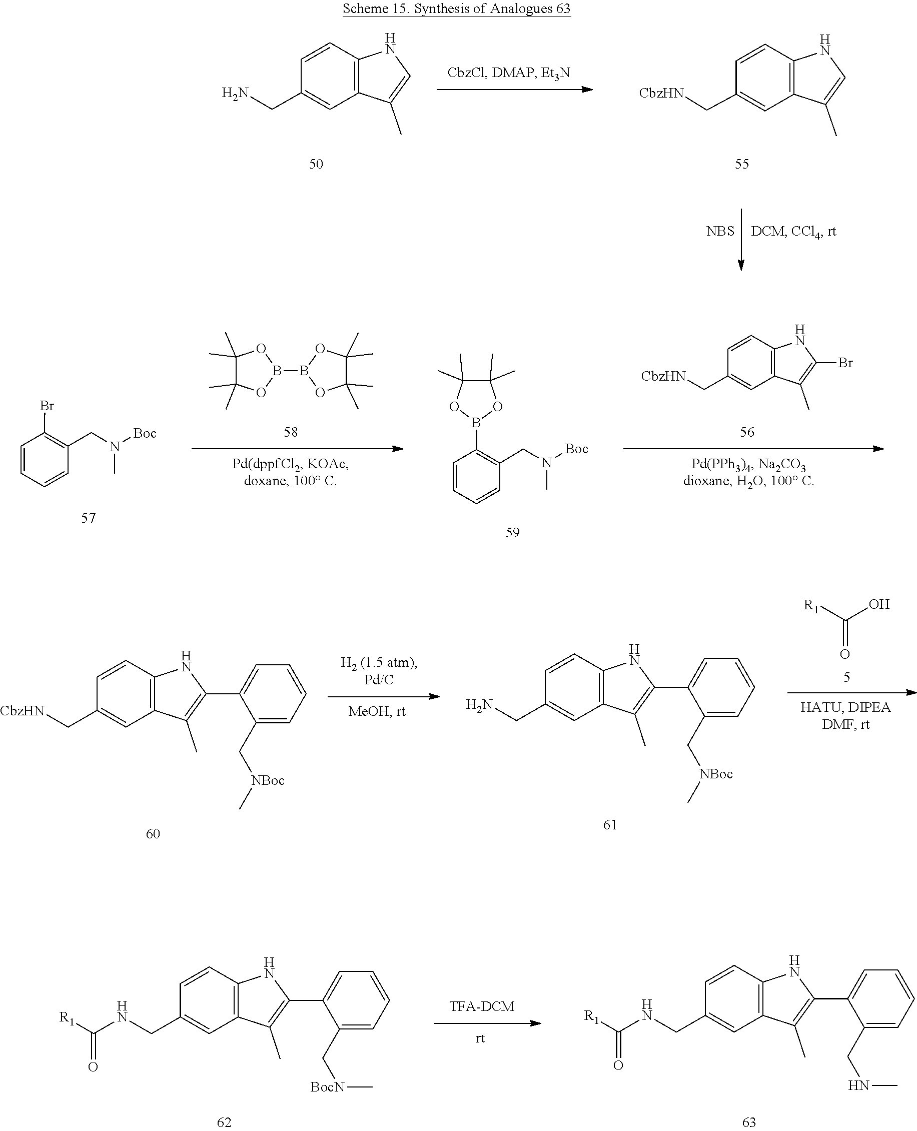

A convergent synthetic approach described in Scheme 15 was employed to prepare the compound analogues 63.

A two-step synthetic sequence related to the first two steps of the synthesis in Scheme 14 was used to synthesize benzyl ((2-bromo-3-methyl-1H-indol-5-yl)methyl)carbamate 56, which had a 2-bromo substituent on the indole ring. Step one of the first sequence involved protection of the amino group of (3-methyl-1H-indol-5-yl)methanamine 50 as a N-Cbz group to afford benzyl ((3-methyl-1H-indol-5-yl)methyl)carbamate 55, using benzyl chloroformate at ambient temperature in the presence of triethylamine and DMAP. Step two of the first sequence involved bromination at the 2-position of the indole ring of intermediate 55 to afford brominated intermediate 56, using N-bromosuccinimide in carbon tetrachloride and DCM at ambient temperature.

In the second sequence, the boronate ester, tert-butyl 2-(4,4,5,5-tetramethyl-1,3,2-dioxaborolan-2-yl)benzylmethylcarbamate 59 was prepared in a single step from commercially available tert-butyl 2-bromobenzyl(methyl)carbamate 57 and bis(pinacolato)diboron 58, using palladium-catalyzed conditions in the presence of potassium acetate at elevated temperature. The heteroaryl bromide intermediate 56 and boronate ester intermediate 58 from the two synthetic sequences were subsequently used in a Suzuki cross-coupling reaction under palladium-catalyzed conditions to generate the key 2-aryl indole intermediate 60.

The N-Cbz protecting group of intermediate 60 was removed under neutral palladium-catalyzed hydrogenation conditions to deliver the benzylic amine intermediate 61. In the penultimate step, HATU amide coupling of the benzylic amine intermediate 61 with a variety of heterocyclic carboxylic acids 5 provided the N-Boc protected 2-arylindole amide intermediates 62. A final N-Boc deprotection step under acidic conditions using trifluoroacetic acid in DCM at ambient temperature furnished the final compound analogues 63.

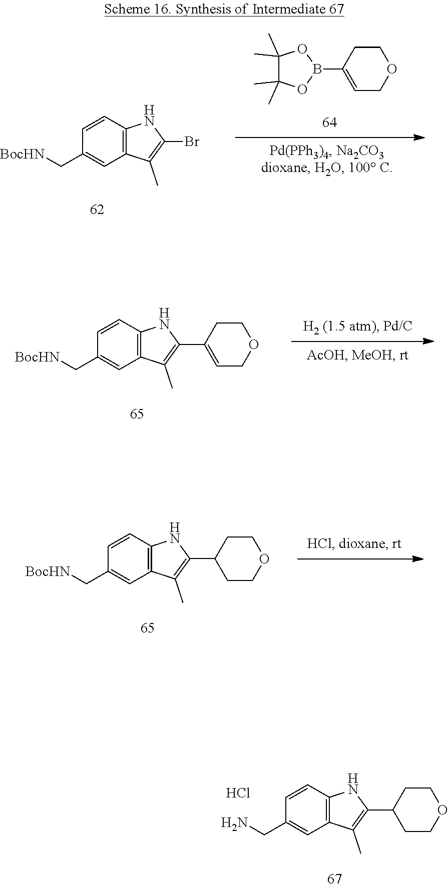 [SMILES: CC1(C)OB(C2=CCOCC2)OC1(C)C ; Cc1c(Br)[nH]c2ccc(CNC(=O)OC(C)(C)C)cc12 ; Cc1c(C2=CCOCC2)[nH]c2ccc(CNC(=O)OC(C)(C)C)cc12 ; Cc1c(C2CCOCC2)[nH]c2ccc(CNC(=O)OC(C)(C)C)cc12 ; Cl ; Cc1c(C2CCOCC2)[nH]c2ccc(CN)cc12]

The heteroaryl bromide intermediate 52, previously described in Scheme 14, was subjected to a Suzuki cross-coupling reaction with commercially available 3,6-dihydro-2H-pyran-4-boronic acid pinacol ester, under palladium-catalyzed conditions at elevated temperature yielding intermediate 65. Subsequently, palladium-catalyzed hydrogenation of intermediate 65 at ambient temperature afforded intermediate 66. The amine intermediate 67 was obtained readily following N-Boc group deprotection of intermediate 66 under acidic conditions. The benzylic amine intermediate 67 was isolated as the hydrochloride salt and then used subsequently in further chemistry.

### Detailed Synthesis of Intermediates of Compounds of the Invention

### Synthesis of 1-fluoro-2-(prop-1-yn-1-yl)benzene

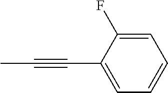

To a solution of 1-ethynyl-2-fluorobenzene (1 mL, 8.82 mmol) in anhydrous THF (20 mL) was added dropwise a solution of n-butyllithium (2.5 M in hexanes, 7 mL, 17.65 mmol) at −70° C. to −60° C. over a period of 20 minutes. After stirring at this temperature for one hour, methyl iodide (2.7 mL, 44.12 mmol) was added dropwise and the reaction mixture was allowed to warm to room temperature. After stirring for 2 hours, the reaction was quenched by the addition of a saturated solution of sodium thiosulfate (10 mL). The organic phase was separated, and the aqueous phase was extracted with iso-hexane (2×20 mL). The combined organic extracts were dried (MgSO₄), filtered and evaporated to furnish 1-fluoro-2-(prop-1-yn-1-yl)benzene as a pale yellow liquid.

Yield 1.25 g (quant). ¹H NMR (400 MHz, CDCl₃) δ 7.41-7.36 (m, 1H), 7.27-7.21 (m, 1H), 7.07 (dd, J=1.2, 4.4 Hz, 1H), 7.03 (dq, J=1.1, 5.0 Hz, 1H), 2.10 (s, 3H).

### Synthesis of 2-(2-fluorophenyl)-3-methyl-1H-indole-5-carbonitrile

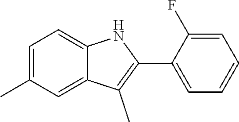 [SMILES: Cc1ccc2[nH]c(-c3ccccc3F)c(C)c2c1]

A sealed tube was charged with 4-amino-3-iodobenzonitrile (1 g, 4.1 mmol), K₂CO₃ (1.13 g, 8.2 mmol), LiCl (0.174 g, 4.1 mmol) and [1,1′-bis(diphenylphosphino)ferrocene]dichloropalladium(II) complex with dichloromethane (0.335 g, 0.41 mmol). To this mixture was added a solution of 1-fluoro-2-(prop-1-yn-1-yl)benzene (0.687 g, 5.12 mmol) in anhydrous DMF (6 mL). The resulting suspension was degassed for a few minutes with nitrogen, the reaction tube sealed and the mixture heated to 100° C. for 4 hours. After cooling to room temperature, the reaction mixture was filtered through celite, rinsed with DMF and concentrated. The residue was partioned between ethyl acetate (100 mL) and water (100 mL). The organic phase was washed with water (50 mL) and brine (30 mL), dried (MgSO₄), filtered and evaporated. The residue was purified by column chromatography on silica gel (5-25% EtOAc in cyclohexane) to give 2-(2-fluorophenyl)-3-methyl-1H-indole-5-carbonitrile as a light brown solid.

Yield 433 mg (42%). ¹H NMR (400 MHz, DMSO) δ 11.79 (s, 1H), 8.14 (d, J=0.4 Hz, 1H), 7.64-7.59 (m, 1H), 7.56-7.44 (m, 3H), 7.42-7.37 (m, 2H), 2.29 (d, J=1.6 Hz, 3H).

### Synthesis of tert-butyl ((2-(2-fluorophenyl)-3-methyl-1H-indol-5-yl)methyl)carbamate

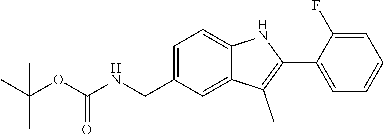 [SMILES: Cc1c(-c2ccccc2F)[nH]c2ccc(CNC(=O)OC(C)(C)C)cc12]

To a suspension of 2-(2-fluorophenyl)-3-methyl-1H-indole-5-carbonitrile (352 mg, 1.41 mmol) in anhydrous MeOH (10 mL) was added nickel(II) chloride hexahydrate (33 mg, 0.14 mmol) and di-tert-butyl dicarbonate (920 mg, 4.22 mmol) at room temperature. To the resulting solution sodium borohydride (426 mg, 11.3 mmol) was added portion wise over a period of 15 minutes. After stirring for 15 minutes at room temperature, the reaction mixture was partitioned between ethyl acetate (50 mL) and a saturated NaHCO₃ solution (50 mL). The organic phase was washed with brine (20 mL), dried (MgSO₄), filtered and evaporated to provide tert-butyl ((2-(2-fluorophenyl)-3-methyl-1H-indol-5-yl)methyl)carbamate as a yellow oil.

Yield 670 mg (quant). ¹H NMR (400 MHz, DMSO) δ 11.11 (s, 1H), 7.65-7.57 (m, 1H), 7.51 (dd, J=5.8, 5.8 Hz, 1H), 7.46-7.32 (m, 5H), 7.09 (d, J=8.1 Hz, 1H), 4.26 (d, J=5.8 Hz, 2H), 2.28 (s, 3H), 1.45 (s, 9H). Sample also contains di-tert-butyl dicarbonate

### Synthesis of (2-(2-fluorophenyl)-3-methyl-1H-indol-5-yl)methanamine hydrochloride

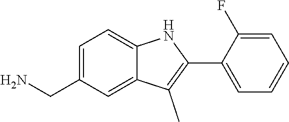 [SMILES: Cc1c(-c2ccccc2F)[nH]c2ccc(CN)cc12]

tert-Butyl ((2-(2-fluorophenyl)-3-methyl-1H-indol-5-yl)methyl)carbamate (675 mg, 1.4 mmol) was treated at room temperature with hydrogen chloride in dioxane (4 M, 19 mL, 76 mmol) for 1.5 hours. The reaction mixture was concentrated, triturated with diethyl ether (3×20 mL) and dried to furnish (2-(2-fluorophenyl)-3-methyl-1H-indol-5-yl)methanamine hydrochloride as a light brown solid.

Yield 376 mg (91%). ¹H NMR (400 MHz, DMSO) δ 11.33 (s, 1H), 8.26 (br s, 3H), 7.73 (s, 1H), 7.64 (dd, J=7.3, 7.3 Hz, 1H), 7.58-7.51 (m, 1H), 7.47-7.38 (m, 3H), 7.28 (d, J=8.6 Hz, 1H), 4.16 (d, J=5.6 Hz, 2H), 2.31 (s, 3H).

### Synthesis of tert-butyl ((3-methyl-2-(o-tolyl)-1H-indol-5-yl)methyl)carbamate

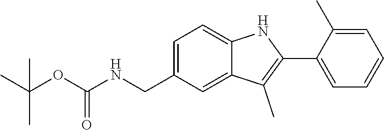 [SMILES: Cc1ccccc1-c1[nH]c2ccc(CNC(=O)OC(C)(C)C)cc2c1C]

To a solution of 3-methyl-2-(o-tolyl)-1H-indole-5-carbonitrile (100 mg, 0.41 mmol) in anhydrous MeOH (4 mL) was added nickel(II) chloride hexahydrate (9.7 mg, 0.04 mmol) and di-tert-butyl dicarbonate (266 mg, 1.22 mmol) at room temperature. Sodium borohydride (123 mg, 3.25 mmol) was then added portion wise over a period of 11 minutes. After stirring for 15 minutes at room temperature, the reaction mixture was partitioned between ethyl acetate (25 mL) and a saturated NaHCO₃ solution (25 mL). The organic phase was washed with brine (20 mL), dried (MgSO₄), filtered and evaporated to provide tert-butyl ((3-methyl-2-(o-tolyl)-1H-indol-5-yl)methyl)carbamate as a yellow oil.

Yield 173 mg (quant). ¹H NMR (400 MHz, DMSO) δ 10.70-10.67 (m, 1H), 7.17-7.14 (m, 3H), 7.11 (s, 3H), 7.05 (d, J=8.3 Hz, 1H), 6.82 (d, J=8.1 Hz, 1H), 4.02 (d, J=5.8 Hz, 2H), 2.04 (s, 3H), 1.92 (s, 3H), 1.22 (s, 9H). Sample also contains di-tert-butyl decarbonate.

### Synthesis of (3-methyl-2-(o-tolyl)-1H-indol-5-yl)methanamine hydrochloride

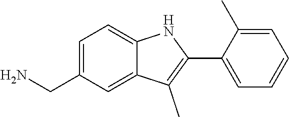 [SMILES: Cc1ccccc1-c1[nH]c2ccc(CN)cc2c1C]

tert-Butyl ((3-methyl-2-(o-tolyl)-1H-indol-5-yl)methyl)carbamate (173 mg, 0.41 mmol) was treated at room temperature with hydrogen chloride in dioxane (4 M, 5 mL, 20 mmol) for 2 hours. The reaction mixture was concentrated, triturated with diethyl ether (3×20 mL) and dried to furnish (3-methyl-2-(o-tolyl)-1H-indol-5-yl)methanamine hydrochloride as a beige solid.

Yield 92 mg (65%). ¹H NMR (400 MHz, DMSO) δ 11.15 (s, 1H), 8.21 (br s, 3H), 7.68 (s, 1H), 7.35-7.45 (m, 5H), 7.24 (d, J=8.3 Hz, 1H), 4.15 (d, J=5.3 Hz, 2H), 2.27 (s, 3H), 2.19 (s, 3H).

### Synthesis of 3-methyl-2-(pyridin-2-yl)-1H-indole-5-carbonitrile

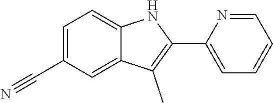 [SMILES: Cc1c(-c2ccccn2)[nH]c2ccc(C#N)cc12]

A sealed tube was charged with 4-amino-3-iodobenzonitrile (439 mg, 1.80 mmol), K₂CO₃ (497 mg, 3.60 mmol), LiCl (76 mg, 1.80 mmol) and [1,1′-bis(diphenylphosphino)ferrocene]dichloropalladium(II) complex with dichloromethane (147 mg, 0.18 mmol). To this mixture was added a solution of 2-(prop-1-yn-1-yl)pyridine (232 mg, 1.98 mmol) in anhydrous DMF (3.5 mL). The resulting suspension was degassed for a few minutes with nitrogen, the reaction tube sealed and the mixture heated to 100° C. for 19 hours. After cooling to room temperature, the reaction mixture was diluted with ethyl acetate (20 mL), brine (10 mL) was added and the organic phase transferred into a separating funnel and partioned between ethyl acetate (50 mL) and water (50 mL). The organic phase was washed with water (30 mL) and brine (20 mL), dried (MgSO₄), filtered and evaporated. The residue was purified by column chromatography on silica gel (5-40% EtOAc in iso-hexane) to give 3-methyl-2-(pyridin-2-yl)-1H-indole-5-carbonitrile as an off-white solid.

Yield 134 mg (32%). ¹H NMR (400 MHz, DMSO) δ 12.01 (s, 1H), 8.78 (d, J=4.3 Hz, 1H), 8.24 (s, 1H), 8.05-7.99 (m, 1H), 7.95 (d, J=7.8 Hz, 1H), 7.62 (d, J=8.6 Hz, 1H), 7.52 (d, J=8.3 Hz, 1H), 7.43 (dd, J=6.1, 6.1 Hz, 1H), 2.66 (s, 3H).

### Synthesis of tert-butyl ((3-methyl-2-(pyridin-2-yl)-1H-indol-5-yl)methyl)carbamate

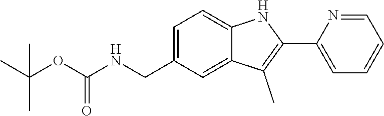 [SMILES: Cc1c(-c2ccccn2)[nH]c2ccc(CNC(=O)OC(C)(C)C)cc12]

To a solution of 3-methyl-2-(pyridin-2-yl)-1H-indole-5-carbonitrile (134 mg, 0.57 mmol) in anhydrous MeOH (6 mL) was added nickel(II) chloride hexahydrate (14 mg, 0.06 mmol) and di-tert-butyl dicarbonate (376 mg, 1.72 mmol) at room temperature. Sodium borohydride (174 mg, 4.60 mmol) was then added portion wise over a period of 14 minutes. After stirring for 30 minutes at room temperature, di-tert-butyl dicarbonate (210 mg, 0.96 mmol) was added, followed by sodium borohydride (150 mg, 3.96 mmol) portion wise over a period of 6 minutes. After stirring for 2.5 hours at room temperature, di-tert-butyl dicarbonate (150 mg, 0.69 mmol) was added, followed by sodium borohydride (80 mg, 2.11 mmol) portion wise over a period of 4 minutes. After stirring for 30 minutes at room temperature, the reaction mixture was partitioned between ethyl acetate (25 mL) and a saturated NaHCO₃ solution (25 mL). The organic phase was washed with brine (20 mL), dried (MgSO₄), filtered and evaporated to provide tert-butyl ((3-methyl-2-(pyridin-2-yl)-1H-indol-5-yl)methyl)carbamate as a yellow oil.

Yield 238 mg (quant). ¹H NMR (400 MHz, DMSO) δ 11.32 (s, 1H), 8.73 (d, J=4.0 Hz, 1H), 7.97-7.92 (m, 1H), 7.88 (d, J=8.1 Hz, 1H), 7.47 (s, 1H), 7.42-7.31 (m, 3H), 7.10 (d, J=7.8 Hz, 1H), 4.28-4.22 (m, 2H), 2.62 (s, 3H), 1.47 (s, 9H). Sample also contains di-tert-butyl dicarbonate

### Synthesis of (3-methyl-2-(pyridin-2-yl)-1H-indol-5-yl)methanamine dihydrochloride

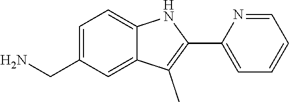 [SMILES: Cc1c(-c2ccccn2)[nH]c2ccc(CN)cc12]

tert-Butyl ((3-methyl-2-(pyridin-2-yl)-1H-indol-5-yl)methyl)carbamate (238 mg, 0.57 mmol) was treated at room temperature with hydrogen chloride in dioxane (4 M, 7 mL, 28 mmol) for 1.5 hours. The reaction mixture was concentrated, triturated with diethyl ether (3×20 mL) and dried to furnish (3-methyl-2-(pyridin-2-yl)-1H-indol-5-yl)methanamine dihydrochloride as a yellow solid.

Yield 161 mg (91%). ¹H NMR (400 MHz, DMSO) δ 11.58 (s, 1H), 8.76 (d, J=4.3 Hz, 1H), 8.28 (br s, 3H), 8.02 (dd, J=7.7, 7.7 Hz, 1H), 7.95 (d, J=8.1 Hz, 1H), 7.78 (s, 1H), 7.51 (d, J=8.3 Hz, 1H), 7.44-7.39 (m, 1H), 7.31 (d, J=7.8 Hz, 1H), 4.16 (d, J=5.6 Hz, 2H), 2.65 (s, 3H).

### Synthesis of 4-methylpyrimidine-5-carbaldehyde (13)

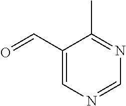

To a solution of (4-methylpyrimidin-5-yl)methanol (250 mg, 2.01 mmol) in anhydrous THF (10 mL) was added manganese(IV) oxide (1.75 g, 20.1 mmol), and the resulting suspension was stirred at 25° C. for 18 hours. The reaction mixture was filtered through celite, rinsed with DCM and evaporated to provide 4-methylpyrimidine-5-carbaldehyde (13) as a yellow oil.

Yield 137 mg (55%). ¹H NMR (400 MHz, DMSO) δ 10.32 (s, 1H), 9.27 (s, 1H), 9.15 (s, 1H), 2.83 (s, 3H).

### Synthesis of 1-methyl-2-(prop-1-yn-1-yl)benzene (36)

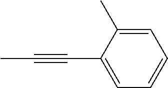

To a solution of 2-ethynyltoluene (1.6 mL, 12.91 mmol) in anhydrous THF (30 mL) was added dropwise a solution of n-butyllithium (2.5 M in hexanes, 7.75 mL, 19.4 mmol) at −78° C. over a period of 20 minutes. After stirring at this temperature for one hour, methyl iodide (1.2 mL, 19.4 mmol) was added dropwise and the reaction mixture was allowed to warm to room temperature. After stirring for 24 hours, the reaction was quenched by the addition of a saturated solution of sodium thiosulfate (20 mL). The organic phase was separated and the aqueous phase was extracted with iso-hexane (2×30 mL). The combined organic extracts were dried (Na₂SO₄), filtered and evaporated to give 1-methyl-2-(prop-1-yn-1-yl)benzene (36) as a pale yellow liquid.

Yield 1.7 g (quant). ¹H NMR (400 MHz, CDCl₃) δ 7.35 (d, J=7.3 Hz, 1H), 7.16 (d, J=4.0 Hz, 2H), 7.14-7.07 (m, 1H), 2.41 (s, 3H), 2.09 (s, 3H).

### Synthesis of 3-methyl-2-(o-tolyl)-1H-indole-5-carbonitrile (37)

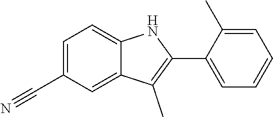 [SMILES: Cc1ccccc1-c1[nH]c2ccc(C#N)cc2c1C]

A sealed tube was charged with 4-amino-3-iodobenzonitrile (1 g, 4.1 mmol), K₂CO₃ (1.13 g, 8.2 mmol), LiCl (0.174 g, 4.1 mmol) and [1,1′-bis(diphenylphosphino)ferrocene]dichloropalladium(II) complex with dichloromethane (0.335 g, 0.41 mmol). To this mixture was added a solution of 1-methyl-2-(prop-1-yn-1-yl)benzene (0.667 g, 5.12 mmol) in anhydrous DMF (6 mL). The resulting suspension was degassed for a few minutes with nitrogen, the reaction tube sealed and the mixture heated to 100° C. for 18 hours. After cooling to room temperature, the reaction mixture was filtered through celite, rinsed with ethyl acetate and concentrated. The residue was partitioned between ethyl acetate (100 mL) and water (100 mL). The layers were separated, and the organic phase was washed with water (50 mL) and brine (30 mL), dried (MgSO₄), filtered and evaporated. The residue was purified by column chromatography on silica gel (0-30% EtOAc in iso-hexane) to give 3-methyl-2-(o-tolyl)-1H-indole-5-carbonitrile (37) as a yellow solid.

Yield 540 mg (53%). ¹H NMR (400 MHz, CDCl₃) δ 8.18-8.15 (br s, 1H), 7.94 (d, J=0.8 Hz, 1H), 7.45-7.29 (m, 6H), 2.26 (s, 3H), 2.20 (s, 3H).

### Synthesis of 1,3-dimethyl-2-(o-tolyl)-1H-indole-5-carbonitrile (38)

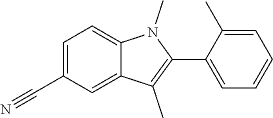 [SMILES: Cc1ccccc1-c1c(C)c2cc(C#N)ccc2n1C]

To a solution of 3-methyl-2-(o-tolyl)-1H-indole-5-carbonitrile (96 mg, 0.39 mmol) in anhydrous DMF at room temperature (2 mL) was added methyl iodide (0.073 mL, 1.17 mmol) followed by sodium hydride (60%, 23 mg, 0.59 mmol). After stirring at room temperature for 1 hour, the reaction mixture was quenched by addition of a saturated aqueous NaHCO₃ solution (1 mL) and partitioned between ethyl acetate (15 mL) and water (15 mL). The layers were separated, and the organic phase was washed with a diluted NaHCO₃ solution (10%, 10 mL) and brine (10 mL), dried (MgSO₄), filtered and evaporated. The resulting residue was triturated with petroleum ether and evaporated to give 1,3-dimethyl-2-(o-tolyl)-1H-indole-5-carbonitrile (38) as an off-white solid.

Yield 86 mg (85%). ¹H NMR (400 MHz, DMSO) δ 8.11 (s, 1H), 7.64 (d, J=8.6 Hz, 1H), 7.53 (d, J=8.3 Hz, 1H), 7.43 (s, 2H), 7.38-7.32 (m, 1H), 7.28 (d, J=7.3 Hz, 1H), 3.31 (s, 3H), 2.07 (s, 3H), 2.05 (s, 3H).

### Synthesis of (1,3-dimethyl-2-(o-tolyl)-1H-indol-5-yl)methanamine (39)

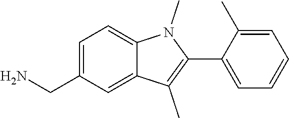 [SMILES: Cc1ccccc1-c1c(C)c2cc(CN)ccc2n1C]

To a solution of 1,3-dimethyl-2-(o-tolyl)-1H-indole-5-carbonitrile (85 mg, 0.33 mmol) in anhydrous THF (3 mL) was added LiAlH₄ (62 mg, 1.63 mmol) portion wise at room temperature. The reaction mixture was heated at 60° C. for 2 hours. After cooling to 0° C. on ice, water (120 μL) and 2N NaOH solution (120 μL) were added dropwise sequentially. The resulting mixture was diluted with ethyl acetate (20 mL), dried (MgSO₄), filtered and evaporated to give (1,3-dimethyl-2-(o-tolyl)-1H-indol-5-yl)methanamine (39) as a pale yellow oil.

Yield 86 mg (quant.). ¹H NMR (400 MHz, DMSO) δ 7.46 (s, 1H), 7.40 (d, J=3.8 Hz, 2H), 7.37-7.30 (m, 2H), 7.24 (d, J=7.1 Hz, 1H), 7.14 (d, J=8.6 Hz, 1H), 3.81 (s, 2H), 3.38 (s, 3H), 2.06 (s, 3H), 2.03 (s, 3H), 1.90 (br s, 2H).

### Synthesis of N-((1H-indol-5-yl)methyl)-4-methylpyrimidine-5-carboxamide

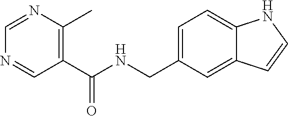 [SMILES: Cc1ncncc1C(=O)NCc1ccc2[nH]ccc2c1]

To a solution of 4-methylpyrimidine-5-carboxylic acid (104 mg, 0.752 mmol) in anhydrous DMF (3 mL) were added sequentially DIPEA (0.18 mL, 1.03 mmol) and HATU (312 mg, 0.821 mmol) and the reaction mixture was stirred at room temperature for 10 minutes. A solution of 5-(aminomethyl)indole (100 mg, 0.684 mmol) in anhydrous DMF (3 mL) was then added and the reaction continued to stir at room temperature for 1.5 hours. The mixture was partitioned between ethyl acetate (50 mL) and a diluted Na₂CO₃ solution (10%, 50 mL). The layers were separated, and the organic phase was washed with a diluted solution of Na₂CO₃ (10%, 25 mL) and brine (10 mL), dried (MgSO₄), filtered and evaporated. The residue was triturated with petroleum ether and evaporated to give N-((1H-indol-5-yl)methyl)-4-methylpyrimidine-5-carboxamide as a beige solid.

Yield 134 mg (73%). ¹H NMR (400 MHz, DMSO) δ 11.04 (s, 1H), 9.10-9.06 (m, 2H), 8.71 (s, 1H), 7.51 (s, 1H), 7.36 (d, J=8.3 Hz, 1H), 7.32 (dd, J=2.5, 2.5 Hz, 1H), 7.10 (d, J=8.3 Hz, 1H), 6.40 (s, 1H), 4.53 (d, J=5.6 Hz, 2H), 2.53 (s, 3H).

### Synthesis of 2-(o-tolyl)-1H-indole-5-carbonitrile (31)

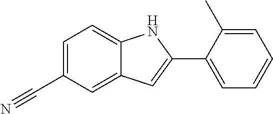 [SMILES: Cc1ccccc1-c1cc2cc(C#N)ccc2[nH]1]

A sealed tube was charged with indole-5-carbonitrile (540 mg, 3.80 mmol), bicyclo[2.2.1]hept-2-ene (715 mg, 7.60 mmol), KHCO₃ (761 mg, 7.60 mmol) and bis(acetonitrile)dichloropalladium(II) (99 mg, 0.38 mmol). A solution of 0.5 M aqueous N,N-dimethylacetamide (18.5 mL) was added at room temperature, followed by addition of 2-iodotoluene (0.97 mL, 7.60 mmol). After stirring at 100° C. for 48 hours, the reaction mixture was diluted with ethyl acetate (120 mL), filtered through MgSO₄ and partitioned between ethyl acetate (50 mL) and water (50 mL). The layers were separated, and the organic phase was washed with water (50 mL) and brine (20 mL), dried (MgSO₄), filtered and evaporated. The residue was purified by column chromatography on silica gel (5-30% EtOAc in cyclohexane) to give 2-(o-tolyl)-1H-indole-5-carbonitrile (31) as a white solid.

Yield 68 mg (8%). ¹H NMR (400 MHz, DMSO) δ 11.89 (s, 1H), 8.10-8.08 (m, 1H), 7.57-7.53 (m, 2H), 7.45 (dd, J=1.6, 8.5 Hz, 1H), 7.39-7.32 (m, 3H), 6.73 (s, 1H), 2.46 (s, 3H).

### Synthesis of 1-methyl-2-(o-tolyl)-1H-indole-5-carbonitrile (32)

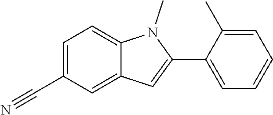 [SMILES: Cc1ccccc1-c1cc2cc(C#N)ccc2n1C]

To a solution of 2-(o-tolyl)-1H-indole-5-carbonitrile (66 mg, 0.284 mmol) in anhydrous DMF at room temperature (2 mL) was added methyl iodide (0.053 mL, 0.852 mmol) followed by sodium hydride (60%, 17 mg, 0.426 mmol). After stirring at room temperature for 2 hours, the reaction mixture was quenched by addition of a saturated aqueous NaHCO₃ solution (1 mL) and partitioned between ethyl acetate (15 mL) and water (15 mL). The layers were separated, and the organic phase was washed with a diluted NaHCO₃ solution (10%, 10 mL) and brine (10 mL), dried (MgSO₄), filtered and evaporated. The resulting residue was triturated with petroleum ether and evaporated to give 1-methyl-2-(o-tolyl)-1H-indole-5-carbonitrile (32) as an off-white solid.

Yield 51 mg (73%). ¹H NMR (400 MHz, DMSO) δ 8.11-8.09 (m, 1H), 7.69 (d, J=8.6 Hz, 1H), 7.54 (dd, J=1.5, 8.6 Hz, 1H), 7.44-7.40 (m, 2H), 7.34-7.32 (m, 2H), 6.59 (s, 1H), 3.54 (s, 3H), 2.15 (s, 3H).

### Synthesis of(1-methyl-2-(o-tolyl)-1H-indol-5-yl)methanamine (33)

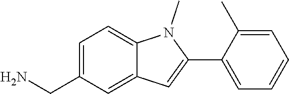 [SMILES: Cc1ccccc1-c1cc2cc(CN)ccc2n1C]

To a solution of 1-methyl-2-(o-tolyl)-1H-indole-5-carbonitrile (56 mg, 0.227 mmol) in anhydrous THF (3 mL) was added LiAlH₄ (43 mg, 1.14 mmol) portion wise at room temperature. The reaction mixture was heated at 60° C. for 2 hours. After cooling to 0° C. on ice, water (120 μL) and an aqueous 2N NaOH solution (120 μL) were added dropwise sequentially. The resulting mixture was diluted with ethyl acetate (20 mL), dried (MgSO₄), filtered and evaporated to give (1-methyl-2-(o-tolyl)-1H-indol-5-yl)methanamine (33) as a white solid.

Yield 57 mg (quant.). ¹H NMR (400 MHz, DMSO) δ 7.48 (s, 1H), 7.41-7.36 (m, 3H), 7.31 (dd, J=3.2, 3.2 Hz, 2H), 7.15 (dd, J=1.4, 8.5 Hz, 1H), 6.34 (s, 1H), 3.79 (s, 2H), 3.46 (s, 3H), 3.30 (br s, 2H), 2.16 (s, 3H).

### Synthesis of 1-methyl-3-(prop-1-yn-1-yl)benzene

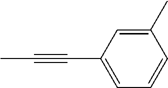

To a solution of 1-ethynyl-3-methylbenzene (1.1 mL, 8.61 mmol) in anhydrous THF (20 mL) was added dropwise a solution of n-butyllithium (2.5 M in hexanes, 5.2 mL, 13 mmol) at −78° C. over a period of 15 minutes. After stirring at this temperature for one hour, methyl iodide (0.80 mL, 12.91 mmol) was added dropwise and the reaction mixture was allowed to warm to room temperature. After stirring for 16 hours, the reaction was quenched by the addition of a saturated solution of sodium thiosulfate (10 mL). The layers were separated, and the organic phase was separated and the aqueous phase was extracted with /so-hexane (2×20 mL). The combined organic extracts were dried (MgSO₄), filtered and evaporated to give 1-methyl-3-(prop-1-yn-1-yl)benzene as a yellow oil.

Yield 1.16 g (quant, trace solvent). ¹H NMR (400 MHz, CDCl₃) δ 7.33-7.14 (m, 3H), 7.07 (d, J=7.1 Hz, 1H), 2.31 (s, 3H), 2.04 (s, 3H).

### Synthesis of 3-(prop-1-yn-1-yl)pyridine

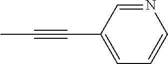

Compound 3-(prop-1-yn-1-yl)pyridine was prepared from 3-ethynylpyridine following a similar procedure to that described for the synthesis of 1-methyl-3-(prop-1-yn-1-yl)benzene except that it was purified by column chromatography on silica gel (0-20% EtOAc in cyclohexane) to give 3-(prop-1-yn-1- yl)pyridine as a yellow oil.

Yield 654 mg (57%). ¹H NMR (400 MHz, CDCl₃) δ 8.62 (d, J=1.0 Hz, 1H), 8.49-8.46 (m, 1H), 7.67-7.65 (m, 1H), 7.20 (dd, J=5.3, 7.6 Hz, 1H), 2.08 (s, 3H).

### Synthesis of 1-methoxy-3-(prop-1-yn-1-yl)benzene

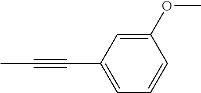

Compound 1-methoxy-3-(prop-1-yn-1-yl)benzene was prepared from 1-ethynyl-3-methoxybenzene following a similar procedure to that described for the synthesis of 1-methyl-3-(prop-1-yn-1-yl)benzene and was obtained as a pale yellow liquid. This was taken on to the next step without purification.

Yield 578 mg (quant). ¹H NMR (400 MHz, CDCl₃) δ 7.19 (dd, J=7.9, 7.9 Hz, 1H), 6.98 (d, J=7.6 Hz, 1H), 6.92 (s, 1H), 6.82 (dd, J=1.8, 8.3 Hz, 1H), 3.79 (s, 3H), 2.04 (s, 3H).

### Synthesis of 1-methoxy-2-(prop-1-yn-1-yl)benzene

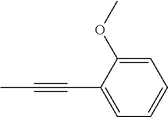

Compound 1-methoxy-2-(prop-1-yn-1-yl)benzene was prepared from 1-ethynyl-2-methoxybenzene following a similar procedure to that described for the synthesis of 1-methyl-3-(prop-1-yn-1-yl)benzene and was obtained as a dark orange liquid. This was taken on to the next step without purification.

Yield 591 mg (quant). ¹H NMR (400 MHz, CDCl₃) δ 7.37 (dd, J=1.3, 7.6 Hz, 1H), 7.27-7.21 (m, 1H), 6.87 (dd, J=7.7, 13.0 Hz, 2H), 3.88 (s, 3H), 2.12 (s, 3H).

### Synthesis of 2-methyl-3-(prop-1-yn-1-yl)pyridine

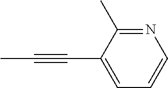

Compound 2-methyl-3-(prop-1-yn-1-yl)pyridine was prepared from 3-ethynyl-2-methylpyridine following a similar procedure to that described for the synthesis of 1-methyl-3-(prop-1-yn-1-yl)benzene except that it was purified by column chromatography on silica gel (0-25% Et₂O in petroleum ether) to give 2- methyl-3-(prop-1-yn-1-yl)pyridine as a colourless oil.

Yield 273 mg (49%). ¹H NMR (400 MHz, CDCl₃) δ 8.37 (dd, J=1.8, 4.9 Hz, 1H), 7.61 (dd, J=1.8, 7.7 Hz, 1H), 7.05 (dd, J=4.9, 7.8 Hz, 1H), 2.65 (s, 3H), 2.11 (s, 3H).

### Synthesis of 3-fluoro-2-(prop-1-yn-1-yl)pyridine

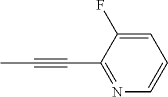

Compound 3-fluoro-2-(prop-1-yn-1-yl)pyridine was prepared from 2-ethynyl-3-fluoropyridine following a similar procedure to that described for the synthesis of 1-methyl-3-(prop-1-yn-1-yl)benzene except that it was purified by column chromatography on silica gel (0-25% Et₂O in petroleum ether) to give 3-fluoro-2-(prop-1-yn-1-yl)pyridine as a colourless liquid.

Yield 300 mg (54%). ¹H NMR (400 MHz, CDCl₃) δ 8.35 (d, J=4.8 Hz, 1H), 7.41-7.35 (m, 1H), 7.25-7.18 (m, 1H), 2.15 (s, 3H).

### Synthesis of tert-butyl 4-(prop-1-yn-1-yl)piperidine-1-carboxylate

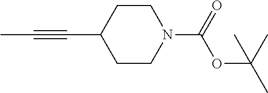 [SMILES: CC#CC1CCN(C(=O)OC(C)(C)C)CC1]

Compound tert-butyl 4-(prop-1-yn-1-yl)piperidine-1-carboxylate was prepared from tert-butyl 4-ethynylpiperidine-1-carboxylate following a similar procedure to that described for the synthesis of 1-methyl-3-(prop-1-yn-1-yl)benzene and was obtained as a pale yellow oil.

Yield 1.1 g (quant). ¹H NMR (400 MHz, CDCl₃) δ 3.75-3.65 (m, 2H), 3.22-3.09 (m, 2H), 2.54-2.46 (m, 1H), 1.80 (d, J=2.4 Hz, 3H), 1.78-1.69 (m, 2H), 1.54-1.47 (m, 2H), 1.46 (s, 9H).

### Synthesis of 4-(prop-1-yn-1-yl)tetrahydro-2H-pyran

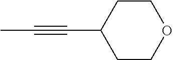

Compound 4-(prop-1-yn-1-yl)tetrahydro-2H-pyran was prepared from 4-ethynyltetrahydro-2H-pyran following a similar procedure to that described for the synthesis of 1-methyl-3-(prop-1-yn-1-yl)benzene except that it was purified by column chromatography on silica gel (0-15% Et₂O in petroleum ether) to give a 9:1 mixture of 4-(prop-1-yn-1-yl)tetrahydro-2H-pyran and 4-ethynyltetrahydro-2H-pyran as a colourless liquid. This was taken on to the next step without further purification.

Yield 209 mg. ¹H NMR (400 MHz, CDCl₃) δ 3.92-3.85 (m, 2H), 3.50-3.43 (m, 2H), 2.59-2.51 (m, 1H), 1.81 (d, J=2.3 Hz, 3H), 1.79-1.75 (m, 2H), 1.66-1.59 (m, 2H).

### Synthesis of 4-methyl-3-(prop-1-yn-1-yl)pyridine

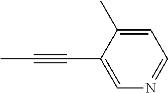

Compound 4-methyl-3-(prop-1-yn-1-yl)pyridine was prepared from 3-ethynyl-4-methylpyridine following a similar procedure to that described for the synthesis of 1-methyl-3-(prop-1-yn-1-yl)benzene except that it was purified by column chromatography on silica gel (0-40% Et₂O in petroleum ether) to give 4- methyl-3-(prop-1-yn-1-yl)pyridine as a colourless liquid.

Yield 175 mg (39%). ¹H NMR (400 MHz, CDCl₃) δ 8.53 (s, 1H), 8.33 (d, J=5.0 Hz, 1H), 7.09 (d, J=5.0 Hz, 1H), 2.40 (s, 3H), 2.12 (s, 3H).

### Synthesis of 1-(prop-1-yn-1-yl)-2-(trifluoromethyl)benzene

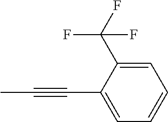

Compound 1-(prop-1-yn-1-yl)-2-(trifluoromethyl)benzene was prepared from 1-ethynyl-2-(trifluoromethyl)benzene following a similar procedure to that described for the synthesis of 1-methyl-3-(prop-1-yn-1-yl)benzene except that it was purified by column chromatography on silica gel (100% cyclohexane) to give 1-(prop-1-yn-1-yl)-2-(trifluoromethyl)benzene as a colourless liquid.

Yield 96 mg (26%). ¹H NMR (400 MHz, CDCl₃) δ 7.61 (d, J=7.8 Hz, 1H), 7.52 (d, J=7.6 Hz, 1H), 7.44 (dd, J=7.6, 7.6 Hz, 1H), 7.34 (dd, J=7.6, 7.6 Hz, 1H), 2.09 (s, 3H).

### Synthesis of 2-(prop-1-yn-1-yl)pyrazine

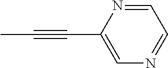

Compound 2-(prop-1-yn-1-yl)pyrazine was prepared from 2-ethynylpyrazine following a similar procedure to that described for the synthesis of 1-methyl-3-(prop-1-yn-1-yl)benzene except that it was purified by column chromatography on silica gel (0-40% Et₂O in petroleum ether) to give 2-(prop-1-yn-1-yl)pyrazine as a white solid.

Yield 333 mg (58%). ¹H NMR (400 MHz, CDCl₃) δ 8.61 (d, J=1.5 Hz, 1H), 8.51-8.49 (m, 1H), 8.44 (d, J=2.5 Hz, 1H), 2.13 (s, 3H).

### Synthesis of 2-(prop-1-yn-1-yl)pyrimidine

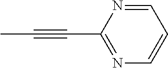

Compound 2-(prop-1-yn-1-yl)pyrimidine was prepared from 2-ethynylpyrimidine following a similar procedure to that described for the synthesis of 1-methyl-3-(prop-1-yn-1-yl)benzene except that it was purified by column chromatography on silica gel (0-100% Et₂O in petroleum ether) to give 2-(prop-1-yn-1-yl)pyrimidine as a pale brown solid.

Yield 236 mg (41%). ¹H NMR (400 MHz, CDCl₃) δ 8.68 (d, J=4.9 Hz, 2H), 7.20 (dd, J=4.9, 4.9 Hz, 1H), 2.12 (s, 3H).

### Synthesis of prop-1-yn-1-ylcyclopentane

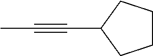

Compound prop-1-yn-1-ylcyclopentane was prepared from ethynylcyclopentane following a similar procedure to that described for the synthesis of 1-methyl-3-(prop-1-yn-1-yl)benzene and was obtained as a yellow oil. This was taken on to the next step without purification.

Yield 407 mg (88%). ¹H NMR (400 MHz, CDCl₃) δ 2.58-2.49 (m, 1H), 1.93-1.82 (m, 2H), 1.79 (d, J=2.5 Hz, 3H), 1.74-1.65 (m, 2H), 1.60-1.50 (m, 4H).

### Synthesis of 3-methyl-2-(prop-1-yn-1-yl)pyridine

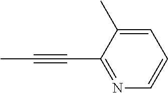

Compound 3-methyl-2-(prop-1-yn-1-yl)pyridine was prepared from 2-ethynyl-3-methylpyridine following a similar procedure to that described for the synthesis of 1-methyl-3-(prop-1-yn-1-yl)benzene except that it was purified by column chromatography on silica gel (0-50% Et₂O in petroleum ether) to give 3- methyl-2-(prop-1-yn-1-yl)pyridine as a yellow oil.

Yield 291 mg (65%). ¹H NMR (400 MHz, CDCl₃) δ 8.36 (dd, J=0.8, 4.0 Hz, 1H), 7.47 (d, J=7.6 Hz, 1H), 7.09 (dd, J=4.8, 7.8 Hz, 1H), 2.41 (s, 3H), 2.13 (s, 3H).

### Synthesis of 1,3-difluoro-2-(prop-1-yn 1-yl)benzene

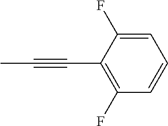

Compound 1,3-difluoro-2-(prop-1-yn-1-yl)benzene was prepared from 2-ethynyl-1,3-difluorobenzene following a similar procedure to that described for the synthesis of 1-methyl-3-(prop-1-yn-1-yl)benzene and was obtained as a pale orange liquid. This was taken on to the next step without purification.

Yield 461 mg (77%). ¹H NMR (400 MHz, CDCl₃) δ 7.24-7.16 (m, 1H), 6.90-6.85 (m, 2H), 2.15 (s, 3H).

### Synthesis of 3-fluoro-4-(prop-1-yn-1-yl)pyridine

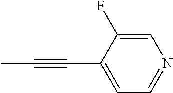

Compound 3-fluoro-4-(prop-1-yn-1-yl)pyridine was prepared from 4-ethynyl-3-fluoropyridine following a similar procedure to that described for the synthesis of 1-methyl-3-(prop-1-yn-1-yl)benzene except that it was purified by column chromatography on silica gel (0-25% Et₂O in petroleum ether) to give 3-fluoro-4-(prop-1-yn-1-yl)pyridine as a white solid. This was taken on to the next step without further purification.

Yield 165 mg (36%). ¹H NMR (400 MHz, CDCl₃) δ 8.44 (s, 1H), 8.32 (d, J=5.1 Hz, 1H), 7.29-7.25 (m, 1H), 2.14-2.08 (m, 3H).

### Synthesis of tert-butyl 3-(prop-1-yn-1-yl)piperidine-1-carboxylate

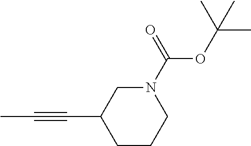 [SMILES: CC#CC1CCCN(C(=O)OC(C)(C)C)C1]

Compound tert-butyl 3-(prop-1-yn-1-yl)piperidine-1-carboxylate was prepared from tert-butyl 3-ethynylpiperidine-1-carboxylate following a similar procedure to that described for the synthesis of 1-methyl-3-(prop-1-yn-1-yl)benzene and was obtained as a pale yellow oil. This was taken on to the next step without further purification.

Yield 590 mg (quant). ¹H NMR (400 MHz, CDCl₃) δ 3.92-3.81 (m, 1H), 3.80-3.70 (m, 1H), 2.99-2.82 (m, 2H), 2.40-2.30 (m, 1H), 1.97-1.88 (m, 1H), 1.78 (d, J=2.0 Hz, 3H), 1.73-1.59 (m, 1H), 1.55-1.35 (m, 11H).

### Synthesis of 1-chloro-2-(prop-1-yn-1-yl)benzene

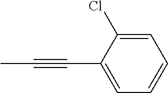

Compound 1-chloro-2-(prop-1-yn-1-yl)benzene was prepared from 1-chloro-2-ethynylbenzene following a similar procedure to that described for the synthesis of 1-methyl-3-(prop-1-yn-1-yl)benzene and was obtained as a yellow oil.

Yield 662 mg (quant). ¹H NMR (400 MHz, CDCl₃) δ 7.44-7.41 (m, 1H), 7.38-7.35 (m, 1H), 7.22-7.15 (m, 2H), 2.12 (s, 3H).

### Synthesis of 3-methyl-4-(prop-1-yn-1-yl)pyridine

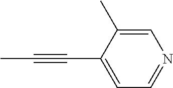

To a suspension of bis(triphenylphosphine)palladium(II) dichloride (102 mg, 0.15 mmol) and 1,4- bis(diphenylphosphino)butane (124 mg, 0.29 mmol) in DMSO (20 mL) was added 4-bromo-3-methylpyridine (500 mg, 2.91 mmol), 2-butynoic acid (293 mg, 3.49 mmol) and 1,8-diazabicyclo[5.4.0]undec-7-ene (1.3 mL, 8.72 mmol). The resulting suspension was degassed for a few minutes with nitrogen, the reaction tube sealed and the mixture heated to 110° C. for 1.5 hours. After cooling to room temperature, the reaction mixture was quenched by the addition of water (10 mL) and extracted with Et₂O (3×30 mL). The combined organic extracts were washed with water (50 mL) and brine (50 mL), then passed through a phase separator and evaporated. The resulting residue was purified by column chromatography on silica gel (0-65% Et₂O in petroleum ether) to give 3-methyl-4-(prop-1-yn-1-yl)pyridine as a pale yellow oil.

Yield 102 mg (27%). ¹H NMR (400 MHz, CDCl₃) δ 8.42 (s, 1H), 8.34 (d, J=5.1 Hz, 1H), 7.19 (d, J=5.1 Hz, 1H), 2.37 (s, 3H), 2.12 (s, 3H).

### Synthesis of 2-fluoro-3-(prop-1-yn-1-yl)pyridine

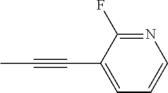

Compound 2-fluoro-3-(prop-1-yn-1-yl)pyridine was prepared from 3-bromo-2-fluoropyridine following a similar procedure to that described for the synthesis of 3-methyl-4-(prop-1-yn-1-yl)pyridine except that it was purified by column chromatography on silica gel (0-10% Et₂O in petroleum ether) to give 2-fluoro-3-(prop-1-yn-1-yl)pyridine as a white solid.

Yield 134 mg (35%). ¹H NMR (400 MHz, CDCl₃) δ 8.10 (d, J=4.5 Hz, 1H), 7.80-7.75 (m, 1H), 7.14-7.10 (m, 1H), 2.11 (s, 3H).

### Synthesis of 1-methyl-5-(prop-1-yn-1-yl)-1H-pyrazole

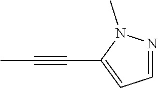

Compound 1-methyl-5-(prop-1-yn-1-yl)-1H-pyrazole was prepared from 5-bromo-1-methyl-1H-pyrazole following a similar procedure to that described for the synthesis of 3-methyl-4-(prop-1-yn-1-yl)pyridine except that it was purified by column chromatography on silica gel (0-40% Et₂O in petroleum ether) to give 1-methyl-5-(prop-1-yn-1-yl)-1H-pyrazole as a colourless oil. This was taken on to the next step without further purification.

Yield 99 mg (88%). ¹H NMR (400 MHz, CDCl₃) δ 7.39 (d, J=2.0 Hz, 1H), 6.31 (d, J=2.0 Hz, 1H), 3.90 (s, 3H), 2.11 (s, 3H).

### Synthesis of 1-ethyl-2-(prop-1-yn-1-yl)benzene

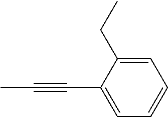

Compound 1-ethyl-2-(prop-1-yn-1-yl)benzene was prepared from 1-bromo-2-ethylbenzene following a similar procedure to that described for the synthesis of 3-methyl-4-(prop-1-yn-1-yl)pyridine except that it was purified by column chromatography on silica gel (100% petroleum ether) to give 1-ethyl-2-(prop-1-yn-1-yl)benzene as a pale yellow liquid.

Yield 302 mg (67%). ¹H NMR (400 MHz, CDCl₃) δ 7.36 (d, J=7.4 Hz, 1H), 7.25-7.15 (m, 2H), 7.13-7.08 (m, 1H), 2.79 (q, J=7.5 Hz, 2H), 2.09 (s, 3H), 1.23 (t, J=7.5 Hz, 3H).

### Synthesis of 2-methyl-4-(prop-1-yn-1-yl)thiazole

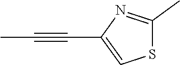

Compound 2-methyl-4-(prop-1-yn-1-yl)thiazole was prepared from 4-bromo-2-methylthiazole following a similar procedure to that described for the synthesis of 3-methyl-4-(prop-1-yn-1-yl)pyridine except that it was purified by column chromatography on silica gel (0-10% Et₂O in petroleum ether) to give 2-methyl-4-(prop-1-yn-1-yl)thiazole as a yellow solid.

Yield 283 mg (73%). ¹H NMR (400 MHz, CDCl₃) δ 7.15 (s, 1H), 2.68 (s, 3H), 2.04 (s, 3H).

### Synthesis of 1-cyclopropyl-2-(prop-1-yn-1-yl)benzene

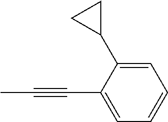

Compound 1-cyclopropyl-2-(prop-1-yn-1-yl)benzene was prepared from 1-bromo-2-cyclopropylbenzene following a similar procedure to that described for the synthesis of 3-methyl-4-(prop-1-yn-1-yl)pyridine except that it was purified by column chromatography on silica gel (isocratic, 100% petroleum ether) to give 1-cyclopropyl-2-(prop-1-yn-1-yl)benzene as a pale yellow liquid.

Yield 230 mg (58%). ¹H NMR (400 MHz, CDCl₃) δ 7.36 (dd, J=1.3, 7.6 Hz, 1H), 7.16 (ddd, J=1.3, 7.5, 7.5 Hz, 1H), 7.05 (ddd, J=1.3, 7.5, 7.5 Hz, 1H), 6.75 (d, J=7.8 Hz, 1H), 2.42-2.34 (m, 1H), 2.11 (s, 3H), 1.03-0.98 (m, 2H), 0.72-0.67 (m, 2H).

### Synthesis of 2-methyl-6-(prop-1-yn-1-yl)pyridine

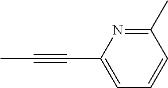

A sealed tube was charged with 2-bromo-6-methylpyridine (0.16 mL, 1.42 mmol), copper(I) iodide (81 mg, 0.43 mmol) and tetrakis(triphenylphosphine)palladium(0) (82 mg, 0.07 mmol), then degassed with nitrogen. Anhydrous THF (2 mL), triethylamine (0.59 mL, 4.26 mmol) and 1-(trimethylsilyl)propyne (0.22 mL, 1.49 mmol) were added at room temperature, followed by dropwise addition of tetrabutylammonium fluoride (1M in THF, 1.5 mL, 1.49 mmol) over 5 minutes. After stirring at room temperature for 16 hours, the reaction mixture was diluted with DCM/MeOH (1:1, 10 mL), dry loaded onto silica and purified by column chromatography on silica gel (0-15% EtOAc in cyclohexane) to give 2-methyl-6-(prop-1-yn-1-yl)pyridine as a yellow oil.

Yield 140 mg (75%). ¹H NMR (400 MHz, CDCl₃) δ 7.49 (dd, J=7.7, 7.7 Hz, 1H), 7.17 (d, J=7.7 Hz, 1H), 7.04 (d, J=7.6 Hz, 1H), 2.53 (s, 3H), 2.07 (s, 3H).

### Synthesis of 2-fluoro-6-(prop-1-yn-1-yl)pyridine

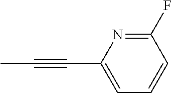

Compound 2-fluoro-6-(prop-1-yn-1-yl)pyridine was prepared from 2-bromo-6-fluoropyridine following a similar procedure to that described for the synthesis of 2-methyl-6-(prop-1-yn-1-yl)pyridine and was obtained as a pale yellow solid.

Yield 136 mg (71%). ¹H NMR (400 MHz, CDCl₃) δ 7.80 (dd, J=7.8, 7.8 Hz, 1H), 7.55 (dd, J=7.8, 7.8 Hz, 2H), 2.10 (s, 3H).

### Synthesis of 1-methyl-2-(prop-1-yn-1-yl)-1H-imidazole

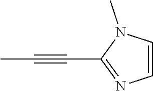

Compound 1-methyl-2-(prop-1-yn-1-yl)-1H-imidazole was prepared from 2-Iodo-1-methyl-1H-imidazole following a similar procedure to that described for the synthesis of 2-methyl-6-(prop-1-yn-1-yl)pyridine except that it was purified by column chromatography on silica gel (0-100% EtOAc in petroleum ether) to give 1-methyl-2-(prop-1-yn-1-yl)-1H-imidazole as a brown liquid. This was taken on to the next step without further purification.

Yield 110 mg (34%). ¹H NMR (400 MHz, CDCl₃) δ 6.98 (s, 1H), 6.83 (s, 1H), 3.68 (s, 3H), 2.10 (s, 3H).

### Synthesis of 4-(prop-1-yn-1-yl)pyrimidine

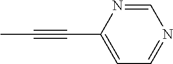

Compound 4-(prop-1-yn-1-yl)pyrimidine was prepared from 4-chloropyrimidine following a similar procedure to that described for the synthesis of 2-methyl-6-(prop-1-yn-1-yl)pyridine except that it was purified by column chromatography on silica gel (0-40% EtOAc in petroleum ether) to give 4-(prop-1-yn-1-yl)pyrimidine as a brown solid. This was taken on to the next step without further purification.

Yield 148 mg (28%). ¹H NMR (400 MHz, CDCl₃) δ 9.14 (s, 1H), 8.67 (d, J=5.1 Hz, 1H), 7.30 (dd, J=1.2, 5.1 Hz, 1H), 2.13 (s, 3H).

### Synthesis of 2-methoxy-6-(prop-1-yn-1-yl)pyridine

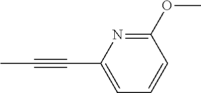

Compound 2-methoxy-6-(prop-1-yn-1-yl)pyridine was prepared from 2-bromo-6-methoxypyridine following a similar procedure to that described for the synthesis of 2-methyl-6-(prop-1-yn-1-yl)pyridine except that it was purified by column chromatography on silica gel (0-5% EtOAc in petroleum ether) to give 2-methoxy-6-(prop-1-yn-1-yl)pyridine as a pale yellow liquid.

Yield 330 mg (84%). ¹H NMR (400 MHz, CDCl₃) δ 7.48 (dd, J=7.3, 8.4 Hz, 1H), 6.98 (d, J=7.3 Hz, 1H), 6.66 (d, J=8.4 Hz, 1H), 3.94 (s, 3H), 2.08 (s, 3H).

### Synthesis of 4-fluoro-2-(prop-1-yn-1-yl)pyridine

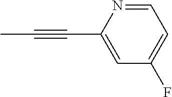

Compound 4-fluoro-2-(prop-1-yn-1-yl)pyridine was prepared from 2-bromo-4-fluoropyridine following a similar procedure to that described for the synthesis of 2-methyl-6-(prop-1-yn-1-yl)pyridine except that it was purified by column chromatography on silica gel (0-30% EtOAc in petroleum ether) to give 4- fluoro-2-(prop-1-yn-1-yl)pyridine as an orange liquid.

Yield 335 mg (82%). ¹H NMR (400 MHz, CDCl₃) δ 8.49 (dd, J=5.7, 8.7 Hz, 1H), 7.09 (dd, J=2.4, 9.3 Hz, 1H), 6.97-6.93 (m, 1H), 2.09 (s, 3H).

### Synthesis of 5-methoxy-2-(prop-1-yn-1-yl)pyridine

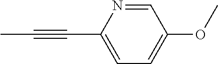

Compound 5-methoxy-2-(prop-1-yn-1-yl)pyridine was prepared from 2-bromo-5-methoxypyridine following a similar procedure to that described for the synthesis of 2-methyl-6-(prop-1-yn-1-yl)pyridine except that it was purified by column chromatography on silica gel (0-30% EtOAc in petroleum ether) to give 5-methoxy-2-(prop-1-yn-1-yl)pyridine as an orange solid.

Yield 379 mg (97%). ¹H NMR (400 MHz, CDCl₃) δ 8.23 (d, J=3.0 Hz, 1H), 7.30 (d, J=8.7 Hz, 1H), 7.11 (dd, J=3.0, 8.7 Hz, 1H), 3.86 (s, 3H), 2.06 (s, 3H).

### Synthesis of 4-methoxy-2-(prop-1-yn-1-yl)pyridine

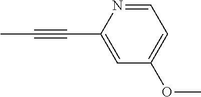

Compound 4-methoxy-2-(prop-1-yn-1-yl)pyridine was prepared from 2-bromo-4-methoxypyridine following a similar procedure to that described for the synthesis of 2-methyl-6-(prop-1-yn-1-yl)pyridine except that it was purified by column chromatography on silica gel (0-50% EtOAc in petroleum ether) to give 4-methoxy-2-(prop-1-yn-1-yl)pyridine as a pale brown liquid.

Yield 391 mg (quant.). ¹H NMR (400 MHz, CDCl₃) δ 8.33 (d, J=5.8 Hz, 1H), 6.90 (d, J=2.4 Hz, 1H), 6.72 (dd, J=2.4, 5.8 Hz, 1H), 3.83 (s, 3H), 2.07 (s, 3H).

### Synthesis of 5-methyl-4-(prop-1-yn-1-yl)pyrimidine

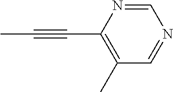

Compound 5-methyl-4-(prop-1-yn-1-yl)pyrimidine was prepared from 4-chloro-5-methylpyrimidine following a similar procedure to that described for the synthesis of 2-methyl-6-(prop-1-yn-1-yl)pyridine except that it was purified by column chromatography on silica gel (0-50% EtOAc in petroleum ether) to give 5-methyl-4-(prop-1-yn-1-yl)pyrimidine as an orange solid. This was taken on to the next step without further purification.

Yield 456 mg (80%). ¹H NMR (400 MHz, DMSO) δ 8.93 (s, 1H), 8.69 (s, 1H), 2.31 (s, 3H), 2.18 (s, 3H).

### Synthesis of 2-(prop-1-yn-1-yl)pyridine

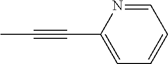

Compound 2-(prop-1-yn-1-yl)pyridine was prepared from 2-bromopyridine following a similar procedure to that described for the synthesis of 2-methyl-6-(prop-1-yn-1-yl)pyridine except that it was purified by column chromatography on silica gel (0-30% EtOAc in iso-hexane) to give 2-(prop-1-yn-1-yl)pyridine as a brown liquid.

Yield 1.32 g (83%). ¹H NMR (400 MHz, CDCl₃) δ 8.54 (d, J=4.3 Hz, 1H), 7.63-7.58 (m, 1H), 7.35 (d, J=7.8 Hz, 1H), 7.20-7.15 (m, 1H), 2.08 (s, 3H).

### Synthesis of 2-(prop-1-yn-1-yl)-6-(trifluoromethyl)pyridine

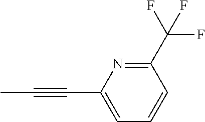

Compound 2-(prop-1-yn-1-yl)-6-(trifluoromethyl)pyridine was prepared from 2-bromo-6-(trifluoromethyl)pyridine following a similar procedure to that described for the synthesis of 2-methyl-6-(prop-1-yn-1-yl)pyridine and was obtained as a brown liquid.

Yield 160 mg (61%). ¹H NMR (400 MHz, CDCl₃) δ 7.80 (dd, J=7.8, 7.8 Hz, 1H), 7.55 (dd, J=7.8, 7.8 Hz, 2H), 2.10 (s, 3H).

### Synthesis of 5-methyl-2-(prop-1-yn-1-yl)pyridine

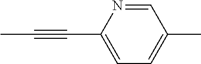

Compound 5-methyl-2-(prop-1-yn-1-yl)pyridine was prepared from 2-bromo-5-methylpyridine following a similar procedure to that described for the synthesis of 2-methyl-6-(prop-1-yn-1-yl)pyridine except that it was purified by column chromatography on silica gel (0-30% EtOAc in petroleum ether) to give 5-methyl-2-(prop-1-yn-1-yl)pyridine as a pale brown liquid.

Yield 358 mg (94%). ¹H NMR (400 MHz, CDCl₃) δ 8.36 (s, 1H), 7.41 (dd, J=2.0, 7.8 Hz, 1H), 7.25 (d, J=7.8 Hz, 1H), 2.32 (s, 3H), 2.07 (s, 3H).

### Synthesis of 5-fluoro-2-(prop-1-yn-1-yl)pyridine

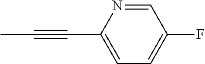

Compound 5-fluoro-2-(prop-1-yn-1-yl)pyridine was prepared from 2-bromo-5-fluoropyridine following a similar procedure to that described for the synthesis of 2-methyl-6-(prop-1-yn-1-yl)pyridine except that it was purified by column chromatography on silica gel (0-20% EtOAc in petroleum ether) to give 5-fluoro-2-(prop-1-yn-1-yl)pyridine as a pale brown liquid.

Yield 291 mg (76%). ¹H NMR (400 MHz, CDCl₃) δ 8.39 (d, J=2.5 Hz, 1H), 7.39-7.30 (m, 2H), 2.07 (s, 3H).

### Synthesis of 4-methyl-3-(prop-1-yn-1-yl)pyridazine

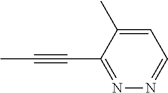

Compound 4-methyl-3-(prop-1-yn-1-yl)pyridazine was prepared from 3-chloro-4-methylpyridazine following a similar procedure to that described for the synthesis of 2-methyl-6-(prop-1-yn-1-yl)pyridine except that it was purified by column chromatography on silica gel (0-100% EtOAc in petroleum ether) to give 4-methyl-3-(prop-1-yn-1-yl)pyridazine as an impure dark brown liquid (impurity=triphenylphosphine oxide). This was taken on to the next step without further purification.

Yield 200 mg (purity=50%, yield=19%). ¹H NMR (400 MHz, DMSO) δ 9.00 (d, J=4.6 Hz, 1H), 2.36 (s, 3H), 2.19 (s, 3H). Other aromatic signal masked by impurity signal. m/z: [ESI⁺] 133 (M+H)⁺.

### Synthesis of 3-methyl-2-(m-tolyl)-1H-indole-5-carbonitrile

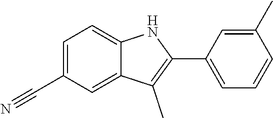 [SMILES: Cc1cccc(-c2[nH]c3ccc(C#N)cc3c2C)c1]

A sealed tube was charged with 4-amino-3-iodobenzonitrile (1.95 g, 7.99 mmol) and 1-methyl-3-(prop-1-yn-1-yl)benzene (1.30 g, 9.99 mmol) in anhydrous DMF (11 mL). To this solution was added K₂CO₃ (2.21 g, 15.98 mmol), LiCl (0.34 g, 7.99 mmol) and [1,1′-bis(diphenylphosphino)ferrocene]dichloropalladium(II) complex with dichloromethane (0.65 g, 0.799 mmol). The resulting suspension was degassed for a few minutes with nitrogen, the reaction tube sealed and the mixture heated to 100° C. for 16 hours. After cooling to room temperature, the reaction mixture was filtered through celite, rinsed with EtOAc and concentrated. The residue was partitioned between ethyl acetate (100 mL) and water (100 mL). The layers were separated, and the organic phase was washed with 4% aqueous LiCl (50 mL) and brine (30 mL), dried (MgSO₄), filtered and evaporated. The residue was purified by column chromatography on silica gel (0-20% EtOAc in cyclohexane) to give 3-methyl-2-(m-tolyl)-1H-indole-5-carbonitrile as a pale brown solid.

Yield 621 mg (32%). ¹H NMR (400 MHz, CDCl₃) δ 8.30 (br s, 1H), 7.94 (s, 1H), 7.44-7.37 (m, 5H), 7.22 (d, J=6.6 Hz, 1H), 2.45 (s, 6H).

### Synthesis of 3-methyl-2-(pyridin-3-yl)-1H-indole-5-carbonitrile

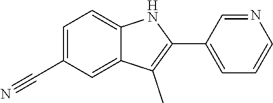 [SMILES: Cc1c(-c2cccnc2)[nH]c2ccc(C#N)cc12]

Compound 3-methyl-2-(pyridin-3-yl)-1H-indole-5-carbonitrile was prepared from 3-(prop-1-yn-1- yl)pyridine following a similar procedure to that described for the synthesis of 3-methyl-2-(m-tolyl)-1H-indole-5-carbonitrile except that it was purified by column chromatography on silica gel (0-20% acetone in DCM) to give 3-methyl-2-(pyridin-3-yl)-1H-indole-5-carbonitrile as a beige solid.

Yield 204 mg (20%). ¹H NMR (400 MHz, CDCl₃) δ 12.01 (br s, 1H), 8.98 (d, J=1.5 Hz, 1H), 8.69-8.66 (m, 1H), 8.23 (s, 1H), 8.16 (d, J=7.8 Hz, 1H), 7.67-7.53 (m, 3H), 2.52 (s, 3H).

### Synthesis of 2-(3-methoxyphenyl)-3-methyl-1H-indole-5-carbonitrile

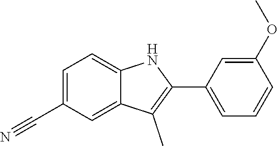 [SMILES: COc1cccc(-c2[nH]c3ccc(C#N)cc3c2C)c1]

Compound 2-(3-methoxyphenyl)-3-methyl-1H-indole-5-carbonitrile was prepared from 1-methoxy-3-(prop-1-yn-1-yl)benzene following a similar procedure to that described for the synthesis of 3-methyl-2- (m-tolyl)-1H-indole-5-carbonitrile except that it was purified by column chromatography on silica gel (0-25% EtOAc in cyclohexane) to give 2-(3-methoxyphenyl)-3-methyl-1H-indole-5-carbonitrile as a yellow oil. This was taken on to the next step without further purification.

Yield 406 mg (51%). ¹H NMR (400 MHz, CDCl₃) δ 8.36 (br s, 1H), 7.94 (s, 1H), 7.45-7.39 (m, 3H), 7.17-7.14 (m, 1H), 7.11-7.07 (m, 1H), 6.97-6.94 (m, 1H), 3.88 (s, 3H), 2.46 (s, 3H).

### Synthesis of 2-(2-methoxyphenyl)-3-methyl-1H-indole-5-carbonitrile

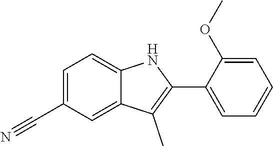 [SMILES: COc1ccccc1-c1[nH]c2ccc(C#N)cc2c1C]

Compound 2-(2-methoxyphenyl)-3-methyl-1H-indole-5-carbonitrile was prepared from 1-methoxy-2-(prop-1-yn-1-yl)benzene following a similar procedure to that described for the synthesis of 3-methyl-2-(m-tolyl)-1H-indole-5-carbonitrile except that it was purified by column chromatography on silica gel (0-15% EtOAc in cyclohexane) to give 2-(2-methoxyphenyl)-3-methyl-1H-indole-5-carbonitrile as an orange solid. This was taken on to the next step without further purification.

Yield 329 mg (42%). ¹H NMR (400 MHz, CDCl₃) δ 8.98 (br s, 1H), 7.94 (s, 1H), 7.53 (dd, J=1.5, 7.6 Hz, 1H), 7.43-7.35 (m, 3H), 7.14-7.03 (m, 2H), 3.91 (s, 3H), 2.42 (s, 3H).

### Synthesis of 3-methyl-2-(2-methylpyridin-3-yl)-1H-indole-5-carbonitrile

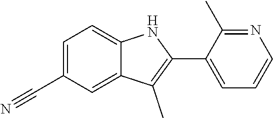 [SMILES: Cc1ncccc1-c1[nH]c2ccc(C#N)cc2c1C]

Compound 3-methyl-2-(2-methylpyridin-3-yl)-1H-indole-5-carbonitrile was prepared from 2-methyl-3-(prop-1-yn-1-yl)pyridine following a similar procedure to that described for the synthesis of 3-methyl-2-(m-tolyl)-1H-indole-5-carbonitrile except that it was purified by column chromatography on silica gel (0-10% acetone in DCM) to give 3-methyl-2-(2-methylpyridin-3-yl)-1H-indole-5-carbonitrile as a brown solid. This was taken on to the next step without further purification.

Yield 141 mg (34%). ¹H NMR (400 MHz, CDCl₃) δ 8.59 (dd, J=1.5, 4.8 Hz, 1H), 8.45 (br s, 1H), 7.96 (s, 1H), 7.65 (dd, J=1.4, 7.7 Hz, 1H), 7.47 (dd, J=1.4, 8.4 Hz, 1H), 7.42 (d, J=8.4 Hz, 1H), 7.28-7.22 (m, 1H), 2.49 (s, 3H), 2.22 (s, 3H).

### Synthesis of 2-(3-fluoropyridin-2-yl)-3-methyl-1H-indole-5-carbonitrile

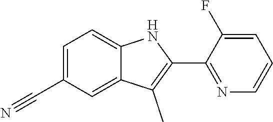 [SMILES: Cc1c(-c2ncccc2F)[nH]c2ccc(C#N)cc12]

Compound 2-(3-fluoropyridin-2-yl)-3-methyl-1H-indole-5-carbonitrile was prepared from 3-fluoro-2-(prop-1-yn-1-yl)pyridine following a similar procedure to that described for the synthesis of 3-methyl-2- (m-tolyl)-1H-indole-5-carbonitrile except that it was purified by column chromatography on silica gel (0-7% acetone in toluene) to give 2-(3-fluoropyridin-2-yl)-3-methyl-1H-indole-5-carbonitrile as a beige solid. This was taken on to the next step without further purification.

Yield 302 mg (68%). ¹H NMR (400 MHz, CDCl₃) δ 9.23 (br s, 1H), 8.53 (d, J=4.3 Hz, 1H), 8.02 (s, 1H), 7.59-7.52 (m, 1H), 7.45 (q, J=8.3 Hz, 2H), 7.35-7.29 (m, 1H), 2.56 (d, J=4.0 Hz, 3H).

### Synthesis of tert-butyl 4-(5-cyano-3-methyl-1H-indol-2-yl)piperidine-1-carboxylate

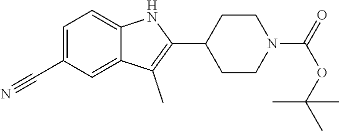 [SMILES: Cc1c(C2CCN(C(=O)OC(C)(C)C)CC2)[nH]c2ccc(C#N)cc12]

Compound tert-butyl 4-(5-cyano-3-methyl-1H-indol-2-yl)piperidine-1-carboxylate was prepared from tert-butyl 4-(prop-1-yn-1-yl)piperidine-1-carboxylate following a similar procedure to that described for the synthesis of 3-methyl-2-(m-tolyl)-1H-indole-5-carbonitrile except that it was purified by column chromatography on silica gel (0-25% EtOAc in cyclohexane) to give tert-butyl 4-(5-cyano-3-methyl-1H-indol-2-yl)piperidine-1-carboxylate as a beige solid. This was taken on to the next step without further purification.

Yield 382 mg (27%). ¹H NMR (400 MHz, CDCl₃) δ 8.83 (s, 1H), 7.83 (s, 1H), 7.35-7.34 (m, 2H), 4.31-4.29 (m, 2H), 3.10-3.00 (m, 1H), 2.87-2.85 (m, 2H), 2.27 (s, 3H), 1.85 (d, J=12.2 Hz, 2H), 1.71 (dd, J=12.2, 12.2 Hz, 2H), 1.51 (s, 9H).

### Synthesis of 3-methyl-2-(tetrahydro-2H-pyran-4-yl)-1H-indole-5-carbonitrile

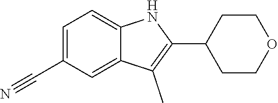 [SMILES: Cc1c(C2CCOCC2)[nH]c2ccc(C#N)cc12]

Compound 3-methyl-2-(tetrahydro-2H-pyran-4-yl)-1H-indole-5-carbonitrile was prepared from 4-(prop-1-yn-1-yl)tetrahydro-2H-pyran following a similar procedure to that described for the synthesis of 3- methyl-2-(m-tolyl)-1H-indole-5-carbonitrile except that it was purified by column chromatography on silica gel (0-40% EtOAc in cyclohexane) to give 3-methyl-2-(tetrahydro-2H-pyran-4-yl)-1H-indole-5-carbonitrile as a pale brown solid. This was taken on to the next step without further purification.

Yield 249 mg (54%). ¹H NMR (400 MHz, CDCl₃) δ 8.08 (s, 1H), 7.84-7.83 (m, 1H), 7.39-7.30 (m, 2H), 4.16-4.10 (m, 2H), 3.62-3.55 (m, 2H), 3.20-3.11 (m, 1H), 2.27 (s, 3H), 1.89-1.80 (m, 4H).

### Synthesis of 3-methyl-2-(2-(trifluoromethyl)phenyl-1H-indole-5-carbonitrile

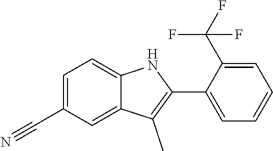 [SMILES: Cc1c(-c2ccccc2C(F)(F)F)[nH]c2ccc(C#N)cc12]

Compound 3-methyl-2-(2-(trifluoromethyl)phenyl)-1H-indole-5-carbonitrile was prepared from 1-(prop-1-yn-1-yl)-2-(trifluoromethyl)benzene following a similar procedure to that described for the synthesis of 3-methyl-2-(m-tolyl)-1H-indole-5-carbonitrile except that it was purified by column chromatography on silica gel (0-25% EtOAc in cyclohexane) to give 3-methyl-2-(2-(trifluoromethyl)phenyl)-1H-indole-5-carbonitrile as a beige solid. This was taken on to the next step without further purification.

Yield 40 mg (32%). ¹H NMR (400 MHz, CDCl₃) δ 8.31 (s, 1H), 7.96 (s, 1H), 7.84 (d, J=7.6 Hz, 1H), 7.69-7.63 (m, 1H), 7.63-7.56 (m, 1H), 7.52-7.43 (m, 2H), 7.43-7.37 (m, 1H), 2.21 (s, 3H).

### Synthesis of 3-methyl-2-(pyrimidin-2-yl)-1H-indole-5-carbonitrile

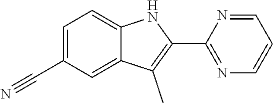 [SMILES: Cc1c(-c2ncccn2)[nH]c2ccc(C#N)cc12]

Compound 3-methyl-2-(pyrimidin-2-yl)-1H-indole-5-carbonitrile was prepared from 2-(prop-1-yn-1- yl)pyrimidine following a similar procedure to that described for the synthesis of 3-methyl-2-(m-tolyl)-1H-indole-5-carbonitrile and was obtained as a beige solid. This was taken on to the next step without further purification.

Yield 120 mg (32%). ¹H NMR (400 MHz, CDCl₃) δ 9.58 (s, 1H), 8.78 (d, J=4.9 Hz, 2H), 8.03 (s, 1H), 7.50-7.46 (m, 1H), 7.43 (dd, J=0.8, 8.5 Hz, 1H), 7.15 (dd, J=4.9, 4.9 Hz, 1H), 2.81 (s, 3H).

### Synthesis of tert-butyl 3-(5-cyano-3-methyl-1H-indol-2-yl)piperidine-1-carboxylate

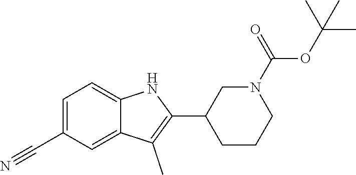 [SMILES: Cc1c(C2CCCN(C(=O)OC(C)(C)C)C2)[nH]c2ccc(C#N)cc12]

Compound tert-butyl 3-(5-cyano-3-methyl-1H-indol-2-yl)piperidine-1-carboxylate was prepared from tert-butyl 3-(prop-1-yn-1-yl)piperidine-1-carboxylate following a similar procedure to that described for the synthesis of 3-methyl-2-(m-tolyl)-1H-indole-5-carbonitrile except that it was purified by reverse-phase column chromatography on RP-C18 silica gel (20-90% acetonitrile in water, 0.1% ammonium bicarbonate) to give tert-butyl 3-(5-cyano-3-methyl-1H-indol-2-yl)piperidine-1-carboxylate as a beige solid. This was taken on to the next step without further purification.

Yield 423 mg (59%). m/z: [ESI⁺] 340 (M+H)⁺ (73% pure).

### Synthesis of (3-methyl-2-(m-tolyl)-1H-indol-5-yl)methanamine

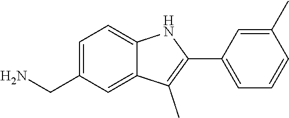 [SMILES: Cc1cccc(-c2[nH]c3ccc(CN)cc3c2C)c1]

To a solution of 3-methyl-2-(m-tolyl)-1H-indole-5-carbonitrile (310 mg, 1.26 mmol) in anhydrous THF (12 mL) was added LiAlH₄ (239 mg, 6.29 mmol) portion wise at room temperature. The reaction mixture was heated at 60° C. for 2 hours. After cooling to 0° C. on ice, water (350 μL) and an aqueous 2N NaOH solution (350 μL) were added dropwise sequentially. The resulting mixture was diluted with ethyl acetate (50 mL), dried (MgSO₄), filtered and evaporated to give (3-methyl-2-(m-tolyl)-1H-indol-5-yl)methanamine as an off-white solid. This was taken on to the next step without further purification.

Yield 309 mg (98%). ¹H NMR (400 MHz, CDCl₃) δ 8.01-7.99 (m, 1H), 7.52 (s, 1H), 7.40-7.32 (m, 4H), 7.18-7.13 (m, 2H), 3.98 (s, 2H), 2.46 (s, 3H), 2.43 (s, 3H). NH₂ protons obscured by residual water peak.

### Synthesis of (3-methyl-2-(pyridin-3-yl)-1H-indol-5-yl)methanamine

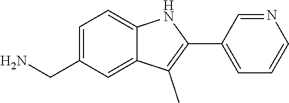 [SMILES: Cc1c(-c2cccnc2)[nH]c2ccc(CN)cc12]

Compound (3-methyl-2-(pyridin-3-yl)-1H-indol-5-yl)methanamine was prepared from 3-methyl-2-(pyridin-3-yl)-1H-indole-5-carbonitrile following a similar procedure to that described for the synthesis of (3-methyl-2-(m-tolyl)-1H-indol-5-yl)methanamine and was obtained as a yellow solid. This was taken on to the next step without further purification.

Yield 223 mg (quant.). ¹H NMR (400 MHz, CDCl₃) δ 11.19 (s, 1H), 8.88 (d, J=1.8 Hz, 1H), 8.55-8.51 (m, 1H), 8.06-8.01 (m, 1H), 7.55-7.47 (m, 2H), 7.29 (d, J=8.1 Hz, 1H), 7.12 (d, J=8.1 Hz, 1H), 3.79 (s, 2H), 2.42 (s, 3H). Two protons obscured by residual water peak.

### Synthesis of (2-(3-methoxyphenyl)-3-methyl-1H-indol-5-yl)methanamine

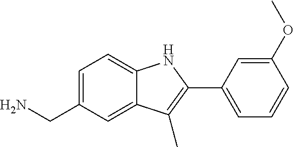 [SMILES: COc1cccc(-c2[nH]c3ccc(CN)cc3c2C)c1]

Compound (2-(3-methoxyphenyl)-3-methyl-1H-indol-5-yl)methanamine was prepared from 2-(3- methoxyphenyl)-3-methyl-1H-indole-5-carbonitrile following a similar procedure to that described for the synthesis of (3-methyl-2-(m-tolyl)-1H-indol-5-yl)methanamine and was obtained as a pale yellow oil. This was taken on to the next step without further purification.

Yield 407 mg (quant.). ¹H NMR (400 MHz, CDCl₃) δ 8.02 (s, 1H), 7.53 (s, 1H), 7.41-7.32 (m, 2H), 7.19-7.10 (m, 3H), 6.93-6.89 (m, 1H), 3.98 (s, 2H), 3.88 (s, 3H), 2.47 (s, 3H). NH₂ protons obscured by residual water peak.

### Synthesis of (2-(2-methoxyphenyl)-3-methyl-1H-indol-5-yl)methanamine

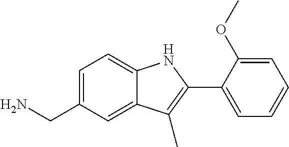 [SMILES: COc1ccccc1-c1[nH]c2ccc(CN)cc2c1C]

Compound (2-(2-methoxyphenyl)-3-methyl-1H-indol-5-yl)methanamine was prepared from 2-(2- methoxyphenyl)-3-methyl-1H-indole-5-carbonitrile following a similar procedure to that described for the synthesis of (3-methyl-2-(m-tolyl)-1H-indol-5-yl)methanamine and was obtained as a pale yellow solid. This was taken on to the next step without further purification.

Yield 318 mg (95%). ¹H NMR (400 MHz, CDCl₃) δ 8.71-8.66 (m, 1H), 7.55-7.51 (m, 2H), 7.37-7.32 (m, 2H), 7.14 (dd, J=1.6, 8.3 Hz, 1H), 7.08 (ddd, J=1.2, 7.5, 7.5 Hz, 1H), 7.03 (dd, J=0.8, 8.3 Hz, 1H), 3.97 (s, 2H), 3.89 (s, 3H), 2.43 (s, 3H). NH₂ protons obscured by residual water peak.

### Synthesis of (3-methyl-2-(2-methylpyridin-3-yl)-1H-indol-5-yl)methanamine

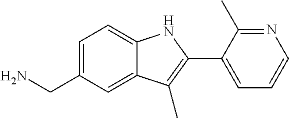 [SMILES: Cc1ncccc1-c1[nH]c2ccc(CN)cc2c1C]

Compound (3-methyl-2-(2-methylpyridin-3-yl)-1H-indol-5-yl)methanamine was prepared from 3-methyl-2-(2-methylpyridin-3-yl)-1H-indole-5-carbonitrile following a similar procedure to that described for the synthesis of (3-methyl-2-(m-tolyl)-1H-indol-5-yl)methanamine and was obtained as a pale yellow oil. This was taken on to the next step without further purification.

Yield 150 mg (quant.). ¹H NMR (400 MHz, CDCl₃) δ 8.57 (dd, J=1.8, 4.8 Hz, 1H), 7.93 (s, 1H), 7.64 (dd, J=1.8, 7.7 Hz, 1H), 7.56-7.54 (m, 1H), 7.34 (d, J=8.3 Hz, 1H), 7.24-7.18 (m, 2H), 4.00 (s, 2H), 2.52 (s, 3H), 2.21 (s, 3H). NH₂ protons obscured by residual water peak.

### Synthesis of (2-(3-fluoropyridin-2-yl)-3-methyl-1H-indol-5-yl)methanamine

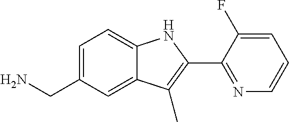 [SMILES: Cc1c(-c2ncccc2F)[nH]c2ccc(CN)cc12]

Compound (2-(3-fluoropyridin-2-yl)-3-methyl-1H-indol-5-yl)methanamine was prepared from 2-(3-fluoropyridin-2-yl)-3-methyl-1H-indole-5-carbonitrile following a similar procedure to that described for the synthesis of (3-methyl-2-(m-tolyl)-1H-indol-5-yl)methanamine and was obtained as a yellow oil. This was taken on to the next step without further purification.

Yield 285 mg. m/z: [ESI+] 239 (fragment: M-NH₂)⁺ (40% pure).

### Synthesis of (3-methyl-2-(1-methylpiperidin-4-yl)-1H-indol-5-yl)methanamine

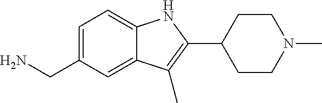 [SMILES: Cc1c(C2CCN(C)CC2)[nH]c2ccc(CN)cc12]

Compound (3-methyl-2-(1-methylpiperidin-4-yl)-1H-indol-5-yl)methanamine was prepared from tertbutyl 4-(5-cyano-3-methyl-1H-indol-2-yl)piperidine-1-carboxylate following a similar procedure to that described for the synthesis of (3-methyl-2-(m-tolyl)-1H-indol-5-yl)methanamine except that the quantity of LiAlH₄ was increased to 10 equivalents (to aid removal of the Boc group). (3-Methyl-2-(1-methylpiperidin-4-yl)-1H-indol-5-yl)methanamine was obtained as a pale pink oil. This was taken on to the next step without further purification.

Yield 273 mg (94%). ¹H NMR (400 MHz, CDCl₃) δ 7.80 (s, 1H), 7.42-7.40 (m, 1H), 7.24 (d, J=7.8 Hz, 1H), 7.06 (dd, J=1.6, 8.2 Hz, 1H), 3.93 (s, 2H), 3.02-2.96 (m, 2H), 2.88-2.78 (m, 1H), 2.34 (s, 3H), 2.25 (s, 3H), 2.13-2.05 (m, 2H), 1.91-1.69 (m, 4H). NH₂ protons obscured by residual water peak.

### Synthesis of (3-methyl-2-(tetrahydro-2H-pyran-4-yl)-1H-indol-5-yl)methanamine

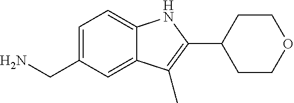 [SMILES: Cc1c(C2CCOCC2)[nH]c2ccc(CN)cc12]

Compound (3-methyl-2-(tetrahydro-2H-pyran-4-yl)-1H-indol-5-yl)methanamine was prepared from 3-methyl-2-(tetrahydro-2H-pyran-4-yl)-1H-indole-5-carbonitrile following a similar procedure to that described for the synthesis of (3-methyl-2-(m-tolyl)-1H-indol-5-yl)methanamine and was obtained as a pale orange solid. This was taken on to the next step without further purification.

Yield 250 mg (quant.). ¹H NMR (400 MHz, DMSO) δ 10.49 (s, 1H), 7.30 (s, 1H), 7.15 (d, J=8.3 Hz, 1H), 6.96 (dd, J=1.7, 8.3 Hz, 1H), 3.96 (dd, J=4.2, 11.0 Hz, 2H), 3.74 (s, 2H), 3.50-3.42 (m, 2H), 3.11-3.02 (m, 1H), 2.18 (s, 3H), 1.94-1.81 (m, 2H), 1.61 (dd, J=2.1, 12.8 Hz, 2H). NH₂ protons obscured under residual water peak.

### Synthesis of (3-methyl-2-(2-(trifluoromethyl)phenyl)-1H-indol-5-yl)methanamine

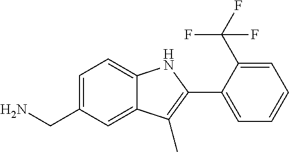 [SMILES: Cc1c(-c2ccccc2C(F)(F)F)[nH]c2ccc(CN)cc12]

Compound (3-methyl-2-(2-(trifluoromethyl)phenyl)-1H-indol-5-yl)methanamine was prepared from 3- methyl-2-(2-(trifluoromethyl)phenyl)-1H-indole-5-carbonitrile following a similar procedure to that described for the synthesis of (3-methyl-2-(m-tolyl)-1H-indol-5-yl)methanamine and was obtained as a yellow oil. This was taken on to the next step without further purification.

Yield 70 mg. m/z: [ESI⁺] 288 (fragment: M-NH₂)⁺ (54% pure).

### Synthesis of (3-methyl-2-(pyrimidin-2-yl)-1H-indol-5-yl)methanamine

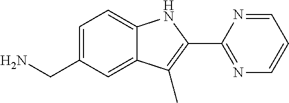 [SMILES: Cc1c(-c2ncccn2)[nH]c2ccc(CN)cc12]

Compound (3-methyl-2-(pyrimidin-2-yl)-1H-indol-5-yl)methanamine was prepared from 3-methyl-2-(pyrimidin-2-yl)-1H-indole-5-carbonitrile following a similar procedure to that described for the synthesis of (3-methyl-2-(m-tolyl)-1H-indol-5-yl)methanamine and was obtained as a brown solid. This was taken on to the next step without further purification.

Yield 114 mg. m/z: [ESI⁺] 222 (fragment: M-NH₂)⁺ (54% pure).

### Synthesis of (3-methyl-2-(1-methylpiperidin-3-yl)-1H-indol-5-yl)methanamine

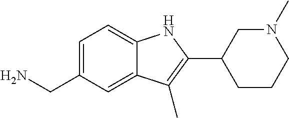 [SMILES: Cc1c(C2CCCN(C)C2)[nH]c2ccc(CN)cc12]

Compound (3-methyl-2-(1-methylpiperidin-3-yl)-1H-indol-5-yl)methanamine was prepared from tertbutyl 3-(5-cyano-3-methyl-1H-indol-2-yl)piperidine-1-carboxylate following a similar procedure to that described for the synthesis of (3-methyl-2-(m-tolyl)-1H-indol-5-yl)methanamine except that the quantity of LiAlH₄ was increased to 10 equivalents (to aid removal of the Boc group). (3-Methyl-2-(1-methylpiperidin-3-yl)-1H-indol-5-yl)methanamine was obtained as a pale pink oil. This was taken on to the next step without further purification.

Yield 331 mg. m/z: [ESI⁺] 258 (M+H)⁺ (100% pure).

### Synthesis of 4-(aminomethyl)-2-iodoaniline dihydrochloride

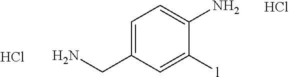

To a solution of 4-amino-3-iodobenzonitrile (3.00 g, 12.29 mmol) in anhydrous THF at room temperature (30 mL) was slowly added BH₃-THF complex (1M in THF, 36.9 mL, 36.88 mmol). After stirring under reflux (external temperature=85° C.) for 3.5 hours, the reaction mixture was allowed to cool to room temperature. After slow addition of aqueous HCl (2M, 8 mL), the reaction mixture was stirred under reflux for 1 hour and was then evaporated to give 4-(aminomethyl)-2-iodoaniline dihydrochloride as a white solid.

Yield 3.95 g (quant.). ¹H NMR (400 MHz, DMSO) δ 8.18-8.08 (br s, 3H), 7.68 (d, J=2.0 Hz, 1H), 7.17 (dd, J=2.0, 8.3 Hz, 1H), 6.74 (d, J=8.3 Hz, 1H), 3.80 (q, J=5.7 Hz, 2H). Three protons obscured by solvent peak.

### Synthesis of N-(4-amino-3-iodobenzyl-4-methylpyrimidine-5-carboxamide

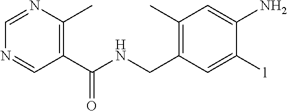

To a solution of 4-methylpyrimidine-5-carboxylic acid (0.66 g, 4.75 mmol) in anhydrous DMF (10 mL) were added sequentially DIPEA (1.23 mL, 7.13 mmol) and HATU (1.99 g, 5.23 mmol) and the reaction mixture was stirred at room temperature for 10 minutes. A solution of 4-(aminomethyl)-2-iodoaniline dihydrochloride (2.21 g, 4.75 mmol) and DIPEA (2.06 mL, 11.88 mmol) in anhydrous DMF (10 mL) was then added and the reaction continued to stir at room temperature for 1.5 hours. The mixture was partitioned between ethyl acetate (200 mL) and a diluted Na₂CO₃ solution (10%, 200 mL). The layers were separated, and the organic phase was washed with a diluted solution of Na₂CO₃ (10%, 100 mL) and brine (50 mL), dried (MgSO₄), filtered and evaporated. The residue was triturated with petroleum ether and evaporated to give N-(4-amino-3-iodobenzyl)-4-methylpyrimidine-5-carboxamide as a yellow solid.

Yield 1.22 g (70%). ¹H NMR (400 MHz, DMSO) δ 9.07 (s, 1H), 9.00 (dd, J=5.7, 5.7 Hz, 1H), 8.69 (s, 1H), 7.54 (d, J=2.0 Hz, 1H), 7.08 (dd, J=2.0, 8.1 Hz, 1H), 6.73 (d, J=8.1 Hz, 1H), 5.15 (s, 2H), 4.28 (d, J=5.8 Hz, 2H), 2.51 (s, 3H).

### Synthesis of (2-(pyridin-2-yl)-1H-benzo[d]imidazol-5-yl)methanamine

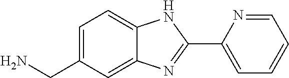 [SMILES: NCc1ccc2[nH]c(-c3ccccn3)nc2c1]

To a solution of 2-(pyridin-2-yl)-1H-benzo[d]imidazole-5-carbonitrile (250 mg, 1.14 mmol) in anhydrous THF (12 mL) was added LiAlH₄ (215 mg, 5.68 mmol) portion wise at room temperature. The reaction mixture was heated at 60° C. for 2 hours. After cooling to 0° C. on ice, water (350 μL) and an aqueous 2N NaOH solution (350 μL) were added dropwise sequentially. The resulting mixture was diluted with ethyl acetate (50 mL), dried (MgSO₄), filtered and evaporated to give (2-(pyridin-2-yl)-1H-benzo[d]imidazol-5-yl)methanamine as an off-white solid. This was taken on to the next step without further purification.

Yield 40 mg (16%). ¹H NMR (400 MHz, DMSO) δ 8.72 (d, J=4.0 Hz, 1H), 8.30 (d, J=7.8 Hz, 1H), 8.01-7.96 (m, 1H), 7.64-7.57 (m, 1H), 7.50 (ddt, J=1.2, 3.7, 4.1 Hz, 2H), 7.47-7.33 (m, 1H), 7.21-7.17 (m, 1H), 3.82 (s, 2H), 3.38 (br s, 2H).

### Synthesis of 3-methyl-2-(pyridin-2-1H-pyrrolo[3,2-b]pyridine-5-carbonitrile (20)

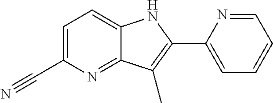 [SMILES: Cc1c(-c2ccccn2)[nH]c2ccc(C#N)nc12]

A sealed tube was charged at room temperature with 5-amino-6-iodopicolinonitrile (1.03 g, 4.22 mmol), K₂CO₃ (1.17 g, 8.43 mmol), LiCl (0.18 g, 4.22 mmol) and [1,1′-bis(diphenylphosphino)ferrocene]dichloropalladium(II) complex with dichloromethane (0.34 g, 0.42 mmol). To this mixture was added a solution of 2-(prop-1-yn-1-yl)pyridine (0.62 g, 5.12 mmol) in anhydrous DMF (12 mL). The resulting suspension was degassed for a few minutes with nitrogen, the reaction tube sealed and the mixture heated to 100° C. for 18 hours. After cooling to room temperature, the reaction mixture was filtered through celite, rinsed with ethyl acetate and concentrated. The residue was partitioned between ethyl acetate (100 mL) and water (100 mL). The layers were separated, and the organic phase was washed with water (50 mL) and brine (30 mL), dried (MgSO₄), filtered and evaporated. The residue was purified by column chromatography on silica gel (0-10% EtOAc in isohexane) to give 3-methyl-2-(pyridin-2-yl)-1H-pyrrolo[3,2-b]pyridine-5-carbonitrile (20) as a yellow solid.

Yield 180 mg. m/z: [ESI⁺] 235 (M+H)⁺ (75% pure).

### Synthesis of tert-butyl ((3-methyl-2-(pyridin-2-yl)-1H-pyrrolo[3,2-b]pyridin-5-yl)methyl)carbamate

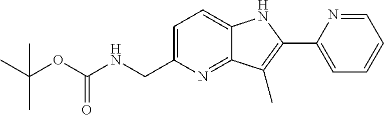 [SMILES: Cc1c(-c2ccccn2)[nH]c2ccc(CNC(=O)OC(C)(C)C)nc12]

To a suspension of 3-methyl-2-(pyridin-2-yl)-1H-pyrrolo[3,2-b]pyridine-5-carbonitrile (180 mg, 0.768 mmol) in anhydrous MeOH (8 mL) was added nickel(II) chloride hexahydrate (18 mg, 0.0768 mmol) and di-tert-butyl dicarbonate (503 mg, 2.31 mmol) at room temperature. To the resulting solution sodium borohydride (233 mg, 6.15 mmol) was added portion wise over a period of 10 minutes. After stirring for 15 minutes at room temperature, the reaction was re-charged with additional di-tert-butyl dicarbonate (167 mg, 0.765 mmol) followed by additional sodium borohydride (233 mg, 6.15 mmol) which was added portion wise over a period of 10 minutes. The reaction was re-charged in this manner a further 4 times until LCMS monitoring showed no starting material remained. The reaction mixture was then partitioned between ethyl acetate (50 mL) and a saturated NaHCO₃ solution (50 mL). The layers were separated, and the organic phase was washed with brine (20 mL), dried (MgSO₄), filtered and evaporated. The residue was purified by column chromatography on silica gel (0-10% EtOAc in iso-hexane) to give tert-butyl ((3-methyl-2-(pyridin-2-yl)-1H-pyrrolo[3,2-b]pyridin-5-yl)methyl)carbamate as a yellow oil. This was taken on to the next step without further purification.

Yield 50 mg. m/z: [ESI⁺] 339 (M+H)⁺ (65% pure). No NMR data due to limited material.

### Synthesis of(3-methyl-2-(pyridin-2-yl)-1H-pyrrolo[3,2-b]pyridin-5-yl)methanamine (21)

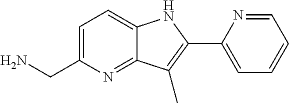 [SMILES: Cc1c(-c2ccccn2)[nH]c2ccc(CN)nc12]

To a solution of tert-butyl ((3-methyl-2-(pyridin-2-yl)-1H-pyrrolo[3,2-b]pyridin-5-yl)methyl)carbamate (50 mg, 0.148 mmol) in 1,4-dioxane (1 mL) was added hydrogen chloride in dioxane (4 M, 0.74 mL, 2.95 mmol). After stirring at room temperature for 16 hours, the reaction mixture was evaporated and the resulting residue was partitioned between DCM (25 mL) and 1M aqueous NaOH (25 mL). The organic phase was dried through phase separating filter paper and evaporated to give (3-methyl-2-(pyridin-2-yl)-1H-pyrrolo[3,2-b]pyridin-5-yl)methanamine (21) as a yellow solid. This was taken on to the next step without further purification.

Yield 20 mg (57%). m/z: [ESI⁺] 239 (M+H)⁺ (90% pure). No NMR data due to limited material.

### Synthesis of N-(4-amino-3-((tetrahydro-2H-pyran-4-yl)ethynyl)benzyl)-4-methylpyrimidine-5-carboxamide

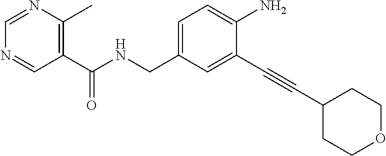 [SMILES: Cc1ncncc1C(=O)NCc1ccc(N)c(C#CC2CCOCC2)c1]

A suspension of N-(4-amino-3-iodobenzyl)-4-methylpyrimidine-5-carboxamide (150 mg, 0.41 mmol), 4-ethynyltetrahydro-2H-pyran (67 mg, 0.61 mmol), bis(triphenylphosphine)palladium(II) dichloride (17 mg, 0.02 mmol) and copper(I) iodide (8 mg, 0.04 mmol) in DMF (1.5 mL) was sparged at room temperature for 5 minutes with nitrogen then diethylamine (63 μL, 0.61 mmol) was added and the mixture was stirred at room temperature for 4 hours. The reaction mixture was diluted in water (20 mL) and extracted with DCM (2×20 mL). The organic phase was passed through a phase separator paper to dry and evaporated. The residue was purified by column chromatography on silica gel (0-10% methanol in DCM) to afford N-(4-amino-3-((tetrahydro-2H-pyran-4-yl)ethynyl)benzyl)-4-methylpyrimidine-5-carboxamide as a yellow gum.

Yield 49 mg. m/z: [ESI⁺] 351 (M+H)⁺ (38% pure).

### Synthesis of ethyl 2-(4-amino-3-iodophenyl)acetate (41)

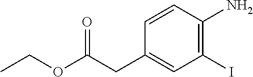

To a solution of ethyl 2-(4-aminophenyl)acetate (10.00 g, 55.80 mmol) in acetonitrile (100 mL) was added N-iodosuccinimide (15.06 g, 66.94 mmol) at room temperature. The resulting mixture was refluxed overnight under a nitrogen atmosphere. After cooling down to room temperature, the resulting mixture was concentrated and the residue was purified by column chromatography on silica gel (1% to 20% ethyl acetate in petroleum ether). The fractions containing the desired product were collected and concentrated under reduced pressure to afford ethyl 2-(4-amino-3-iodophenyl)acetate (41) as a light yellow oil.

Yield 15.31 g (90%). ¹H NMR (400 MHz, DMSO) δ 7.45 (s, 1H), 6.97 (d, J=8.2 Hz, 1H), 6.70 (d, J=8.2 Hz, 1H), 5.13 (br s, 2H), 4.05 (t, J=7.2 Hz, 2H), 3.45 (s, 2H), 1.18 (t, J=7.2 Hz, 3H). m/z: [ESI⁺]306 (M+H)⁺.

### Synthesis of ethyl 2-(3-methyl-2-(tetrahydro-2H-pyran-4-yl)-1H-indol-5-yl)acetate

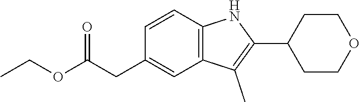 [SMILES: CCOC(=O)Cc1ccc2[nH]c(C3CCOCC3)c(C)c2c1]

Compound ethyl 2-(3-methyl-2-(tetrahydro-2H-pyran-4-yl)-1H-indol-5-yl)acetate was prepared from ethyl 2-(4-amino-3-iodophenyl)acetate (400 mg, 1.311 mmol) following a similar procedure to that described for the synthesis of 3-methyl-2-(m-tolyl)-1H-indole-5-carbonitrile to give ethyl 2-(3-methyl-2-(tetrahydro-2H-pyran-4-yl)-1H-indol-5-yl)acetate as a yellow oil.

Yield 180 mg (46%). ¹H NMR (400 MHz, CD₃OD) δ 7.31 (d, J=1.6 Hz, 1H), 7.22 (d, J=8.4 Hz, 1H), 6.95 (dd, J=1.6, 8.4 Hz, 1H), 4.14 (q, J=7.2 Hz, 2H), 4.07 (dd, J=4.0, 11.2 Hz, 2H), 3.67 (s, 2H), 3.61 (dt, J=2.0, 12.0 Hz, 2H), 3.16 (tt, J=3.6, 12.0 Hz, 1H), 2.25 (s, 3H), 1.95 (dq, J=4.4, 12.8 Hz, 2H), 1.74 (ddd, J=2.0, 4.0, 13.2 Hz, 2H), 1.25 (t, J=7.2 Hz, 3H). m/z: [ESI⁺] 302 (M+H)⁺.

### Synthesis of 2-(3-methyl-2-(tetrahydro-2H-pyran-4-yl)-1H-indol-5-yl)acetic acid

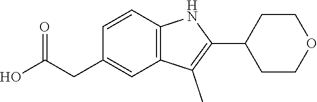 [SMILES: Cc1c(C2CCOCC2)[nH]c2ccc(CC(=O)O)cc12]

A solution of ethyl 2-(3-methyl-2-(tetrahydro-2H-pyran-4-yl)-1H-indol-5-yl)acetate (170 mg, 0.564 mmol) in THF (4 mL) and H₂O (4 mL) was treated with LiOH (135 mg, 5.637 mmol) overnight at 50° C. The resulting mixture was cooled down to room temperature and acidified to pH 5 with aqueous HCl (1N). To the above mixture was added brine (30 mL) and the resulting mixture was extracted with ethyl acetate (3×10 mL). The combined organic layers were washed with brine (2×5 mL) and dried over anhydrous Na₂SO₄. After filtration, the filtrate was concentrated under reduced pressure to give 2-(3-methyl-2-(tetrahydro-2H-pyran-4-yl)-1H-indol-5-yl)acetic acid as a yellow oil, which was used in the next step directly without further purification.

Yield 110 mg (71%). ¹H NMR (400 MHz, DMSO) δ 10.58 (s, 1H), 7.24 (d, J=1.6 Hz, 1H), 7.17 (d, J=8.4 Hz, 1H), 6.90 (dd, J=1.6, 8.4 Hz, 1H), 3.97 (dd, J=4.0, 11.2 Hz, 2H), 3.55 (s, 2H), 3.47 (dt, J=2.0, 12.0 Hz, 2H), 3.09 (tt, J=3.6, 12.0 Hz, 1H), 2.18 (s, 3H), 1.89 (dq, J=4.4, 12.8 Hz, 2H), 1.62 (dd, J=4.0, 13.2 Hz, 2H). COOH was not observed. m/z: [ESI⁺] 274 (M+H)⁺.

### Synthesis of 4-(aminomethyl)-5-fluoro-2-iodoaniline

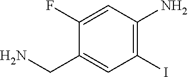 [SMILES: NCc1cc(I)c(N)cc1F]

Compound 4-(aminomethyl)-5-fluoro-2-iodoaniline was prepared from 4-amino-2-fluoro-5-iodobenzonitrile (2.00 g, 7.63 mmol) following a similar procedure to that described for the synthesis of 4-(aminomethyl)-2-iodoaniline dihydrochloride except that after concentration, the mixture was basified to pH 8 with saturated aqueous NaHCO₃. To the above mixture was added brine (100 mL). The resulting mixture was stirred for additional 20 min at room temperature. The resulting mixture was extracted with ethyl acetate (3×40 mL). The combined organic layers were washed with brine (2×20 mL) and dried over anhydrous Na₂SO₄. After filtration, the filtrate was concentrated under reduced pressure to give 4-(aminomethyl)-5-fluoro-2-iodoaniline as a light yellow solid.

Yield 2.00 g (98%). ¹H NMR (400 MHz, DMSO) δ 7.75 (d, J=8.4 Hz, 1H), 6.61 (d, J=8.4 Hz, 1H), 6.12 (br s, 2H), 3.84 (d, J=5.6 Hz, 2H). aliphatic NH₂ was not observed. m/z: [ESI⁺] 250 (M-NH₂)⁺

### Synthesis of N-(4-amino-2-fluoro-5-iodobenzyl)-4-methylpyrimidine-5-carboxamide

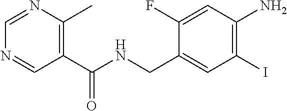 [SMILES: Cc1ncncc1C(=O)NCc1cc(I)c(N)cc1F]

Compound N-(4-amino-2-fluoro-5-iodobenzyl)-4-methylpyrimidine-5-carboxamide was prepared from 4-methylpyrimidine-5-carboxylic acid (260 mg, 1.879 mmol) and 4-(aminomethyl)-5-fluoro-2-iodoaniline (500 mg, 1.879 mmol) following a similar procedure to that described for the synthesis of N-(4-amino-3-iodobenzyl)-4-methylpyrimidine-5-carboxamide to give N-(4-amino-2-fluoro-5-iodobenzyl)-4-methylpyrimidine-5-carboxamide as a yellow solid.

Yield 200 mg (28%). ¹H NMR (400 MHz, DMSO) δ 9.08 (d, J=2.4 Hz, 1H), 8.98 (t, J=5.6 Hz, 1H), 8.67 (d, J=2.4 Hz, 1H), 7.58 (d, J=8.4 Hz, 1H), 6.56 (d, J=12.4 Hz, 1H), 5.47 (br s, 2H), 4.32 (d, J=5.6 Hz, 2H), 2.49 (s, 3H). m/z: [ESI⁺] 387 (M+H)⁺.

### Synthesis of N-(4-amino-3-fluoro-5-iodobenzyl)-4-methylpyrimidine-5-carboxamide

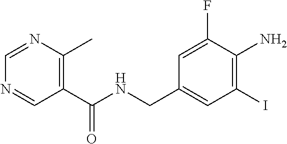 [SMILES: Cc1ncncc1C(=O)NCc1cc(F)c(N)c(I)c1]

Compound N-(4-amino-3-fluoro-5-iodobenzyl)-4-methylpyrimidine-5-carboxamide was prepared from 4-methylpyrimidine-5-carboxylic acid (0.65 g, 4.71 mmol) and 4-(aminomethyl)-2-fluoro-6-iodoaniline (1.26 g, 4.74 mmol) following a similar procedure to that described for the synthesis of N-(4-amino-3-iodobenzyl)-4-methylpyrimidine-5-carboxamide to give N-(4-amino-3-fluoro-5-iodobenzyl)-4-methylpyrimidine-5-carboxamide as a light yellow solid.

Yield 0.60 g (33%). m/z: [ESI⁺] 387 (M+H)⁺

### Synthesis of tert-butyl ((3-methyl-1H-indol-5-yl)methyl)carbamate (51)

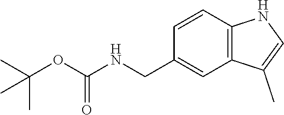 [SMILES: Cc1c[nH]c2ccc(CNC(=O)OC(C)(C)C)cc12]

A mixture of 1-(3-methyl-1H-indol-5-yl)methanamine (50) (10.50 g, 65.53 mmol) and di-tert-butyl dicarbonate (17.16 g, 78.63 mmol) in acetonitrile (100 mL) was stirred overnight at room temperature. The resulting mixture was concentrated under reduced pressure. The residue was diluted with water (200 mL) and extracted with DCM (2×100 mL). The combined organic layers were washed with brine (50 mL) and dried over anhydrous Na₂SO₄. After filtration, the filtrate was concentrated under reduced pressure. The residue was purified by silica gel column chromatography, eluting with 9% of ethyl acetate in DCM to afford tert-butyl ((3-methyl-1H-indol-5-yl)methyl)carbamate (51) as a brown solid.

Yield 6.43 g (38%). ¹H NMR (400 MHz, CDCl₃) δ 8.07 (s, 1H), 7.50 (d, J=1.6 Hz, 1H), 7.31 (d, J=8.4 Hz, 1H), 7.14 (dd, J=1.6, 8.4 Hz, 1H), 6.99 (s, 1H), 4.87 (br s, 1H), 4.45 (s, 2H), 2.35 (s, 3H), 1.52 (s, 9H). m/z: [ESI⁻] 259 (M−H)⁻.

### Synthesis of tert-butyl ((2-bromo-3-methyl-1H-indol-5-yl)methyl)carbamate (52)

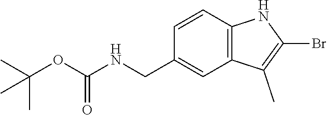 [SMILES: Cc1c(Br)[nH]c2ccc(CNC(=O)OC(C)(C)C)cc12]

To a solution of tert-butyl ((3-methyl-1H-indol-5-yl)methyl)carbamate (51) (6.43 g, 24.70 mmol) in carbon tetrachloride (60 mL) and DCM (60 mL) was added NBS (4.84 g, 27.19 mmol) in several portions at 0° C. The resulting mixture was stirred for 2 h at 0° C. The reaction was diluted with water (120 mL) at room temperature. The resulting mixture was extracted with DCM (2×60 mL). The combined organic layers were washed with brine (60 mL) and dried over anhydrous Na₂SO₄. After filtration, the filtrate was concentrated under reduced pressure. The residue was purified by column chromatography on silica gel (17% of ethyl acetate in DCM) to afford tert-butyl ((2-bromo-3-methyl-1H-indol-5-yl)methyl)carbamate (52) as a yellow liquid.

Yield 6.40 g (76%). ¹H NMR (400 MHz, CDCl₃) δ 8.02 (s, 1H), 7.40 (d, J=1.6 Hz, 1H), 7.24 (d, J=8.4 Hz, 1H), 7.11 (dd, J=1.6, 8.4 Hz, 1H), 4.84 (br s, 1H), 4.42 (s, 2H), 2.26 (s, 3H), 1.50 (s, 9H). m/z: [ESI⁺] 339, 341 (M+H)⁺.

### Synthesis of tert-butyl ((2-(3,6-dihydro-2H-pyran-4-yl)-3-methyl-1H-indol-5-yl)methyl)carbamate (65)

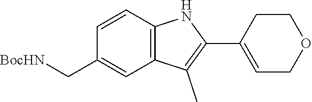 [SMILES: Cc1c(C2=CCOCC2)[nH]c2ccc(CNC(=O)OC(C)(C)C)cc12]

To a stirred mixture of tert-butyl ((2-bromo-3-methyl-1H-indol-5-yl)methyl)carbamate (52) (6.00 g, 17.69 mmol) in 1,4-dioxane (100 mL) and H₂O (10 mL) were added Na₂CO₃ (2.81 g, 26.53 mmol), 2-(3,6-dihydro-2H-pyran-4-yl)-4,4,5,5-tetramethyl-1,3,2-dioxaborolane (64) (11.15 g, 53.08 mmol) and Pd(PPh₃)₄ (3.07 g, 2.66 mmol) respectively at room temperature under a nitrogen atmosphere. The resulting mixture was purged with nitrogen gas for three times and stirred overnight at 100° C. under a nitrogen atmosphere. The mixture was allowed to cool down to room temperature and diluted with water (200 mL). The resulting mixture was extracted with DCM (2×100 mL). The combined organic layers were washed with brine (100 mL) and dried over anhydrous Na₂SO₄. After filtration, the filtrate was concentrated under reduced pressure. The residue was purified by column chromatography on silica gel (50% of ethyl acetate in DCM) to afford tert-butyl ((2-(3,6-dihydro-2H-pyran-4-yl)-3-methyl-1H-indol-5-yl)methyl)carbamate (65) as a yellow liquid.

Yield 3.60 g (59%). ¹H NMR (400 MHz, CDCl₃) δ 8.07 (s, 1H), 7.44 (d, J=1.6 Hz, 1H), 7.26 (d, J=8.4 Hz, 1H), 7.10 (dd, J=1.6, 8.4 Hz, 1H), 6.08-5.99 (m, 1H), 4.87 (br s, 1H), 4.42 (d, J=5.6 Hz, 2H), 4.39 (q, J=2.9 Hz, 2H), 3.96 (t, J=5.4 Hz, 2H), 2.60 (qd, J=2.6, 4.8 Hz, 2H), 2.39 (s, 3H), 1.50 (s, 9H). m/z: [ESI⁺] 343 (M+H)⁺.

### Synthesis of tert-butyl ((3-methyl-2-(tetrahydro-2H-pyran-4-yl)-1H-indol-5-yl)methyl)carbamate (66)

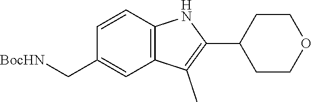 [SMILES: Cc1c(C2CCOCC2)[nH]c2ccc(CNC(=O)OC(C)(C)C)cc12]

To a stirred solution of tert-butyl ((2-(3,6-dihydro-2H-pyran-4-yl)-3-methyl-1H-indol-5-yl)methyl)carbamate (65) (3.60 g, 10.51 mmol) in methanol (50 mL) and AcOH (50 mL) was added 10% palladium on activated carbon (0.60 g) in portions at room temperature under a nitrogen atmosphere. The resulting mixture was stirred overnight at room temperature under a hydrogen atmosphere. The resulting mixture was filtered. The filtered cake was washed with methanol (5×30 mL). The combined filtrates were concentrated under reduced pressure to afford tert-butyl ((3-methyl-2-(tetrahydro-2H-pyran-4-yl)-1H-indol-5-yl)methyl)carbamate (66) as a yellow liquid.

Yield 2.00 g (55%). ¹H NMR (400 MHz, CDCl₃) δ 7.93 (s, 1H), 7.43 (d, J=1.6 Hz, 1H), 7.28 (d, J=8.4 Hz, 1H), 7.10 (dd, J=1.6, 8.4 Hz, 1H), 4.81 (br s, 1H), 4.42 (d, J=5.6 Hz, 2H), 4.19-4.09 (m, 2H), 3.61 (dt, J=2.4, 11.6 Hz, 2H), 3.16 (tt, J=4.0, 12.4 Hz, 1H), 2.28 (s, 3H), 1.98-1.85 (m, 2H), 1.82 (ddd, J=1.6, 4.0, 12.4 Hz, 2H), 1.50 (s, 9H). m/z: [ESI⁺] 345 (M+H)⁺.

### Synthesis of (3-methyl-2-(tetrahydro-2H-pyran-4-yl)-1H-indol-5-yl)methanamine hydrochloride (67)

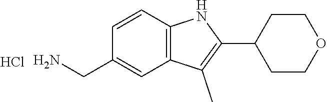 [SMILES: Cl ; Cc1c(C2CCOCC2)[nH]c2ccc(CN)cc12]

Compound (3-methyl-2-(tetrahydro-2H-pyran-4-yl)-1H-indol-5-yl)methanamine hydrochloride (67) was prepared from tert-butyl ((3-methyl-2-(tetrahydro-2H-pyran-4-yl)-1H-indol-5-yl)methyl)carbamate (2.61 g, 7.58 mmol) following a similar procedure to that described for the synthesis of (2-(2-fluorophenyl)-3-methyl-1H-indol-5-yl)methanamine hydrochloride to afford (3-methyl-2-(tetrahydro-2H-pyran-4-yl)-1H-indol-5-yl)methanamine hydrochloride (67) as a dark green solid.

Yield 1.00 g (47%). ¹H NMR (400 MHz, DMSO) δ 10.90 (s, 1H), 8.44 (br s, 3H, NH₃⁺), 7.53 (d, J=1.6 Hz, 1H), 7.29 (d, J=8.4 Hz, 1H), 7.14 (dd, J=1.6, 8.4 Hz, 1H), 4.03 (dd, J=3.2, 6.0 Hz, 2H), 3.97 (dd, J=4.0, 11.6 Hz, 2H), 3.53-3.43 (m, 2H), 3.11 (tt, J=3.6, 12.0 Hz, 1H), 2.21 (s, 3H), 1.91 (dq, J=4.4, 12.6 Hz, 2H), 1.63 (dd, J=2.8, 12.0 Hz, 2H). m/z: [ESI⁺] 228 (M-NH₂)⁺.

### Synthesis of benzyl ((3-methyl-1H-indol-5-yl)methyl)carbamate (55)

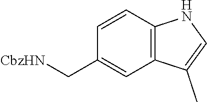 [SMILES: Cc1c[nH]c2ccc(CNC(=O)OCc3ccccc3)cc12]

To a solution of 1-(3-methyl-1H-indol-5-yl)methanamine (50) (2.00 g, 12.48 mmol) in DCM (20 mL) was added triethylamine (3.79 g, 37.45 mmol), DMAP (0.15 g, 1.228 mmol) and benzyl chloroformate (4.26 g, 24.97 mmol). The resulting mixture was stirred for 3 h at room temperature under a nitrogen atmosphere. The resulting mixture was diluted with water (30 mL) and extracted with DCM (3×50 mL). The combined organic layers were washed with brine (30 mL) and dried over anhydrous Na₂SO₄. After filtration, the filtrate was concentrated under reduced pressure. The residue was purified by column chromatography on silica gel (50% of ethyl acetate in petroleum ether) to afford benzyl ((3-methyl-1H-indol-5-yl)methyl)carbamate (55) as a yellow solid.

Yield 1.50 g (41%). ¹H NMR (400 MHz, CDCl₃) δ 7.92 (s, 1H), 7.51 (s, 1H), 7.42-7.37 (m, 4H), 7.37-7.34 (m, 1H), 7.34-7.31 (m, 1H), 7.15 (d, J=8.4 Hz, 1H), 7.00 (s, 1H), 5.18 (s, 2H), 5.05 (br s, 1H), 4.52 (d, J=5.6 Hz, 2H), 2.34 (s, 3H). m/z: [ESI⁺] 295 (M+H)⁺.

### Synthesis of benzyl ((2-bromo-3-methyl-1H-indol-5-yl)methyl)carbamate (56)

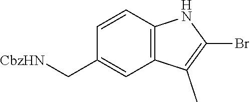 [SMILES: Cc1c(Br)[nH]c2ccc(CNC(=O)OCc3ccccc3)cc12]

Compound benzyl ((2-bromo-3-methyl-1H-indol-5-yl)methyl)carbamate (56) was prepared from benzyl ((3-methyl-1H-indol-5-yl)methyl)carbamate (55) (1.50 g, 5.10 mmol) following a similar procedure to that described for the synthesis of tert-butyl ((2-bromo-3-methyl-1H-indol-5-yl)methyl)carbamate (52) to afford benzyl ((2-bromo-3-methyl-1H-indol-5-yl)methyl)carbamate as a white solid (56).

Yield 1.39 g (73%). ¹H NMR (400 MHz, CDCl₃) δ 7.99 (s, 1H), 7.42-7.33 (m, 6H), 7.24 (dd, J=0.8, 8.4 Hz, 1H), 7.12 (d, J=8.4 Hz, 1H), 5.18 (s, 2H), 5.06 (br s, 1H), 4.49 (s, 2H), 2.26 (s, 3H). m/z: [ESI⁺] 373, 375 (M+H)⁺.

### Synthesis of tert-butyl methyl(2-(4,4,5,5-tetramethyl-1,3,2-dioxaborolan-2-yl)benzyl)carbamate (59)

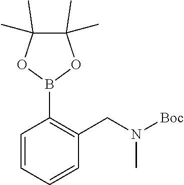 [SMILES: CN(Cc1ccccc1B1OC(C)(C)C(C)(C)O1)C(=O)OC(C)(C)C]

To a stirred mixture of tert-butyl (2-bromobenzyl)(methyl)carbamate (57) (1.00 g, 3.33 mmol) and bis(pinacolato)diboron (58) (1.30 g, 5.12 mmol) in 1,4-dioxane (10 mL) were added potassium acetate (0.98 g, 9.99 mmol) and [1′1-bis(diphenylphosphino)ferrocene]dichloro palladium(II) (0.12 g, 0.16 mmol) at room temperature under a nitrogen atmosphere. The resulting mixture was purged with nitrogen consecutively three times and was stirred for 3 h at 100° C. under a nitrogen atmosphere. The resulting mixture was cooled down to room temperature and filtered. The filtered cake was washed with DCM (3×10 mL). The combined filtrates were concentrated under reduced pressure. The residue was purified by column chromatography on silica gel (9% of ethyl acetate in petroleum ether) to afford tert-butyl methyl(2-(4,4,5,5-tetramethyl-1,3,2-dioxaborolan-2-yl)benzyl)carbamate (59) as a light yellow oil.

Yield 1.10 g (95%). ¹H NMR (400 MHz, CDCl₃) δ 7.83 (dd, J=1.6, 7.6 Hz, 1H), 7.44 (dt, J=1.6, 7.6 Hz, 1H), 7.28-7.22 (m, 2H), 4.79 (s, 2H), 2.85 (s, 3H), 1.36 (s, 12H), 1.28 (s, 9H). m/z: [ESI⁺] 348 (M+H)⁺.

### Synthesis of tert-butyl (2-(5-((((benzyloxy)carbonyl)amino)methyl)-3-methyl-1H-indol-2-yl)benzyl)(methyl)carbamate (60)

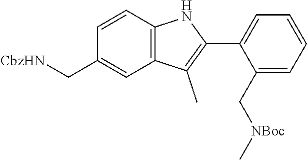 [SMILES: Cc1c(-c2ccccc2CN(C)C(=O)OC(C)(C)C)[nH]c2ccc(CNC(=O)OCc3ccccc3)cc12]

Compound tert-butyl (2-(5-((((benzyloxy)carbonyl)amino)methyl)-3-methyl-1H-indol-2-yl)benzyl)(methyl)carbamate (60) was prepared from benzyl ((2-bromo-3-methyl-1H-indol-5-yl)methyl)carbamate (56) (1.23 g, 3.30 mmol) and tert-butyl methyl(2-(4,4,5,5-tetramethyl-1,3,2-dioxaborolan-2-yl)benzyl)carbamate (59) (2.15 g, 6.19 mmol) following a similar procedure to that described for the synthesis of tert-butyl ((2-(3,6-dihydro-2H-pyran-4-yl)-3-methyl-1H-indol-5-yl)methyl)carbamate (65) to afford tert-butyl (2-(5-((((benzyloxy)carbonyl)amino)methyl)-3-methyl-1H-indol-2-yl)benzyl)(methyl)carbamate (60) as a yellow oil.

Yield 0.44 g (26%). ¹H NMR (400 MHz, CDCl₃) δ 8.63 (s, 1H), 7.52 (s, 1H), 7.47-7.31 (m, 10H), 7.17 (d, J=8.4 Hz, 1H), 5.18 (s, 2H), 5.10 (br s, 1H), 4.53 (d, J=5.6 Hz, 2H), 4.38 (s, 2H), 2.78 (s, 3H), 2.21 (s, 3H), 1.46 (s, 9H). m/z: [ESI⁺] 532 (M+H+H₂O)⁺.

### Synthesis of tert-butyl (2-(5-(aminomethyl)-3-methyl-1H-indol-2-yl)benzyl)(methyl)carbamate (61)

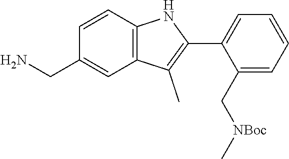 [SMILES: Cc1c(-c2ccccc2CN(C)C(=O)OC(C)(C)C)[nH]c2ccc(CN)cc12]

To a stirred solution of tert-butyl (2-(5-((((benzyloxy)carbonyl)amino)methyl)-3-methyl-1H-indol-2-yl)benzyl)(methyl)carbamate (60) (438 mg, 0.853 mmol) in methanol (5 mL) was added 10% palladium on activated carbon (50 mg) at room temperature under a nitrogen atmosphere. The resulting mixture was purged with hydrogen gas consecutively three times and was stirred for 16 h at room temperature under a hydrogen atmosphere. The resulting mixture was filtered. The filtered cake was washed with methanol (3×10 mL). The combined filtrates were concentrated under reduced pressure to afford tert-butyl (2-(5-(aminomethyl)-3-methyl-1H-indol-2-yl)benzyl)(methyl)carbamate (61) as a white solid.

Yield 244 mg (75%). ¹H NMR (400 MHz, CDCl₃) δ 8.73 (s, 1H), 7.54 (s, 1H), 7.44 (ddd, J=2.0, 6.0, 8.4 Hz, 1H), 7.41-7.32 (m, 4H), 7.18 (dd, J=1.6, 8.4 Hz, 1H), 4.40 (s, 2H), 4.00 (s, 2H), 2.77 (s, 3H), 2.23 (s, 3H), 1.46 (s, 9H). m/z: [ESI⁺] 363 (M-NH₂)⁺.

### Synthesis of tert-butyl methyl(2-(3-methyl-5-((4-methylpyrimidine-5-carboxamido)methyl)-1H-indol-2-yl)benzyl)carbamate

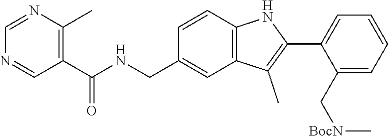 [SMILES: Cc1ncncc1C(=O)NCc1ccc2[nH]c(-c3ccccc3CN(C)C(=O)OC(C)(C)C)c(C)c2c1]

Compound tert-butyl methyl(2-(3-methyl-5-((4-methylpyrimidine-5-carboxamido)methyl)-1H-indol-2-yl)benzyl)carbamate was prepared from 4-methylpyrimidine-5-carboxylic acid (88 mg, 0.637 mmol) and tert-butyl (2-(5-(aminomethyl)-3-methyl-1H-indol-2-yl)benzyl)(methyl)carbamate (61) (220 mg, 0.580 mmol) following a similar procedure to that described for the synthesis of N-(4-amino-3-iodobenzyl)-4-methylpyrimidine-5-carboxamide to afford tert-butyl methyl(2-(3-methyl-5-((4-methylpyrimidine-5-carboxamido)methyl)-1H-indol-2-yl)benzyl)carbamate as a red oil.

Yield 210 mg (73%). ¹H NMR (400 MHz, CDCl₃) δ 9.12 (s, 1H), 8.74 (s, 1H), 7.59 (s, 1H), 7.44 (dd, J=4.4, 7.8 Hz, 1H), 7.40-7.34 (m, 5H), 7.22 (dd, J=1.6, 8.4 Hz, 1H), 6.40 (br s, 1H), 4.79 (d, J=5.2 Hz, 2H), 4.37 (s, 2H), 2.80 (s, 3H), 2.77 (s, 3H), 2.23 (s, 3H), 1.45 (s, 9H). m/z: [ESI⁺] 500 (M+H)⁺.

### Synthesis of tert-butyl ((2-(2-(hydroxymethyl)phenyl)-3-methyl-1H-indol-5-yl)methyl)carbamate

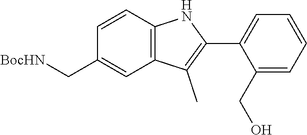 [SMILES: Cc1c(-c2ccccc2CO)[nH]c2ccc(CNC(=O)OC(C)(C)C)cc12]

Compound tert-butyl ((2-(2-(hydroxymethyl)phenyl)-3-methyl-1H-indol-5-yl)methyl)carbamate was prepared from tert-butyl ((2-bromo-3-methyl-1H-indol-5-yl)methyl)carbamate (52) (1.00 g, 2.95 mmol) and (2-(hydroxymethyl)phenyl)boronic acid (1.34 g, 8.84 mmol) following a similar procedure to that described for the synthesis of tert-butyl ((2-(3,6-dihydro-2H-pyran-4-yl)-3-methyl-1H-indol-5-yl)methyl)carbamate (65) to afford tert-butyl ((2-(2-(hydroxymethyl)phenyl)-3-methyl-1H-indol-5-yl)methyl)carbamate as a white solid.

Yield 1.00 g (93%), ¹H NMR (400 MHz, CDCl₃) δ 9.57 (s, 1H), 7.57 (dd, J=1.6, 7.6 Hz, 1H), 7.54 (s, 1H), 7.52-7.44 (m, 2H), 7.42 (dd, J=1.6, 7.6 Hz, 1H), 7.37 (d, J=8.4 Hz, 1H), 7.17 (d, J=8.4 Hz, 1H), 4.85 (br s, 1H), 4.63 (s, 2H), 4.46 (d, J=5.4 Hz, 2H), 2.38 (s, 3H), 2.15 (br s, 1H), 1.51 (s, 9H). m/z: [ESI⁺] 367 (M+H)⁺.

### Synthesis of(2-(5-(aminomethyl)-3-methyl-1H-indol-2-yl)phenyl)methanol 2,2,2-trifluoroacetate

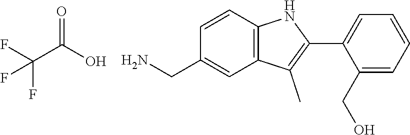 [SMILES: O=C(O)C(F)(F)F ; Cc1c(-c2ccccc2CO)[nH]c2ccc(CN)cc12]

Compound (2-(5-(aminomethyl)-3-methyl-1H-indol-2-yl)phenyl)methanol 2,2,2-trifluoroacetate was prepared from tert-butyl ((2-(2-(hydroxymethyl)phenyl)-3-methyl-1H-indol-5-yl)methyl)carbamate (1.00 g, 2.73 mmol) following a similar procedure to that described for the synthesis of (2-(2-fluorophenyl)-3-methyl-1H-indol-5-yl)methanamine hydrochloride, however trifluoroacetic acid was used instead of hydrogen chloride in dioxane to afford (2-(5-(aminomethyl)-3-methyl-1H-indol-2-yl)phenyl)methanol 2,2,2-trifluoroacetate as a brown solid.

Yield 0.70 g (67%). ¹H NMR (400 MHz, CDCl₃) δ 8.34 (br s, 3H, NH₃⁺), 8.31 (s, 1H), 7.62-7.58 (m, 1H), 7.57 (d, J=1.6 Hz, 1H), 7.52-7.48 (m, 2H), 7.41-7.38 (m, 1H), 7.33 (d, J=8.4 Hz, 1H), 7.14 (dd, J=1.6, 8.4 Hz, 1H), 5.21 (s, 2H), 4.16 (d, J=6.4 Hz, 2H), 2.18 (s, 3H). m/z: [ESI⁺] 250 (M-NH₂)+.

### Synthesis of tert-butyl ((2-(2-(methoxymethyl)phenyl)-3-methyl-1H-indol-5-yl)methyl)carbamate

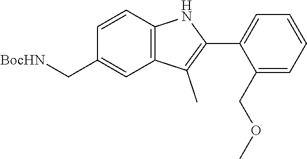 [SMILES: COCc1ccccc1-c1[nH]c2ccc(CNC(=O)OC(C)(C)C)cc2c1C]

Compound tert-butyl ((2-(2-(methoxymethyl)phenyl)-3-methyl-1H-indol-5-yl)methyl)carbamate was prepared from tert-butyl ((2-bromo-3-methyl-1H-indol-5-yl)methyl)carbamate (52) (80 mg, 0.236 mmol) and (2-(methoxymethyl)phenyl)boronic acid (47 mg, 0.283 mmol) following a similar procedure to that described for the synthesis of tert-butyl ((2-(3,6-dihydro-2H-pyran-4-yl)-3-methyl-1H-indol-5-yl)methyl)carbamate (65) to afford tert-butyl ((2-(2-(methoxymethyl)phenyl)-3-methyl-1H-indol-5-yl)methyl)carbamate as an off-white solid.

Yield 60 mg (67%). ¹H NMR (400 MHz, CDCl₃) δ 9.51 (s, 1H), 7.61-7.56 (m, 2H), 7.53-7.46 (m, 2H), 7.43 (dd, J=1.6, 7.6 Hz, 1H), 7.37 (d, J=8.4 Hz, 1H), 7.19 (dd, J=1.6, 8.4 Hz, 1H), 4.95 (br s, 1H), 4.49 (d, J=5.6 Hz, 2H), 4.37 (s, 2H), 3.50 (s, 3H), 2.41 (s, 3H), 1.54 (s, 9H). m/z: [ESI⁺] 381 (M+H)⁺.

### Synthesis of (2-(2-(methoxymethyl)phenyl)-3-methyl-1H-indol-5-yl)methanamine hydrochloride

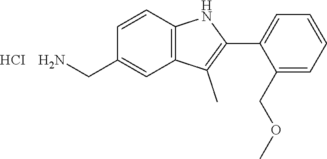 [SMILES: "HCI" ; COCc1ccccc1-c1[nH]c2ccc(CN)cc2c1C]

Compound (2-(2-(methoxymethyl)phenyl)-3-methyl-1H-indol-5-yl)methanamine hydrochloride was prepared from tert-butyl ((2-(2-(methoxymethyl)phenyl)-3-methyl-1H-indol-5-yl)methyl)carbamate (60 mg, 0.158 mmol) following a similar procedure to that described for the synthesis of (2-(2-fluorophenyl)-3-methyl-1H-indol-5-yl)methanamine hydrochloride to afford (2-(2-(methoxymethyl)phenyl)-3-methyl-1H-indol-5-yl)methanamine hydrochloride as an off-white solid.

Yield 45 mg (90%). m/z: [ESI⁺] 264 (M-NH₂)⁺.

### Synthetic Details for Compounds of the Invention

### Synthesis of N-((2-(2-fluorophenyl)-3-methyl-1H-indol-5-yl)methyl)benzamide (Compound 100)

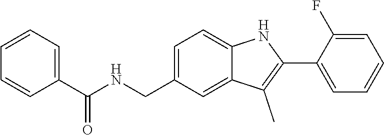 [SMILES: Cc1c(-c2ccccc2F)[nH]c2ccc(CNC(=O)c3ccccc3)cc12]

To a solution of benzoic acid (20 mg, 0.163 mmol) in anhydrous DMF (0.5 mL) were added sequentially DIPEA (0.039 mL, 0.222 mmol) and HATU (67 mg, 0.177 mmol) and the reaction mixture was stirred at room temperature for 10 minutes. A solution of (2-(2-fluorophenyl)-3-methyl-1H-indol-5-yl)methanamine hydrochloride (43 mg, 0.148 mmol) in anhydrous DMF (1.5 mL) and DIPEA (0.039 mL, 0.222 mmol) was then added and the reaction continued to stir at room temperature for 2 hours. The mixture was partioned between ethyl acetate (25 mL) and a diluted Na₂CO₃ solution (10%, 25 mL). The organic phase was washed with a diluted solution of Na₂CO₃ (10%, 25 mL) and brine (10 mL), dried (MgSO₄), filtered and evaporated. The residue was purified by preparative HPLC to give N-((2-(2-fluorophenyl)-3-methyl-1H-indol-5-yl)methyl)benzamide (100) as an off-white solid.

Yield 32 mg (60%). ¹H NMR (400 MHz, DMSO) δ 11.12 (s, 1H), 9.05 (dd, J=5.4, 5.4 Hz, 1H), 7.96 (d, J=7.1 Hz, 2H), 7.60-7.48 (m, 6H), 7.43-7.34 (m, 3H), 7.19 (d, J=8.3 Hz, 1H), 4.63 (d, J=5.8 Hz, 2H), 2.28 (s, 3H). m/z: [ESI⁺] 359 (M+H)⁺, (C₂₃H₁₉FN₂O).

### Synthesis of N-((2-(2-fluorophenyl)-3-methyl-1H-indol-5-yl)methyl)-4-methylpyrimidine-5-carboxamide (Compound 101)

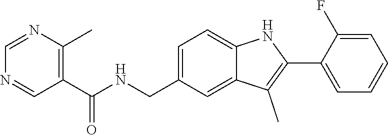 [SMILES: Cc1ncncc1C(=O)NCc1ccc2[nH]c(-c3ccccc3F)c(C)c2c1]

Compound N-((2-(2-fluorophenyl)-3-methyl-1H-indol-5-yl)methyl)-4-methylpyrimidine-5-carboxamide (101) was prepared from 4-methylpyrimidine-5-carboxylic acid following a procedure similar to that described for the synthesis of N-((2-(2-fluorophenyl)-3-methyl-1H-indol-5-yl)methyl)benzamide, and was isolated as a white solid.

Yield 25 mg (48%). ¹H NMR (400 MHz, DMSO) δ 11.16 (s, 1H), 9.19 (t, J=5.0 Hz, 1H), 9.14 (s, 1H), 8.78 (s, 1H), 7.62 (dd, J=7.7, 7.7 Hz, 1H), 7.57 (s, 1H), 7.55-7.49 (m, 1H), 7.41 (dd, J=7.2, 7.2 Hz, 3H), 7.21 (d, J=8.3 Hz, 1H), 4.63 (d, J=5.8 Hz, 2H), 2.60 (s, 3H), 2.30 (s, 3H). m/z: [ESI⁺] 375 (M+H)⁺, (C₂₂H₁₉FN₄O).

### Synthesis of N-((2-(2-fluorophenyl)-3-methyl-1H-indol-5-yl)methyl)nicotinamide (Compound 102)

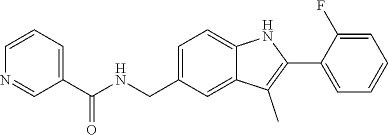 [SMILES: Cc1c(-c2ccccc2F)[nH]c2ccc(CNC(=O)c3cccnc3)cc12]

Compound N-((2-(2-fluorophenyl)-3-methyl-1H-indol-5-yl)methyl)nicotinamide (102) was prepared from nicotinic acid following a procedure similar to that described for the synthesis of N-((2-(2-fluorophenyl)-3-methyl-1H-indol-5-yl)methyl)benzamide, and was isolated as a white solid.

Yield 26 mg (53%). ¹H NMR (400 MHz, DMSO) δ 11.14 (s, 1H), 9.28-9.22 (m, 1H), 9.11 (d, J=1.5 Hz, 1H), 8.77-8.74 (m, 1H), 8.29 (d, J=7.8 Hz, 1H), 7.62-7.50 (m, 4H), 7.39 (dd, J=8.0, 12.5 Hz, 3H), 7.20 (d, J=9.1 Hz, 1H), 4.65 (d, J=5.6 Hz, 2H), 2.28 (s, 3H). m/z: [ESI⁺] 360 (M+H)⁺, (C₂₂H₁₈FN₃O).

### Synthesis of N-((2-(2-fluorophenyl)-3-methyl-1H-indol-5-yl)methyl)-4-methylnicotinamide (Compound 103)

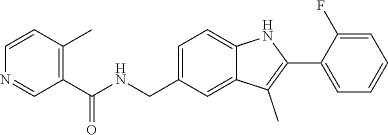 [SMILES: Cc1ccncc1C(=O)NCc1ccc2[nH]c(-c3ccccc3F)c(C)c2c1]

Compound N-((2-(2-fluorophenyl)-3-methyl-1H-indol-5-yl)methyl)-4-methylnicotinamide (103) was prepared from 4-methylnicotinic acid following a procedure similar to that described for the synthesis of N-((2-(2-fluorophenyl)-3-methyl-1H-indol-5-yl)methyl)benzamide, and was isolated as a white solid.

Yield 24 mg (47%). ¹H NMR (400 MHz, DMSO) δ 11.12 (s, 1H), 9.01 (dd, J=5.9, 5.9 Hz, 1H), 8.53 (s, 1H), 8.48 (d, J=5.0 Hz, 1H), 7.61-7.55 (m, 1H), 7.53-7.45 (m, 2H), 7.39-7.30 (m, 4H), 7.17 (dd, J=1.6, 8.3 Hz, 1H), 4.58 (d, J=6.0 Hz, 2H), 2.39 (s, 3H), 2.26 (s, 3H). m/z: [ESI⁺] 374 (M+H)⁺, (C₂₃H₂₀FN₃O).

### Synthesis of N-((2-(2-fluorophenyl)-3-methyl-1H-indol-5-yl)methyl)pyrimidine-5-carboxamide (Compound 104)

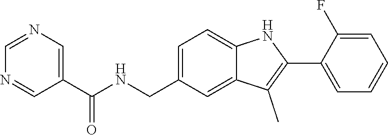 [SMILES: Cc1c(-c2ccccc2F)[nH]c2ccc(CNC(=O)c3cncnc3)cc12]

Compound N-((2-(2-fluorophenyl)-3-methyl-1H-indol-5-yl)methyl)pyrimidine-5-carboxamide (104) was prepared from pyrimidine-5-carboxylic acid following a procedure similar to that described for the synthesis of N-((2-(2-fluorophenyl)-3-methyl-1H-indol-5-yl)methyl)benzamide, and was isolated as a white solid.

Yield 28 mg (56%). H NMR (400 MHz, DMSO) δ11.12 (s, 1H), 9.37 (dd, J=5.8, 5.8 Hz, 1H), 9.33 (s, 1H), 9.23 (s, 2H), 7.60-7.53 (m, 2H), 7.52-7.45 (m, 1H), 7.41-7.33 (m, 3H), 7.17 (dd, J=1.6, 8.3 Hz, 1H), 4.63 (d, J=5.8 Hz, 2H), 2.25 (d, J=1.6 Hz, 3H). m/z: [ESI⁺] 361 (M+H)⁺, (C₂₁H₁₇FN₄O).

### Synthesis of N-((2-(2-fluorophenyl)-3-methyl-1H-indol-5-yl)methyl)-2-methylnicotinamide (Compound 105)

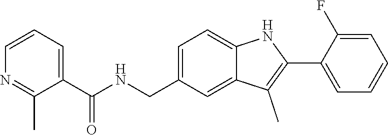 [SMILES: Cc1ncccc1C(=O)NCc1ccc2[nH]c(-c3ccccc3F)c(C)c2c1]

Compound N-((2-(2-fluorophenyl)-3-methyl-1H-indol-5-yl)methyl)-2-methylnicotinamide (105) was prepared from 2-methylnicotinic acid following a procedure similar to that described for the synthesis of N-((2-(2-fluorophenyl)-3-methyl-1H-indol-5-yl)methyl)benzamide, and was isolated as a white solid.

Yield 29 mg (57%). H NMR (400 MHz, DMSO) δ 11.15 (s, 1H), 8.99 (dd, J=5.7, 5.7 Hz, 1H), 8.54 (d, J=3.5 Hz, 1H), 7.78 (d, J=7.6 Hz, 1H), 7.65-7.59 (m, 1H), 7.57-7.49 (m, 2H), 7.40 (dd, J=8.8, 8.8 Hz, 3H), 7.33 (dd, J=4.9, 7.5 Hz, 1H), 7.20 (d, J=8.3 Hz, 1H), 4.60 (d, J=6.1 Hz, 2H), 2.30 (s, 3H). m/z: [ESI⁺] 374 (M+H)⁺, (C₂₃H₂₀FN₃O).

### Synthesis of N-((2-(2-fluorophenyl)-3-methyl-1H-indol-5-yl)methyl) isonicotinamide (Compound 106)

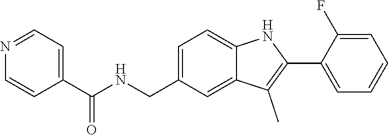 [SMILES: Cc1c(-c2ccccc2F)[nH]c2ccc(CNC(=O)c3ccncc3)cc12]

Compound N-((2-(2-fluorophenyl)-3-methyl-1H-indol-5-yl)methyl) isonicotinamide (106) was prepared from isonicotinic acid following a procedure similar to that described for the synthesis of N-((2-(2-fluorophenyl)-3-methyl-1H-indol-5-yl)methyl)benzamide, and was isolated as a white solid.

Yield 25 mg (51%). ¹H NMR (400 MHz, DMSO) δ 11.14 (s, 1H), 9.34 (dd, J=5.4, 5.4 Hz, 1H), 8.78 (d, J=5.6 Hz, 2H), 7.86 (d, J=5.6 Hz, 2H), 7.61 (dd, J=7.1, 7.1 Hz, 1H), 7.57-7.49 (m, 2H), 7.39 (dd, J=7.8, 13.4 Hz, 3H), 7.19 (d, J=8.1 Hz, 1H), 4.64 (d, J=5.8 Hz, 2H), 2.29 (s, 3H). m/z: [ESI⁺] 360 (M+H)⁺, (C₂₂H₁₈FN₃O).

### Synthesis of 4-methyl-N-((3-methyl-2-(o-tolyl)-1H-indol-5-yl)methyl)pyrimidine-5-carboxamide (Compound 109)

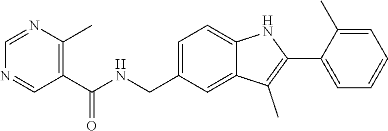 [SMILES: Cc1ccccc1-c1[nH]c2ccc(CNC(=O)c3cncnc3C)cc2c1C]

Compound 4-methyl-N-((3-methyl-2-(o-tolyl)-1H-indol-5-yl)methyl)pyrimidine-5-carboxamide (109) was prepared from 4-methylpyrimidine-5-carboxylic acid and (3-methyl-2-(o-tolyl)-1H-indol-5-yl)methanamine hydrochloride following a procedure similar to that described for the synthesis of N-((2-(2-fluorophenyl)-3-methyl-1H-indol-5-yl)methyl)benzamide, and was isolated as a white solid.

Yield 38 mg (62%). ¹H NMR (400 MHz, DMSO) δ 10.98 (s, 1H), 9.20-9.12 (m, 2H), 8.78 (s, 1H), 7.54 (s, 1H), 7.43-7.39 (m, 2H), 7.39-7.32 (m, 3H), 7.17 (d, J=8.1 Hz, 1H), 4.63 (d, J=5.6 Hz, 2H), 2.61 (s, 3H), 2.28 (s, 3H), 2.17 (s, 3H). m/z: [ESI⁺] 371 (M+H)⁺, (C₂₃H₂₂N₄O).

### Synthesis of 4-methyl-N-((3-methyl-2-(pyridin-2-yl)-1H-indol-5-yl)methyl)pyrimidine-5-carboxamide (Compound 110)

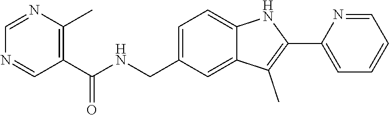 [SMILES: Cc1ncncc1C(=O)NCc1ccc2[nH]c(-c3ccccn3)c(C)c2c1]

Compound 4-methyl-N-((3-methyl-2-(pyridin-2-yl)-1H-indol-5-yl)methyl)pyrimidine-5-carboxamide (110) was prepared from 4-methylpyrimidine-5-carboxylic acid and (3-methyl-2-(pyridin-2-yl)-1H-indol-5-yl)methanamine dihydrochloride following a procedure similar to that described for the synthesis of N-((2-(2-fluorophenyl)-3-methyl-1H-indol-5-yl)methyl)benzamide, and was isolated as an off-white solid.

Yield 26 mg (33%). ¹H NMR (400 MHz, DMSO) δ 11.38 (s, 1H), 9.21-9.12 (m, 2H), 8.79 (s, 1H), 8.74 (d, J=4.0 Hz, 1H), 7.99-7.94 (m, 1H), 7.89 (d, J=8.1 Hz, 1H), 7.61 (s, 1H), 7.46 (d, J=8.3 Hz, 1H), 7.36 (dd, J=5.3, 6.6 Hz, 1H), 7.22 (d, J=8.6 Hz, 1H), 4.63 (d, J=5.6 Hz, 2H), 2.64 (s, 3H), 2.60 (s, 3H). m/z: [ESI⁺] 358 (M+H)⁺, (C₂₁H₁₉N₅O).

### Synthesis of 1-(2-(2-fluorophenyl)-3-methyl-1H-indol-5-yl)-N-((4-methylpyrimidin-5-yl)methyl)methanamine hydrochloride (Compound 111)

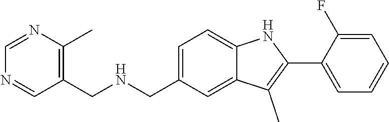 [SMILES: Cc1ncncc1CNCc1ccc2[nH]c(-c3ccccc3F)c(C)c2c1]

To a solution of (2-(2-fluorophenyl)-3-methyl-1H-indol-5-yl)methanamine hydrochloride (41 mg, 0.141 mmol), triethylamine (24 μL, 0.169 mmol) and 4-methylpyrimidine-5-carbaldehyde (13)(17 mg, 0.141 mmol) in anhydrous DCM (3 mL) was added at room temperature sodium triacetoxyborohydride (75 mg, 0.35 mmol) followed by acetic acid (16 μL, 0.282 mmol). The reaction was stirred at room temperature for 4 hours. The reaction mixture was partitioned between ethyl acetate (20 mL) and a diluted solution of Na₂CO₃ (50%, 20 mL). The organic phase was washed with brine (10 mL), dried (MgSO₄), filtered and evaporated. The residue was purified by preparative HPLC to give the free base of the title compound as a colourless glass (18 mg). This material was dissolved in MeOH (0.5 mL), treated with 0.5 M HCl in MeOH (0.25 mL) and dried to give 1-(2-(2-fluorophenyl)-3-methyl-1H-indol-5-yl)-N-((4-methylpyrimidin-5-yl)methyl)methanamine hydrochloride (111) as a yellow solid.

Yield 21 mg (37%). ¹H NMR (400 MHz, DMSO) δ 11.39 (s, 1H), 9.54 (br s, 2H), 9.08 (s, 1H), 8.86 (s, 1H), 7.84 (s, 1H), 7.67-7.62 (m, 1H), 7.56-7.52 (m, 1H), 7.50-7.45 (m, 1H), 7.41 (dd, J=7.6, 14.4 Hz, 3H), 4.44 (s, 2H), 4.30 (dd, J=5.2, 5.2 Hz, 2H), 2.60 (s, 3H), 2.33 (s, 3H). m/z: [ESI⁺] 361 (M+H)⁺, (C₂₂H₂₁FN₄).

### Synthesis of N-[[1,3-dimethyl-2-(o-tolyl)indol-5-yl]methyl-4-methyl-pyrimidine-5-carboxamide (Compound 112)

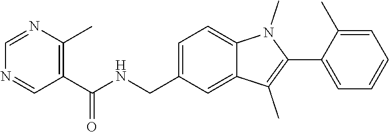 [SMILES: Cc1ccccc1-c1c(C)c2cc(CNC(=O)c3cncnc3C)ccc2n1C]

To a solution of 4-methylpyrimidine-5-carboxylic acid (51 mg, 0.366 mmol) in anhydrous DMF (1 mL) were added sequentially DIPEA (0.087 mL, 0.499 mmol) and HATU (152 mg, 0.399 mmol) and the reaction mixture was stirred at room temperature for 10 minutes. A solution of (1,3-dimethyl-2-(o-tolyl)-1H-indol-5-yl)methanamine (88 mg, 0.333 mmol) in anhydrous DMF (2 mL) was then added and the reaction continued to stir at room temperature for 2 hours. The mixture was partitioned between ethyl acetate (25 mL) and a diluted Na₂CO₃ solution (10%, 25 mL). The layers were separated, and the organic phase was washed with a diluted solution of Na₂CO₃ (10%, 25 mL) and brine (10 mL), dried (MgSO₄), filtered and evaporated. The residue was purified by preparative HPLC to give N-[[1,3- dimethyl-2-(o-tolyl)indol-5-yl]methyl]-4-methyl-pyrimidine-5-carboxamide (112) as a white solid.

Yield 81 mg (63%). ¹H NMR (400 MHz, DMSO) δ 9.16-9.10 (m, 1H), 9.08 (s, 1H), 8.72 (s, 1H), 7.52-7.50 (m, 1H), 7.43-7.40 (m, 3H), 7.36-7.31 (m, 1H), 7.25 (d, J=7.3 Hz, 1H), 7.20 (dd, J=1.8, 8.3 Hz, 1H), 4.59 (d, J=5.8 Hz, 2H), 3.40 (s, 3H), 2.55 (s, 3H), 2.06 (s, 3H), 2.04 (s, 3H). m/z: [ESI⁺] 385 (M+H)⁺, (C₂₄H₂₄N₄O).

### Synthesis of 4-methyl-N-[[2-(o-tolyl)-1H-indol-5-yl]methyl]pyrimidine-5-carboxamide (Compound 114)

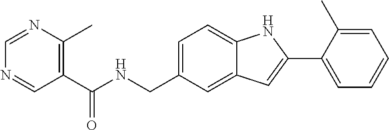 [SMILES: Cc1ccccc1-c1cc2cc(CNC(=O)c3cncnc3C)ccc2[nH]1]

A sealed tube was charged at room temperature with N-((1H-indol-5-yl)methyl)-4-methylpyrimidine-5-carboxamide (137 mg, 0.514 mmol), bicyclo[2.2.1]hept-2-ene (97 mg, 1.03 mmol), K₂CO₃ (142 mg, 1.03 mmol) and bis(acetonitrile)dichloropalladium(II) (13 mg, 0.051 mmol). A solution of 0.5M aqueous N,N-dimethylacetamide (2.6 mL) was added, followed by addition of 2-iodotoluene (0.131 mL, 1.03 mmol). After stirring at 100° C. for 48 hours, the reaction mixture was diluted with ethyl acetate (20 mL), filtered through celite and partitioned between ethyl acetate (25 mL) and water (25 mL). The layers were separated, and the organic phase was washed with water (20 mL) and brine (20 mL), dried (MgSO₄), filtered and evaporated. The residue was purified by preparative HPLC to give 4-methyl-N-[[2-(o-tolyl)-1H-indol-5-yl]methyl]pyrimidine-5-carboxamide (114) as an off-white solid.

Yield 10 mg (6%). ¹H NMR (400 MHz, DMSO) δ 11.25 (s, 1H), 9.12 (t, J=5.8 Hz, 1H), 9.08 (s, 1H), 8.73 (s, 1H), 7.53 (dd, J=4.4, 4.4 Hz, 2H), 7.39-7.27 (m, 4H), 7.13 (dd, J=1.5, 8.3 Hz, 1H), 6.57 (d, J=1.3 Hz, 1H), 4.55 (d, J=5.8 Hz, 2H), 2.54 (s, 3H), 2.47 (s, 3H). m/z: [ESI⁺] 357 (M+H)⁺, (C₂₂H₂₀N₄O).

### Synthesis of 4-methyl-N-[[1-methyl-2-(o-tolyl)indol-5-yl]methyl]pyrimidine-5-carboxamide (Compound 117)

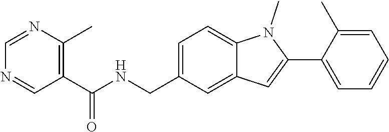 [SMILES: Cc1ccccc1-c1cc2cc(CNC(=O)c3cncnc3C)ccc2n1C]

To a solution of 4-methylpyrimidine-5-carboxylic acid (33 mg, 0.242 mmol) in anhydrous DMF (1 mL) were added sequentially DIPEA (0.057 mL, 0.330 mmol) and HATU (100 mg, 0.264 mmol) and the reaction mixture was stirred at room temperature for 10 minutes. A solution of (1-methyl-2-(o-tolyl)-1H-indol-5-yl)methanamine (33)(55 mg, 0.220 mmol) in anhydrous DMF (1.5 mL) was then added and the reaction continued to stir at room temperature for 1.5 hours. The mixture was partitioned between ethyl acetate (25 mL) and a diluted Na₂CO₃ solution (10%, 25 mL). The layers were separated, and the organic phase was washed with a diluted solution of Na₂CO₃ (10%, 25 mL) and brine (10 mL), dried (MgSO₄), filtered and evaporated. The residue was purified by preparative HPLC to give 4-methyl-N-[[1-methyl-2-(o-tolyl)indol-5-yl]methyl]pyrimidine-5-carboxamide (117) as a white solid.

Yield 50 mg (61%). ¹H NMR (400 MHz, DMSO) δ 9.14 (t, J=5.9 Hz, 1H), 9.08 (s, 1H), 8.73 (s, 1H), 7.55-7.54 (m, 1H), 7.46 (d, J=8.4 Hz, 1H), 7.40-7.37 (m, 2H), 7.33-7.30 (m, 2H), 7.20 (dd, J=1.6, 8.4 Hz, 1H), 6.40 (s, 1H), 4.57 (d, J=5.9 Hz, 2H), 3.48 (s, 3H), 2.54 (s, 3H), 2.16 (s, 3H). m/z: [ESI⁺]371 (M+H)⁺, (C₂₃H₂₂N₄O).

### Synthesis of 4-methyl-N-[[3-methyl-2-(m-tolyl)-1H-indol-5-yl]methyl]pyrimidine-5-carboxamide (Compound 113)

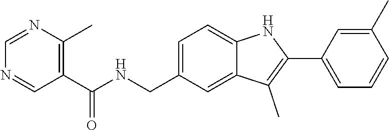 [SMILES: Cc1cccc(-c2[nH]c3ccc(CNC(=O)c4cncnc4C)cc3c2C)c1]

To a solution of 4-methylpyrimidine-5-carboxylic acid (36 mg, 0.264 mmol) in anhydrous DMF (1 mL) were added sequentially DIPEA (0.063 mL, 0.359 mmol) and HATU (109 mg, 0.288 mmol) and the reaction mixture was stirred at room temperature for 10 minutes. A solution of (3-methyl-2-(m-tolyl)-1H-indol-5-yl)methanamine (60 mg, 0.240 mmol) in anhydrous DMF (2 mL) was then added and the reaction continued to stir at room temperature for 16 hours. The mixture was partitioned between ethyl acetate (25 mL) and a diluted Na₂CO₃ solution (10%, 25 mL). The layers were separated, and the organic phase was washed with a diluted solution of Na₂CO₃ (10%, 25 mL) and brine (10 mL), dried (MgSO₄), filtered and evaporated. The residue was purified by preparative HPLC to give 4-methyl-N-[[3-methyl-2-(m-tolyl)-1H-indol-5-yl]methyl]pyrimidine-5-carboxamide (113) as an off-white solid.

Yield 54 mg (61%). ¹H NMR (400 MHz, DMSO) δ 11.09 (s, 1H), 9.12 (t, J=5.8 Hz, 1H), 9.08 (s, 1H), 8.72 (s, 1H), 7.50-7.44 (m, 3H), 7.39 (dd, J=7.6, 7.6 Hz, 1H), 7.32 (d, J=8.2 Hz, 1H), 7.17 (d, J=7.6 Hz, 1H), 7.12 (dd, J=1.6, 8.2 Hz, 1H), 4.57 (d, J=5.8 Hz, 2H), 2.55 (s, 3H), 2.40 (s, 3H), 2.39 (s, 3H). m/z: [ESI⁺] 371 (M+H)⁺, (C₂₃H₂₂N₄O).

### Synthesis of 4-methyl-N-[[3-methyl-2-(3-pyridyl)-1H-indol-5-yl]methyl]pyrimidine-5-carboxamide (Compound 115)

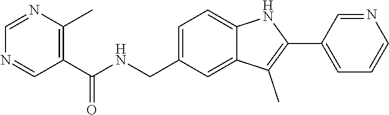 [SMILES: Cc1ncncc1C(=O)NCc1ccc2[nH]c(-c3cccnc3)c(C)c2c1]

Compound 4-methyl-N-[[3-methyl-2-(3-pyridyl)-1H-indol-5-yl]methyl]pyrimidine-5-carboxamide (115) was prepared from (3-methyl-2-(pyridin-3-yl)-1H-indol-5-yl)methanamine following a similar procedure to that described for the synthesis of 4-methyl-N-[[3-methyl-2-(m-tolyl)-1H-indol-5-yl]methyl]pyrimidine-5-carboxamide and was obtained as a yellow solid.

Yield 15 mg (17%). ¹H NMR (400 MHz, DMSO) δ 11.30 (s, 1H), 9.14 (t, J=6.1 Hz, 1H), 9.08 (s, 1H), 8.90-8.88 (m, 1H), 8.73 (s, 1H), 8.55 (dd, J=1.6, 4.7 Hz, 1H), 8.07-8.03 (m, 1H), 7.55-7.51 (m, 2H), 7.36 (d, J=8.3 Hz, 1H), 7.17 (dd, J=1.8, 8.3 Hz, 1H), 4.58 (d, J=6.1 Hz, 2H), 2.54 (s, 3H), 2.43 (s, 3H). m/z: [ESI⁺] 358 (M+H)⁺, (C₂₁H₁₉N₅O).

### Synthesis of N-[[2-(3-methoxyphenyl)-3-methyl-1H-indol-5-yl]methyl]-4-methyl-pyrimidine-5-carboxamide (Compound 116)

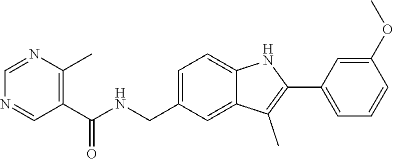 [SMILES: COc1cccc(-c2[nH]c3ccc(CNC(=O)c4cncnc4C)cc3c2C)c1]

Compound N-[[2-(3-methoxyphenyl)-3-methyl-1H-indol-5-yl]methyl]-4-methyl-pyrimidine-5-carboxamide (116) was prepared from (2-(3-methoxyphenyl)-3-methyl-1H-indol-5-yl)methanamine following a similar procedure to that described for the synthesis of 4-methyl-N-[[3-methyl-2-(m-tolyl)-1H-indol-5-yl]methyl]pyrimidine-5-carboxamide and was obtained as an off-white solid.

Yield 46 mg (25%). ¹H NMR (400 MHz, DMSO) δ 11.14 (s, 1H), 9.13 (t, J=5.9 Hz, 1H), 9.08 (s, 1H), 8.72 (s, 1H), 7.51 (s, 1H), 7.42 (dd, J=8.0, 8.0 Hz, 1H), 7.33 (d, J=8.3 Hz, 1H), 7.27-7.23 (m, 1H), 7.22-7.19 (m, 1H), 7.14 (dd, J=1.6, 8.3 Hz, 1H), 6.95-6.91 (m, 1H), 4.57 (d, J=5.9 Hz, 2H), 3.84 (s, 3H), 2.55 (s, 3H), 2.42 (s, 3H). m/z: [ESI⁺] 387 (M+H)⁺, (C₂₃H₂₂N₄O₂).

### Synthesis of N-[[2-(2-methoxyphenyl)-3-methyl-1H-indol-5-yl]methyl]-4-methyl-pyrimidine-5-carboxamide (Compound 120)

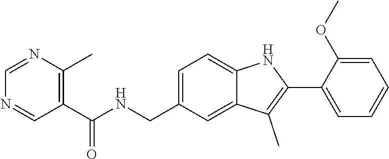 [SMILES: COc1ccccc1-c1[nH]c2ccc(CNC(=O)c3cncnc3C)cc2c1C]

Compound N-[[2-(2-methoxyphenyl)-3-methyl-1H-indol-5-yl]methyl]-4-methyl-pyrimidine-5-carboxamide (120) was prepared from (2-(2-methoxyphenyl)-3-methyl-1H-indol-5-yl)methanamine following a similar procedure to that described for the synthesis of 4-methyl-N-[[3-methyl-2-(m-tolyl)-1H-indol-5-yl]methyl]pyrimidine-5-carboxamide and was obtained as a beige solid.

Yield 60 mg (50%). ¹H NMR (400 MHz, DMSO) δ 10.83 (s, 1H), 9.12 (t, J=5.8 Hz, 1H), 9.08 (s, 1H), 8.72 (s, 1H), 7.46 (s, 1H), 7.42-7.35 (m, 2H), 7.30 (d, J=8.5 Hz, 1H), 7.15 (d, J=7.9 Hz, 1H), 7.12-7.04 (m, 2H), 4.57 (d, J=5.8 Hz, 2H), 3.80 (s, 3H), 2.54 (s, 3H), 2.19 (s, 3H). m/z: [ESI⁺] 387 (M+H)⁺, (C₂₃H₂₂N₄O₂).

### Synthesis of 4-methyl-N-[[3-methyl-2-(2-methyl-3-pyridyl)-1H-indol-5-yl]methyl]pyrimidine-5-carboxamide (Compound 119)

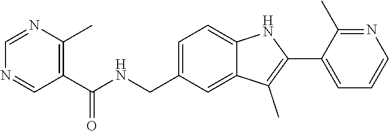 [SMILES: Cc1ncncc1C(=O)NCc1ccc2[nH]c(-c3cccnc3C)c(C)c2c1]

Compound 4-methyl-N-[[3-methyl-2-(2-methyl-3-pyridyl)-1H-indol-5-yl]methyl]pyrimidine-5-carboxamide (119) was prepared from (3-methyl-2-(2-methylpyridin-3-yl)-1H-indol-5-yl)methanamine following a similar procedure to that described for the synthesis of 4-methyl-N-[[3-methyl-2-(m-tolyl)-1H-indol-5-yl]methyl]pyrimidine-5-carboxamide and was obtained as an off-white solid.

Yield 29 mg (25%). ¹H NMR (400 MHz, DMSO) δ 11.05 (s, 1H), 9.13 (t, J=5.9 Hz, 1H), 9.08 (s, 1H), 1.72 (s, 1H), 8.52 (dd, J=1.7, 4.8 Hz, 1H), 7.73 (dd, J=1.7, 8.0 Hz, 1H), 7.51 (s, 1H), 7.37-7.30 (m, 2H), 7.15 (dd, J=1.7, 8.0 Hz, 1H), 4.58 (d, J=5.9 Hz, 2H), 2.55 (s, 3H), 2.42 (s, 3H), 2.13 (s, 3H). m/z: [ESI⁺] 372 (M+H)⁺, (C₂₂H₂₁N₅O).

### Synthesis of N-[[2-(3-fluoro-2-pyridyl)-3-methyl-1H-indol-5-yl]methyl]-4-methyl-pyrimidine-5-carboxamide (Compound 118)

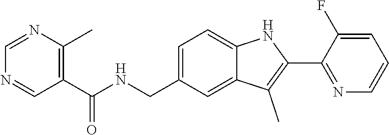 [SMILES: Cc1ncncc1C(=O)NCc1ccc2[nH]c(-c3ncccc3F)c(C)c2c1]

Compound N-[[2-(3-fluoro-2-pyridyl)-3-methyl-1H-indol-5-yl]methyl]-4-methyl-pyrimidine-5-carboxamide (118) was prepared from (2-(3-fluoropyridin-2-yl)-3-methyl-1H-indol-5-yl)methanamine following a similar procedure to that described for the synthesis of 4-methyl-N-[[3-methyl-2-(m-tolyl)-1H-indol-5-yl]methyl]pyrimidine-5-carboxamide and was obtained as an off-white solid.

Yield 13 mg (3%). ¹H NMR (400 MHz, DMSO) δ 11.22 (s, 1H), 9.14 (t, J=5.8 Hz, 1H), 9.08 (s, 1H), 8.73 (s, 1H), 8.59-8.56 (m, 1H), 7.90-7.84 (m, 1H), 7.56 (s, 1H), 7.51-7.46 (m, 1H), 7.41 (d, J=8.5 Hz, 1H), 7.19 (dd, J=1.6, 8.5 Hz, 1H), 4.58 (d, J=5.8 Hz, 2H), 2.55 (s, 3H), 2.40 (d, J=2.8 Hz, 3H). m/z: [ESI⁺] 376 (M+H)⁺, (C₂₁H₁₈FN₅O).

### Synthesis of 4-methyl-N-[[3-methyl-2-(1-methyl-4-piperidyl)-1H-indol-5-yl]methyl]pyrimidine-5-carboxamide (Compound 121)

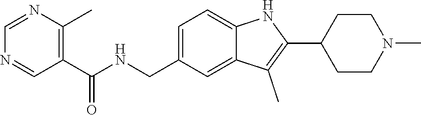 [SMILES: Cc1ncncc1C(=O)NCc1ccc2[nH]c(C3CCN(C)CC3)c(C)c2c1]

Compound 4-methyl-N-[[3-methyl-2-(1-methyl-4-piperidyl)-1H-indol-5-yl]methyl]pyrimidine-5-carboxamide (121) was prepared from (3-methyl-2-(1-methylpiperidin-4-yl)-1H-indol-5-yl)methanamine following a similar procedure to that described for the synthesis of 4-methyl-N-[[3-methyl-2-(m-tolyl)-1H-indol-5-yl]methyl]pyrimidine-5-carboxamide and was obtained as a white solid.

Yield 7 mg (4%). ¹H NMR (400 MHz, DMSO) δ 10.58 (s, 1H), 9.10-9.05 (m, 2H), 8.69 (s, 1H), 7.34 (s, 1H), 7.21 (d, J=8.5 Hz, 1H), 7.01 (dd, J=1.6, 8.5 Hz, 1H), 4.52 (d, J=5.9 Hz, 2H), 2.91-2.85 (m, 2H), 2.78-2.70 (m, 1H), 2.52 (s, 3H), 2.20 (s, 3H), 2.17 (s, 3H), 2.01-1.80 (m, 4H), 1.67-1.62 (m, 2H). m/z: [ESI⁺] 378 (M+H)⁺, (C₂₂H₂₇N₅O).

### Synthesis of 4-methyl-N-[(3-methyl-2-tetrahydropyran-4-yl-1H-indol-5-yl)methyl]pyrimidine-5-carboxamide (Compound 124)

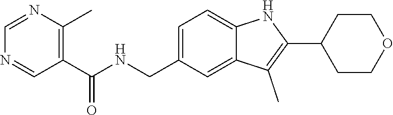 [SMILES: Cc1ncncc1C(=O)NCc1ccc2[nH]c(C3CCOCC3)c(C)c2c1]

Compound 4-methyl-N-[(3-methyl-2-tetrahydropyran-4-yl-1H-indol-5-yl)methyl]pyrimidine-5-carboxamide (124) was prepared from (3-methyl-2-(tetrahydro-2H-pyran-4-yl)-1H-indol-5-yl)methanamine following a similar procedure to that described for the synthesis of 4-methyl-N-[[3-methyl-2-(m-tolyl)-1H-indol-5-yl]methyl]pyrimidine-5-carboxamide and was obtained as an off-white solid.

Yield 60 mg (34%). ¹H NMR (400 MHz, DMSO) δ 10.63 (s, 1H), 9.11-9.07 (m, 2H), 8.69 (s, 1H), 7.36 (s, 1H), 7.22 (d, J=8.3 Hz, 1H), 7.02 (dd, J=1.5, 8.3 Hz, 1H), 4.52 (d, J=5.7 Hz, 2H), 3.96 (dd, J=3.8, 11.0 Hz, 2H), 3.50-3.44 (m, 2H), 3.14-3.04 (m, 1H), 2.53 (s, 3H), 2.19 (s, 3H), 1.94-1.82 (m, 2H), 1.65-1.58 (m, 2H). m/z: [ESI⁺] 365 (M+H)⁺, (C₂₁H₂₄N₄O₂).

### Synthesis of 4-methyl-N-[[3-methyl-2-[2-(trifluoromethyl)phenyl]-1H-indol-5-yl]methyl]pyrimidine-5-carboxamide (Compound 122)

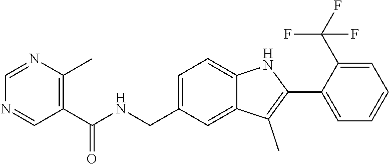 [SMILES: Cc1ncncc1C(=O)NCc1ccc2[nH]c(-c3ccccc3C(F)(F)F)c(C)c2c1]

Compound 4-methyl-N-[[3-methyl-2-[2-(trifluoromethyl)phenyl]-1H-indol-5-yl]methyl]pyrimidine-5-carboxamide (122) was prepared from (3-methyl-2-(2-(trifluoromethyl)phenyl)-1H-indol-5-yl)methanamine following a similar procedure to that described for the synthesis of 4-methyl-N-[[3-methyl-2-(m-tolyl)-1H-indol-5-yl]methyl]pyrimidine-5-carboxamide and was obtained as a white solid.

Yield 6 mg (6%). ¹H NMR (400 MHz, DMSO) δ 11.04 (s, 1H), 9.12 (t, J=5.8 Hz, 1H), 9.08 (s, 1H), 8.72 (s, 1H), 7.89 (d, J=7.3 Hz, 1H), 7.78 (dd, J=7.3, 7.6 Hz, 1H), 7.70 (dd, J=7.6, 7.6 Hz, 1H), 7.53 (d, J=7.3 Hz, 1H), 7.49 (s, 1H), 7.30 (d, J=8.3 Hz, 1H), 7.14 (dd, J=1.8, 8.3 Hz, 1H), 4.57 (d, J=5.8 Hz, 2H), 2.55 (s, 3H), 2.04 (s, 3H). m/z: [ESI⁺] 425 (M+H)⁺, (C₂₃H₁₉F₃N₄O).

### Synthesis of 4-methyl-N-[(3-methyl-2-pyrimidin-2-yl-1H-indol-5-yl)methyl]pyrimidine-5- carboxamide (Compound 126)

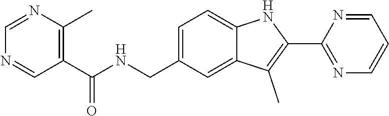 [SMILES: Cc1ncncc1C(=O)NCc1ccc2[nH]c(-c3ncccn3)c(C)c2c1]

Compound 4-methyl-N-[(3-methyl-2-pyrimidin-2-yl-1H-indol-5-yl)methyl]pyrimidine-5-carboxamide (126) was prepared from (3-methyl-2-(pyrimidin-2-yl)-1H-indol-5-yl)methanamine following a similar procedure to that described for the synthesis of 4-methyl-N-[[3-methyl-2-(m-tolyl)-1H-indol-5-yl]methyl]pyrimidine-5-carboxamide and was obtained as an off-white solid.

Yield 4 mg (2%). ¹H NMR (400 MHz, DMSO) δ 11.42 (s, 1H), 9.13 (t, J=5.8 Hz, 1H), 9.08 (s, 1H), 8.87 (d, J=5.1 Hz, 2H), 8.73 (s, 1H), 7.58 (s, 1H), 7.43 (d, J=8.6 Hz, 1H), 7.33 (d, J=5.1 Hz 1H), 7.21 (d, J=8.6 Hz, 1H), 4.57 (d, J=5.8 Hz, 2H), 2.72 (s, 3H), 2.55 (s, 3H). m/z: [ESI⁺] 359 (M+H)⁺, (C₂₀H₁₆N₆O).

### Synthesis of both enantiomers of 4-methyl-N-[[3-methyl-2-(1-methyl-3-piperidyl)-1H-indol-5-yl]methyl]pyrimidine-5-carboxamide (Compound 134 (formate) and Compound 132 (free base))

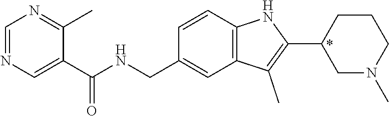 [SMILES: Cc1ncncc1C(=O)NCc1ccc2[nH]c(C3CCCN(C)C3)c(C)c2c1]

Compounds 4-methyl-N-[[3-methyl-2-(1-methyl-3-piperidyl)-1H-indol-5-yl]methyl]pyrimidine-5-carboxamide (134, 132) (formate and free base) were prepared from (3-methyl-2-(1-methylpiperidin-3-yl)-1H-indol-5-yl)methanamine following a similar procedure to that described for the synthesis of 4-methyl-N-[[3-methyl-2-(m-tolyl)-1H-indol-5-yl]methyl]pyrimidine-5-carboxamide except that after purification by preparative HPLC the enantiomers were resolved by chiral SFC.

Isomer 1 (Compound 134, formate) was obtained as a dark yellow solid.

Yield 15 mg (3%). ¹H NMR (400 MHz, DMSO) δ 10.58 (s, 1H), 9.10-9.05 (m, 2H), 8.69 (s, 1H), 8.24 (s, 1H), 7.36 (s, 1H), 7.23 (d, J=8.3 Hz, 1H), 7.02 (dd, J=1.5, 8.3 Hz, 1H), 4.52 (d, J=5.8 Hz, 2H), 3.13-3.04 (m, 1H), 2.83-2.73 (m, 2H), 2.53 (s, 3H), 2.26-2.14 (m, 7H), 2.05-1.98 (m, 1H), 1.79-1.56 (m, 4H). m/z: [ESI⁺] 378 (M+H)⁺, (C₂₂H₂₇N₅O), SFC: 98.6% ee.

Isomer 2 (Compound 132, free base) was obtained as a dark yellow solid.

Yield 15 mg (3%). ¹H NMR (400 MHz, DMSO) δ 10.58 (s, 1H), 9.11-9.06 (m, 2H), 8.69 (s, 1H), 7.36 (s, 1H), 7.23 (d, J=8.2 Hz, 1H), 7.01 (dd, J=1.5, 8.2 Hz, 1H), 4.52 (d, J=5.8 Hz, 2H), 3.11-3.02 (m, 1H), 2.77-2.70 (m, 2H), 2.53 (s, 3H), 2.21-2.10 (m, 7H), 2.01-1.94 (m, 1H), 1.78-1.55 (m, 4H). m/z: [ESI⁺] 378 (M+H)⁺, (C₂₂H₂₇N₅O), SFC: 98.3% ee.

### Synthesis of 4-methyl-N-[[3-methyl-2-(4-methyl-3-pyridyl)-1H-indol-5-yl]methyl]pyrimidine-5-carboxamide (Compound 123)

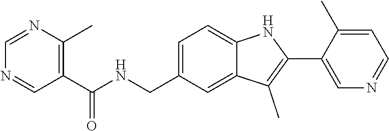 [SMILES: Cc1ccncc1-c1[nH]c2ccc(CNC(=O)c3cncnc3C)cc2c1C]

A sealed tube was charged at room temperature with N-(4-amino-3-iodobenzyl)-4-methylpyrimidine-5- carboxamide (109 mg, 0.296 mmol) and 4-methyl-3-(prop-1-yn-1-yl)pyridine (49 mg, 0.370 mmol) in anhydrous DMF (1 mL). To this solution was added K₂CO₃ (82 mg, 0.592 mmol), LiCl (13 mg, 0.296 mmol) and [1,1′-bis(diphenylphosphino)ferrocene]dichloropalladium(II) complex with dichloromethane (24 mg, 0.0296 mmol). The resulting suspension was degassed for a few minutes with nitrogen, the reaction tube sealed and the mixture heated to 100° C. for 16 hours. After cooling to room temperature, the reaction mixture was filtered through celite, rinsed with EtOAc and concentrated. The residue was partitioned between ethyl acetate (15 mL) and water (15 mL). The layers were separated, and the organic phase was washed with 4% aqueous LiCl (10 mL) and brine (10 mL), dried (MgSO₄), filtered and evaporated. The residue was purified by preparative HPLC to give 4-methyl-N-[[3-methyl-2-(4-methyl-3-pyridyl)-1H-indol-5-yl]methyl]pyrimidine-5-carboxamide (123) as an off-white solid.

Yield 25 mg (23%). ¹H NMR (400 MHz, DMSO) δ 11.06 (s, 1H), 9.13 (t, J=5.8 Hz, 1H), 9.08 (s, 1H), 8.72 (s, 1H), 8.49-8.47 (m, 2H), 7.52 (s, 1H), 7.40 (d, J=5.1 Hz, 1H), 7.32 (d, J=8.3 Hz, 1H), 7.16 (dd, J=1.8, 8.3 Hz, 1H), 4.58 (d, J=5.8 Hz, 2H), 2.55 (s, 3H), 2.26 (s, 3H), 2.14 (s, 3H). m/z: [ESI⁺] 372 (M+H)⁺, (C₂₂H₂₁N₅O).

### Synthesis of 4-methyl-N-[(3-methyl-2-pyrazin-2-yl-1H-indol-5-yl)methyl]pyrimidine-5-carboxamide (Compound 125)

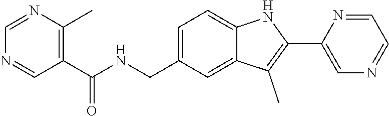 [SMILES: Cc1ncncc1C(=O)NCc1ccc2[nH]c(-c3cnccn3)c(C)c2c1]

Compound 4-methyl-N-[(3-methyl-2-pyrazin-2-yl-1H-indol-5-yl)methyl]pyrimidine-5-carboxamide (125) was prepared from 2-(prop-1-yn-1-yl)pyrazine following a similar procedure to that described for the synthesis of 4-methyl-N-[[3-methyl-2-(4-methyl-3-pyridyl)-1H-indol-5-yl]methyl]pyrimidine-5-carboxamide except that it was also purified by preparative SFC to give 4-methyl-N-[(3-methyl-2- pyrazin 2-yl-1H-indol-5-yl)methyl]pyrimidine-5-carboxamide as an off-white solid.

Yield 28 mg (26%). ¹H NMR (400 MHz, DMSO) δ 11.50 (s, 1H), 9.14 (t, J=5.8 Hz, 1H), 9.10 (m, 1H), 9.08 (s, 1H), 8.73 (s, 1H), 8.72 (dd, J=1.7, 2.5 Hz, 1H), 8.53 (d, J=2.5 Hz, 1H), 7.59 (s, 1H), 7.42 (d, J=8.5 Hz, 1H), 7.21 (d, J=8.5 Hz, 1H), 4.58 (d, J=5.8 Hz, 2H), 2.62 (s, 3H), 2.55 (s, 3H). m/z: [ESI⁺]359 (M+H)⁺, (C₂₀H₁₅N₆O).

### Synthesis of N-[(2-cyclopentyl-3-methyl-1H-indol-5-yl)methyl]-4-methyl-pyrimidine-5-carboxamide (Compound 133)

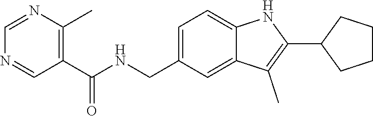 [SMILES: Cc1ncncc1C(=O)NCc1ccc2[nH]c(C3CCCC3)c(C)c2c1]

Compound N-[(2-cyclopentyl-3-methyl-1H-indol-5-yl)methyl]-4-methyl-pyrimidine-5-carboxamide (133) was prepared from prop-1-yn-1-ylcyclopentane following a similar procedure to that described for the synthesis of 4-methyl-N-[[3-methyl-2-(4-methyl-3-pyridyl)-1H-indol-5-yl]methyl]pyrimidine-5-carboxamide and was obtained as a beige solid.

Yield 2 mg (2%). ¹H NMR (400 MHz, DMSO) δ 10.52 (s, 1H), 9.10-9.03 (m, 2H), 8.69 (s, 1H), 7.33 (s, 1H), 7.21 (d, J=8.2 Hz, 1H), 7.00 (dd, J=1.6, 8.2 Hz, 1H), 4.52 (d, J=5.8 Hz, 2H), 3.28-3.18 (m, 1H), 2.53 (s, 3H), 2.17 (s, 3H), 2.00-1.92 (m, 2H), 1.87-1.77 (m, 2H), 1.74-1.62 (m, 4H). m/z: [ESI⁺]349 (M+H)⁺, (C₂₁H₂₄N₄O).

### Synthesis of 4-methyl-N-[[3-methyl-2-(3-methyl-4-pyridyl)-1H-indol-5-yl]methyl]pyrimidine-5-carboxamide (Compound 131)

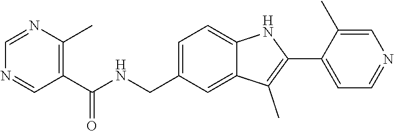 [SMILES: Cc1cnccc1-c1[nH]c2ccc(CNC(=O)c3cncnc3C)cc2c1C]

Compound 4-methyl-N-[[3-methyl-2-(3-methyl-4-pyridyl)-1H-indol-5-yl]methyl]pyrimidine-5-carboxamide (131) was prepared from 3-methyl-4-(prop-1-yn-1-yl)pyridine following a similar procedure to that described for the synthesis of 4-methyl-N-[[3-methyl-2-(4-methyl-3-pyridyl)-1H-indol-5-yl]methyl]pyrimidine-5-carboxamide except that it was also purified by preparative SFC to give 4-methyl-N-[[3-methyl-2-(3-methyl-4-pyridyl)-1H-indol-5-yl]methyl]pyrimidine-5-carboxamide (131) as a yellow solid.

Yield 50 mg (41%). ¹H NMR (400 MHz, DMSO) δ 11.08 (s, 1H), 9.14 (t, J=5.8 Hz, 1H), 9.08 (s, 1H), 8.72 (s, 1H), 8.57 (s, 1H), 8.49 (d, J=4.9 Hz, 1H), 7.53 (s, 1H), 7.35-7.32 (m, 2H), 7.17 (dd, J=1.6, 8.3 Hz, 1H), 4.58 (d, J=5.8 Hz, 2H), 2.55 (s, 3H), 2.27 (s, 3H), 2.17 (s, 3H). m/z: [ESI⁺] 372 (M+H)⁺, (C₂₂H₂₁N₅O).

### Synthesis of 4-methyl-N-[[3-methyl-2-(3-methyl-2-pyridyl)-1H-indol-5-yl]methyl]pyrimidine-5-carboxamide (Compound 130)

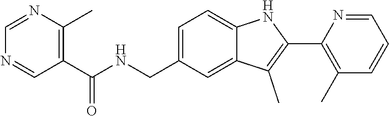 [SMILES: Cc1cccnc1-c1[nH]c2ccc(CNC(=O)c3cncnc3C)cc2c1C]

Compound 4-methyl-N-[[3-methyl-2-(3-methyl-2-pyridyl)-1H-indol-5-yl]methyl]pyrimidine-5-carboxamide (130) was prepared from 3-methyl-2-(prop-1-yn-1-yl)pyridine following a similar procedure to that described for the synthesis of 4-methyl-N-[[3-methyl-2-(4-methyl-3-pyridyl)-1H-indol-5-yl]methyl]pyrimidine-5-carboxamide except that it was also purified by preparative SFC to give 4-methyl-N-[[3-methyl-2-(3-methyl-2-pyridyl)-1H-indol-5-yl]methyl]pyrimidine-5-carboxamide (130) as a white solid.

Yield 20 mg (17%). ¹H NMR (400 MHz, DMSO) δ 11.03 (s, 1H), 9.13 (t, J=5.8 Hz, 1H), 9.08 (s, 1H), 8.72 (s, 1H), 8.53 (dd, J=1.1, 4.7 Hz, 1H), 7.77 (d, J=8.3 Hz, 1H), 7.52 (s, 1H), 7.36-7.32 (m, 2H), 7.15 (d, J=8.3 Hz, 1H), 4.58 (d, J=5.8 Hz, 2H), 2.55 (s, 3H), 2.29 (s, 3H), 2.18 (s, 3H). m/z: [ESI⁺] 372 (M+H)⁺, (C₂₂H₂₁N₅O).

### Synthesis of N-[[2-(2,6-difluorophenyl)-3-methyl-1H-indol-5-yl]methyl]-4-methyl-pyrimidine-5-carboxamide (Compound 128)

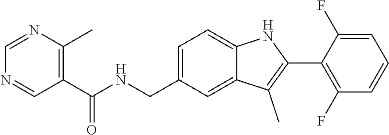 [SMILES: Cc1ncncc1C(=O)NCc1ccc2[nH]c(-c3c(F)cccc3F)c(C)c2c1]

Compound N-[[2-(2,6-difluorophenyl)-3-methyl-1H-indol-5-yl]methyl]-4-methyl-pyrimidine-5-carboxamide (128) was prepared from 1,3-difluoro-2-(prop-1-yn-1-yl)benzene following a similar procedure to that described for the synthesis of 4-methyl-N-[[3-methyl-2-(4-methyl-3-pyridyl)-1H-indol-5-yl]methyl]pyrimidine-5-carboxamide except that it was also purified by preparative SFC to give N-[[2- (2,6-difluorophenyl)-3-methyl-1H-indol-5-yl]methyl]-4-methyl-pyrimidine-5-carboxamide (128) as an off-white solid.

Yield 20 mg (19%). ¹H NMR (400 MHz, DMSO) δ 11.18 (s, 1H), 9.13 (t, J=5.8 Hz, 1H), 9.08 (s, 1H), 8.72 (s, 1H), 7.59-7.52 (m, 2H), 7.34 (d, J=8.3 Hz, 1H), 7.32-7.24 (m, 2H), 7.17 (d, J=8.3 Hz, 1H), 4.58 (d, J=5.8 Hz, 2H), 2.54 (s, 3H), 2.15 (s, 3H). ¹⁹F NMR (376.5 MHz, DMSO) δ=−111 (s). m/z: [ESI⁺] 393 (M+H)⁺, (C₂₂H₁₈F₂N₄O).

### Synthesis of N-[[2-(2-fluoro-3-pyridyl)-3-methyl-1H-indol-5-yl]methyl]-4-methyl-pyrimidine-5-carboxamide (Compound 129)

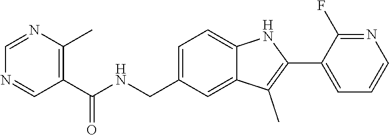 [SMILES: Cc1ncncc1C(=O)NCc1ccc2[nH]c(-c3cccnc3F)c(C)c2c1]

Compound N-[[2-(2-fluoro-3-pyridyl)-3-methyl-1H-indol-5-yl]methyl]-4-methyl-pyrimidine-5-carboxamide (129) was prepared from 2-fluoro-3-(prop-1-yn-1-yl)pyridine following a similar procedure to that described for the synthesis of 4-methyl-N-[[3-methyl-2-(4-methyl-3-pyridyl)-1H indol-5-yl]methyl]pyrimidine-5-carboxamide except that it was also purified by preparative SFC to give N-[[2-(2-fluoro-3-pyridyl)-3-methyl-1H-indol-5-yl]methyl]-4-methyl-pyrimidine-5-carboxamide (129) as an off-white solid.

Yield 15 mg (12%). ¹H NMR (400 MHz, DMSO) δ 11.22 (s, 1H), 9.14 (t, J=6.1 Hz, 1H), 9.08 (s, 1H), 8.72 (s, 1H), 8.30-8.27 (m, 1H), 8.15-8.09 (m, 1H), 7.55-7.50 (m, 2H), 7.37 (d, J=8.3 Hz, 1H), 7.18 (d, J=8.3 Hz, 1H), 4.58 (d, J=6.1 Hz, 2H), 2.54 (s, 3H), 2.27 (d, J=1.5 Hz, 3H). ¹⁹F NMR (376.5 MHz, DMSO) δ=−68.5 (s). m/z: [ESI⁺] 376 (M+H)⁺, (C₂₁H₁₈FN₅O).

### Synthesis of N-[[2-(3-fluoro-4-pyridyl)-3-methyl-1H-indol-5-yl]methyl]-4-methyl-pyrimidine-5-carboxamide (Compound 127)

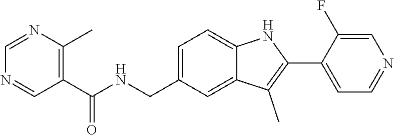 [SMILES: Cc1ncncc1C(=O)NCc1ccc2[nH]c(-c3ccncc3F)c(C)c2c1]

Compound N-[[2-(3-fluoro-4-pyridyl)-3-methyl-1H-indol-5-yl]methyl]-4-methyl-pyrimidine-5-carboxamide (127) was prepared from 3-fluoro-4-(prop-1-yn-1-yl)pyridine following a similar procedure to that described for the synthesis of 4-methyl-N-[[3-methyl-2-(4-methyl-3-pyridyl)-1H-indol-5-yl]methyl]pyrimidine-5-carboxamide except that it was also purified by preparative SFC to give N-[[2-(3-fluoro-4-pyridyl)-3-methyl-1H-indol-5-yl]methyl]-4-methyl-pyrimidine-5-carboxamide (127) as a beige solid.

Yield 26 mg (21%). ¹H NMR (400 MHz, DMSO) δ 11.31 (s, 1H), 9.15 (t, J=5.8 Hz, 1H), 9.08 (s, 1H), 8.72 (s, 1H), 8.71 (d, J=2.4 Hz, 1H), 8.54 (dd, J=1.1, 5.1 Hz, 1H), 7.63 (dd, J=5.1, 6.6 Hz, 1H), 7.57 (s, 1H), 7.40 (d, J=8.3 Hz, 1H), 7.22 (dd, J=1.6, 8.3 Hz, 1H), 4.58 (d, J=5.8 Hz, 2H), 2.54 (s, 3H), 2.32 (d, J=2.0 Hz, 3H). ¹⁹F NMR (376.5 MHz, DMSO) δ=−129 (s). m/z: [ESI⁺] 376 (M+H)⁺, (C₂₁H₁₈FN₅O).

### Synthesis of 4-methyl-N-[[3-methyl-2-(2-methylpyrazol-3-yl)-1H-indol-5-yl]methyl]pyrimidine-5-carboxamide (Compound 141)

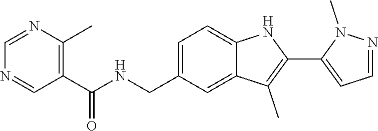 [SMILES: Cc1ncncc1C(=O)NCc1ccc2[nH]c(-c3ccnn3C)c(C)c2c1]

Compound 4-methyl-N-[[3-methyl-2-(2-methylpyrazol-3-yl)-1H-indol-5 yl]methyl]pyrimidine-5-carboxamide (141) was prepared from 1-methyl-5-(prop-1-yn-1-yl)-1H-pyrazole following a similar procedure to that described for the synthesis of 4-methyl-N-[[3-methyl-2-(4-methyl-3-pyridyl)-1H-indol-5-yl]methyl]pyrimidine-5-carboxamide except that it was also purified by preparative SFC to give 4-methyl-N-[[3-methyl-2-(2-methylpyrazol-3-yl)-1H-indol-5-yl]methyl]pyrimidine-5-carboxamide (141) as a white solid.

Yield 11 mg (9%). 1H NMR (400 MHz, DMSO) δ 11.15 (s, 1H), 9.14 (t, J=5.8 Hz, 1H), 9.08 (s, 1H), 8.72 (s, 1H), 7.55 (d, J=2.0 Hz, 1H), 7.54-7.52 (m, 1H), 7.34 (d, J=8.5 Hz, 1H), 7.18 (dd, J=1.6, 8.5 Hz, 1H), 6.45 (d, J=2.0 Hz, 1H), 4.57 (d, J=5.8 Hz, 2H), 3.81 (s, 3H), 2.54 (s, 3H), 2.22 (s, 3H). m/z: [ESI⁺] 361 (M+H)⁺, (C₂₀H₂₀N₆O).

### Synthesis of N-[[2-(2-chlorophenyl)-3-methyl-1H-indol-5-yl]methyl]-4-methyl-pyrimidine-5-carboxamide (Compound 140)

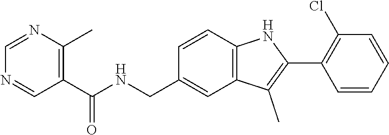 [SMILES: Cc1ncncc1C(=O)NCc1ccc2[nH]c(-c3ccccc3Cl)c(C)c2c1]

Compound N-[[2-(2-chlorophenyl)-3-methyl-1H-indol-5-yl]methyl]-4-methyl-pyrimidine-5-carboxamide (140) was prepared from 1-chloro-2-(prop-1-yn-1-yl)benzene following a similar procedure to that described for the synthesis of 4-methyl-N-[[3-methyl-2-(4-methyl-3-pyridyl)-1H-indol-5-yl]methyl]pyrimidine-5- carboxamide and was obtained as an off-white solid.

Yield 28 mg (22%). ¹H NMR (400 MHz, DMSO) δ 11.08 (s, 1H), 9.13 (t, J=5.8 Hz, 1H), 9.08 (s, 1H), 8.72 (s, 1H), 7.65-7.59 (m, 1H), 7.53-7.45 (m, 4H), 7.32 (d, J=8.3 Hz, 1H), 7.15 (d, J=8.3 Hz, 1H), 4.57 (d, J=5.8 Hz, 2H), 2.55 (s, 3H), 2.15 (s, 3H). m/z: [ESI⁺] 391 (M+H)⁺, (C₂₂H₁₉ClN₄O).

### Synthesis of N-[[2-(2-ethylphenyl)-3-methyl-1H-indol-5-yl]methyl]-4-methyl-pyrimidine-5-carboxamide (Compound 1391

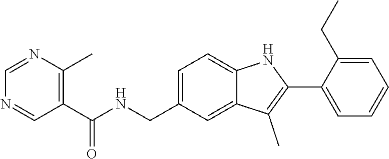 [SMILES: CCc1ccccc1-c1[nH]c2ccc(CNC(=O)c3cncnc3C)cc2c1C]

Compound N-[[2-(2-ethylphenyl)-3-methyl-1H-indol-5-yl]methyl]-4-methyl-pyrimidine-5-carboxamide (139) was prepared from 1-ethyl-2-(prop-1-yn-1-yl)benzene following a similar procedure to that described for the synthesis of 4-methyl-N-[[3-methyl-2-(4-methyl-3-pyridyl)-1H-indol-5-yl]methyl]pyrimidine-5-carboxamide except that it was also purified by preparative SFC to give N-[[2-(2-ethylphenyl)-3-methyl-1H-indol-5-yl]methyl]-4-methyl-pyrimidine-5-carboxamide (139) as an off-white solid.

Yield 17 mg (14%). ¹H NMR (400 MHz, DMSO) δ 10.94 (s, 1H), 9.12 (t, J=5.8 Hz, 1H), 9.08 (s, 1H), 8.72 (s, 1H), 7.47 (s, 1H), 7.40-7.38 (m, 2H), 7.33-7.27 (m, 3H), 7.11 (d, J=8.3 Hz, 1H), 4.57 (d, J=5.8 Hz, 2H), 2.59-2.52 (m, 5H), 2.09 (s, 3H), 0.98 (t, J=7.6 Hz, 3H). m/z: [ESI⁺] 385 (M+H)⁺, (C₂₄H₂₄N₄O).

### Synthesis of 4-methyl-N-[[3-methyl-2-(6-methyl-2-pyridyl)-1H-indol-5-yl]methyl]pyrimidine-5-carboxamide (Compound 137) and 4-methyl-N-[[2-methyl-3-(6-methyl-2-pyridyl)-1H-indol-5-yl]methyl]pyrimidine-5-carboxamide (Compound 138)

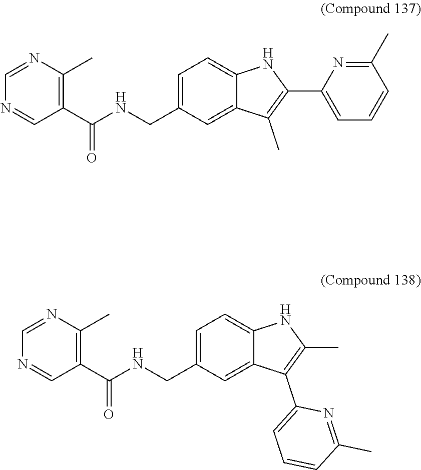 [SMILES: Cc1cccc(-c2[nH]c3ccc(CNC(=O)c4cncnc4C)cc3c2C)n1 ; Cc1cccc(-c2c(C)[nH]c3ccc(CNC(=O)c4cncnc4C)cc23)n1]

Compounds 4-methyl-N-[[3-methyl-2-(6-methyl-2-pyridyl)-1H-indol-5-yl]methyl]pyrimidine-5-carboxamide (137) and 4-methyl-N-[[2-methyl-3-(6-methyl-2-pyridyl)-1H-indol-5-yl]methyl]pyrimidine-5- carboxamide (138) were prepared from 2-methyl-6-(prop-1-yn-1-yl)pyridine following a similar procedure to that described for the synthesis of 4-methyl-N-[[3-methyl-2-(4-methyl-3-pyridyl)-1H-indol-5- yl]methyl]pyrimidine-5-carboxamide.

4-Methyl-N-[[3-methyl-2-(6-methyl-2-pyridyl)-1H-indol-5-yl]methyl]pyrimidine-5-carboxamide (Compound 137) was obtained as a brown solid.

Yield 52 mg (43%). ¹H NMR (400 MHz, DMSO) δ 11.16 (s, 1H), 9.13 (t, J=5.6 Hz, 1H), 9.08 (s, 1H), 8.72 (s, 1H), 7.78 (dd, J=7.8, 7.8 Hz, 1H), 7.63 (d, J=7.8 Hz, 1H), 7.55 (s, 1H), 7.42 (d, J=7.8 Hz, 1H), 7.16 (d, J=7.8 Hz, 2H), 4.57 (d, J=5.6 Hz, 2H), 2.58 (s, 3H), 2.56 (s, 3H), 2.55 (s, 3H). m/z: [ESI⁺] 372 (M+H)⁺, (C₂₂H₂₁N₅O).

4-Methyl-N-[[2-methyl-3-(6-methyl-2-pyridyl)-1H-indol-5-yl]methyl]pyrimidine-5-carboxamide (Compound 138) was obtained as an off-white solid.

Yield 6 mg (5%). ¹H NMR (400 MHz, DMSO) δ 11.27 (s, 1H), 9.13 (t, J=5.8 Hz, 1H), 9.07 (s, 1H), 8.71 (s, 1H), 7.96 (s, 1H), 7.69 (dd, J=7.8, 7.8 Hz, 1H), 7.38 (d, J=7.8 Hz, 1H), 7.31 (d, J=8.2 Hz, 1H), 7.09 (d, J=8.2 Hz, 1H), 7.04 (d, J=7.8 Hz, 1H), 4.56 (d, J=5.8 Hz, 2H), 2.62 (s, 3H), 2.52 (s, 3H), 2.49 (s, 3H). m/z: [ESI⁺] 372 (M+H)⁺, (C₂₂H₂₁N₅O).

### Synthesis of 4-methyl-N-[[3-methyl-2-(2-methylthiazol-4-yl)-1H-indol-5-yl]methyl]pyrimidine-5-carboxamide (Compound 136)

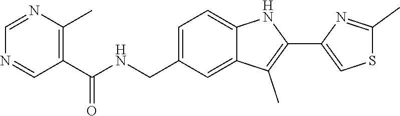 [SMILES: Cc1nc(-c2[nH]c3ccc(CNC(=O)c4cncnc4C)cc3c2C)cs1]

Compound 4-methyl-N-[[3-methyl-2-(2-methylthiazol-4-yl)-1H-indol-5-yl]methyl]pyrimidine-5-carboxamide (136) was prepared from 2-methyl-4-(prop-1-yn-1-yl)thiazole following a similar procedure to that described for the synthesis of 4-methyl-N-[[3-methyl-2-(4-methyl-3-pyridyl)-1H-indol-5-yl]methyl]pyrimidine-5-carboxamide except that it was also purified by preparative SFC to give 4-methyl-N-[[3-methyl-2-(2-methylthiazol-4-yl)-1H-indol-5-yl]methyl]pyrimidine-5-carboxamide (136) as an off-white solid.

Yield 23 mg (19%). ¹H NMR (400 MHz, DMSO) δ 11.16 (s, 1H), 9.12 (t, J=5.8 Hz, 1H), 9.08 (s, 1H), 8.72 (s, 1H), 7.69 (s, 1H), 7.50 (s, 1H), 7.34 (d, J=8.3 Hz, 1H), 7.12 (dd, J=1.6, 8.3 Hz, 1H), 4.56 (d, J=5.8 Hz, 2H), 2.75 (s, 3H), 2.54 (s, 3H), 2.51 (s, 3H). m/z: [ESI⁺] 378 (M+H)⁺, (C₂₀H₁₉N₅OS).

### Synthesis of N-[[2-(6-fluoro-2-pyridyl)-3-methyl-1H-indol-5-yl]methyl]-4-methyl-pyrimidine-5-carboxamide (Compound 135)

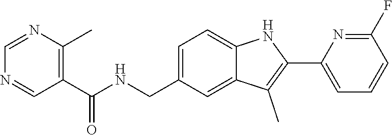 [SMILES: Cc1ncncc1C(=O)NCc1ccc2[nH]c(-c3cccc(F)n3)c(C)c2c1]

Compound N-[[2-(6-fluoro-2-pyridyl)-3-methyl-1H-indol-5-yl]methyl]-4-methyl-pyrimidine-5-carboxamide (135) was prepared from 2-fluoro-6-(prop-1-yn-1-yl)pyridine following a similar procedure to that described for the synthesis of 4-methyl-N-[[3-methyl-2-(4-methyl-3-pyridyl)-1H-indol-5-yl]methyl]pyrimidine-5-carboxamide and was obtained as a brown solid.

Yield 10 mg (8%). ¹H NMR (400 MHz, DMSO) δ 11.37 (s, 1H), 9.14 (t, J=5.9 Hz, 1H), 9.08 (s, 1H), 8.73 (s, 1H), 8.09 (q, J=8.2 Hz, 1H), 7.77 (dd, J=2.6, 7.7 Hz, 1H), 7.57 (s, 1H), 7.41 (d, J=8.5 Hz, 1H), 7.19 (d, J=8.4 Hz, 1H), 7.06 (d, J=8.4 Hz, 1H), 4.57 (d, J=5.9 Hz, 2H), 2.59 (s, 3H), 2.55 (s, 3H). ¹⁹F NMR (376.5 MHz, DMSO) δ=−73 (s). m/z: [ESI⁺] 376 (M+H)⁺, (C₂₁H₁₈FN₅O).

### Synthesis of N-[[2-(5-methoxy-2-pyridyl)-3-methyl-1H-indol-5-yl]methyl]-4-methyl-pyrimidine-5-carboxamide (Compound 142)

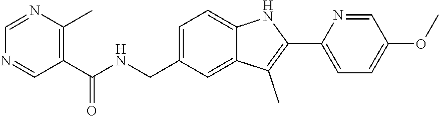 [SMILES: COc1ccc(-c2[nH]c3ccc(CNC(=O)c4cncnc4C)cc3c2C)nc1]

Compound N-[[2-(5-methoxy-2-pyridyl)-3-methyl-1H-indol-5-yl]methyl]-4-methyl-pyrimidine-5-carboxamide (142) was prepared from 5-methoxy-2-(prop-1-yn-1-yl)pyridine following a similar procedure to that described for the synthesis of 4-methyl-N-[[3-methyl-2-(4-methyl-3-pyridyl)-1H-indol-5-yl]methyl]pyrimidine-5-carboxamide and was obtained as a brown solid.

Yield 35 mg (28%). ¹H NMR (400 MHz, DMSO) δ 11.20 (s, 1H), 9.12 (t, J=5.8 Hz, 1H), 9.08 (s, 1H), 8.72 (s, 1H), 8.40 (d, J=2.8 Hz, 1H), 7.79 (d, J=8.8 Hz, 1H), 7.55-7.51 (m, 2H), 7.37 (d, J=8.3 Hz, 1H), 7.13 (d, J=8.3 Hz, 1H), 4.56 (d, J=5.8 Hz, 2H), 3.89 (s, 3H), 2.54 (s, 3H), 2.53 (s, 3H). m/z: [ESI⁺] 388 (M+H)⁺, (C₂₂H₂₁N₅O₂).

### Synthesis of N-[[2-(4-fluoro-2-pyridyl)-3-methyl-1H-indol-5-yl]methyl]-4-methyl-pyrimidine-5-carboxamide (Compound 143)

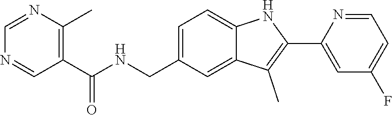 [SMILES: Cc1ncncc1C(=O)NCc1ccc2[nH]c(-c3cc(F)ccn3)c(C)c2c1]

Compound N-[[2-(4-fluoro-2-pyridyl)-3-methyl-1H-indol-5-yl]methyl]-4-methyl-pyrimidine-5-carboxamide (143) was prepared from 4-fluoro-2-(prop-1-yn-1-yl)pyridine following a similar procedure to that described for the synthesis of 4-methyl-N-[[3-methyl-2-(4-methyl-3-pyridyl)-1H-indol-5-yl]methyl]pyrimidine-5-carboxamide and was obtained as a beige solid.

Yield 64 mg (52%). ¹H NMR (400 MHz, DMSO) δ 11.39 (s, 1H), 9.13 (t, J=5.8 Hz, 1H), 9.08 (s, 1H), 8.72 (s, 1H), 8.70 (dd, J=5.6, 9.1 Hz, 1H), 7.68 (dd, J=2.3, 11.1 Hz, 1H), 7.58 (s, 1H), 7.41 (d, J=8.3 Hz, 1H), 7.26-7.18 (m, 2H), 4.58 (d, J=5.8 Hz, 2H), 2.60 (s, 3H), 2.55 (s, 3H). ¹⁹F NMR (376.5 MHz, DMSO) δ=−103 (s). m/z: [ESI⁺] 376 (M+H)⁺, (C₂₁H₁₈FN₅O).

### Synthesis of N-[[2-(4-methoxy-2-pyridyl)-3-methyl-1H-indol-5-yl]methyl]-4-methyl-pyrimidine-5-carboxamide (Compound 146)

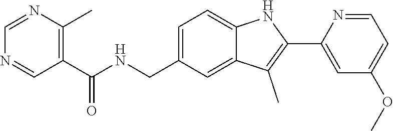 [SMILES: COc1ccnc(-c2[nH]c3ccc(CNC(=O)c4cncnc4C)cc3c2C)c1]

Compound N-[[2-(4-methoxy-2-pyridyl)-3-methyl-1H-indol-5-yl]methyl]-4-methyl-pyrimidine-5-carboxamide (146) was prepared from 4-methoxy-2-(prop-1-yn-1-yl)pyridine following a similar procedure to that described for the synthesis of 4-methyl-N-[[3-methyl-2-(4-methyl-3-pyridyl)-1H-indol-5- yl]methyl]pyrimidine-5-carboxamide and was obtained as a beige solid.

Yield 44 mg (37%). ¹H NMR (400 MHz, DMSO) δ 11.29 (s, 1H), 9.13 (t, J=5.8 Hz, 1H), 9.08 (s, 1H), 8.72 (s, 1H), 8.49 (d, J=5.7 Hz, 1H), 7.55 (s, 1H), 7.39 (d, J=8.3 Hz, 1H), 7.35 (d, J=2.3 Hz, 1H), 7.16 (d, J=8.3 Hz, 1H), 6.91 (dd, J=2.4, 5.7 Hz, 1H), 4.57 (d, J=5.8 Hz, 2H), 3.92 (s, 3H), 2.59 (s, 3H), 2.55 (s, 3H). m/z: [ESI⁺] 388 (M+H)⁺, (C₂₂H₂₁N₅O₂).

### Synthesis of 4-methyl-N-[(3-methyl-2-pyrimidin-4-yl-1H-indol-5-yl)methyl]pyrimidine-5-carboxamide (Compound 145)

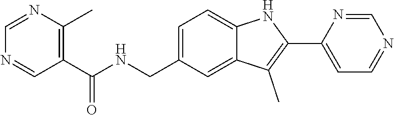 [SMILES: Cc1ncncc1C(=O)NCc1ccc2[nH]c(-c3ccncn3)c(C)c2c1]

Compound 4-methyl-N-[(3-methyl-2-pyrimidin-4-yl-1H-indol-5-yl)methyl]pyrimidine-5-carboxamide (145) was prepared from 4-(prop-1-yn-1-yl)pyrimidine following a similar procedure to that described for the synthesis of 4-methyl-N-[[3-methyl-2-(4-methyl-3-pyridyl)-1H-indol-5-yl]methyl]pyrimidine-5-carboxamide except that it was also purified by preparative SFC to give 4-methyl-N-[(3-methyl-2-pyrimidin-4-yl-1H-indol-5-yl)methyl]pyrimidine-5-carboxamide (145) as an off-white solid.

Yield 15 mg (13%). ¹H NMR (400 MHz, DMSO) δ 11.59 (s, 1H), 9.20 (d, J=1.3 Hz, 1H), 9.14 (t, J=5.8 Hz, 1H), 9.08 (s, 1H), 8.82 (d, J=5.6 Hz, 1H), 8.74 (s, 1H), 7.88 (dd, J=1.3, 5.6 Hz, 1H), 7.62 (s, 1H), 7.44 (d, J=8.3 Hz, 1H), 7.25 (dd, J=1.3, 8.3 Hz, 1H), 4.57 (d, J=5.8 Hz, 2H), 2.66 (s, 3H), 2.55 (s, 3H). m/z: [ESI⁺] 359 (M+H)⁺, (C₂₀H₁₈N₆O).

### Synthesis of 4-methyl-N-[[3-methyl-2-(1-methylimidazol-2-yl)-1H-indol-5-yl]methyl]pyrimidine-5-carboxamide formate (Compound 144)

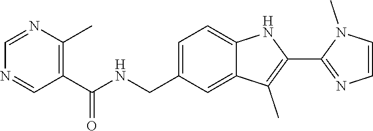 [SMILES: Cc1ncncc1C(=O)NCc1ccc2[nH]c(-c3nccn3C)c(C)c2c1]

Compound 4-methyl-N-[[3-methyl-2-(1-methylimidazol-2-yl)-1H-indol-5-yl]methyl]pyrimidine-5-carboxamide (144) formate was prepared from 1-methyl-2-(prop-1-yn-1-yl)-1H-imidazole following a similar procedure to that described for the synthesis of 4-methyl-N-[[3-methyl-2-(4-methyl-3-pyridyl)-1H-indol-5-yl]methyl]pyrimidine-5-carboxamide and was obtained as a beige solid.

Yield 9 mg (7%). ¹H NMR (400 MHz, DMSO) δ 11.13 (s, 1H), 9.14 (t, J=5.8 Hz, 1H), 9.08 (s, 1H), 8.72 (s, 1H), 8.35 (s, 0.5H), 7.53 (s, 1H), 7.36-7.31 (m, 2H), 7.18 (dd, J=1.5, 8.3 Hz, 1H), 7.07 (d, J=1.5 Hz, 1H), 4.58 (d, J=5.8 Hz, 2H), 3.66 (s, 3H), 2.55 (s, 3H), 2.28 (s, 3H). m/z: [ESI⁺] 361 (M+H)⁺, (C₂₀H₂₀N₆O).

### Synthesis of N-[[2-(6-methoxy-2-pyridyl)-3-methyl-1H-indol-5-yl]methyl]-4-methyl-pyrimidine-5-carboxamide (Compound 155) and N-[[3-(6-methoxy-2-pyridyl)-2-methyl-1H-indol-5-yl]methyl]-4-methyl-pyrimidine-5-carboxamide (Compound 153)

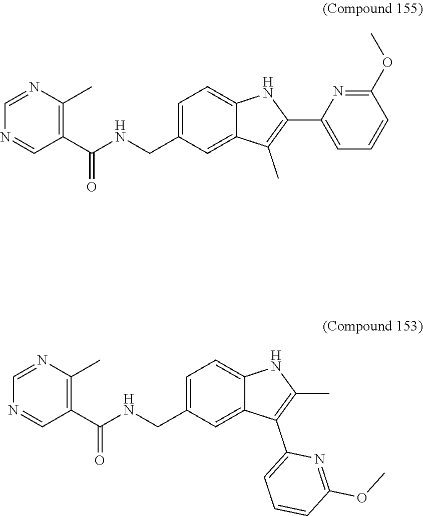 [SMILES: COc1cccc(-c2[nH]c3ccc(CNC(=O)c4cncnc4C)cc3c2C)n1 ; COc1cccc(-c2c(C)[nH]c3ccc(CNC(=O)c4cncnc4C)cc23)n1]

Compounds N-[[2-(6-methoxy-2-pyridyl)-3-methyl-1H-indol-5-yl]methyl]-4-methyl-pyrimidine-5-carboxamide (155) and N-[[3-(6-methoxy-2-pyridyl)-2-methyl-1H-indol-5-yl]methyl]-4-methyl-pyrimidine-5- carboxamide (153) were prepared from 2-methoxy-6-(prop-1-yn-1-yl)pyridine following a similar procedure to that described for the synthesis of 4-methyl-N-[[3-methyl-2-(4-methyl-3-pyridyl)-1H-indol-5-yl]methyl]pyrimidine-5-carboxamide except that they were also purified by preparative SFC.

N-[[2-(6-methoxy-2-pyridyl)-3-methyl-1H-indol-5-yl]methyl]-4-methyl-pyrimidine-5-carboxamide (Compound 155) was obtained as a brown solid.

Yield 5 mg (4%). ¹H NMR (400 MHz, DMSO) δ 11.19 (s, 1H), 9.14 (t, J=5.8 Hz, 1H), 9.08 (s, 1H), 8.73 (s, 1H), 7.79 (dd, J=7.8, 10.0 Hz, 1H), 7.56 (s, 1H), 7.42 (dd, J=7.8, 10.0 Hz, 2H), 7.18 (d, J=8.3 Hz, 1H), 6.71 (d, J=8.3 Hz, 1H), 4.57 (d, J=5.8 Hz, 2H), 4.00 (s, 3H), 2.62 (s, 3H), 2.55 (s, 3H). m/z: [ESI⁺]388 (M+H)⁺, (C₂₂H₂₁N₅O₂).

N-[[3-(6-methoxy-2-pyridyl)-2-methyl-1H-indol-5-yl]methyl]-4-methyl-pyrimidine-5-carboxamide (Compound 153) was obtained as a beige solid.

Yield 8 mg (6%). ¹H NMR (400 MHz, DMSO) δ 11.33 (s, 1H), 9.13 (t, J=5.8 Hz, 1H), 9.07 (s, 1H), 8.69 (s, 1H), 8.01 (s, 1H), 7.72 (d, J=7.8 Hz, 1H), 7.32 (d, J=7.8 Hz, 1H), 7.21 (d, J=6.8 Hz, 1H), 7.11 (d, J=7.8 Hz, 1H), 6.59 (d, J=7.8 Hz, 1H), 4.56 (d, J=5.8 Hz, 2H), 3.90 (s, 3H), 2.67 (s, 3H), 2.51 (s, 3H). m/z: [ESI⁺] 388 (M+H)⁺, (C₂₂H₂₁N₅O₂).

### Synthesis of 4-methyl-N-[[3-methyl-2-(5-methylpyrimidin-4-yl)-1H-indol-5-yl]methyl]pyrimidine-5-carboxamide (Compound 151)

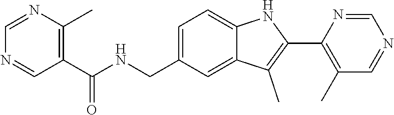 [SMILES: Cc1cncnc1-c1[nH]c2ccc(CNC(=O)c3cncnc3C)cc2c1C]

Compound 151 was prepared from 5-methyl-4-(prop-1-yn-1-yl)pyrimidine following a similar procedure to that described for the synthesis of 4-methyl-N-[[3-methyl-2-(4-methyl-3-pyridyl)-1H-indol-5-yl]methyl]pyrimidine-5-carboxamide and was obtained as a pale yellow solid.

Yield 20 mg (17%). ¹H NMR (400 MHz, DMSO) δ 11.24 (s, 1H), 9.20 (t, J=5.8 Hz, 1H), 9.16 (s, 1H), 9.14 (s, 1H), 8.82 (s, 1H), 8.79 (s, 1H), 7.63 (s, 1H), 7.45 (d, J=8.3 Hz, 1H), 7.28 (dd, J=1.5, 8.3 Hz, 1H), 4.64 (d, J=5.8 Hz, 2H), 2.61 (s, 3H), 2.41 (s, 3H), 2.36 (s, 3H). m/z: [ESI⁺] 373 (M+H)⁺, (C₂₁H₂₀N₆O).

### Synthesis of 4-methyl-N-[[3-methyl-2-[6-(trifluoromethyl)-2-pyridyl]-1H-indol-5-yl]methyl]pyrimidine-5-carboxamide (Compound 149)

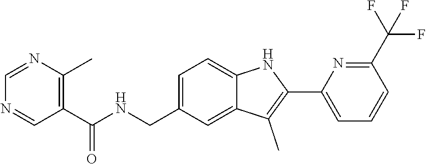 [SMILES: Cc1ncncc1C(=O)NCc1ccc2[nH]c(-c3cccc(C(F)(F)F)n3)c(C)c2c1]

Compound 4-methyl-N-[[3-methyl-2-[6-(trifluoromethyl)-2-pyridyl]-1H-indol-5-yl]methyl]pyrimidine-5-carboxamide (149) was prepared from 2-(prop-1-yn-1-yl)-6-(trifluoromethyl)pyridine following a similar procedure to that described for the synthesis of 4-methyl-N-[[3-methyl-2-(4-methyl-3-pyridyl)-1H-indol-5-yl]methyl]pyrimidine-5-carboxamide and was obtained as a brown solid.

Yield 24 mg (17%). ¹H NMR (400 MHz, DMSO) δ 11.39 (s, 1H), 9.14 (t, J=5.8 Hz, 1H), 9.08 (s, 1H), 8.74 (s, 1H), 8.18 (dd, J=7.8, 7.8 Hz, 1H), 8.11 (d, J=7.8 Hz, 1H), 7.76 (d, J=7.3 Hz, 1H), 7.60 (s, 1H), 7.45 (d, J=8.3 Hz, 1H), 7.22 (dd, J=1.5, 8.3 Hz, 1H), 4.58 (d, J=5.8 Hz, 2H), 2.63 (s, 3H), 2.55 (s, 3H). ¹⁹F NMR (376.5 MHz, DMSO) δ=−67 (s). m/z: [ESI⁺] 426 (M+H)⁺, (C₂₂H₁₈F₃N₅O).

### Synthesis of 4-methyl-N-[[3-methyl-2-(5-methyl-2-pyridyl)-1H-indol-5-yl]methyl]pyrimidine-5-carboxamide (Compound 148)

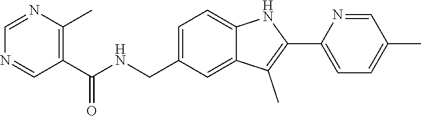 [SMILES: Cc1ccc(-c2[nH]c3ccc(CNC(=O)c4cncnc4C)cc3c2C)nc1]

Compound 4-methyl-N-[[3-methyl-2-(5-methyl-2-pyridyl)-1H-indol-5-yl]methyl]pyrimidine-5-carboxamide (148) was prepared from 5-methyl-2-(prop-1-yn-1-yl)pyridine following a similar procedure to that described for the synthesis of 4-methyl-N-[[3-methyl-2-(4-methyl-3-pyridyl)-1H-indol-5-yl]methyl]pyrimidine-5-carboxamide and was obtained as a grey solid.

Yield 22 mg (18%). ¹H NMR (400 MHz, DMSO) δ 11.27 (s, 1H), 9.12 (t, J=5.8 Hz, 1H), 9.08 (s, 1H), 8.73 (s, 1H), 8.52 (s, 1H), 7.76-7.70 (m, 2H), 7.54 (s, 1H), 7.38 (d, J=8.3 Hz, 1H), 7.14 (dd, J=1.5, 8.3 Hz, 1H), 4.57 (d, J=5.8 Hz, 2H), 2.55 (s, 3H), 2.55 (s, 3H), 2.35 (s, 3H). m/z: [ESI⁺] 372 (M+H)⁺, (C₂₂H₂₁N₅O).

### Synthesis of N-[[2-(5-fluoro-2-pyridyl)-3-methyl-1H-indol-5-yl]methyl]-4-methyl-pyrimidine-5-carboxamide (Compound 147)

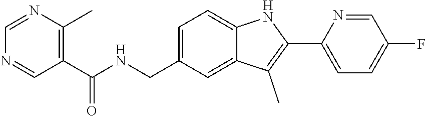 [SMILES: Cc1ncncc1C(=O)NCc1ccc2[nH]c(-c3ccc(F)cn3)c(C)c2c1]

Compound N-[[2-(5-fluoro-2-pyridyl)-3-methyl-1H-indol-5-yl]methyl]-4-methyl-pyrimidine-5-carboxamide (147) was prepared from 5-fluoro-2-(prop-1-yn-1-yl)pyridine following a similar procedure to that described for the synthesis of 4-methyl-N-[[3-methyl-2-(4-methyl-3-pyridyl)-1H-indol-5-yl]methyl] pyrimidine-5-carboxamide and was obtained as a beige solid.

Yield 52 mg (43%). ¹H NMR (400 MHz, DMSO) δ 11.33 (s, 1H), 9.13 (t, J=5.8 Hz, 1H), 9.08 (s, 1H), 8.73 (s, 1H), 8.68 (d, J=2.8 Hz, 1H), 7.92-7.82 (m, 2H), 7.55 (s, 1H), 7.39 (d, J=8.3 Hz, 1H), 7.17 (dd, J=1.5, 8.3 Hz, 1H), 4.57 (d, J=5.8 Hz, 2H), 2.56 (s, 3H), 2.55 (s, 3H). ¹⁹F NMR (376.5 MHz, DMSO) δ=−130 (s). m/z: [ESI⁺] 376 (M+H)⁺, (C₂₁H₁₈FN₅O).

### Synthesis of N-[[2-(2-cyclopropylphenyl)-3-methyl-1H-indol-5-yl]methyl]-4-methyl-pyrimidine-5-carboxamide (Compound 154)

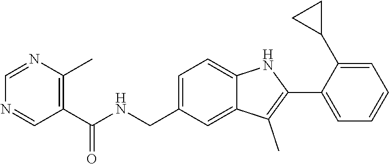 [SMILES: Cc1ncncc1C(=O)NCc1ccc2[nH]c(-c3ccccc3C3CC3)c(C)c2c1]

Compound N-[[2-(2-cyclopropylphenyl)-3-methyl-1H-indol-5-yl]methyl]-4-methyl-pyrimidine-5-carboxamide (154) was prepared from 1-cyclopropyl-2-(prop-1-yn-1-yl)benzene following a similar procedure to that described for the synthesis of 4-methyl-N-[[3-methyl-2-(4-methyl-3-pyridyl)-1H-indol-5-yl]methyl]pyrimidine-5-carboxamide except that it was also purified by preparative SFC to give N-[[2-(2-cyclopropylphenyl)-3-methyl-1H-indol-5-yl]methyl]-4-methyl-pyrimidine-5-carboxamide (154) as an off-white solid.

Yield 5 mg (5%). ¹H NMR (400 MHz, DMSO) δ 10.98 (s, 1H), 9.12 (t, J=5.8 Hz, 1H), 9.08 (s, 1H), 8.72 (s, 1H), 7.48 (s, 1H), 7.36-7.21 (m, 4H), 7.11 (dd, J=1.6, 8.0 Hz, 1H), 6.94 (d, J=8.0 Hz, 1H), 4.57 (d, J=5.8 Hz, 2H), 2.55 (s, 3H), 2.14 (s, 3H), 1.89-1.82 (m, 1H), 0.87-0.82 (m, 2H), 0.69-0.64 (m, 2H). m/z: [ESI⁺] 397 (M+H)⁺, (C₂₅H₂₄N₄O).

### Synthesis of 4-methyl-N-((3-methyl-2-(4-methylpyridazin-3-yl)-1H-indol-5-yl)methyl)pyrimidine-5-carboxamide (Compound 157)

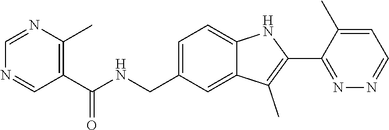 [SMILES: Cc1ccnnc1-c1[nH]c2ccc(CNC(=O)c3cncnc3C)cc2c1C]

Compound 4-methyl-N-((3-methyl-2-(4-methylpyridazin-3-yl)-1H-indol-5-yl)methyl)pyrimidine-5-carboxamide (157) was prepared from 4-methyl-3-(prop-1-yn-1-yl)pyridazine following a similar procedure to that described for the synthesis of 4-methyl-N-[[3-methyl-2-(4-methyl-3-pyridyl)-1H-indol-5- yl]methyl]pyrimidine-5-carboxamide and was obtained as an off-white solid.

Yield 24 mg (9%). ¹H NMR (400 MHz, DMSO) δ 11.28 (s, 1H), 9.18-9.12 (m, 2H), 9.10 (s, 1H), 8.75 (s, 1H), 7.71 (d, J=5.3 Hz, 1H), 7.59 (s, 1H), 7.40 (d, J=8.0 Hz, 1H), 7.22 (d, J=8.0 Hz, 1H), 4.61 (d, J=5.8 Hz, 2H), 2.57 (s, 3H), 2.33 (s, 3H), 2.23 (s, 3H). m/z: [ESI⁺] 373 (M+H)⁺, (C₂₁H₂₀N₆O).

### Synthesis of 4-methyl-N-[[2-(2-pyridyl)-1H-benzimidazol-5-yl]methyl]pyrimidine-5-carboxamide (Compound 152)

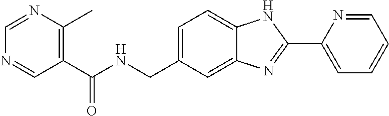 [SMILES: Cc1ncncc1C(=O)NCc1ccc2[nH]c(-c3ccccn3)nc2c1]

To a solution of 4-methylpyrimidine-5-carboxylic acid (21 mg, 0.152 mmol) in anhydrous DMF (0.25 mL) were added sequentially DIPEA (0.018 mL, 0.103 mmol) and HATU (63 mg, 0.166 mmol) and the reaction mixture was stirred at room temperature for 10 minutes. A solution of (2-(pyridin-2-yl)-1H-benzo[d]imidazol-5-yl)methanamine (31 mg, 0.138 mmol) in anhydrous DMF (0.25 mL) and DIPEA (0.018 mL, 0.103 mmol) was then added and the reaction continued to stir at room temperature for 16 hours. The mixture was partitioned between ethyl acetate (25 mL) and 1M aqueous NaOH (25 mL). The layers were separated, and the organic phase was washed with brine (10 mL), dried (MgSO₄), filtered and evaporated. The residue was purified by preparative HPLC to give 4-methyl-N-[[2-(2-pyridyl)-1H-benzimidazol-5-yl]methyl]pyrimidine-5-carboxamide (152) as a green solid.

Yield 10 mg (21%). ¹H NMR (400 MHz, DMSO) δ 13.07 (s, 1H), 9.25-9.17 (m, 1H), 9.09 (d, J=4.5 Hz, 1H), 8.76-8.73 (m, 2H), 8.32 (d, J=7.8 Hz, 1H), 8.00 (dd, J=7.2, 7.2 Hz, 1H), 7.70-7.66 (m, 1H), 7.56-7.49 (m, 2H), 7.25 (dd, J=8.4, 21.2 Hz, 1H), 4.60 (d, J=3.2 Hz, 2H), 2.55 (s, 3H). m/z: [ESI⁺]345 (M+H)⁺, (C₁₉H₁₆N₆O).

### Synthesis of 4-methyl-N-[[3-methyl-2-(2-pyridyl)-1H-pyrrolo[3,2-b]pyridin-5-yl]methyl]pyrimidine-5-carboxamide (Compound 150)

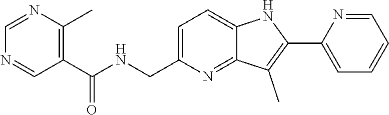 [SMILES: Cc1ncncc1C(=O)NCc1ccc2[nH]c(-c3ccccn3)c(C)c2n1]

To a solution of 4-methylpyrimidine-5-carboxylic acid (13 mg, 0.0923 mmol) in anhydrous DMF (0.25 mL) were added sequentially DIPEA (0.022 mL, 0.126 mmol) and HATU (38 mg, 0.101 mmol) and the reaction mixture was stirred at room temperature for 10 minutes. A solution of (3-methyl-2-(pyridin-2-yl)-1H-pyrrolo[3,2-b]pyridin-5-yl)methanamine (20 mg, 0.0839 mmol) in anhydrous DMF (0.25 mL) was then added and the reaction continued to stir at room temperature for 16 hours. The mixture was partitioned between ethyl acetate (25 mL) and 1 M aqueous NaOH (25 mL). The layers were separated, and the organic phase was washed with brine (10 mL), dried (MgSO₄), filtered and evaporated. The residue was purified by preparative HPLC to give 4-methyl-N-[[3-methyl-2-(2-pyridyl)-1H-pyrrolo[3,2-b]pyridin-5-yl]methyl]pyrimidine-5-carboxamide (150) as a white solid.

Yield 9 mg (30%). ¹H NMR (400 MHz, DMSO) δ 11.56 (s, 1H), 9.25 (t, J=5.9 Hz, 1H), 9.09 (s, 1H), 8.80 (s, 1H), 8.73-8.70 (m, 1H), 7.98-7.90 (m, 2H), 7.76 (d, J=8.3 Hz, 1H), 7.39-7.35 (m, 1H), 7.21 (d, J=8.3 Hz, 1H), 4.68 (d, J=5.9 Hz, 2H), 2.63 (s, 3H), 2.60 (s, 3H). m/z: [ESI⁺] 359 (M+H)⁺, (C₂₀H₁₈N₆O).

### Synthesis of 4-methyl-N-((2-(tetrahydro-2H-pyran-4-yl)-1H-indol-5-yl)methyl)pyrimidine-5-carboxamide (Compound 156)

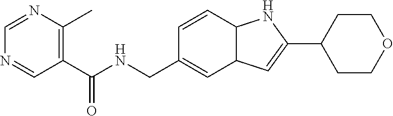 [SMILES: Cc1ncncc1C(=O)NCC1=CC2C=C(C3CCOCC3)NC2C=C1]

### Method A:

To a solution of N-(4-amino-3-((tetrahydro-2H-pyran-4-yl)ethynyl)benzyl)-4-methylpyrimidine-5-carboxamide (49 mg, 0.14 mmol) in EtOH (3 mL) was added AuCl₃ (2 mg, 0.007 mmol) and the mixture was heated to 70° C. and stirred for 18 hours. The reaction mixture was concentrated and purified by column chromatography on silica gel (0-10% methanol in DCM) followed by preparative HPLC to afford 4-methyl-N-((2-(tetrahydro-2H-pyran-4-yl)-1H-indol-5-yl)methyl)pyrimidine-5-carboxamide (156) as a white solid.

Yield 8 mg (16%). ¹H NMR (400 MHz, DMSO) δ 10.94 (s, 1H), 9.11-9.08 (m, 2H), 8.71 (s, 1H), 7.42 (s, 1H), 7.28 (d, J=8.3 Hz 1H), 7.04 (d, J=8.3 Hz, 1H), 6.15 (s, 1H), 4.52 (d, J=5.9 Hz, 2H), 3.96 (dd, J=2.1, 12.9 Hz, 2H), 3.51-3.43 (m, 2H), 3.02-2.92 (m, 1H), 2.53 (s, 3H), 1.93 (dd, J=2.1, 12.9 Hz, 2H), 1.79-1.66 (m, 2H). m/z: [ESI⁺] 351 (M+H)⁺, (C₂₀H₂₂N₄O₂).

### Method B:

Compound 4-methyl-N-((2-(tetrahydro-2H-pyran-4-yl)-1H-indol-5-yl)methyl)pyrimidine-5-carboxamide (156) was prepared from 4-methylpyrimidine-5-carboxylic acid (198 mg, 1.433 mmol) and (2-(tetrahydro-2H-pyran-4-yl)-1H-indol-5-yl)methanamine (300 mg, 1.303 mmol) following a similar procedure to that described for the synthesis of 4-methyl-N-[[3-methyl-2-(m-tolyl)-1H-indol-5-yl]methyl]pyrimidine-5-carboxamide and was obtained as a white solid.

Yield 100 mg (22%). ¹H NMR (400 MHz, DMSO) δ 10.93 (s, 1H), 9.10 (t, J=6.0 Hz, 1H), 9.08 (s, 1H), 8.71 (s, 1H), 7.42 (d, J=1.6 Hz, 1H), 7.27 (d, J=8.4 Hz, 1H), 7.04 (dd, J=1.6, 8.4 Hz, 1H), 6.15 (dd, J=0.8, 2.0 Hz, 1H), 4.52 (d, J=6.0 Hz, 2H), 4.01 (dd, J=4.0, 11.2 Hz, 2H), 3.47 (dt, J=2.0, 11.6 Hz, 2H), 2.97 (tt, J=4.0, 11.6 Hz, 1H), 2.53 (s, 3H), 1.92 (qd, J=2.4, 12.4 Hz, 2H), 1.72 (dq, J=4.4, 12.0 Hz, 2H). m/z: [ESI⁺] 351 (M+H)⁺, (C₂₀H₂₂N₄O₂).

### Synthesis of 4-methyl-N-((3-methyl-2-(2-((methylamino)methyl)phenyl)-1H-indol-5-yl)methyl)pyrimidine-5-carboxamide (Compound 217)

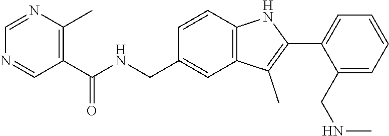 [SMILES: CNCc1ccccc1-c1[nH]c2ccc(CNC(=O)c3cncnc3C)cc2c1C]

A mixture of tert-butyl methyl(2-(3-methyl-5-((4-methylpyrimidine-5-carboxamido)methyl)-1H-indol-2-yl)benzyl)carbamate (210 mg, 0.420 mmol) in DCM (4 mL) and 2,2,2-trifluoroacetic acid (4 mL) were stirred for 2 h at room temperature under a nitrogen atmosphere. The resulting solution was concentrated under reduced pressure. The crude product was purified by Prep-HPLC with the following conditions: Column: Sunfire prep C18 column, 30×150 mm, 5 m; Mobile Phase A: water (plus 10 mM formic acid), Mobile Phase B: ACN; How rate: 60 mL/min; Gradient: 20% B to 55% B in 8 min; Detector: UV 254/220 nm. The fractions containing the desired product were concentrated under reduced pressure and lyophilized to afford 4-methyl-N-((3-methyl-2-(2-((methylamino)methyl)phenyl)-1H-indol-5-yl)methyl)pyrimidine-5-carboxamide (217) as a red solid.

Yield 60 mg (36%). ¹H NMR (400 MHz, DMSO) δ 11.71 (s, 1H), 9.15 (t, J=5.6 Hz, 1H), 9.09 (s, 1H), 8.73 (s, 1H), 8.22 (s, 0.71H, formic acid), 7.63-7.55 (m, 1H), 7.52 (d, J=1.6 Hz, 1H), 7.44 (m, 3H), 7.33 (d, J=8.4 Hz, 1H), 7.15 (dd, J=1.6, 8.4 Hz, 1H), 4.58 (d, J=5.6 Hz, 2H), 3.68 (s, 2H), 2.56 (s, 3H), 2.31 (s, 3H), 2.21 (s, 3H). m/z: [ESI⁺] 400 (M+H)⁺, (C₂₄H₂₅N₅O).

### Synthesis of 1-methyl-N-((3-methyl-2-(pyridin-2-yl)-1H-indol-5-yl)methyl)-1H-pyrazole-5-carboxamide (Compound 185)

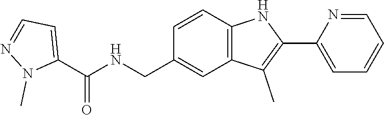 [SMILES: Cc1c(-c2ccccn2)[nH]c2ccc(CNC(=O)c3ccnn3C)cc12]

Compound 1-methyl-N-((3-methyl-2-(pyridin-2-yl)-1H-indol-5-yl)methyl)-1H-pyrazole-5-carboxamide (185) was prepared from 1-methyl-1H-pyrazole-5-carboxylic acid (150 mg, 1.189 mmol) and (3-methyl-2-(pyridin-2-yl)-1H-indol-5-yl)methanamine (420 mg, 1.770 mmol) following a similar procedure to that described for the synthesis of 4-methyl-N-[[3-methyl-2-(m-tolyl)-1H-indol-5-yl]methyl]pyrimidine-5-carboxamide and was obtained as a yellow solid.

Yield 12 mg (3%). ¹H NMR (400 MHz, DMSO) δ 11.31 (s, 1H), 8.97 (t, J=5.6 Hz, 1H), 8.68 (ddd, J=1.0, 1.8, 4.8 Hz, 1H), 7.91 (dt, J=1.6, 7.6 Hz, 1H), 7.84 (td, J=1.2, 6.8 Hz, 1H), 7.52 (s, 1H), 7.46 (d, J=2.0 Hz, 1H), 7.38 (d, J=8.4 Hz, 1H), 7.30 (ddd, J=1.2, 4.8, 7.4 Hz, 1H), 7.13 (dd, J=1.6, 8.4 Hz, 1H), 6.91 (d, J=2.0 Hz, 1H), 4.53 (d, J=5.6 Hz, 2H), 4.09 (s, 3H), 2.58 (s, 3H). m/z: [ESI⁺] 346 (M+H)⁺, (C₂₀H₁₉N₅O).

### Synthesis of N-((2-(2-(hydroxymethyl)phenyl)-3-methyl-1H-indol-5-yl)methyl)-3-methylpyridazine-4-carboxamide (Compound 215)

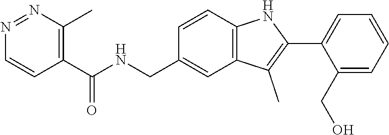 [SMILES: Cc1nnccc1C(=O)NCc1ccc2[nH]c(-c3ccccc3CO)c(C)c2c1]

Compound N-((2-(2-(hydroxymethyl)phenyl)-3-methyl-1H-indol-5-yl)methyl)-3-methylpyridazine-4-carboxamide (215) was prepared from 3-methylpyridazine-4-carboxylic acid (630 mg, 4.561 mmol) and (2-(5-(aminomethyl)-3-methyl-1H-indol-2-yl)phenyl)methanol 2,2,2-trifluoroacetate (1.57 g, 4.13 mmol) following a similar procedure to that described for the synthesis of 4-methyl-N-[[3-methyl-2-(m-tolyl)-1H-indol-5-yl]methyl]pyrimidine-5-carboxamide and was obtained as a white solid.

Yield 350 mg (22%). ¹H NMR (400 MHz, DMSO) δ 10.91 (s, 1H), 9.20 (d, J=5.0 Hz, 1H), 9.19 (t, J=5.4 Hz, 1H), 7.65 (dd, J=1.2, 7.8 Hz, 1H), 7.60 (d, J=5.0 Hz, 1H), 7.49 (d, J=1.6 Hz, 1H), 7.46 (dd, J=1.6, 7.4 Hz, 1H), 7.37 (dt, J=1.6, 7.4 Hz, 1H), 7.34 (dd, J=1.8, 7.8 Hz, 1H), 7.28 (d, J=8.4 Hz, 1H), 7.13 (dd, J=1.6, 8.4 Hz, 1H), 5.24 (t, J=5.4 Hz, 1H), 4.57 (d, J=5.8 Hz, 2H), 4.43 (d, J=5.4 Hz, 2H), 2.68 (s, 3H), 2.14 (s, 3H). m/z: [ESI⁺] 387 (M+H)⁺, (C₂₃H₂₂N₄O₂).

### Synthesis of 3-methyl-N-((3-methyl-2-(tetrahydro-2H-pyran-4-yl)-1H-indol-5-yl)methyl)pyridazine-4-carboxamide (Compound 194)

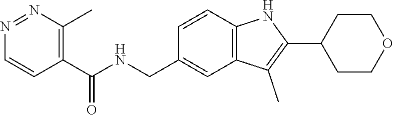 [SMILES: Cc1nnccc1C(=O)NCc1ccc2[nH]c(C3CCOCC3)c(C)c2c1]

Compound 3-methyl-N-((3-methyl-2-(tetrahydro-2H-pyran-4-yl)-1H-indol-5-yl)methyl)pyridazine-4-carboxamide (194) was prepared from 3-methylpyridazine-4-carboxylic acid (62 mg, 0.449 mmol) and (3-methyl-2-(tetrahydro-2H-pyran-4-yl)-1H-indol-5-yl)methanamine (100 mg, 0.409 mmol) following a similar procedure to that described for the synthesis of 4-methyl-N-[[3-methyl-2-(m-tolyl)-1H-indol-5-yl]methyl]pyrimidine-5-carboxamide and was obtained as an off-white solid.

Yield 7 mg (5%). ¹H NMR (400 MHz, DMSO) δ 10.62 (s, 1H), 9.17 (t, J=5.2 Hz, 1H), 9.13 (dd, J=1.6, 5.6 Hz, 1H), 7.55 (d, J=5.2 Hz, 1H), 7.33 (d, J=1.6 Hz, 1H), 7.24-7.16 (d, J=8.4 Hz, 1H), 6.99 (dd, J=1.6, 8.4 Hz, 1H), 4.50 (d, J=5.6 Hz, 2H), 3.94 (dd, J=4.0, 11.2 Hz, 2H), 3.45 (dd, J=10.4, 12.4 Hz, 2H), 3.07 (tdd, J=3.6, 7.4, 12.0 Hz, 1H), 2.63 (s, 3H), 2.17 (s, 3H), 1.86 (qd, J=4.4, 12.8 Hz, 2H), 1.60 (d, J=12.8 Hz, 2H). m/z: [ESI⁺] 365 (M+H)⁺, (C₂₁H₂₄N₄O₂).

### Synthesis of 2-cyano-N-((3-methyl-2-(tetrahydro-2H-pyran-4-yl)-1H-indol-5-yl)methyl)pyrimidine-5-carboxamide (Compound 21

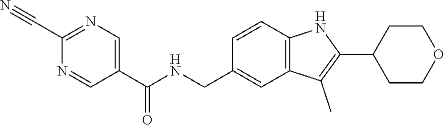 [SMILES: Cc1c(C2CCOCC2)[nH]c2ccc(CNC(=O)c3cnc(C#N)nc3)cc12]

Compound 2-cyano-N-((3-methyl-2-(tetrahydro-2H-pyran-4-yl)-1H-indol-5-yl)methyl)pyrimidine-5-carboxamide (216) was prepared from 2-cyanopyrimidine-5-carboxylic acid (101 mg, 0.677 mmol) and (3-methyl-2-(tetrahydro-2H-pyran-4-yl)-1H-indol-5-yl)methanamine (150 mg, 0.614 mmol) following a similar procedure to that described for the synthesis of 4-methyl-N-[[3-methyl-2-(m-tolyl)-1H-indol-5-yl]methyl]pyrimidine-5-carboxamide and was obtained as a white solid.

Yield 20 mg (9%). ¹H NMR (400 MHz, DMSO) δ 10.63 (s, 1H), 9.48 (t, J=5.6 Hz, 1H), 9.35 (s, 2H), 7.38 (d, J=1.6 Hz, 1H), 7.21 (d, J=8.4 Hz, 1H), 7.02 (dd, J=1.6, 8.4 Hz, 1H), 4.58 (d, J=5.6 Hz, 2H), 3.96 (dd, J=4.4, 11.2 Hz, 2H), 3.47 (dt, J=2.0, 11.6 Hz, 2H), 3.09 (tt, J=3.6, 12.4 Hz, 1H), 2.19 (s, 3H), 1.87 (dq, J=4.4, 12.8 Hz, 2H), 1.60 (d, J=12.8 Hz, 2H). m/z: [ESI⁺] 376 (M+H)⁺, (C₂₁H₂₁N₅O₂).

### Synthesis of N-((3-methyl-2-(tetrahydro-2H-pyran-4-yl)-1H-indol-5-yl)methyl)-2-(trifluoromethyl)pyrimidine-5-carboxamide (Compound 213)

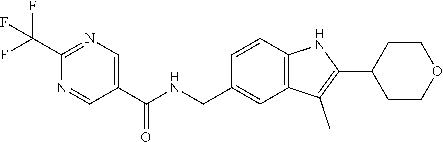 [SMILES: Cc1c(C2CCOCC2)[nH]c2ccc(CNC(=O)c3cnc(C(F)(F)F)nc3)cc12]

Compound N-((3-methyl-2-(tetrahydro-2H-pyran-4-yl)-1H-indol-5-yl)methyl)-2-(trifluoromethyl)pyrimidine-5-carboxamide (213) was prepared from 2-(trifluoromethyl)pyrimidine-5-carboxylic acid (40 mg, 0.208 mmol) and (3-methyl-2-(tetrahydro-2H-pyran-4-yl)-1H-indol-5-yl)methanamine (50 mg, 0.205 mmol) following a similar procedure to that described for the synthesis of 4-methyl-N-[[3-methyl-2-(m-tolyl)-1H-indol-5-yl]methyl]pyrimidine-5-carboxamide and was obtained as a yellow solid.

Yield 16 mg (19%). ¹H NMR (400 MHz, DMSO) δ 10.63 (s, 1H), 9.46 (t, J=5.6 Hz, 1H), 9.42 (s, 2H), 7.39 (d, J=1.6 Hz, 1H), 7.22 (d, J=8.4 Hz, 1H), 7.04 (dd, J=1.6, 8.4 Hz, 1H), 4.59 (d, J=5.6 Hz, 2H), 3.96 (dd, J=4.4, 11.2 Hz, 2H), 3.48 (t, J=11.6 Hz, 2H), 3.09 (tt, J=3.6, 12.4 Hz, 1H), 2.19 (s, 3H), 1.86 (dq, J=4.4, 12.8 Hz, 2H), 1.62 (d, J=12.8 Hz, 2H). m/z: [ESI⁺] 419 (M+H)⁺, (C₂₁H₂₁F₃N₄O₂).

### Synthesis of N-((3-methyl-2-(tetrahydro-2H-pyran-4-yl)-1H-indol-5-yl)methyl)pyridazine-4-carboxamide (Compound 225

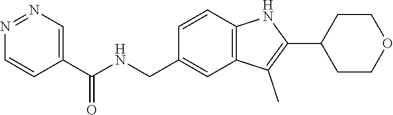 [SMILES: Cc1c(C2CCOCC2)[nH]c2ccc(CNC(=O)c3ccnnc3)cc12]

Compound N-((3-methyl-2-(tetrahydro-2H-pyran-4-yl)-1H-indol-5-yl)methyl)pyridazine-4-carboxamide (225) was prepared from pyridazine-4-carboxylic acid (73 mg, 0.588 mmol) and (3-methyl-2-(tetrahydro-2H-pyran-4-yl)-1H-indol-5-yl)methanamine (130 mg, 0.532 mmol) following a similar procedure to that described for the synthesis of 4-methyl-N-[[3-methyl-2-(m-tolyl)-1H-indol-5-yl]methyl]pyrimidine-5-carboxamide and was obtained as a red solid.

Yield 23 mg (12%). ¹H NMR (400 MHz, DMSO) δ 10.64 (s, 1H), 9.58 (t, J=2.4 Hz, 1H), 9.48 (dd, J=1.6, 5.6 Hz, 1H), 9.43 (dd, J=1.2, 5.2 Hz, 1H), 8.03 (dd, J=2.4, 5.2 Hz, 1H), 7.37 (d, J=1.6 Hz, 1H), 7.22 (d, J=8.4 Hz, 1H), 7.02 (dd, J=1.6, 8.4 Hz, 1H), 4.57 (d, J=5.6 Hz, 2H), 3.97 (dd, J=4.0, 11.6 Hz, 2H), 3.48 (t, J=11.6 Hz, 2H), 3.09 (tt, J=3.6, 12.0 Hz, 1H), 2.19 (s, 3H), 1.88 (dq, J=4.4, 12.8 Hz, 2H), 1.63 (d, J=12.8 Hz, 2H). m/z: [ESI⁺] 351 (M+H)⁺, (C₂₀H₂₂N₄O₂).

### Synthesis of 4-methyl-N-((3-methyl-2-(tetrahydro-2H-pyran-4-yl)-1H-indol-5-yl)methyl)-1,2,3-thiadiazole-5-carboxamide (Compound 220)

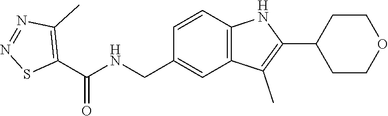 [SMILES: Cc1nnsc1C(=O)NCc1ccc2[nH]c(C3CCOCC3)c(C)c2c1]

Compound 4-methyl-N-((3-methyl-2-(tetrahydro-2H-pyran-4-yl)-1H-indol-5-yl)methyl)-1,2,3-thiadiazole-5-carboxamide (220) was prepared from 4-methyl-1,2,3-thiadiazole-5-carboxylic acid (52 mg, 0.361 mmol) and (3-methyl-2-(tetrahydro-2H-pyran-4-yl)-1H-indol-5-yl)methanamine (80 mg, 0.327 mmol) following a similar procedure to that described for the synthesis of 4-methyl-N-[[3-methyl-2-(m-tolyl)-1H-indol-5-yl]methyl]pyrimidine-5-carboxamide and was obtained as a white solid.

Yield 27 mg (22%). ¹H NMR (400 MHz, DMSO) δ 10.65 (s, 1H), 9.29 (t, J=5.6 Hz, 1H), 7.35 (d, J=1.6 Hz, 1H), 7.22 (d, J=8.4 Hz, 1H), 7.01 (dd, J=1.6, 8.4 Hz, 1H), 4.52 (d, J=5.6 Hz, 2H), 3.97 (dd, J=4.4, 11.2 Hz, 2H), 3.48 (dt, J=2.0, 11.6 Hz, 2H), 3.10 (tt, J=3.6, 12.0 Hz, 1H), 2.78 (s, 3H), 2.19 (s, 3H), 1.89 (dq, J=4.4, 12.8 Hz, 2H), 1.63 (d, J=12.8 Hz, 2H). m/z: [ESI⁺] 371 (M+H)⁺, (C₁₉H₂₂N₄O₂S).

### Synthesis of 1-methyl-N-((3-methyl-2-(tetrahydro-2H-pyran-4-yl)-1H-indol-5-yl)methyl)-1H-pyrazole-5-carboxamide (Compound 186)

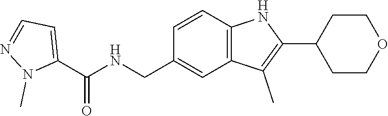 [SMILES: Cc1c(C2CCOCC2)[nH]c2ccc(CNC(=O)c3ccnn3C)cc12]

Compound 1-methyl-N-((3-methyl-2-(tetrahydro-2H-pyran-4-yl)-1H-indol-5-yl)methyl)-1H-pyrazole-5-carboxamide (186) was prepared from 1-methyl-1H-pyrazole-5-carboxylic acid (85 mg, 0.674 mmol) and (3-methyl-2-(tetrahydro-2H-pyran-4-yl)-1H-indol-5-yl)methanamine (150 mg, 0.614 mmol) following a similar procedure to that described for the synthesis of 4-methyl-N-[[3-methyl-2-(m-tolyl)-1H-indol-5-yl]methyl]pyrimidine-5-carboxamide and was obtained as an off-white solid.

Yield 44 mg (20%). 1H NMR (400 MHz, DMSO) δ 10.61 (s, 1H), 8.92 (t, J=5.6 Hz, 1H), 7.45 (d, J=2.0 Hz, 1H), 7.33 (d, J=1.6 Hz, 1H), 7.20 (d, J=8.4 Hz, 1H), 6.99 (dd, J=1.6, 8.4 Hz, 1H), 6.88 (d, J=2.0 Hz, 1H), 4.48 (d, J=5.6 Hz, 2H), 4.07 (s, 3H), 3.96 (dd, J=4.0, 11.2 Hz, 2H), 3.47 (dt, J=2.0, 11.6 Hz, 2H), 3.09 (tt, J=3.6, 12.0 Hz, 1H), 2.18 (s, 3H), 1.88 (dq, J=4.4, 12.8 Hz, 2H), 1.62 (d, J=12.8 Hz, 2H). m/z: [ESI⁺] 353 (M+H)⁺, (C₂₀H₂₄N₄O₂).

### Synthesis of N-((3-methyl-2-(tetrahydro-2H-pyran-4-yl)-1H-indol-5-yl)methyl) isothiazole-5-carboxamide (Compound 223)

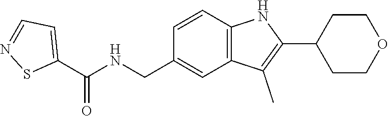 [SMILES: Cc1c(C2CCOCC2)[nH]c2ccc(CNC(=O)c3ccns3)cc12]

Compound N-((3-methyl-2-(tetrahydro-2H-pyran-4-yl)-1H-indol-5-yl)methyl) isothiazole-5-carboxamide (223) was prepared from isothiazole-5-carboxylic acid (29 mg, 0.225 mmol) and (3-methyl-2-(tetrahydro-2H-pyran-4-yl)-1H-indol-5-yl)methanamine (50 mg, 0.205 mmol) following a similar procedure to that described for the synthesis of 4-methyl-N-[[3-methyl-2-(m-tolyl)-1H-indol-5-yl]methyl]pyrimidine-5-carboxamide and was obtained as a white solid.

Yield 30 mg (41%). ¹H NMR (400 MHz, DMSO) δ 10.63 (s, 1H), 9.34 (t, J=5.6 Hz, 1H), 8.65 (d, J=2.0 Hz, 1H), 7.94 (d, J=2.0 Hz, 1H), 7.35 (d, J=1.6 Hz, 1H), 7.22 (d, J=8.4 Hz, 1H), 7.01 (dd, J=1.6, 8.4 Hz, 1H), 4.52 (d, J=5.6 Hz, 2H), 3.96 (dd, J=3.6, 11.6 Hz, 2H), 3.47 (dt, J=2.0, 11.6 Hz, 2H), 3.09 (tt, J=3.6, 12.0 Hz, 1H), 2.19 (s, 3H), 1.88 (dq, J=4.4, 12.8 Hz, 2H), 1.62 (ddd, J=1.6, 4.0, 12.8 Hz, 2H). m/z: [ESI⁺] 356 (M+H)⁺, (C₁₉H₂₁N₃O₂S).

### Synthesis of 1,4-dimethyl-N-((3-methyl-2-(tetrahydro-2H-pyran-4-yl)-1H-indol-5-yl)methyl)-1H-pyrazole-5-carboxamide (Compound 222)

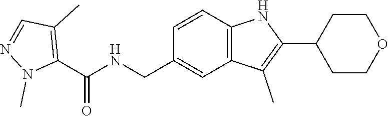 [SMILES: Cc1cnn(C)c1C(=O)NCc1ccc2[nH]c(C3CCOCC3)c(C)c2c1]

Compound 1,4-dimethyl-N-((3-methyl-2-(tetrahydro-2H-pyran-4-yl)-1H-indol-5-yl)methyl)-1H-pyrazole-5-carboxamide (222) was prepared from 1,4-dimethyl-1H-pyrazole-5-carboxylic acid (50 mg, 0.357 mmol) and (3-methyl-2-(tetrahydro-2H-pyran-4-yl)-1H-indol-5-yl)methanamine (80 mg, 0.327 mmol) following a similar procedure to that described for the synthesis of 4-methyl-N-[[3-methyl-2-(m-tolyl)-1H-indol-5-yl]methyl]pyrimidine-5-carboxamide and was obtained as a white solid.

Yield 47 mg (39%). ¹H NMR (400 MHz, DMSO) δ 10.63 (s, 1H), 8.65 (t, J=6.0 Hz, 1H), 7.35 (d, J=1.6 Hz, 1H), 7.25 (s, 1H), 7.22 (d, J=8.4 Hz, 1H), 7.01 (dd, J=1.6, 8.4 Hz, 1H), 4.52 (d, J=6.0 Hz, 2H), 3.97 (dd, J=4.4, 11.2 Hz, 2H), 3.85 (s, 3H), 3.54-3.42 (m, 2H), 3.09 (tt, J=3.6, 12.0 Hz, 1H), 2.19 (s, 3H), 2.10 (s, 3H), 1.89 (dq, J=4.4, 12.4 Hz, 2H), 1.63 (ddd, J=1.6, 4.0, 12.8 Hz, 2H). m/z: [ESI⁺] 367 (M+H)⁺, (C₂₁H₂₆N₄O₂).

### Synthesis of 1,3-dimethyl-N-((3-methyl-2-(tetrahydro-2H-pyran-4-yl)-1H-indol-5-yl)methyl)-1H-pyrazole-5-carboxamide (Compound 221)

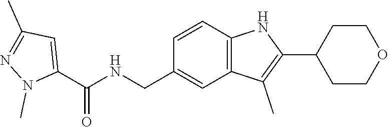 [SMILES: Cc1cc(C(=O)NCc2ccc3[nH]c(C4CCOCC4)c(C)c3c2)n(C)n1]

Compound 1,3-dimethyl-N-((3-methyl-2-(tetrahydro-2H-pyran-4-yl)-1H-indol-5-yl)methyl)-1H-pyrazole-5-carboxamide (221) was prepared from 1,3-dimethyl-1H-pyrazole-5-carboxylic acid (82 mg, 0.585 mmol) and (3-methyl-2-(tetrahydro-2H-pyran-4-yl)-1H-indol-5-yl)methanamine (130 mg, 0.532 mmol) following a similar procedure to that described for the synthesis of 4-methyl-N-[[3-methyl-2-(m-tolyl)-1H-indol-5-yl]methyl]pyrimidine-5-carboxamide and was obtained as a white solid.

Yield 9 mg (5%). ¹H NMR (400 MHz, DMSO) δ 10.61 (s, 1H), 8.84 (t, J=6.0 Hz, 1H), 7.31 (d, J=1.6 Hz, 1H), 7.20 (d, J=8.4 Hz, 1H), 6.98 (dd, J=1.6, 8.4 Hz, 1H), 6.64 (s, 1H), 4.46 (d, J=6.0 Hz, 2H), 3.98 (s, 3H), 3.96 (dd, J=4.4, 11.2 Hz, 2H), 3.47 (dt, J=2.0, 11.6 Hz, 2H), 3.09 (tt, J=3.6, 12.0 Hz, 1H), 2.18 (s, 3H), 2.14 (s, 3H), 1.88 (dq, J=4.4, 12.8 Hz, 2H), 1.62 (d, J=12.8 Hz, 2H). m/z: [ESI⁺] 367 (M+H)⁺, (C₂₁H₂₆N₄O₂).

### Synthesis of 3-methoxy-1-methyl-N-((3-methyl-2-(tetrahydro-2H-pyran-4-yl)-1H-indol-5-yl)methyl)-1H-pyrazole-5-carboxamide (Compound 224)

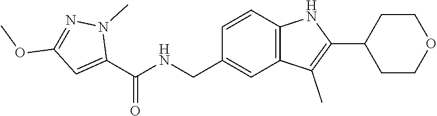 [SMILES: COc1cc(C(=O)NCc2ccc3[nH]c(C4CCOCC4)c(C)c3c2)n(C)n1]

Compound 3-methoxy-1-methyl-N-((3-methyl-2-(tetrahydro-2H-pyran-4-yl)-1H-indol-5-yl)methyl)-1H-pyrazole-5-carboxamide (224) was prepared from 3-methoxy-1-methyl-1H-pyrazole-5-carboxylic acid (70 mg, 0.448 mmol) and (3-methyl-2-(tetrahydro-2H-pyran-4-yl)-1H-indol-5-yl)methanamine (100 mg, 0.409 mmol) following a similar procedure to that described for the synthesis of 4-methyl-N-[[3-methyl-2-(m-tolyl)-1H-indol-5-yl]methyl]pyrimidine-5-carboxamide and was obtained as a white solid.

Yield 70 mg (45%). ¹H NMR (400 MHz, DMSO) δ 10.62 (s, 1H), 8.87 (d, J=6.0 Hz, 1H), 7.32 (d, J=1.6 Hz, 1H), 7.21 (d, J=8.4 Hz, 1H), 6.99 (dd, J=1.6, 8.4 Hz, 1H), 6.30 (s, 1H), 4.46 (d, J=6.0 Hz, 2H), 3.97 (dd, J=4.0, 11.2 Hz, 2H), 3.93 (s, 3H), 3.76 (s, 3H), 3.48 (dt, J=2.0, 12.0 Hz, 2H), 3.09 (tt, J=3.6, 12.4 Hz, 1H), 2.19 (s, 3H), 1.88 (dq, J=4.4, 12.4 Hz, 2H), 1.62 (dd, J=2.0, 12.8 Hz, 2H). m/z: [ESI⁺] 383 (M+H)⁺, (C₂₁H₂₆N₄O₃).

### Synthesis of 1-methyl-N-((3-methyl-2-(tetrahydro-2H-pyran-4-yl)-1H-indol-5-yl)methyl)-1H-1,2,3-triazole-5-carboxamide (Compound 218)

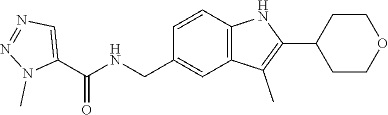 [SMILES: Cc1c(C2CCOCC2)[nH]c2ccc(CNC(=O)c3cnnn3C)cc12]

Compound 1-methyl-N-((3-methyl-2-(tetrahydro-2H-pyran-4-yl)-1H-indol-5-yl)methyl)-1H-1,2,3-triazole-5-carboxamide (218) was prepared from 1-methyl-1H-1,2,3-triazole-5-carboxylic acid (74 mg, 0.582 mmol) and (3-methyl-2-(tetrahydro-2H-pyran-4-yl)-1H-indol-5-yl)methanamine (130 mg, 0.532 mmol) following a similar procedure to that described for the synthesis of 4-methyl-N-[[3-methyl-2-(m-tolyl)-1H-indol-5-yl]methyl]pyrimidine-5-carboxamide and was obtained as a white solid.

Yield 48 mg (26%). ¹H NMR (400 MHz, DMSO) δ 10.63 (s, 1H), 9.20 (t, J=6.0 Hz, 1H), 8.23 (s, 1H), 7.34 (d, J=1.6 Hz, 1H), 7.21 (d, J=8.4 Hz, 1H), 7.00 (dd, J=1.6, 8.4 Hz, 1H), 4.51 (d, J=6.0 Hz, 2H), 4.23 (s, 3H), 3.96 (dd, J=4.0, 11.2 Hz, 2H), 3.47 (dt, J=2.0, 11.6 Hz, 2H), 3.09 (tt, J=3.6, 12.0 Hz, 1H), 2.19 (s, 3H), 1.88 (dq, J=4.4, 12.8 Hz, 2H), 1.62 (dd, J=3.6, 12.8 Hz, 2H). m/z: [ESI⁺] 354 (M+H)⁺, (C₁₉H₂₃N₅O₂).

### Synthesis of 1-methyl-N-((3-methyl-2-(tetrahydro-2H-pyran-4-yl)-1H-indol-5-yl)methyl)-1H-1,2,4-triazole-5-carboxamide (Compound 219)

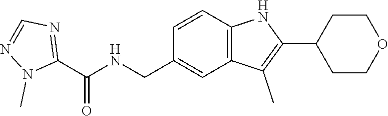 [SMILES: Cc1c(C2CCOCC2)[nH]c2ccc(CNC(=O)c3ncnn3C)cc12]

Compound 1-methyl-N-((3-methyl-2-(tetrahydro-2H-pyran-4-yl)-1H-indol-5-yl)methyl)-1H-1,2,4-triazole-5-carboxamide (219) was prepared from 1-methyl-1H-1,2,4-triazole-5-carboxylic acid (86 mg, 0.677 mmol) and (3-methyl-2-(tetrahydro-2H-pyran-4-yl)-1H-indol-5-yl)methanamine (150 mg, 0.614 mmol) following a similar procedure to that described for the synthesis of 4-methyl-N-[[3-methyl-2-(m-tolyl)-1H-indol-5-yl]methyl]pyrimidine-5-carboxamide and was obtained as a light yellow solid.

Yield 40 mg (18%). ¹H NMR (400 MHz, DMSO) δ 10.61 (s, 1H), 9.26 (t, J=6.0 Hz, 1H), 8.05 (s, 1H), 7.35 (d, J=1.6 Hz, 1H), 7.20 (d, J=8.4 Hz, 1H), 7.02 (dd, J=1.6, 8.4 Hz, 1H), 4.48 (d, J=6.0 Hz, 2H), 4.15 (s, 3H), 3.96 (dd, J=4.0, 10.8 Hz, 2H), 3.47 (dt, J=2.0, 11.6 Hz, 2H), 3.09 (tt, J=3.6, 12.0 Hz, 1H), 2.18 (s, 3H), 1.88 (dq, J=4.4, 12.8 Hz, 2H), 1.62 (d, J=12.8 Hz, 2H). m/z: [ESI⁺] 354 (M+H)⁺, (C₁₉H₂₃N₅O₂).

### Synthesis of N-((2-(2-(methoxymethyl)phenyl)-3-methyl-1H-indol-5-yl)methyl)-3-methylpyridazine-4-carboxamide (Compound 214)

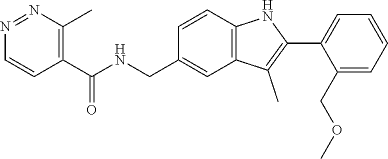 [SMILES: COCc1ccccc1-c1[nH]c2ccc(CNC(=O)c3ccnnc3C)cc2c1C]

Compound N-((2-(2-(methoxymethyl)phenyl)-3-methyl-1H-indol-5-yl)methyl)-3-methylpyridazine-4-carboxamide (214) was prepared from 3-methylpyridazine-4-carboxylic acid (27 mg, 0.195 mmol) and (2-(2-(methoxymethyl)phenyl)-3-methyl-1H-indol-5-yl)methanamine hydrochloride (56 mg, 0.177 mmol) following a similar procedure to that described for the synthesis of 4-methyl-N-[[3-methyl-2-(m-tolyl)-1H-indol-5-yl]methyl]pyrimidine-5-carboxamide and was obtained as a white solid.

Yield 36 mg (51%). ¹H NMR (400 MHz, DMSO) δ 10.92 (s, 1H), 9.21 (d, J=5.0 Hz, 1H), 9.20 (t, J=6.0 Hz, 1H), 7.61 (d, J=5.0 Hz, 1H), 7.58 (dd, J=1.6, 7.6 Hz, 1H), 7.50 (d, J=1.6 Hz, 1H), 7.46 (dd, J=1.6, 7.6 Hz, 1H), 7.42 (dd, J=1.6, 7.2 Hz, 1H), 7.37 (dd, J=1.6, 7.6 Hz, 1H), 7.32 (d, J=8.4 Hz, 1H), 7.13 (dd, J=1.6, 8.4 Hz, 1H), 4.58 (d, J=6.0 Hz, 2H), 4.34 (s, 2H), 3.20 (s, 3H), 2.69 (s, 3H), 2.13 (s, 3H). m/z: [ESI⁺] 401 (M+H)⁺, (C₂₄H₂₄N₄O₂).

### Synthesis of 2-(3-methyl-2-(tetrahydro-2H-pyran-4-yl)-1H-indol-5-yl)-N-(4-methylpyrimidin-5-yl)acetamide (Compound 202)

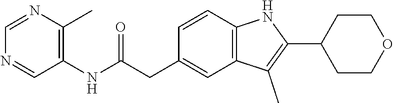 [SMILES: Cc1ncncc1NC(=O)Cc1ccc2[nH]c(C3CCOCC3)c(C)c2c1]

Compound 2-(3-methyl-2-(tetrahydro-2H-pyran-4-yl)-1H-indol-5-yl)-N-(4-methylpyrimidin-5-yl)acetamide (202) was prepared from 2-(3-methyl-2-(tetrahydro-2H-pyran-4-yl)-1H-indol-5-yl)acetic acid (50 mg, 0.183 mmol) and 4-methylpyrimidin-5-amine (20 mg, 0.183 mmol) following a similar procedure to that described for the synthesis of 4-methyl-N-[[3-methyl-2-(m-tolyl)-1H-indol-5-yl]methyl]pyrimidine-5-carboxamide and was obtained as an off-white solid.

Yield 14 mg (21%). ¹H NMR (400 MHz, DMSO) δ 10.62 (s, 1H), 9.82 (br s, 1H), 8.81 (s, 1H), 8.73 (s, 1H), 7.37 (d, J=1.6 Hz, 1H), 7.21 (d, J=8.4 Hz, 1H), 7.02 (dd, J=1.6, 8.4 Hz, 1H), 3.97 (dd, J=4.4, 11.2 Hz, 2H), 3.75 (s, 2H), 3.48 (t, J=11.6 Hz, 2H), 3.08 (tt, J=4.0, 11.6 Hz, 1H), 2.38 (s, 3H), 2.20 (s, 3H), 1.89 (dq, J=4.4, 12.8 Hz, 2H), 1.62 (d, J=12.8 Hz, 2H). m/z: [ESI⁺] 365 (M+H)⁺, (C₂₁H₂₄N₄O₂).

### Synthesis of N-((6-fluoro-3-methyl-2-(tetrahydro-2H-pyran-4-yl)-1H-indol-5-yl)methyl)-4-methylpyrimidine-5-carboxamide (Compound 166)

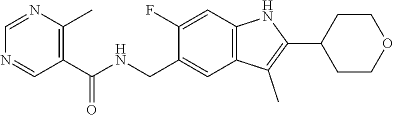 [SMILES: Cc1ncncc1C(=O)NCc1cc2c(C)c(C3CCOCC3)[nH]c2cc1F]

Compound N-((6-fluoro-3-methyl-2-(tetrahydro-2H-pyran-4-yl)-1H-indol-5-yl)methyl)-4-methylpyrimidine-5-carboxamide (166) was prepared from N-(4-amino-2-fluoro-5-iodobenzyl)-4-methylpyrimidine-5-carboxamide (200 mg, 0.518 mmol) and 4-(prop-1-yn-1-yl)tetrahydro-2H-pyran (96 mg, 0.773 mmol) following a similar procedure to that described for the synthesis of 2-(2-fluorophenyl)-3-methyl-1H-indole-5-carbonitrile and was obtained as an off-white solid.

Yield 40 mg (20%). ¹H NMR (400 MHz, DMSO) δ 10.76 (s, 1H), 9.08 (s, 1H), 9.06 (t, J=5.6 Hz, 1H), 8.69 (s, 1H), 7.40 (d, J=7.4 Hz, 1H), 7.02 (d, J=11.0 Hz, 1H), 4.56 (d, J=5.6 Hz, 2H), 3.96 (dd, J=3.6, 11.2 Hz, 2H), 3.47 (dt, J=2.4, 12.0 Hz, 2H), 3.08 (tt, J=3.6, 12.0 Hz, 1H), 2.53 (s, 3H), 2.18 (s, 3H), 1.86 (dq, J=4.4, 12.4 Hz, 2H), 1.62 (dd, J=2.0, 12.8 Hz, 2H). ¹⁹F NMR (376 MHz, DMSO) δ 128.26. m/z: [ESI⁺] 383 (M+H)⁺, (C₂₁H₂₃FN₄O₂).

### Synthesis of N-((7-fluoro-3-methyl-2-(tetrahydro-2H-pyran-4-yl)-1H-indol-5-yl)methyl)-4-methylpyrimidine-5-carboxamide (Compound 163)

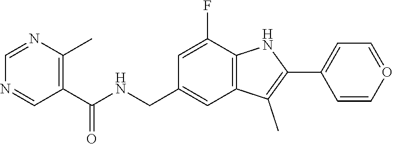

Compound N-((7-fluoro-3-methyl-2-(tetrahydro-2H-pyran-4-yl)-1H-indol-5-yl)methyl)-4-methylpyrimidine-5-carboxamide (163) was prepared from N-(4-amino-3-fluoro-5-iodobenzyl)-4-methylpyrimidine-5-carboxamide (100 mg, 0.259 mmol) and 4-(prop-1-yn-1-yl)tetrahydro-2H-pyran (48 mg, 0.387 mmol) following a similar procedure to that described for the synthesis of 2-(2-fluorophenyl)-3-methyl-1H-indole-5-carbonitrile and was obtained as an off-white solid.

Yield 15 mg (15%). ¹H NMR (400 MHz, DMSO) δ 11.00 (s, 1H), 9.11 (t, J=6.0 Hz, 1H), 9.06 (s, 1H), 8.70 (s, 1H), 7.18 (s, 1H), 6.84 (dd, J=1.2, 12.4 Hz, 1H), 4.50 (d, J=6.0 Hz, 2H), 3.94 (dd, J=3.6, 11.2 Hz, 2H), 3.44 (dt, J=2.4, 12.0 Hz, 2H), 3.08 (tdd, J=3.6, 7.2, 12.0 Hz, 1H), 2.51 (s, 3H), 2.18 (s, 3H), 1.96 (dq, J=4.4, 12.8 Hz, 2H), 1.57 (dd, J=2.0, 12.8 Hz, 2H). ¹⁹F NMR (376 MHz, DMSO) δ 133.42. m/z: [ESI⁺] 383 (M+H)⁺, (C₂₁H₂₃FN₄O₂).

### Example 2

### Biological Activity of Compounds of the Invention

The biological activity results of all compounds of the invention are summarized in Table 2.

TABLE 2
Cellular LogEC₅₀ values of compounds of the invention
in the WI-38 collagen 1 inhibition assay.

| | COL1 Efficacy |
|---|---|
| | (LogEC₅₀) |
| | −: >−4 |
| | +: −4 to −5 |
| | ++: −5 to −6 |
| Compound No. | +++: <−6 |
| 100 | ++ |
| 101 | +++ |
| 102 | ++ |
| 103 | +++ |
| 104 | +++ |
| 105 | +++ |
| 106 | +++ |
| 109 | +++ |
| 110 | +++ |
| 111 | +++ |
| 112 | +++ |
| 113 | ++ |
| 114 | +++ |
| 115 | ++ |
| 116 | ++ |
| 117 | +++ |
| 118 | +++ |
| 119 | ++ |
| 120 | ++ |
| 121 | + |
| 122 | +++ |
| 123 | +++ |
| 124 | +++ |
| 125 | ++ |
| 126 | ++ |
| 127 | +++ |
| 128 | +++ |
| 129 | ++ |
| 130 | +++ |
| 131 | +++ |
| 132 | ++ |
| 133 | +++ |
| 134 | ++ |
| 135 | ++ |
| 136 | ++ |
| 137 | ++ |
| 138 | ++ |
| 139 | +++ |
| 140 | +++ |
| 141 | ++ |
| 142 | ++ |
| 143 | + |
| 144 | − |
| 145 | − |
| 146 | ++ |
| 147 | ++ |
| 148 | + |
| 149 | − |
| 150 | − |
| 151 | ++ |
| 152 | − |
| 153 | ++ |
| 154 | + |
| 155 | − |
| 156 | ++ |
| 157 | − |
| 158 | +++ |
| 159 | ++ |
| 160 | ++ |
| 161 | ++ |
| 162 | + |
| 163 | ++ |
| 166 | ++ |
| 174 | + |
| 182 | − |
| 184 | + |
| 185 | ++ |
| 186 | +++ |
| 188 | + |
| 190 | + |
| 192 | − |
| 194 | ++ |
| 196 | − |
| 200 | + |
| 201 | + |
| 202 | ++ |
| 206 | + |
| 213 | ++ |
| 214 | +++ |
| 215 | ++ |
| 216 | ++ |
| 217 | ++ |
| 218 | ++ |
| 219 | ++ |
| 220 | ++ |
| 221 | +++ |
| 222 | +++ |
| 223 | ++ |
| 224 | +++ |
| 225 | ++ |
| 227 | − |
| 229 | − |
| 230 | − |
| 231 | − |
| 232 | − |
| 233 | − |
| 234 | − |
| 235 | − |
| 236 | − |
| 237 | − |
| 238 | − |
| 239 | − |
| 240 | − |
| 241 | − |
| 242 | − |
| 243 | − |
| 244 | − |
| 245 | − |
| 246 | − |
| 247 | − |
| 248 | − |
| 249 | − |

### Example 3

### Experimental Methods

### High Content Screen for the Identification of Collagen I Modulators

Compound effect on translation of Collagen I in WI38, human lung fibroblast cell line was conducted using specific PSM assay using tRNAgly and tRNApro isoacceptors, as described herein below. A library of diverse small molecules, 90,000 compounds, was used at a final concentration of 30 uM. Image and data analyses were conducted using Anima's proprietary algorithms. False positive and toxic compounds were eliminated. Compounds which increased or decreased the FRET signal generated by ribosomes during collagen I translation were identified as hits.

Positive hits were re-screened in the specific PSM assay, using tRNAPro and tRNAGly, and counter-screened to eliminate general translation inhibitors in bulk tRNA PSM assay and in a metabolic labeling assay [Click-IT™, L-Azidohomoalanine (AHA)]; collagen-specific regulators were assays using anti-Collagen I immunofluorescence; all assays were run on activated WI38 cells. Hits were scored using Anima's proprietary algorithms, and 360 compounds which selectively inhibited specific PSM assay and reduced collagen I as detected by immunofluorescence were selected as confirmed hits. These compounds were purchased as powder to confirm activity. Re-purchased hits were tested in the specific PSM assay (tRNApro-tRNAgly) and anti-Collagen I immunofluorescence, and in counter assays to eliminate global translation modulators: (1) bulk tRNA and metabolic labeling using Click-IT™ AHA (L-Azidohomoalanine).

### Cell Culture

WI-38 cells (ATCC® CCL-75™) were maintained in MEM EAGLE (NEAA) W. GLUTAMIN (Biological Industries, Cat. 06-1040-15-1A) containing 10% fetal bovine serum (FBS) and 1% Penicillin-Streptomycin Solution. To synchronize the cells (cell cycle synchronization) prior to induction of collagen synthesis, the cells were starved using DMEM-low glucose supplemented with 0.25% FBS for two hours and then without FBS for 24 hours. Then, to induce collagen synthesis, the cells were treated with a collagen induction cocktail for the indicated time. Compounds were added with induction.

Primary human pulmonary fibroblasts (HPF, PromoCell C-12360) were maintained in fibroblast growth medium 2 (PromoCell C-23020) according to manufacture instruction. Collagen synthesis was inducted using the same cocktail as for the WI-38 cells.

Primary human dermal fibroblasts (HDF) (PromoCell C-12302) were maintained in PromoCell's proprietary Fibroblast Growth Medium 2 (ready-to-use, Cat. C-23020). For collagen synthesis induction, cells were seeded on experimental plates for 24 hours followed by addition of collagen induction cocktail. Tested compounds were added together with induction.

### Protein Synthesis Monitoring (PSM) Assays

Cy3 and Cy5 Labeled tRNA, bulk or specific, are transfected with 0.4 μl HiPerFect (Qiagen) per 384 well. First, HiPerFect is mixed with DMEM and incubated for 5 minutes; next, 8 nanograms Cy3-labeled tRNAPro and 8 ng Cy5-labeled tRNAGly (or 8 ng each Cy3 and Cy5-labelled bulk tRNA are diluted in 1×PBS and then added to the HiPerFect:DMEM cocktail and incubated at room temperature for 20 minutes. The transfection mixture is dispersed automatically into 384-well black plates. Cells are then seeded at 3,500 cells per well in DMEM-10% FBS-1% penicillin-Streptomycin-1% L-Glutamine. Plates are incubated at 37° C., 5% CO₂ overnight. Twenty-four hours after transfection collagen production is stimulated with collagen induction cocktail, and then compounds are added at a final concentration of 30 uM. After an additional 24 hours incubation, cells are fixed with 4% paraformaldehyde and images are captured with Operetta microscope (Perkin Elmer) using ×20 high NA objective lens.

### Metabolic Labeling Assay

Synchronized WI-38 cells are seeded at 3,500 cells per well in DMEM-10% FBS-1% penicillin-Streptomycin-1% L-Glutamine. Plates are incubated at 37° C., 5% CO₂ overnight. The collagen production is stimulated with collagen induction cocktail, and then compounds are added at a final concentration of 30 uM. After 20 hours of incubation, the growth medium is aspirated, and cell washed twice with HBSS. Metabolic labeling medium DMEM (-Cys -Met)-10% dialyzed FBS-1% penicillin-Streptomycin-1% L-Glutamine was added to the cells for 30 minutes. Then medium was replaced by metabolic labeling medium containing 25 μM L-Azidohomoalanine (AHA, ThermoFisher) and incubated for 4 hours at 37° C., 5% CO₂. Cells are washed by HBSS at 37° C. for 15 minutes before fixing with 4% paraformaldehyde. Cells are washed twice with 3% BSA in PBS before permeabilization with 0.5% Triton X-100 in PBS for 20 minutes. The AHA staining with Alexa Fluor™ 555 alkyne is performed according to the manufacture instruction. Images are captured with Operetta microscope (Perkin Elmer) using ×20 high NA objective lens.

### Collagen-I Immunofluorescence Assay

Cells in 96-well or 384-well plates were fixed for 20 min in 4% paraformaldehyde (PFA, ENCO, Cat. sc-281692). Following two washes with 1×PBS, cells were treated with hydrogen peroxide (Acros, Cat: 7722-84-1) for 10 minutes and then washed twice with 1×PBS. Cells were then incubated over-night at 4° C. with Anti-Collagen I (Sigma-Aldrich, Cat: C 2456) antibody and washed three times with 1×PBS. Cells were then incubated with a suitable secondary fluorescently-tagged antibody and nuclei stained with DAPI, for 1 hour, and then washed 3 times with 1×PBS.

Cell images were taken with Operetta (Perkin Elmer, USA), a wide-field fluorescence microscope at 20× magnification. After acquisition, the images were transferred to Columbus software (Perkin-Elmer) for image analysis. In Columbus, cells were identified by their nucleus, using the “Find Nuceli” module and cytoplasm was detected based on the secondary antibody channel. Subsequently, the fluorescent signal was enumerated in the identified cell region. Data was exported to a data analysis and visualization software, Tibco Spotfire, USA.

### Fluorescent In Situ Hybridization (FISH) Assay

WI-38 cells were grown in 384-wells plates (Perkin Elmer, Cat. 6057300) and fixed for 20 min in 4% paraformaldehyde (PFA, ENCO, Cat. sc-281692), and left overnight in 70% ethanol at 4° C. The next day, the cells were washed with 1×PBS and then incubated for 10 min in 10% formamide in 10% saline-sodium citrate (SSC). Fluorescently labeled DNA probes that target the COL1 (Cy5, Biosearch Technologies, Cat. SMF-1063-5) and GAPDH (Cy3, Biosearch Technologies, Cat. VSMF-2150-5) mRNAs were hybridized overnight at 37° C. in a dark chamber in 10% formamide. The next day, cells were washed twice with 10% formamide for 30 min. Next, nuclei were counterstained with DAPI (SIGMA, Cat. 5MG-D9542) and then washed twice with 1×PBS. FISH experiments were performed according to the probes manufacturer's protocol for adherent cells.

Following RNA FISH experiments, images of cells were taken with Operetta (Perkin Elmer, USA), a wide-field fluorescence microscope at 20× magnification. After acquisition, the images were transferred to Columbus software for image analysis. In Columbus, cells were identified by their nucleus, using the “Find Nuceli” module, cytoplasm was detected based on the FISH-channel, and single mRNAs in the cytoplasm and transcription sites in the nucleus were detected using “Find Spots” module. Subsequently, fluorescent signals were collected for each channel in the identified regions, nucleus, cytoplasm and spots. Data was exported to a data analysis and visualization software, Tibco Spotfire, USA.

1-53. (canceled)

54. A compound represented by the structure of formula (I): 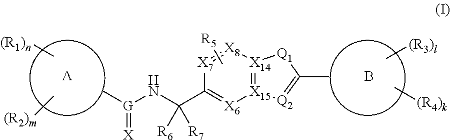
wherein
A and B rings are each independently a single or fused aromatic or heteroaromatic ring system, or a single or fused C₃-C₁₀ cycloalkyl or a single or fused C₃-C₁₀ heterocyclic ring;
R₁ and R₂ are each independently H, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl), C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy, optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, substituted or unsubstituted C₃-C₈ heterocyclic ring, substituted or unsubstituted aryl, or substituted or unsubstituted benzyl;
or R₂ and R₁ are joint together to form a 5 or 6 membered substituted or unsubstituted, aliphatic or aromatic, carbocyclic or heterocyclic ring;
R₃ and R₄ are each independently H, F, Cl, Br, I, OH, SH, R₈—OH, R₈—SH, —R₈—O—R₁₀, —O—R₈—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy, optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, substituted or unsubstituted C₃-C₈ heterocyclic ring, substituted or unsubstituted aryl, or substituted or unsubstituted benzyl;
or R₃ and R₄ are joint together to form a 5 or 6 membered substituted or unsubstituted, aliphatic or aromatic, carbocyclic or heterocyclic ring;
R₅ is absent or is H, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy, optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, substituted or unsubstituted C₃-C₈ heterocyclic ring, substituted or unsubstituted aryl, or substituted or unsubstituted benzyl;
R₆ and R₇ are each independently H, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy, optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, substituted or unsubstituted C₃-C₈ heterocyclic ring, substituted or unsubstituted aryl, or substituted or unsubstituted benzyl;
or R₆ and R₇ are joint together to form a 3 to 6 membered substituted or unsubstituted, aliphatic or aromatic, carbocyclic or heterocyclic ring;
Q₁ is NH, N(R), N—OH or N—OMe;
Q₂ is N, or C—CH₃;
R is H, F, Cl, Br, I, OH, SH, OH, N(R)₂, CF₃, CN, NO₂, C₁-C₅ linear or branched, substituted or unsubstituted alkyl, C₁-C₅ linear or branched alkoxy, C₁-C₅ linear or branched haloalkyl, R₈-aryl, —R₈—O—R₈—O—R₁₀, —R₈—O—R₁₀, —R₈—R₁₀, substituted or unsubstituted aryl, or substituted or unsubstituted heteroaryl;
G=X is C═O, C═S, S═O, SO₂, CH₂, CHR, or C(R)₂;
each R₈ is independently [CH₂]ₚ
wherein p is between 1 and 10;
R₉ is [CH]_{q}, [C]_{q}
wherein q is between 2 and 10;
R₁₀ and R₁₁ are each independently H, C₁-C₈ substituted or unsubstituted linear or branched alkyl, C₁-C₅ linear or branched alkoxy, C(O)R, or S(O)₂R; or R₁₀ and R₁₁ are joint to form a substituted or unsubstituted C₃-C₈ heterocyclic ring,
m, n, l and k are each independently an integer between 0 and 4;
X₆, X₇ and X₈ are each independently C or N;
X₁₄ and X₁₅ are each independently C or N;
or its pharmaceutically acceptable salt, optical isomer, tautomer, hydrate, N-oxide, isotopic variant, pharmaceutical product or any combination thereof;
wherein R₅ can only be attached to X₆, X₇ and/or X₈ that are C.

55. The compound of claim 54, represented by the structure of formula I(a): 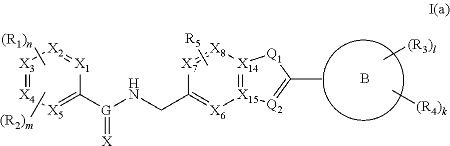
wherein
X₁, X₂, X₃, X₄, and X₅, are each independently C or N;
by the structure of formula I(b): 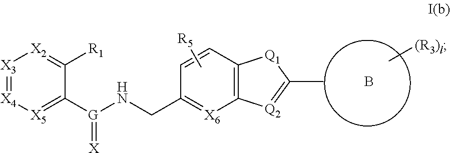
by the structure of formula I(c) 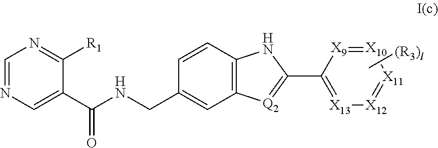
wherein
X₉, X₁₀, X₁₁, X₁₂, and X₁₃ are each independently C or N;
by the structure of formula I(d): 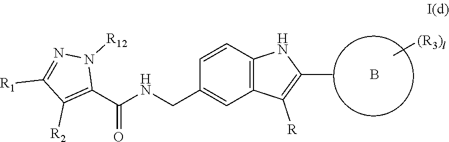
wherein
R₁₂ is H, a C₁-C₅ linear or branched, substituted or unsubstituted alkyl, F, Cl, Br, I, OH, SH, OH, N(R)₂, CF₃, CN, NO₂, C₁-C₅ linear or branched alkoxy, C₁-C₅ linear or branched haloalkyl or C₃-C₈ cycloalkyl;
by the structure of formula I(e): 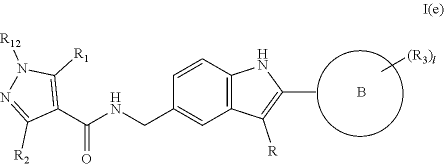
by the structure of formula I(f): 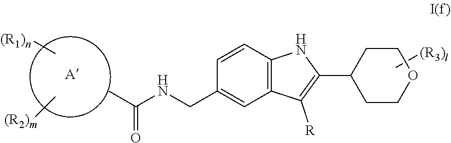
wherein
A′ ring is a 5 (five) membered heteroaromatic, or a heterocyclic ring;
by the structure of formula I(d(i)): 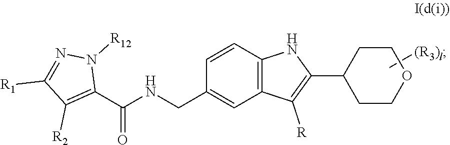
or by the structure of formula I(e(i)):

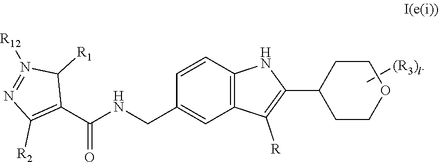

56. The compound of claim 54, selected from the following:

| Compound | |
|---|---|
| Number | Compound Structure |
| 100 | 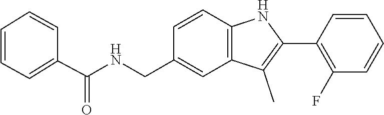 [SMILES: Cc1c(-c2ccccc2F)[nH]c2ccc(CNC(=O)c3ccccc3)cc12] |
| 102 | 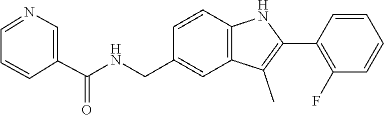 [SMILES: Cc1c(-c2ccccc2F)[nH]c2ccc(CNC(=O)c3cccnc3)cc12] |
| 103 | 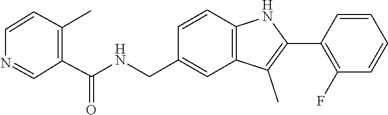 [SMILES: Cc1ccncc1C(=O)NCc1ccc2[nH]c(-c3ccccc3F)c(C)c2c1] |
| 104 | 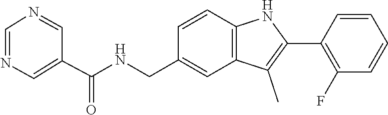 [SMILES: Cc1c(-c2ccccc2F)[nH]c2ccc(CNC(=O)c3cncnc3)cc12] |
| 105 | 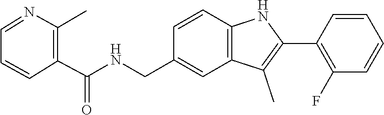 [SMILES: Cc1ncccc1C(=O)NCc1ccc2[nH]c(-c3ccccc3F)c(C)c2c1] |
| 106 | 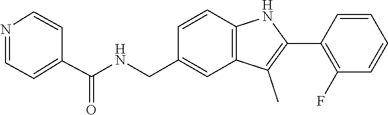 [SMILES: Cc1c(-c2ccccc2F)[nH]c2ccc(CNC(=O)c3ccncc3)cc12] |
| 109 | 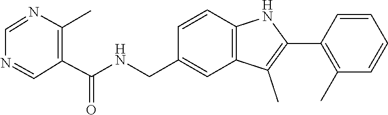 [SMILES: Cc1ccccc1-c1[nH]c2ccc(CNC(=O)c3cncnc3C)cc2c1C] |
| 110 | 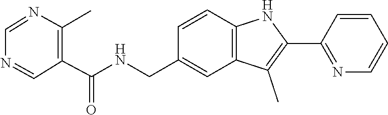 [SMILES: Cc1ncncc1C(=O)NCc1ccc2[nH]c(-c3ccccn3)c(C)c2c1] |
| 111 | 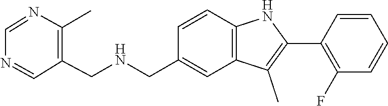 [SMILES: Cc1ncncc1CNCc1ccc2[nH]c(-c3ccccc3F)c(C)c2c1] |
| 112 | 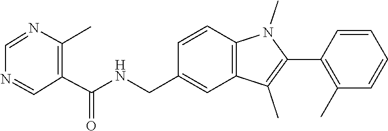 [SMILES: Cc1ccccc1-c1c(C)c2cc(CNC(=O)c3cncnc3C)ccc2n1C] |
| 113 | 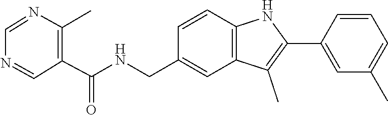 [SMILES: Cc1cccc(-c2[nH]c3ccc(CNC(=O)c4cncnc4C)cc3c2C)c1] |
| 115 | 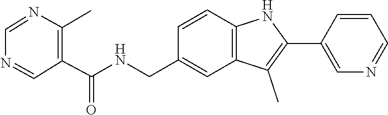 [SMILES: Cc1ncncc1C(=O)NCc1ccc2[nH]c(-c3cccnc3)c(C)c2c1] |
| 116 | 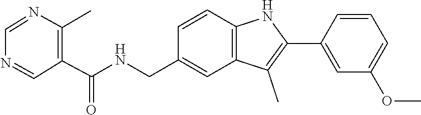 [SMILES: COc1cccc(-c2[nH]c3ccc(CNC(=O)c4cncnc4C)cc3c2C)c1] |
| 118 | 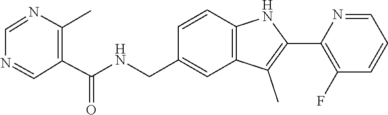 [SMILES: Cc1ncncc1C(=O)NCc1ccc2[nH]c(-c3ncccc3F)c(C)c2c1] |
| 119 | 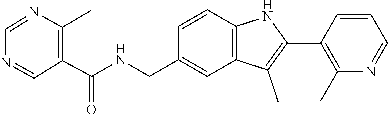 [SMILES: Cc1ncncc1C(=O)NCc1ccc2[nH]c(-c3cccnc3C)c(C)c2c1] |
| 120 | 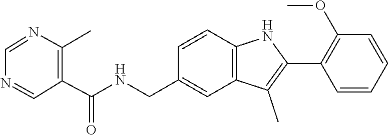 [SMILES: COc1ccccc1-c1[nH]c2ccc(CNC(=O)c3cncnc3C)cc2c1C] |
| 121 | 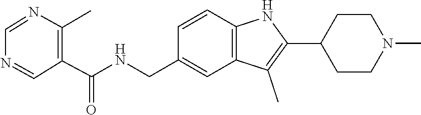 [SMILES: Cc1ncncc1C(=O)NCc1ccc2[nH]c(C3CCN(C)CC3)c(C)c2c1] |
| 122 | 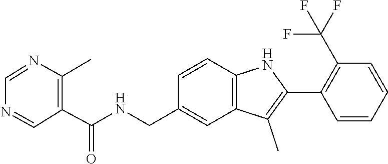 [SMILES: Cc1ncncc1C(=O)NCc1ccc2[nH]c(-c3ccccc3C(F)(F)F)c(C)c2c1] |
| 123 | 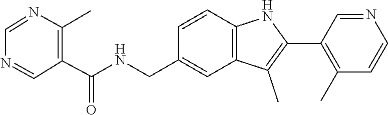 [SMILES: Cc1ccncc1-c1[nH]c2ccc(CNC(=O)c3cncnc3C)cc2c1C] |
| 124 | 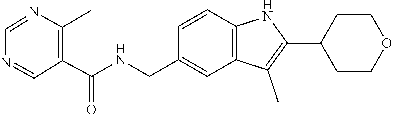 [SMILES: Cc1ncncc1C(=O)NCc1ccc2[nH]c(C3CCOCC3)c(C)c2c1] |
| 125 | 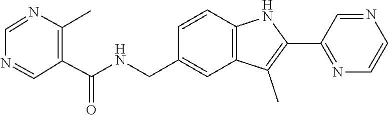 [SMILES: Cc1ncncc1C(=O)NCc1ccc2[nH]c(-c3cnccn3)c(C)c2c1] |
| 126 | 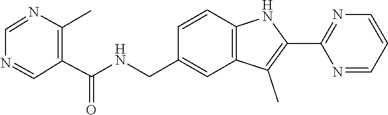 [SMILES: Cc1ncncc1C(=O)NCc1ccc2[nH]c(-c3ncccn3)c(C)c2c1] |
| 127 | 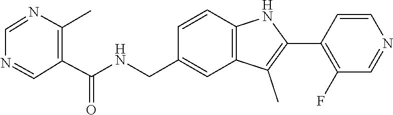 [SMILES: Cc1ncncc1C(=O)NCc1ccc2[nH]c(-c3ccncc3F)c(C)c2c1] |
| 128 | 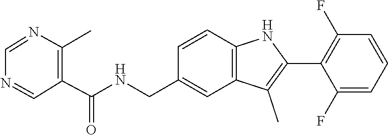 [SMILES: Cc1ncncc1C(=O)NCc1ccc2[nH]c(-c3c(F)cccc3F)c(C)c2c1] |
| 129 | 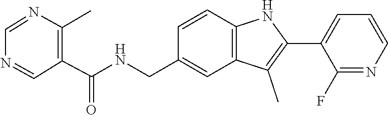 [SMILES: Cc1ncncc1C(=O)NCc1ccc2[nH]c(-c3cccnc3F)c(C)c2c1] |
| 130 | 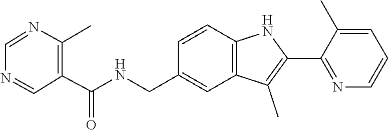 [SMILES: Cc1cccnc1-c1[nH]c2ccc(CNC(=O)c3cncnc3C)cc2c1C] |
| 131 | 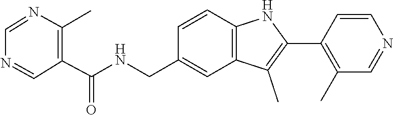 [SMILES: Cc1cnccc1-c1[nH]c2ccc(CNC(=O)c3cncnc3C)cc2c1C] |
| 132 (Isomer 2) | 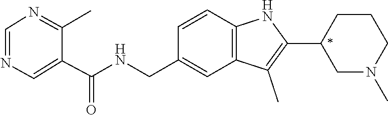 [SMILES: Cc1ncncc1C(=O)NCc1ccc2[nH]c(C3CCCN(C)C3)c(C)c2c1] |
| 133 | 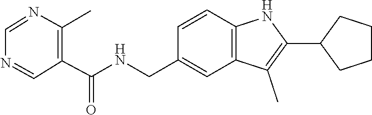 [SMILES: Cc1ncncc1C(=O)NCc1ccc2[nH]c(C3CCCC3)c(C)c2c1] |
| 134 (Isomer 1) | 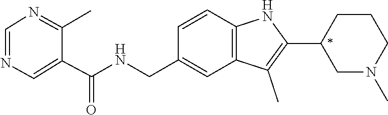 [SMILES: Cc1ncncc1C(=O)NCc1ccc2[nH]c(C3CCCN(C)C3)c(C)c2c1] |
| 135 | 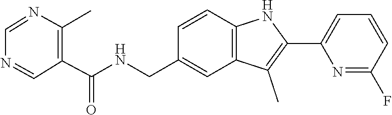 [SMILES: Cc1ncncc1C(=O)NCc1ccc2[nH]c(-c3cccc(F)n3)c(C)c2c1] |
| 136 | 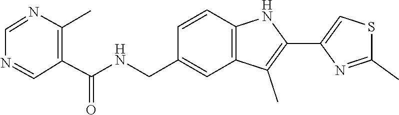 [SMILES: Cc1nc(-c2[nH]c3ccc(CNC(=O)c4cncnc4C)cc3c2C)cs1] |
| 137 | 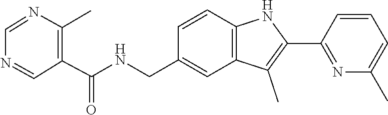 [SMILES: Cc1cccc(-c2[nH]c3ccc(CNC(=O)c4cncnc4C)cc3c2C)n1] |
| 139 | 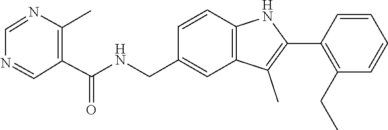 [SMILES: CCc1ccccc1-c1[nH]c2ccc(CNC(=O)c3cncnc3C)cc2c1C] |
| 140 | 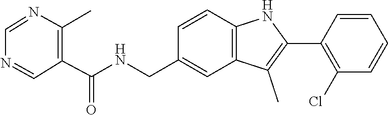 [SMILES: Cc1ncncc1C(=O)NCc1ccc2[nH]c(-c3ccccc3Cl)c(C)c2c1] |
| 141 | 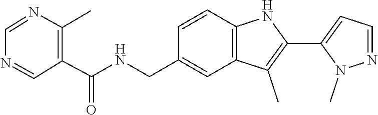 [SMILES: Cc1ncncc1C(=O)NCc1ccc2[nH]c(-c3ccnn3C)c(C)c2c1] |
| 142 | 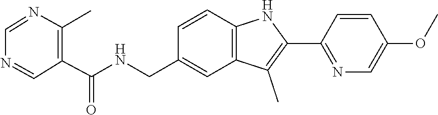 [SMILES: COc1ccc(-c2[nH]c3ccc(CNC(=O)c4cncnc4C)cc3c2C)nc1] |
| 143 | 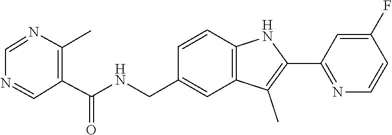 [SMILES: Cc1ncncc1C(=O)NCc1ccc2[nH]c(-c3cc(F)ccn3)c(C)c2c1] |
| 144 | 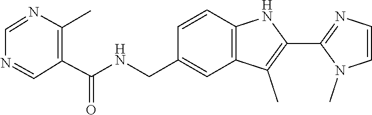 [SMILES: Cc1ncncc1C(=O)NCc1ccc2[nH]c(-c3nccn3C)c(C)c2c1] |
| 145 | 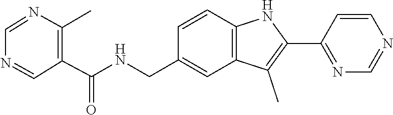 [SMILES: Cc1ncncc1C(=O)NCc1ccc2[nH]c(-c3ccncn3)c(C)c2c1] |
| 146 | 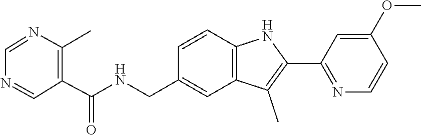 [SMILES: COc1ccnc(-c2[nH]c3ccc(CNC(=O)c4cncnc4C)cc3c2C)c1] |
| 147 | 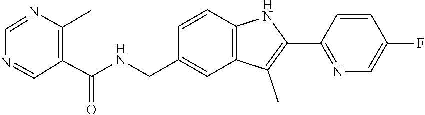 [SMILES: Cc1ncncc1C(=O)NCc1ccc2[nH]c(-c3ccc(F)cn3)c(C)c2c1] |
| 148 | 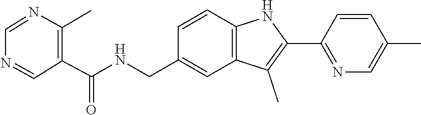 [SMILES: Cc1ccc(-c2[nH]c3ccc(CNC(=O)c4cncnc4C)cc3c2C)nc1] |
| 149 | 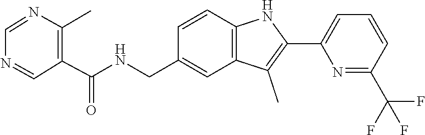 [SMILES: Cc1ncncc1C(=O)NCc1ccc2[nH]c(-c3cccc(C(F)(F)F)n3)c(C)c2c1] |
| 150 | 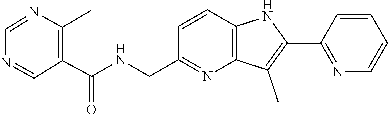 [SMILES: Cc1ncncc1C(=O)NCc1ccc2[nH]c(-c3ccccn3)c(C)c2n1] |
| 151 | 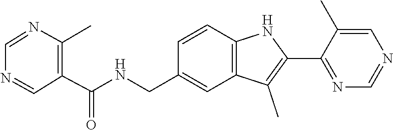 [SMILES: Cc1cncnc1-c1[nH]c2ccc(CNC(=O)c3cncnc3C)cc2c1C] |
| 152 | 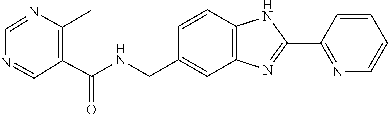 [SMILES: Cc1ncncc1C(=O)NCc1ccc2[nH]c(-c3ccccn3)nc2c1] |
| 154 | 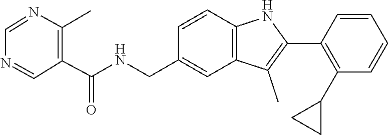 [SMILES: Cc1ncncc1C(=O)NCc1ccc2[nH]c(-c3ccccc3C3CC3)c(C)c2c1] |
| 155 | 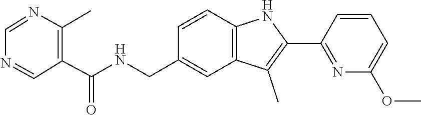 [SMILES: COc1cccc(-c2[nH]c3ccc(CNC(=O)c4cncnc4C)cc3c2C)n1] |
| 157 | 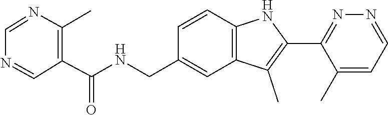 [SMILES: Cc1ccnnc1-c1[nH]c2ccc(CNC(=O)c3cncnc3C)cc2c1C] |
| 158 | 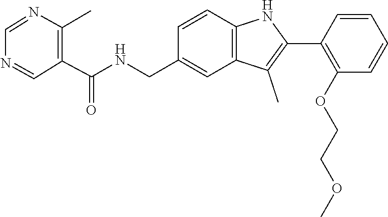 [SMILES: COCCOc1ccccc1-c1[nH]c2ccc(CNC(=O)c3cncnc3C)cc2c1C] |
| 159 | 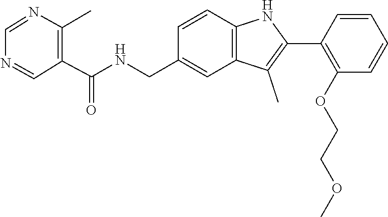 [SMILES: COCCOc1ccccc1-c1[nH]c2ccc(CNC(=O)c3cncnc3C)cc2c1C] |
| 161 | 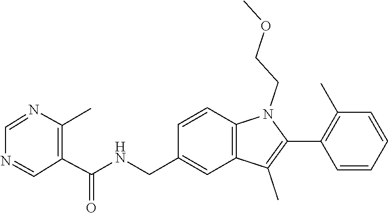 [SMILES: COCCn1c(-c2ccccc2C)c(C)c2cc(CNC(=O)c3cncnc3C)ccc21] |
| 162 | 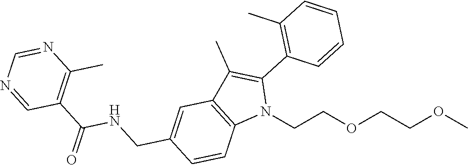 [SMILES: COCCOCCn1c(-c2ccccc2C)c(C)c2cc(CNC(=O)c3cncnc3C)ccc21] |
| 163 | 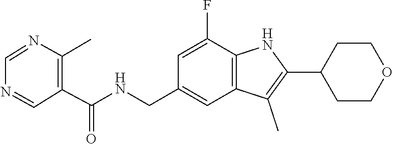 [SMILES: Cc1ncncc1C(=O)NCc1cc(F)c2[nH]c(C3CCOCC3)c(C)c2c1] |
| 164 | 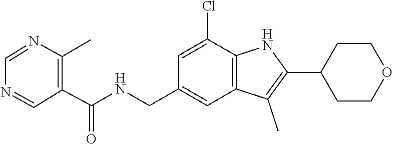 [SMILES: Cc1ncncc1C(=O)NCc1cc(Cl)c2[nH]c(C3CCOCC3)c(C)c2c1] |
| 165 | 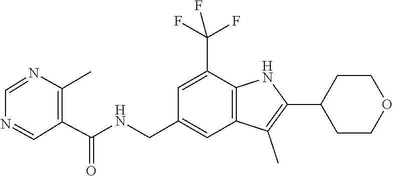 [SMILES: Cc1ncncc1C(=O)NCc1cc(C(F)(F)F)c2[nH]c(C3CCOCC3)c(C)c2c1] |
| 166 | 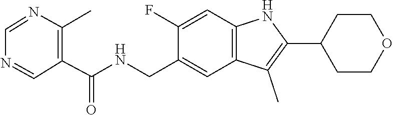 [SMILES: Cc1ncncc1C(=O)NCc1cc2c(C)c(C3CCOCC3)[nH]c2cc1F] |
| 167 | 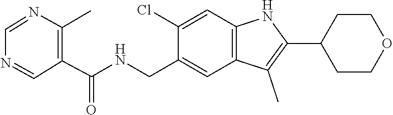 [SMILES: Cc1ncncc1C(=O)NCc1cc2c(C)c(C3CCOCC3)[nH]c2cc1Cl] |
| 168 | 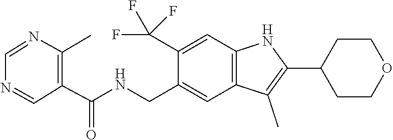 [SMILES: Cc1ncncc1C(=O)NCc1cc2c(C)c(C3CCOCC3)[nH]c2cc1C(F)(F)F] |
| 169 | 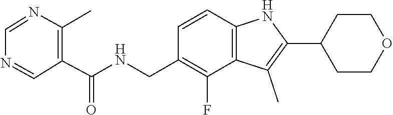 [SMILES: Cc1ncncc1C(=O)NCc1ccc2[nH]c(C3CCOCC3)c(C)c2c1F] |
| 170 | 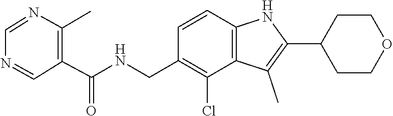 [SMILES: Cc1ncncc1C(=O)NCc1ccc2[nH]c(C3CCOCC3)c(C)c2c1Cl] |
| 171 | 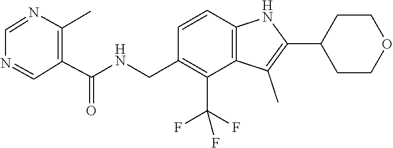 [SMILES: Cc1ncncc1C(=O)NCc1ccc2[nH]c(C3CCOCC3)c(C)c2c1C(F)(F)F] |
| 173 | 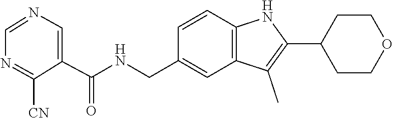 [SMILES: Cc1c(C2CCOCC2)[nH]c2ccc(CNC(=O)c3cncnc3C#N)cc12] |
| 174 | 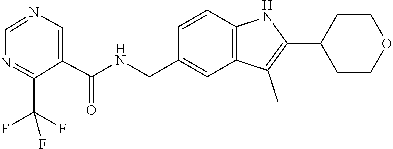 [SMILES: Cc1c(C2CCOCC2)[nH]c2ccc(CNC(=O)c3cncnc3C(F)(F)F)cc12] |
| 175 | 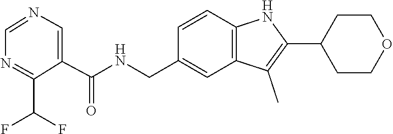 [SMILES: Cc1c(C2CCOCC2)[nH]c2ccc(CNC(=O)c3cncnc3C(F)F)cc12] |
| 176 | 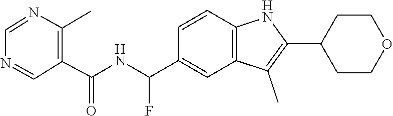 [SMILES: Cc1ncncc1C(=O)NC(F)c1ccc2[nH]c(C3CCOCC3)c(C)c2c1] |
| 177 | 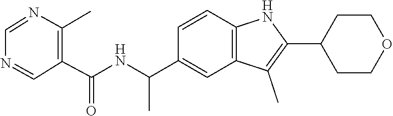 [SMILES: Cc1ncncc1C(=O)NC(C)c1ccc2[nH]c(C3CCOCC3)c(C)c2c1] |
| 178 | 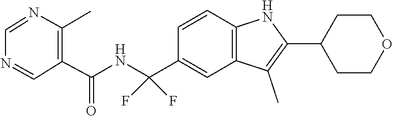 [SMILES: Cc1ncncc1C(=O)NC(F)(F)c1ccc2[nH]c(C3CCOCC3)c(C)c2c1] |
| 179 | 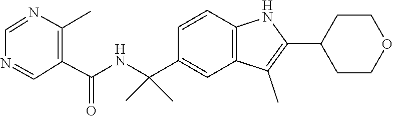 [SMILES: Cc1ncncc1C(=O)NC(C)(C)c1ccc2[nH]c(C3CCOCC3)c(C)c2c1] |
| 180 | 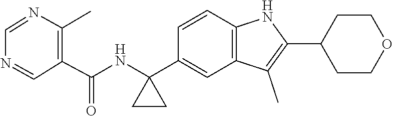 [SMILES: Cc1ncncc1C(=O)NC1(c2ccc3[nH]c(C4CCOCC4)c(C)c3c2)CC1] |
| 181 | 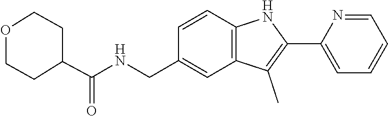 [SMILES: Cc1c(-c2ccccn2)[nH]c2ccc(CNC(=O)C3CCOCC3)cc12] |
| 182 | 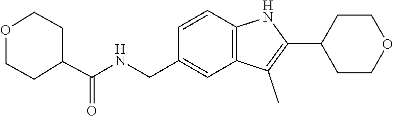 [SMILES: Cc1c(C2CCOCC2)[nH]c2ccc(CNC(=O)C3CCOCC3)cc12] |
| 183 | 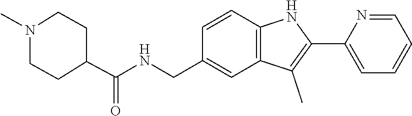 [SMILES: Cc1c(-c2ccccn2)[nH]c2ccc(CNC(=O)C3CCN(C)CC3)cc12] |
| 184 | 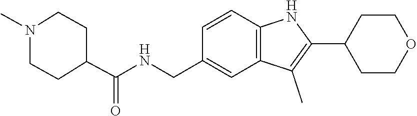 [SMILES: Cc1c(C2CCOCC2)[nH]c2ccc(CNC(=O)C3CCN(C)CC3)cc12] |
| 185 | 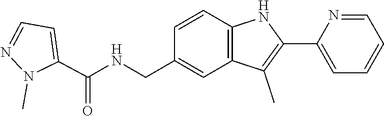 [SMILES: Cc1c(-c2ccccn2)[nH]c2ccc(CNC(=O)c3ccnn3C)cc12] |
| 186 | 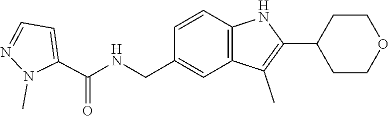 [SMILES: Cc1c(C2CCOCC2)[nH]c2ccc(CNC(=O)c3ccnn3C)cc12] |
| 187 | 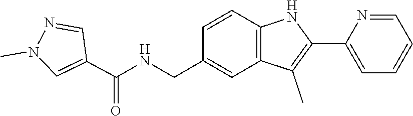 [SMILES: Cc1c(-c2ccccn2)[nH]c2ccc(CNC(=O)c3cnn(C)c3)cc12] |
| 188 | 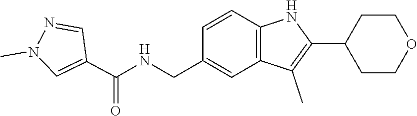 [SMILES: Cc1c(C2CCOCC2)[nH]c2ccc(CNC(=O)c3cnn(C)c3)cc12] |
| 189 | 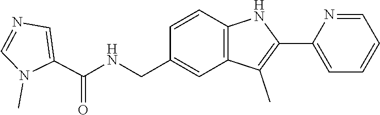 [SMILES: Cc1c(-c2ccccn2)[nH]c2ccc(CNC(=O)c3cncn3C)cc12] |
| 190 | 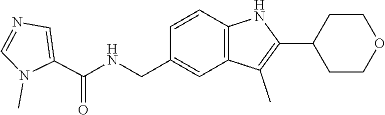 [SMILES: Cc1c(C2CCOCC2)[nH]c2ccc(CNC(=O)c3cncn3C)cc12] |
| 191 | 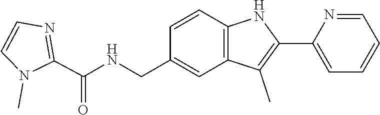 [SMILES: Cc1c(-c2ccccn2)[nH]c2ccc(CNC(=O)c3nccn3C)cc12] |
| 192 | 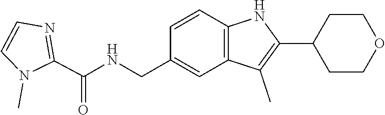 [SMILES: Cc1c(C2CCOCC2)[nH]c2ccc(CNC(=O)c3nccn3C)cc12] |
| 193 | 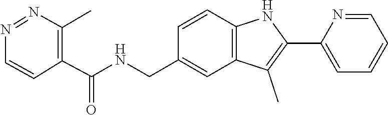 [SMILES: Cc1nnccc1C(=O)NCc1ccc2[nH]c(-c3ccccn3)c(C)c2c1] |
| 194 | 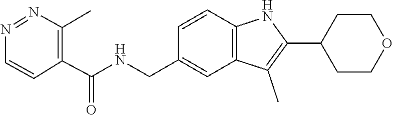 [SMILES: Cc1nnccc1C(=O)NCc1ccc2[nH]c(C3CCOCC3)c(C)c2c1] |
| 195 | 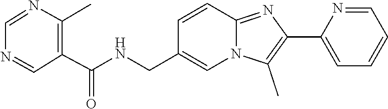 [SMILES: Cc1ncncc1C(=O)NCc1ccc2nc(-c3ccccn3)c(C)n2c1] |
| 196 | 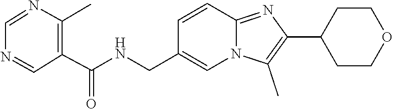 [SMILES: Cc1ncncc1C(=O)NCc1ccc2nc(C3CCOCC3)c(C)n2c1] |
| 197 | 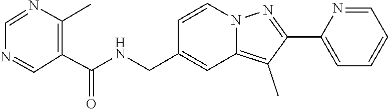 [SMILES: Cc1ncncc1C(=O)NCc1ccn2nc(-c3ccccn3)c(C)c2c1] |
| 198 | 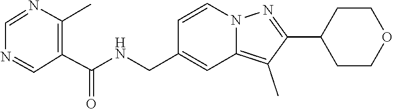 [SMILES: Cc1ncncc1C(=O)NCc1ccn2nc(C3CCOCC3)c(C)c2c1] |
| 201 | 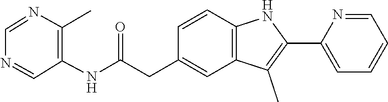 [SMILES: Cc1ncncc1NC(=O)Cc1ccc2[nH]c(-c3ccccn3)c(C)c2c1] |
| 202 | 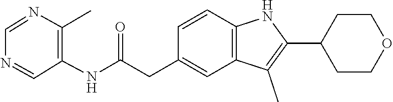 [SMILES: Cc1ncncc1NC(=O)Cc1ccc2[nH]c(C3CCOCC3)c(C)c2c1] |
| 203 | 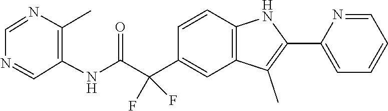 [SMILES: Cc1ncncc1NC(=O)C(F)(F)c1ccc2[nH]c(-c3ccccn3)c(C)c2c1] |
| 204 | 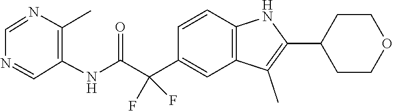 [SMILES: Cc1ncncc1NC(=O)C(F)(F)c1ccc2[nH]c(C3CCOCC3)c(C)c2c1] |
| 205 | 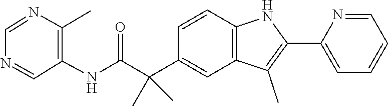 [SMILES: Cc1ncncc1NC(=O)C(C)(C)c1ccc2[nH]c(-c3ccccn3)c(C)c2c1] |
| 206 | 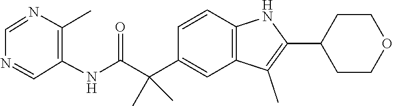 [SMILES: Cc1ncncc1NC(=O)C(C)(C)c1ccc2[nH]c(C3CCOCC3)c(C)c2c1] |
| 207 | 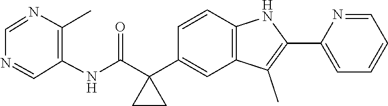 [SMILES: Cc1ncncc1NC(=O)C1(c2ccc3[nH]c(-c4ccccn4)c(C)c3c2)CC1] |
| 208 | 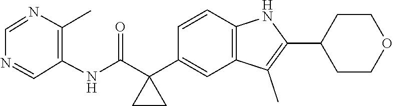 [SMILES: Cc1ncncc1NC(=O)C1(c2ccc3[nH]c(C4CCOCC4)c(C)c3c2)CC1] |
| 209 | 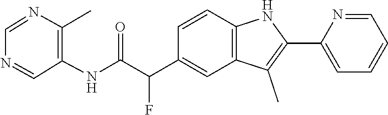 [SMILES: Cc1ncncc1NC(=O)C(F)c1ccc2[nH]c(-c3ccccn3)c(C)c2c1] |
| 210 | 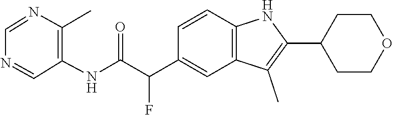 [SMILES: Cc1ncncc1NC(=O)C(F)c1ccc2[nH]c(C3CCOCC3)c(C)c2c1] |
| 211 | 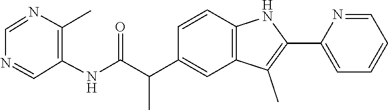 [SMILES: Cc1ncncc1NC(=O)C(C)c1ccc2[nH]c(-c3ccccn3)c(C)c2c1] |
| 212 | 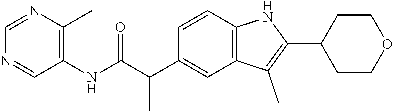 [SMILES: Cc1ncncc1NC(=O)C(C)c1ccc2[nH]c(C3CCOCC3)c(C)c2c1] |
| 213 | 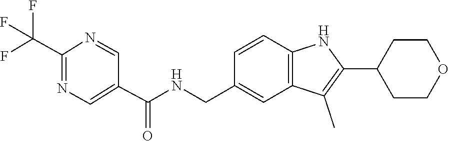 [SMILES: Cc1c(C2CCOCC2)[nH]c2ccc(CNC(=O)c3cnc(C(F)(F)F)nc3)cc12] |
| 214 | 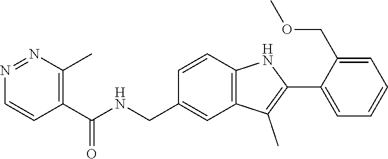 [SMILES: COCc1ccccc1-c1[nH]c2ccc(CNC(=O)c3ccnnc3C)cc2c1C] |
| 215 | 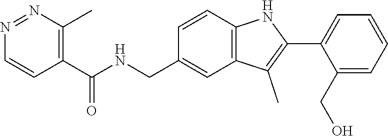 [SMILES: Cc1nnccc1C(=O)NCc1ccc2[nH]c(-c3ccccc3CO)c(C)c2c1] |
| 216 | 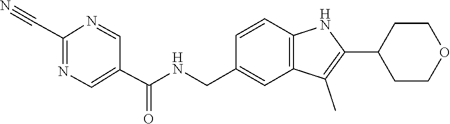 [SMILES: Cc1c(C2CCOCC2)[nH]c2ccc(CNC(=O)c3cnc(C#N)nc3)cc12] |
| 217 | 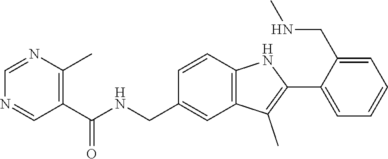 [SMILES: CNCc1ccccc1-c1[nH]c2ccc(CNC(=O)c3cncnc3C)cc2c1C] |
| 218 | 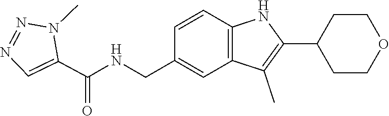 [SMILES: Cc1c(C2CCOCC2)[nH]c2ccc(CNC(=O)c3cnnn3C)cc12] |
| 219 | 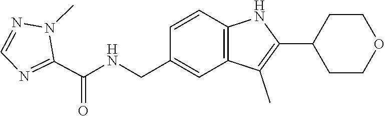 [SMILES: Cc1c(C2CCOCC2)[nH]c2ccc(CNC(=O)c3ncnn3C)cc12] |
| 220 | 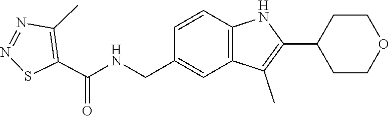 [SMILES: Cc1nnsc1C(=O)NCc1ccc2[nH]c(C3CCOCC3)c(C)c2c1] |
| 221 | 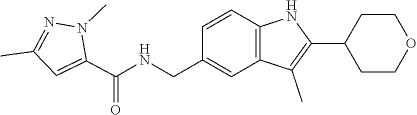 [SMILES: Cc1cc(C(=O)NCc2ccc3[nH]c(C4CCOCC4)c(C)c3c2)n(C)n1] |
| 222 | 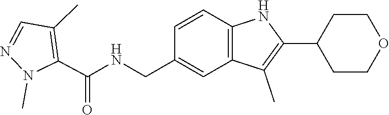 [SMILES: Cc1cnn(C)c1C(=O)NCc1ccc2[nH]c(C3CCOCC3)c(C)c2c1] |
| 223 | 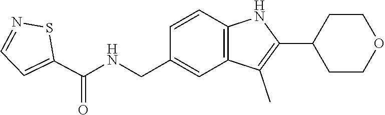 [SMILES: Cc1c(C2CCOCC2)[nH]c2ccc(CNC(=O)c3ccns3)cc12] |
| 224 | 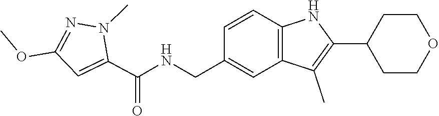 [SMILES: COc1cc(C(=O)NCc2ccc3[nH]c(C4CCOCC4)c(C)c3c2)n(C)n1] |
| 225 | 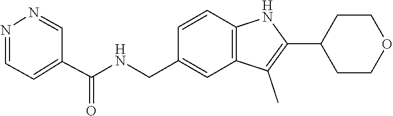 [SMILES: Cc1c(C2CCOCC2)[nH]c2ccc(CNC(=O)c3ccnnc3)cc12] |
| 227 | 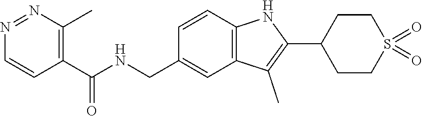 [SMILES: Cc1nnccc1C(=O)NCc1ccc2[nH]c(C3CCS(=O)(=O)CC3)c(C)c2c1] |
| 229 | 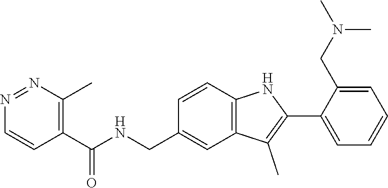 [SMILES: Cc1nnccc1C(=O)NCc1ccc2[nH]c(-c3ccccc3CN(C)C)c(C)c2c1] |
| 230 | 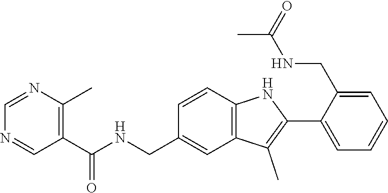 [SMILES: CC(=O)NCc1ccccc1-c1[nH]c2ccc(CNC(=O)c3cncnc3C)cc2c1C] |
| 231 | 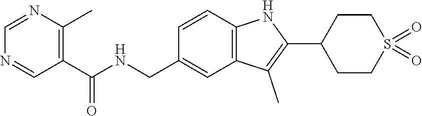 [SMILES: Cc1ncncc1C(=O)NCc1ccc2[nH]c(C3CCS(=O)(=O)CC3)c(C)c2c1] |
| 232 | 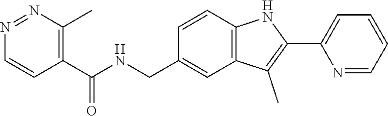 [SMILES: Cc1nnccc1C(=O)NCc1ccc2[nH]c(-c3ccccn3)c(C)c2c1] |
| 233 | 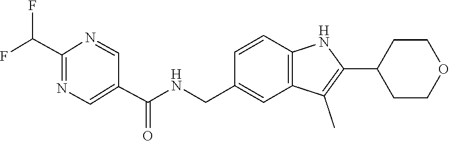 [SMILES: Cc1c(C2CCOCC2)[nH]c2ccc(CNC(=O)c3cnc(C(F)F)nc3)cc12] |
| 234 | 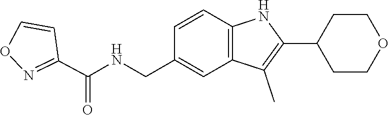 [SMILES: Cc1c(C2CCOCC2)[nH]c2ccc(CNC(=O)c3ccon3)cc12] |
| 235 | 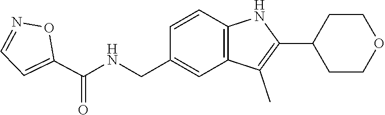 [SMILES: Cc1c(C2CCOCC2)[nH]c2ccc(CNC(=O)c3ccno3)cc12] |
| 236 | 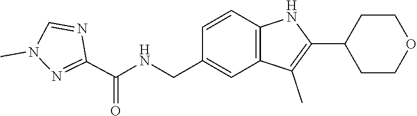 [SMILES: Cc1c(C2CCOCC2)[nH]c2ccc(CNC(=O)c3ncn(C)n3)cc12] |
| 237 | 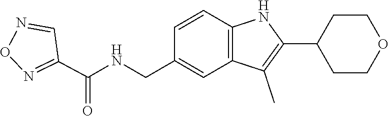 [SMILES: Cc1c(C2CCOCC2)[nH]c2ccc(CNC(=O)c3cnon3)cc12] |
| 238 | 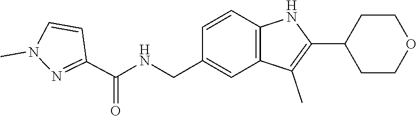 [SMILES: Cc1c(C2CCOCC2)[nH]c2ccc(CNC(=O)c3ccn(C)n3)cc12] |
| 239 | 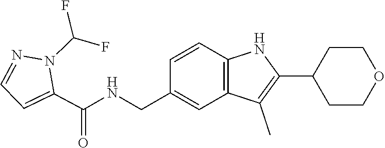 [SMILES: Cc1c(C2CCOCC2)[nH]c2ccc(CNC(=O)c3ccnn3C(F)F)cc12] |
| 240 | 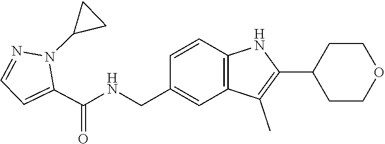 [SMILES: Cc1c(C2CCOCC2)[nH]c2ccc(CNC(=O)c3ccnn3C3CC3)cc12] |
| 241 | 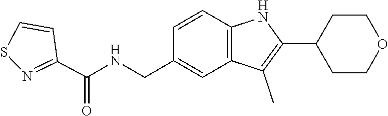 [SMILES: Cc1c(C2CCOCC2)[nH]c2ccc(CNC(=O)c3ccsn3)cc12] |
| 242 | 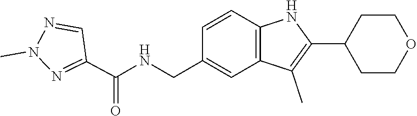 [SMILES: Cc1c(C2CCOCC2)[nH]c2ccc(CNC(=O)c3cnn(C)n3)cc12] |
| 243 | 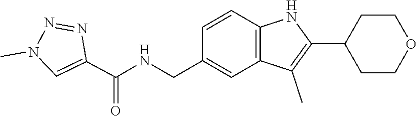 [SMILES: Cc1c(C2CCOCC2)[nH]c2ccc(CNC(=O)c3cn(C)nn3)cc12] |
| 244 | 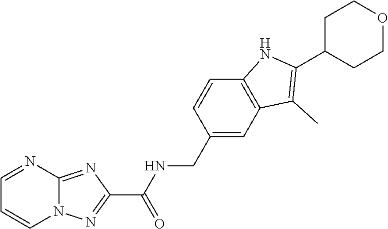 [SMILES: Cc1c(C2CCOCC2)[nH]c2ccc(CNC(=O)c3nc4ncccn4n3)cc12] |
| 245 | 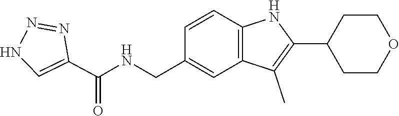 [SMILES: Cc1c(C2CCOCC2)[nH]c2ccc(CNC(=O)c3c[nH]nn3)cc12] |
| 246 | 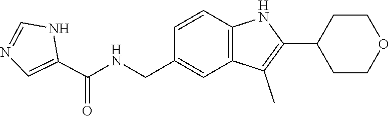 [SMILES: Cc1c(C2CCOCC2)[nH]c2ccc(CNC(=O)c3cnc[nH]3)cc12] |
| 247 | 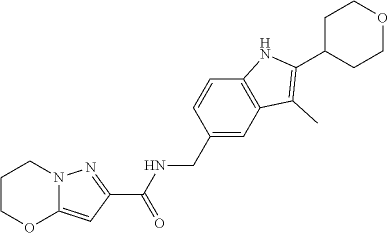 [SMILES: Cc1c(C2CCOCC2)[nH]c2ccc(CNC(=O)c3cc4n(n3)CCCO4)cc12] |
| 248 | 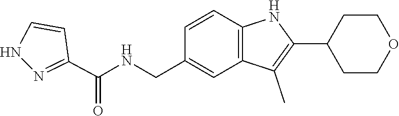 [SMILES: Cc1c(C2CCOCC2)[nH]c2ccc(CNC(=O)c3cc[nH]n3)cc12] |
| 249 | 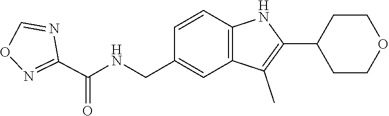 [SMILES: Cc1c(C2CCOCC2)[nH]c2ccc(CNC(=O)c3ncon3)cc12] |

57. The compound according to claim 54, wherein B is tetrahydropyrane, pyridinyl or phenyl; l is 1 and k is 0; R₃ is H, CH₃ or CF₃; A is pyrimidine or pyrazolyl; n is 1 and m is 0; R₁ is CH₃; Q₁ is NH or N(R); G=X is C═O; or any combination thereof.

58. The compound according to claim 54, wherein the compound is a collagen translation inhibitor.

59. A pharmaceutical composition comprising a compound according to claim 54 and a pharmaceutically acceptable carrier.

60. A compound represented by the structure of formula (I): 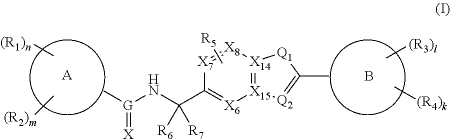
wherein
A is a single or fused aromatic or heteroaromatic ring system, or a single or fused C₃-C₁₀ cycloalkyl or a single or fused C₃-C₁₀ heterocyclic ring;
B ring is a single or fused aromatic ring system, or a single or fused C₃-C₁₀ cycloalkyl or a single or fused C₃-C₁₀ non-aromatic heterocyclic ring;
R₁ and R₂ are each independently H, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl), C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy, optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, substituted or unsubstituted C₃-C₈ heterocyclic ring, substituted or unsubstituted aryl, or substituted or unsubstituted benzyl;
or R₂ and R₁ are joint together to form a 5 or 6 membered substituted or unsubstituted, aliphatic or aromatic, carbocyclic or heterocyclic ring;
R₃ and R₄ are each independently H, F, Cl, Br, I, OH, SH, R₈—OH, R₈—SH, —R₈—O—R₁₀, —O—R₈—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy, optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, substituted or unsubstituted C₃-C₈ heterocyclic ring, substituted or unsubstituted aryl, or substituted or unsubstituted benzyl;
or R₃ and R₄ are joint together to form a 5 or 6 membered substituted or unsubstituted, aliphatic or aromatic, carbocyclic or heterocyclic ring;
R₅ is absent or is H, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy, optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, substituted or unsubstituted C₃-C₈ heterocyclic ring, substituted or unsubstituted aryl, or substituted or unsubstituted benzyl;
R₆ and R₇ are each independently H, F, Cl, Br, I, OH, SH, R₈—OH, R₈—SH, —R₈—O—R₁₀, R₈—(C₃-C₈ cycloalkyl), R₈—(C₃-C₈ heterocyclic ring), CF₃, CD₃, OCD₃, CN, NO₂, —CH₂CN, —R₈CN, NH₂, NHR, N(R)₂, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy, optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, substituted or unsubstituted C₃-C₈ heterocyclic ring, substituted or unsubstituted aryl, or substituted or unsubstituted benzyl;
or R₆ and R₇ are joint together to form a 3 to 6 membered substituted or unsubstituted, aliphatic or aromatic, carbocyclic or heterocyclic ring;
Q₁ is NH, N(R), N—OH or N—OMe;
Q₂ is N or C(R);
R is H, F, Cl, Br, I, OH, SH, OH, N(R)₂, CF₃, CN, NO₂, C₁-C₅ linear or branched, substituted or unsubstituted alkyl, C₁-C₅ linear or branched alkoxy, C₁-C₅ linear or branched haloalkyl, R₈-aryl, —R₈—O—R₈—O—R₁₀, —R₈—O—R₁₀, —R₈—R₁₀, substituted or unsubstituted aryl, or substituted or unsubstituted heteroaryl;
G=X is C═O, C═S, S═O, SO₂, CH₂, CHR, or C(R)₂;
each R₈ is independently [CH₂]ₚ
wherein p is between 1 and 10;
R₉ is [CH]_{q}, [C]_{q}
wherein q is between 2 and 10;
R₁₀ and R₁₁ are each independently H, C₁-C₅ substituted or unsubstituted linear or branched alkyl, C₁-C₅ linear or branched alkoxy, C(O)R, or S(O)₂R; or R₁₀ and R₁₁ are joint to form a substituted or unsubstituted C₃-C₈ heterocyclic ring,
m, n, l and k are each independently an integer between 0 and 4;
X₆, X₇ and X₈ are each independently C or N;
X₁₄ and X₁₅ are each independently C or N;
or its pharmaceutically acceptable salt, optical isomer, tautomer, hydrate, N-oxide, isotopic variant, pharmaceutical product or any combination thereof;
wherein R₅ can only be attached to X₆, X₇ and/or X₈ that are C.

61. The compound of claim 60, represented by the structure of the following compounds:

| Com- | |
|---|---|
| pound | |
| Num- | |
| ber | Compound Structure |
| 114 | 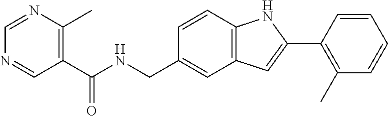 [SMILES: Cc1ccccc1-c1cc2cc(CNC(=O)c3cncnc3C)ccc2[nH]1] |
| 117 | 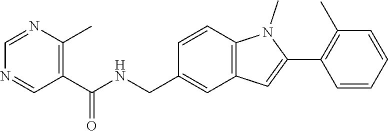 [SMILES: Cc1ccccc1-c1cc2cc(CNC(=O)c3cncnc3C)ccc2n1C] |
| 156 | 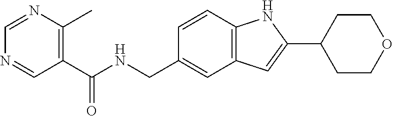 [SMILES: Cc1ncncc1C(=O)NCc1ccc2[nH]c(C3CCOCC3)cc2c1] |
| 160 | 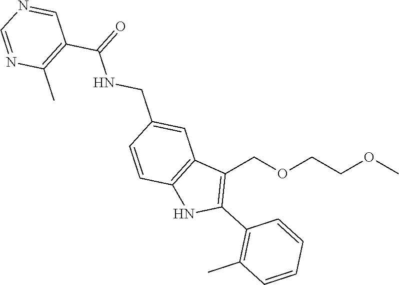 [SMILES: COCCOCc1c(-c2ccccc2C)[nH]c2ccc(CNC(=O)c3cncnc3C)cc12] |

62. The compound according to claim 60, wherein B is tetrahydropyrane, or phenyl; l is 1 and k is 0; R₃ is H, CH₃ or CF₃; A is pyrimidine or pyrazolyl; n is 1 and m is 0; R₁ is CH₃; Q₁ is NH or N(R); Q₂ is CH; G=X is C═O; or any combination thereof.

63. A compound represented by the structure of the following compounds:

| Com- | |
|---|---|
| pound | |
| Num- | |
| ber | Compound Structure |
| 138 | 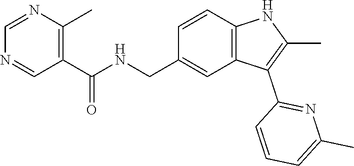 [SMILES: Cc1cccc(-c2c(C)[nH]c3ccc(CNC(=O)c4cncnc4C)cc23)n1] |
| 153 | 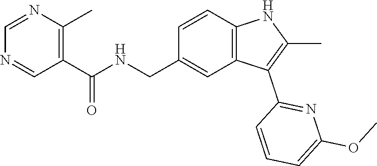 [SMILES: COc1cccc(-c2c(C)[nH]c3ccc(CNC(=O)c4cncnc4C)cc23)n1] |
| 199 | 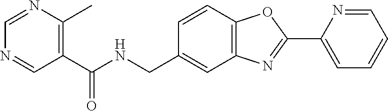 [SMILES: Cc1ncncc1C(=O)NCc1ccc2oc(-c3ccccn3)nc2c1] |
| 200 | 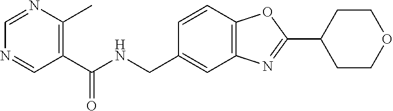 [SMILES: Cc1ncncc1C(=O)NCc1ccc2oc(C3CCOCC3)nc2c1] |

64. A method of treating, suppressing, reducing the severity, reducing the risk of developing or inhibiting fibrosis in a subject, comprising administering a compound according to claim 54 to a subject suffering from fibrosis under conditions effective to treat, suppress, reduce the severity, reduce the risk of developing, or inhibit the fibrosis in said subject.

65. The method of claim 64, wherein said fibrosis is a systemic fibrotic disease;
wherein said fibrosis is primary or secondary fibrosis;
wherein said fibrosis is a result of systemic sclerosis, graft-versus host disease (GVHD), pulmonary fibrosis, autoimmune disorder, tissue injury, inflammation, oxidative stress or any combination thereof;
wherein the fibrosis is hepatic fibrosis, lung fibrosis or dermal fibrosis;
or any combination thereof.

66. The method of claim 65, wherein said systemic fibrotic disease is systemic sclerosis, multifocal fibrosclerosis (IgG4-associated fibrosis), nephrogenic systemic fibrosis, sclerodermatous graft vs. host disease, or any combination thereof; or wherein said fibrosis is an organ-specific fibrotic disease.

67. The method of claim 66, wherein said organ-specific fibrotic disease is lung fibrosis, cardiac fibrosis, kidney fibrosis, pulmonary fibrosis, liver and portal vein fibrosis, radiation-induced fibrosis, bladder fibrosis, intestinal fibrosis, peritoneal sclerosis, diffuse fasciitis, wound healing, scaring, or any combination thereof.

68. The method of claim 67,
wherein said lung fibrosis is Idiopathic pulmonary fibrosis (IPF);
wherein said cardiac fibrosis is hypertension-associated cardiac fibrosis, Post-myocardial infarction, Chagas disease-induced myocardial fibrosis or any combination thereof;
wherein said kidney fibrosis is diabetic and hypertensive nephropathy, urinary tract obstruction-induced kidney fibrosis, inflammatory/autoimmune-induced kidney fibrosis, aristolochic acid nephropathy, polycystic kidney disease, or any combination thereof;
wherein said pulmonary fibrosis is idiopathic pulmonary fibrosis, silica-induced pneumoconiosis (silicosis), asbestos-induced pulmonary fibrosis (asbestosis), chemotherapeutic agent-induced pulmonary fibrosis, or any combination thereof;
wherein said liver and portal vein fibrosis is alcoholic and nonalcoholic liver fibrosis, hepatitis C-induced liver fibrosis, primary biliary cirrhosis, parasite-induced liver fibrosis (schistosomiasis), or any combination thereof;
wherein said diffuse fasciitis is localized scleroderma, keloids, dupuytren's disease, peyronie's disease, myelofibrosis, oral submucous fibrosis, or any combination thereof;
or any combination thereof.

69. The method of claim 64, wherein said subject has a liver cirrhosis.

70. The method of claim 65, wherein the dermal fibrosis is scleroderma; or wherein the dermal fibrosis is a result of a localized or generalized morphea, keloids, hypertrophic scars, familial cutaneous collagenoma, connective tissue nevi of the collagen type, or any combination thereof.

71. The method of claim 65, wherein the hepatic fibrosis is a result of hepatic scarring or chronic liver injury.

72. The method of claim 71, wherein the chronic liver injury results from alcoholism, malnutrition, hemochromatosis, exposure to poisons, toxins or drugs.

73. A method for treating, suppressing, reducing the severity, reducing the risk of developing or inhibiting a disease or condition selected from: lung fibrosis, Idiopathic pulmonary fibrosis (IPF), hepato-fibrotic disorder, cirrhosis, alcoholic steatohepatitis (ASH), non-alcoholic steatohepatitis (NASH), alcoholic fatty liver disease (AFLD), non alcoholic fatty liver disease (NAFLD), or an autoimmune disease or disorder in a subject, comprising administering a compound according to claim 54 to a subject suffering from said disease or condition under conditions effective to treat, suppress, reduce the severity, reduce the risk of developing, or inhibit the disease or condition in said subject.

74. The method of claim 73,
wherein the lung fibrosis is Idiopathic pulmonary fibrosis (IPF);
wherein the hepato-fibrotic disorder is a portal hypertension, cirrhosis, congenital hepatic fibrosis or any combination thereof; or
wherein the cirrhosis is a result of hepatitis or alcoholism.

75. A method of treating, suppressing, reducing the severity, reducing the risk of developing or inhibiting a disease or condition in a subject, comprising administering a compound to a subject suffering from said disease or condition under conditions effective to treat, suppress, reduce the severity, reduce the risk of developing, or inhibit the disease or condition in said subject, wherein the compound is represented by the following structure: 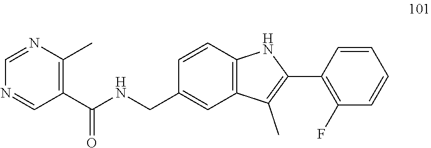 [SMILES: Cc1ncncc1C(=O)NCc1ccc2[nH]c(-c3ccccc3F)c(C)c2c1]
and the disease or condition is selected from: fibrosis, lung fibrosis, Idiopathic pulmonary fibrosis (IPF), hepato-fibrotic disorder, cirrhosis, alcoholic steatohepatitis (ASH), non-alcoholic steatohepatitis (NASH), alcoholic fatty liver disease (AFLD), non alcoholic fatty liver disease (NAFLD), an autoimmune disease or disorder, or any combination thereof.
